(19) United States
(12) Patent Application Publication
(10) Pub. No.: US 2004/0203014 A1
(43) Pub. Date: Oct. 14, 2004

(54) Neurotransmisson-associated proteins

(76) Inventors: Cynthia D. Honchell, San Francisco, CA (US); Bridget A. Warren, San Marcos, CA (US); Mark L. Borowsky, Needham, MA (US); Jennifer A. Griffin, Fremont, CA (US); Joana X. Li, Millbrae, CA (US); Soo Yeun Lee, Mountain View, CA (US); Henry Yue, Sunnyvale, CA (US); Ian J. Forsythe, Edmonton (CA); Joseph P. Marquis, San Jose, CA (US); Kimberly J. Gietzen, San Jose, CA (US); Mariah R. Baughn, Los Angeles, CA (US); Uyen K. Tran, San Jose, CA (US); Patricia M. Lehr-Mason, Morgan Hill, CA (US); Y. Tom Tang, San Jose, CA (US); Jayalaxmi Ramkumar, Fremont, CA (US); Brooke M. Emerling, Chicago, IL (US); Ernestine A. Lee, Kensington, CA (US); Vicki S. Elliott, San Jose, CA (US); April J.A. Hafalia, Daly City, CA (US); Brendan M. Duggan, Sunnyvale, CA (US); Narinder K. Chawla, Union City, CA (US); Amy E. Kable, Silver Spring, MD (US); Hsin-Ru Chang, Belmont, CA (US); Reena Khare, Saratoga, CA (US); Shanya D. Becha, San Francisco, CA (US); Pei Jin, Palo Alto, CA (US); Sally Lee, San Jose, CA (US)

Correspondence Address: FOLEY AND LARDNER, SUITE 500, 3000 K STREET NW, WASHINGTON, DC, 20007, US

(21) Appl. No.: 10/489,372

(22) PCT Filed: Sep. 12, 2002
(86) PCT No.: PCT/US02/29219

(30) Foreign Application Priority Data
Sep. 14, 2001 (US) 60322180
Sep. 28, 2001 (US) 60326096
Oct. 4, 2001 (US) 60327446
Oct. 26, 2001 (US) 60345837
Nov. 2, 2001 (US) 60343903
Nov. 27, 2001 (US) 60334020
Dec. 7, 2001 (US) 60340226
Jan. 4, 2002 (US) 60345008
Mar. 18, 2002 (US) 60365645
May 10, 2002 (US) 60379887

(51) Int. Cl. C12Q 1/68; C07H 21/04; C12N 15/00; C07K 14/705

(52) U.S. Cl. 435/6; 435/69.1; 435/320.1; 435/325; 530/350; 536/23.5

(57) ABSTRACT

Various embodiments of the invention provide human neurotransmission-associated proteins (NTRAN) and polynucleotides which identify and encode NTRAN. Embodiments of the invention also provide expression vectors, host cells, antibodies, agonists, and antagonists. Other embodiments provide methods for diagnosing, treating, or preventing disorders associated with aberrant expression of NTRAN.

### TECHNICAL FIELD

The invention relates to novel nucleic acids, neurotransmission-associated proteins encoded by these nucleic acids, and to the use of these nucleic acids and proteins in the diagnosis, treatment, and prevention of autoimmune/inflammatory, cardiovascular, neurological, developmental, cell proliferative, transport, psychiatric, metabolic, and endocrine disorders. The invention also relates to the assessment of the effects of exogenous compounds on the expression of nucleic acids and neurotransmission-associated proteins.

### BACKGROUND OF THE INVENTION

The human nervous system, which regulates all bodily functions, is composed of the central nervous system (CNS), consisting of the brain and spinal cord, and the peripheral nervous system (PNS), consisting of afferent neural pathways for conducting nerve impulses from sensory organs to the CNS, and efferent neural pathways for conducting motor impulses from the CNS to effector organs. The PNS can be further divided into the somatic nervous system, which regulates voluntary motor activity such as for skeletal muscle, and the autonomic nervous system, which regulates involuntary motor activity for internal organs such as the heart, lungs, and viscera. CNS-associated proteins function in neuronal signaling, cell adhesion, nerve regeneration, axon guidance, neurogenesis, and other processes.

The cerebral cortex or higher brain is the largest structure, consisting of a right and a left hemisphere interconnected by the corpus callosum. The cerebral cortex is involved in sensory, motor, and integrative functions related to perception, voluntary musculoskeletal movements, and the broad range of activities associated with consciousness, language, emotions, and memory. The cerebrum functions in association with the lower centers of the nervous system. The lower areas of the brain such as the medulla, pons, mesencephalon, cerebellum, basal ganglia, substantia nigra, hypothalamus, and thalamus control unconscious activities including arterial pressure and respiration, equilibrium, and feeding reflexes, such as salivation.

The central nervous system (CNS) is composed of more than 100 billion neurons at the spinal cord level, the lower brain level, and the higher brain or cortical level. Neurons transmit electric or chemical signals between cells. The spinal cord, a thin, tubular extension of the central nervous system within the bony spinal canal, contains ascending sensory and descending motor pathways, and is covered by membranes continuous with those of the brainstem and cerebral hemispheres. The spinal cord contains almost the entire motor output and sensory input systems of the trunk and limbs, and neuronal circuits in the cord also control rhythmic movements, such as walking, and a variety of reflexes. The lower areas of the brain such as the medulla, pons, mesencephalon, cerebellum, basal ganglia, substantia nigra, hypothalamus, and thalamus control unconscious activities including arterial pressure and respiration, equilibrium, and feeding reflexes, such as salivation. Emotions, such as anger, excitement, sexual response, and reaction to pain or pleasure, originate in the lower brain. The cerebral cortex or higher brain is the largest structure, consisting of a right and a left hemisphere interconnected by the corpus callosum. The cerebral cortex is involved in sensory, motor, and integrative functions related to perception, voluntary musculoskeletal movements, and the broad range of activities associated with consciousness, language, emotions, and memory. The cerebrum functions in association with the lower centers of the nervous system.

Nervous System Organization and Development

A nerve cell (neuron) contains four regions, the cell body, axon, dendrites, and axon terminal. The cell body contains the nucleus and other organelles. The dendrites are processes which extend outward from the cell body and receive signals from sense organs or from the axons of other neurons. These signals are converted to electrical impulses and transmitted to the cell body. The axon, whose size can range from one millimeter to more than one meter, is a single process that conducts the nerve impulse away from the cell body. Cytoskeletal fibers, including microtubules and neurofilaments, run the length of the axon and function in transporting proteins, membrane vesicles, and other macromolecules from the cell body along the axon to the axon terminal Some axons are surrounded by a myelin sheath made up of membranes from either an oligodendrocyte cell (CNS) or a Schwann cell (PNS). Myelinated axons conduct electrical impulses faster than unmyelinated ones of the same diameter. The axon terminal is at the tip of the axon away from the cell body. (See Lodish, H. et al. (1986) Molecular Cell Biology Scientific American Books New York N.Y., pp. 715-719.)

CNS-associated proteins have roles in neuronal signaling, cell adhesion, nerve regeneration, axon guidance, neurogenesis, and other functions. Certain CNS-associated proteins form an integral part of a membrane or are attached to a membrane. For example, neural membrane protein 35 (NMP35) is closely associated with neuronal membranes and is known to be highly expressed in the rat adult nervous system (Schweitzer, B. et al. (1998) Mol. Cell. Neurosci. 11:260-273). Synaptophysin (SY) is a major integral membrane protein of small synaptic vesicles. The chromosomal location of SY in human and mouse is on the X chromosome in subbands Xp11.22-p11.23. This region has been implicated in several inherited diseases including Wiskott-Aldrich syndrome, three forms of X-linked hypercalciuric nephrolithiaisis, and the eye disorders retinitis pigmentosa 2, congenital stationary night blindness, and Aland Island eye disease (Fisher, S. E. et al. (1997) Genomics 45:340-347). Peripherin, or retinal degeneration slow protein (rds), is an integral membrane glycoprotein that is present in the rims of photoreceptor outer segment disks. In mammals, rds is thought to stabilize the disk rim through heterophilic interactions with related nonglycosylated proteins. Rds is a mouse neurological mutation that is characterized by abnormal development of rod and cone photoreceptors followed by their slow degeneration (Kedzierski, W. J. et al. (1999) Neurochem. 72:430-438).

Each of over a trillion neurons in adult humans connects with over a thousand target cells (Tessier-Lavigne, M. et al. (1996) Science 274:1123-1133). These neuronal connections form during embryonic development. Each differentiating neuron sends out an axon tipped at the leading edge by a growth cone. Aided by molecular guidance cues, the growth cone migrates through the embryonic environment to its synaptic target. Progressive axon outgrowth occurs during neural development but not in the mature mammalian CNS. Following CNS injury, expression of growth-inhibiting molecules is enhanced while availability of their growth-promoting counterparts diminishes. Proteins governing developmental axon guidance contribute to the failure of injured central neurons to regenerate. These proteins include Semaphorin3A and the Semaphorin3A receptor proteins neuropilin-1 and plexin-A1 (Pasterkamp, R. J. and J. Verhaagen (2001) Brain Res. Brain Res. Rev. 35:36-54).

Semaphorins function during embryogenesis by providing local signals to specify territories inaccessible to growing axons (Puschel, A. W. et al. (1995) Neuron 14:941-948). They consist of at least 30 different members and are found in vertebrates, invertebrates, and even certain viruses. All semaphorins contain the sema domain which is approximately 500 amino acids in length. Neuropilin, a semaphorin receptor, has been shown to promote neurite outgrowth in vitro. The extracellular region of neuropilins consists of three different domains: CUB, discoidin, and MAM domains. The CUB and the MAM motifs of neuropilin have been suggested to have roles in protein-protein interactions and are thought to be involved in the binding of semaphorins through the sema and the C-terminal domains (reviewed in Raper, J. A. (2000) Curr. Opin. Neurobiol. 10:88-94).

The guidance of axons during development involves both positive and negative effects (i.e., chemoattraction and chemorepulsion). The Slit family of proteins have been implicated in promoting axon branching, elongation, and repulsion. Members of the Slit family have been identified in a variety of organisms, including insects, amphibians, birds, rodents and humans (Guthrie, S. (1999) Current Biology 9:R432-R435). Slit proteins are ligands for the repulsive guidance receptor, Roundabout (Robo); however, Slit proteins also cause elongation in some assays. A post-translationally processed form of Slit appears to be the active form of the protein (Guthrie, S. supra; and Brose, K. et al. (1999) Cell 96:795-806).

Axon growth is also guided in part by contact-mediated mechanisms involving cell surface and extracellular matrix (ECM) molecules. Many ECM molecules, including fibronectin, vitronectin, members of the laminin, tenascin, collagen, and thrombospondin families, and a variety of proteoglycans, can act either as promoters or inhibitors of neurite outgrowth and extension (Tessier-Lavigne et al., supra). Receptors for ECM molecules include integrins, immunoglobulin superfamily members, and proteoglycans. ECM molecules and their receptors have also been implicated in the adhesion, maintenance, and differentiation of neurons (Reichardt, L. F. et al. (1991) Ann. Rev. Neurosci. 14:531-571). The proteoglycan testican is localized to the post-synaptic area of pyramidal cells of the hippocampus and may play roles in receptor activity, neuromodulation, synaptic plasticity, and neurotransmission (Bonnet, F. et al. (1996) J. Biol. Chem. 271:4373-4380).

Neurotrophins regulate development, maintenance, and function of vertebrate nervous systems. Neurotrophins activate two different classes of receptors, the Trk family of receptor tyrosine kinases and p75NTR, a member of the TNF receptor superfamily. Through these receptors, neurotrophins activate many signaling pathways, including those mediated by ras and members of the cdc-42/ras/rho G protein families, and by the MAP kinase, PI-3 kinase, and Jun kinase cascades. During development, limiting amounts of neurotrophins function as survival factors to ensure a match between the number of surviving neurons and the requirement for appropriate target innervation. They also regulate cell fate decisions, axon growth, dendrite pruning, the patterning of innervation, and the expression of proteins crucial for normal neuronal function, such as neurotransmitters and ion channels. These proteins also regulate many aspects of neural function. In the mature nervous system, they control synaptic function and synaptic plasticity, while continuing to modulate neuronal survival (Huang, E. J. and L. P. Reichardt (2001) Ann. Rev. Neurosci. 24:677-736). Neuritin is a protein induced by neural activity and by neurotrophins which promote neuritogenesis.

The neurexophilins are neuropeptide-like proteins which are proteolytically processed after synthesis. They are ligands for the neuron-specific cell surface proteins, the α-neurexins. Neurexophilins and neurexins may participate in a neuron signaling pathway (Missler, M. and T. C. Sudhof (1998) J. Neurosci. 18:3630-3638; Missler, M. et al. (1998) J. Biol. Chem. 273:34716-34723). Ninjurin is a neuron cell surface protein which plays a role in cell adhesion and in nerve regeneration following injury. Ninjurin is up-regulated after nerve injury in dorsal root ganglion neurons and in Schwann cells (Araki, T. and J. Milbrandt (1996) Neuron 17:353-361). Ninjurin2 is expressed in mature sensory and enteric neurons and promotes neurite outgrowth. Ninjurin2 is upregulated in Schwann cells surrounding the distal segment of injured nerve with a time course similar to that of ninjurin1, neural CAM, and Li (Araki, T. and J. Milbrandt (2000) J. Neurosci. 20:187-195).

Neurexin IV is essential for axonal insulation in the PNS in embryos and larvae. Axonal insulation is of key importance for the proper propagation of action potentials. Caspr, a vertebrate homolog of Neurexin IV—also named paranodin—is found in septate-like junctional structures localized to the paranodal region of the nodes of Ranvier, between axons and Schwann cells. Caspr/paranodin is implicated in blood-brain barrier formation, and linkage of neuronal membrane components with the axonal cytoskeletal network (Bellen, H. J. et al. (1998) Trends Neurosci. 21:444-449).

Mammalian Numb is a phosphotyrosine-binding (PM) domain-containing protein which may be involved in cortical neurogenesis and cell fate decisions in the mammalian nervous system. Numb's binding partner, the LNX protein, contains four PDZ domains and a ring finger domain and may participate in a signaling pathway involving Numb. PDZ domains have been found in proteins which act as adaptors in the assembly of multifunctional protein complexes involved in signaling events at surfaces of cell membranes (Ponting, C. P. (1997) Bioessays 19:469-479). LNX contains a tyrosine phosphorylation site which maybe important for the binding of other PTB-containing proteins such as SHC, an adaptor protein which associates with tyrosine-phosphorylated growth factor receptors and downstream effectors (Dho, S. E. et al. (1998) J. Biol. Chem. 273:9179-9187).

Nogo has been identified as a component of the central nervous system myelin that prevents axonal regeneration in adult vertebrates. Cleavage of the Nogo-66 receptor and other glycophosphatidylinositol-linked proteins from axonal surfaces renders neurons insensitive to Nogo-66, facilitating potential recovery from CNS damage (Fournier, A. E. et al. (2001) Nature 409:341-346).

Homeobox transcription factors direct nerve-cell associated tissue patterning and differentiation. The presence and function of these proteins appears to be ubiquitous in nematodes, arthropods, and vertebrates. One example of these proteins is DRG11, a homeobox transcription factor expressed in mammalian sensory neurons, and which appears to be involved in neural crest development (Saito, T. et al. (1995) Mol. Cell Neurosci. 6:280-292). Cutaneous sensory neurons that detect noxious stimuli project to the dorsal horn of the spinal cord, while those innervating muscle stretch receptors project to the ventral horn. DRG11 is required for die formation of spatio-temporally appropriate projections from nociceptive sensory neurons to their central targets in the dorsal horn of the spinal cord (Chen, Z. F. et al. (2001) Neuron 31:59-73).

Synapses

Contact between one neuron and another occurs at a specialized site called the synapse. Many nervous system functions are regulated by diverse synaptic proteins such as synaptophysin, the synapsins, growth associated protein 43 (GAP-43), SV-2, and p65, which are distributed in subcellular compartments of the synapse. Synaptic terminals also contain many other proteins involved in calcium transport, neurotransmission, signaling, growth, and plasticity. At this site, the axon terminal from one neuron (the presynaptic cell) sends a signal to another neuron (the postsynaptic cell). Synapses may be connected either electrically or chemically. An electrical synapse consists of gap junctions connecting the two neurons, allowing electrical impulses to pass directly from the presynaptic to the postsynaptic cell. In a chemical synapse, the axon terminal of the presynaptic cell contains membrane vesicles containing a particular neurotransmitter molecule. A change in electrical potential at the nerve terminal results in the influx of calcium ions through voltage-gated channels which triggers the release of the neurotransmitter from the synaptic vesicle by exocytosis. The neurotransmitter rapidly diffuses across the synaptic cleft separating the presynaptic nerve cell from the postsynaptic cell. The neurotransmitter then binds receptors and opens transmitter-gated ion channels located in the plasma membrane of the postsynaptic cell, provoking a change in the cell's electrical potential. This change in membrane potential of the postsynaptic cell may serve either to excite or inhibit further transmission of the nerve impulse.

Presynaptic calcium channel activity is modulated by cysteine-string proteins (CSPs). CSPs are secretory vesicle proteins that function in neurotransmission as well as in exocytosis in other cell-types. CSPs belong to the DnaJ/hsp40 (heat shock protein) chaperone family. The effect of CSPs on calcium levels is likely to be downstream of calcium release and is likely to involve exocytosis, possibly in connection with G-proteins (Braun, J. E. et al. (1995) Neuropharmacology 34:1361-9136; Magga, J. M. et al. (2000) Neuron 28:195-204; Dawson-Scully, K. et al. (2000) J. Neurosci. 20:6039-6047; and Chamberlain, L. H. et al. (2001) J. Cell Sci. 114:445-455). Neuregulins (NRGs) mediate between the electrical neural activity and molecular components by regulating the expression of ion channel receptors or transmitter release in synapses. NRGs may also be signaling factors involved in tuning locomotion or other higher functions by coordinating excitatory and inhibitory neurons (Ozaki, M. (2001) Neuroscientist 7:146-154).

N- and P/Q-type Ca²⁺ channels are localized in high density in presynaptic nerve terminals and are crucial elements in neuronal excitation-secretion coupling. In addition to mediating Ca²⁺ entry to initiate transmitter release, they are thought to interact directly with proteins of the synaptic vesicle docking/fusion machinery. N-type and P/Q-type Ca²⁺ channels are colocalized with syntaxin in high-density clusters in nerve terminals. The synaptic protein interaction (synprint) sites in the intracellular loop II-III (LII-III) of both alpha 1B and alpha 1A subunits of N-type and P/Q-type Ca²⁺ channels bind to syntaxin, SNAP-25, and synaptotagmin. Presynaptic Ca²⁺ channels not only provide the Ca²⁺ signal required by the exocytotic machinery, but also contain structural elements that are integral to vesicle docking, priming, and fusion processes (Catterall, W. A. (1999) Ann. NY Acad. Sci. 868:144-159). Synaptotagmins are a large family of proteins involved in both regulated and constitutive vesicular trafficking. They include a neuronal type (synaptotagmin I-V, X, and XI) and a ubiquitous type (synaptotagmin VI-IX). Ca²⁺-dependent synaptotagmin activation is involved in neurite outgrowth (Mikoshiba, K. et al. (1999) Chem. Phys. Lipids 98:59-67).

Proteins associated with the membranes of synaptic vesicles include vamp (synaptobrevin), rab3A, synaptophysin, synaptotagmin (p65) and SV2. These membrane proteins function in regulated exocytosis by regulating neurotransmitter uptake, vesicle targeting, and fusion with the presynaptic plasma membrane (Elferink, L. A. and R. H. Scheller (1993) J. Cell Sci. Suppl. 17:75-79).

Physophilin, also known as the Ac39 subunit of the V-ATPase, is an oligomeric protein that binds the synaptic vesicle protein synaptophysin, constituting a complex that may form the exocytotic fusion pore. Ac39 is present in a synaptosomal complex which, in addition to synaptophysin, includes the bulk of synaptobrevin II, and subunits c and Ac115 of the V0 sector of the V-ATPase. In situ hybridization in rat brain reveals a largely neuronal distribution of Ac39/physophilin mRNA which correlates spatio-temporally with those of subunit c and synaptophysin. Immunohistochemical analysis shows that Ac39/physophllin is mostly concentrated in the neuropil with a pattern identical to subunit A and very similar to synaptophysin. Double-labeling immunofluorescence shows a complete colocalization of Ac39/physophilin with subunit A and a partial colocalization with synaptophysin in the neuropil (Carrion-Vazquez M. et al. (1998) Eur. J. Neurosci.10:1153-1166).

The plasma membrane dopamine transporter (DAT) is essential for the reuptake of released dopamine from the synapse. Uptake of dopamine is temperature- and time-dependent, and is inhibited by a variety of compounds, such as cocaine. DAT-knockout mice have been shown to exhibit extreme hyperactivity and resistance to both cocaine and amphetamine, consistent with the primary action of cocaine on DAT (Giros, B. et al. (1996) Nature 379:606-612). The perturbation of the tightly regulated DAT also predisposes neurons to damage by a variety of insults. Most notable is the selective degeneration of DAT-expressing dopamine nerve terminals in the striatum thought to underlie Parkinson's disease. DAT expression can predict the selective vulnerability of neuronal populations, which suggests that therapeutic strategies aimed at altering DAT function could have significant benefits in a variety of disorders (Gary, W. M. et al. (1999) Trends Pharmacol. Sci. 20:424-429).

43 KD postsynaptic protein or acetylcholine receptor-associated 43 KD protein (RAPSYN) is thought to play a role in anchoring or stabilizing the nicotinic acetylcholine receptor at synaptic sites. RAPSYN is involved in membrane association and may link the nicotinic acetylcholine receptor to the underlying postsynaptic cytoskeleton (Buckel, A. et al. (1996) Genomics 35:613-616). Neuritin is a protein whose gene is known to be induced by neural activity and by neurotrophins which promote neuritogenesis. Neuraxin is a structural protein of the rat central nervous system that is believed to be immunologically related to microtubule-associated protein 5 (MAP5). Neuraxin is a novel type of neuron-specific protein which is characterized by an unusual amino acid composition, 12 central heptadecarepeats and putative protein and membrane interaction sites. The gene encoding neuraxin is unique in the haploid rat genome and is conserved in higher vertebrates. Neuraxin is implicated in neuronal membrane-microtubule interactions and is expressed throughout the rodent CNS (Rienitz, A. et al. (1989) EMBO J. 8:2879-2888).

Neurotransmitters and Neurotransmitter Transport Proteins

Neurotransmitters comprise a diverse group of some 30 small molecules which include acetylcholine, monoamines such as serotonin, dopamine, and histamine, and amino acids such as gamma-aminobutyric acid (GABA), glutamate, and aspartate, and neuropeptides such as endorphins and enkephalins (McCance, K. L. and S. E. Huether (1994) PATHOPHYSIOLOGY, The Biologic Basis for Disease in Adults and Children, 2nd edition, Mosby, St. Louis, Mo., pp. 403-404). Many of these molecules have more than one function and the effects may be excitatory, e.g. to depolarize the postsynaptic cell plasma membrane and stimulate nerve impulse transmission, or inhibitory, e.g. to hyperpolarize the plasma membrane and inhibit nerve impulse transmission.

Neurotransmitters and their receptors are targets of pharmacological agents aimed at controlling neurological function. For example, GABA is the major inhibitory neurotransmitter in the CNS, and GABA receptors are the principal target of sedatives such as benzodiazepines and barbiturates which act by enhancing GABA-mediated effects (Katzung, B. G. (1995) Basic and Clinical Pharmacology, 6th edition, Appleton & Lange, Norwalk, Conn., pp. 338-339).

Two major classes of neurotransmitter transporters are essential to the function of the nervous system. The first class is uptake carriers in the plasma membrane of neurons and glial cells, which pump neurotransmitters from the extracellular space into the cell. This process relies on the Na⁺ gradient across the plasma membrane, particularly the co-transport of Na⁺. Two families of proteins have been identified. One family includes the transporters for GABA, monoamines such as noradrenaline, dopamine, and serotonin, and amino acids such as glycine and proline. Common structural components include twelve putative transmembrane a-helical domains, cytoplasmic N- and C-termini, and a large glycosylated extracellular loop separating transmembrane domains three and four. This family of homologous proteins derives their energy from the co-transport of Na⁺ and Cl⁻ ions with the neurotransmitter into the cell (Na⁺/Cl⁻ neurotransmitter transporters). The second family includes transporters for excitatory amino acids such as glutamate. Common structural components include 6-10 putative transmembrane domains, cytoplasmic N- and C-termini, and glycosylations in the extracellular loops. The excitatory amino acid transporters are not dependent on Cl⁻, and may require intracellular K⁺ ions (Na⁺/K⁺-neurotransmitter transporters) (Liu, Y. et al. (1999) Trends Cell Biol. 9:356-363).

The second class of neurotransmitter transporters is present in the vesicle membrane, and concentrates neurotransmitters from the cytoplasm into the vesicle, before exocytosis of the vesicular contents during synaptic transmission. Vesicular transport uses the electrochemical gradient across the vesicular membrane generated by a H⁺-ATPase. Two families of proteins are involved in the transport of neurotransmitters into vesicles. One family uses primarily proton exchange to drive transport into secretory vesicles and includes the transporters for monoamines and acetylcholine. For example, the monoamine transporters exchange two luminal protons for each molecule of cytoplasmic transmitter. The second family includes the GABA transporter, which relies on the positive charge inside synaptic vesicles. The two classes of vesicular transporters show no sequence similarity to each other and have structures distinct from those of the plasma membrane carriers (Schloss, P. et al., (1994) Curr. Opin. Cell Biol. 6:595-599; Liu et al., supra).

GABA is the predominant inhibitory neurotransmitter and is widely distributed in the mammalian nervous system. GABA is cleared from the synaptic cleft by specific, high-affinity, Na⁺- and Cl⁻-dependent transporters, which are thought to be localized to both pre- and postsynaptic neurons, as well as to surrounding glial cells. At least four GABA transporters (GAT1-GAT4) have been cloned (Liu, Q.-R. et al. (1993) J. Biol. Chem. 268:2106-2112). Studies of [³H]-GABA uptake into cultured cells and plasma-membrane vesicles isolated from various tissues revealed considerable differences in GABA transporter heterogeneity. GABA transporters exhibit differences in substrate affinity and specificity, distinct blocker pharmacologies, and different tissue localization. For example, the Kₘ values of GABA uptake of the expressed GAT1 to GAT4 are 6, 79, 18, and 0.8 mM, respectively. In addition to transporting GABA, GAT2 also transports betaine; GAT3 and GAT4 also transport β-alanine and taurine. Pharmacological studies revealed that GABA transport by GAT1 and GAT4 is more sensitive to 2,4-diaminobutyric acid and guavicine than that by GAT2 and GAT3. In situ hybridization showed that GAT1 and GAT4 expression is brain specific. GAT2 and GAT3 mRNAs were detected in tissues such as liver and kidney (Schloss et al., supra; Borden, L. A. (1996) Neurochem. Int. 29:335-356; Nelson, N. (1998) J. Neurochem. 71:1785-1803).

Human studies indicated that GABA transporter function is reduced in epileptic hippocampi. Decreased GABAergic neurotransmission has also been implicated in the pathophysiology of schizophrenia (Simpson, M. D. et al. (1992) Psychiatry Res. 42:273-282).

Diazepam binding inhibitor (DBI), also known as endozepine and acyl-Coenzyme (CoA)-binding protein, is an endogenous GABA receptor ligand which is thought to down-regulate the effects of GABA. DBI binds medium- and long-chain acyl-CoA esters with very high affinity and may function as an intracellular carrier of acyl-CoA esters (*125950 Diazepam Binding Inhibitor; DBI, Online Mendelian Inheritance in Man (OMIM); PROSITE PDOC00686 Acyl-CoA-binding protein signature).

Glycine serves as one of the major inhibitory neurotransmitters in the mammalian nervous system by activating chloride-channel receptors, which are members of a ligand-gated ion-channel superfamily (Betz, H (1990) Neuron 5:383-392). Glycine also facilitates excitatory transmission through an allosteric activation of the N-methyl-D-aspartate (NMDA) receptor (Johnson, J. W. and P. Ascher (1987) Nature 325:529-531). Forms of glycine transporter include GLYT 1 and GLYT 2. Variants of GLYT1 (GLYT1 a/b) are generated by alternative splicing (Liu, Q.-R. et al. (1993) J. Biol. Chem. 268:22802-22808). GLYT1a is transcribed in both neural and non-neural tissues, whereas GLYT1b was detected only in neural tissues (Borowsky, B. et al. (1993) Neuron 10:851-863). High levels of GLYT1a/b mRNA were found in hippocampus and cortex, implying its involvement in the regulation of excitatory synaptic transmission. It is not clear whether GLYT1a is expressed in neurons, in glia or in both. In contrast, GLYT1b is found almost exclusively in fiber tracts, suggesting its localization in glial cells (Schloss et al., supra). GLYT2 is expressed mainly in brainstem and spinal cord (Schloss et al., supra).

The second identified glycine transporter (GLYT2) differs from GLYT1a/b by its extended intracellular amino terminus. The predominant localization of its mRNA in brainstem and spinal cord and its insensitivity to N-methyl-aminoacetic acid suggests that GLYT2 terminates signal transduction at the strychnine-sensitive inhibitory glycine receptor. It has been proposed that, upon depolarization of cells harboring GLYT1b, the transporter runs backwards and releases glycine to act as a neuromodulatory amino acid at the NMDA receptor (Attwell, D. and M. Bouvier (1992) Curr. Biol. 2:541-543). Such a Ca²⁺-independent, non-vesicular release of neurotransmitters by reverse transport was demonstrated for glutamate and serotonin. This evidence suggests that the transmitter transporters may be important for both the initiation and termination of neurotransmitter action (Schloss et al., supra).

Creatine transporters are strongly related to transporters for GABA. The primary sequence identity between creatine transporter species homologs is very high (98-99%). Pharmacological characterization demonstrated high affinity creatine uptake (27-43 mM), which was blocked by creatine analogs with high affinity. Creatine transporters are widely expressed in a variety of mammalian tissues, including brain, adrenal gland, intestine, colon, prostate, thymus, ovary, spleen, pancreas, placenta, umbilical cord, thyroid, tongue, pharynx, vertebral discs, jaw, and nasal epithelium. Genetic mapping in the mouse localizes the creatine transporter to a region on the X chromosome in linkage conservation with the human region Xq28, the location of the genes for several neuromuscular diseases (Nash, S. R. et al. (1994) Receptors Channels 2:165-174).

The substrates of a number of cDNA clones encoding proteins of the Na⁺/Cl⁻-dependent transporter families are still not identified. These are orphan transporters. Identification of the substrates for orphan transporters has been difficult because in situ hybridization and immunohistochemistry indicate that the transporters are synthesized by phenotypically different neuronal populations, for example glutaminergic, GABAergic, histaminergic, or serotoninergic neurons. One of the transporters, NTT4, exhibits the highest homology to the creatine transporter. It differs structurally from other members of this family in having an unusually long loop between transmembranes seven and eight (Liu, Q.-R. et al. (1993) FEBS Lett. 315:114-118; Schloss et al., supra).

Glutamate is a major excitatory neurotransmitter in the mammalian central nervous system. Electrogenic (Na⁺/K⁺)-coupled glutamate transporters, located in the plasma membranes of nerve terminals and glial cells, mediate removal of glutamate released at excitatory synapses and maintain extracellular concentrations below neurototoxic levels. Glutamate transporters achieve this process by co-transport with three sodium ions and one proton, followed by translocation of a potassium ion in the opposite direction (Zerangue, N. and M. P. Kavanaugh (1996) Nature 383:634-637).

The membrane topology of the glutamate transporters reveals six membrane-spanning helices in the N-terminal part of the proteins (Slotboom, D. J. et al. (1999) Microbiol. Mol. Biol. Rev. 63:293-307). The C-terminal half of the glutamate transporters is well conserved and constitutes a major part of the translocation pathway and contains the binding sites for the substrate and co-transported ions (Zhang, Y. and B. I. Kanner (1999) Proc. Natl. Acad. Sci. USA 96:1710-1715).

Impaired re-uptake of synaptic glutamate, and a reduced expression of glutamate transporters have been found in the motor cortex of patients with amyotrophic lateral sclerosis (ALS). Inhibition of the synthesis of each glutamate transporter subtype using chronic antisense oligonucleotide administration, in vitro and in vivo, selectively and specifically reduced the protein expression and function of glutamate transporters. The loss of glial glutamate transporters produced elevated extracellular glutamate levels, neurodegeneration characteristic of excitotoxicity, and a progressive paralysis. The loss of the neuronal glutamate transporter did not elevate extracellular glutamate in the striatum but produced mild neurotoxicity and resulted in epilepsy (Rothstein, J. D. et al. (1996) Neuron 16:675-686).

Human diseases caused by defects in neurotransmitter transporters include schizophrenia, Tourette's syndrome, Parkinson's disease, brain ischemia, amyotrophic lateral scerlosis, depression, and epilepsy. For example, decreased GABAergic neurotransmission has been implicated in the pathophysiology of CNS disorders such as epilepsy and schizophrenia. Impaired re-uptake of synaptic glutamate, and a reduced expression of the glutamate transporter have been found in the motor cortex of patients with amyotrophic lateral sclerosis (ALS). The loss of glial glutamate transporters produces elevated extracellular glutamate levels, neurodegeneration characteristic of excitotoxicity, and a progressive paralysis. The loss of neuronal glutamate transporters produces mild neurotoxicity and result in epilepsy (Rothstein, J. D. et al. supra).

The vesicular monoamine transporters (VMAT) package cytoplasmic monoamine neurotransmitters into secretory vesicles for regulated exocytotic release. VMAT acts as an electrogenic exchanger of protons and monoamines, using a proton electrochemical gradient. VMAT transporters include VMAT1 and VMAT2. The VMAT proteins possess twelve transmembrane segments, with both extremities lying on the cytoplasmic side. VMAT proteins are associated with distinct vesicle populations in neurons and neuroendocrine cells (Henry, J.-P. et al. (1994) J. Exp. Biol. 196:251-262).

Vesicular transport is inhibited by the antihypertensive drug reserpine and the related but more centrally acting drug tetrabenazine. The mechanism of transport and the biochemistry of VMAT have been analyzed with these drugs, using mainly the chromaffin granules from bovine adrenal glands as a source of transporters (Peter, D. et al. (1994) J. Biol. Chem. 269:7231-7237).

Human studies indicated that reserpine can cause a syndrome resembling depression, indicating the importance of vesicular transport activity for the control of mood and behavior. The psychostimulant amphetamine also disrupts the storage of amines in secretory vesicles, further indicating that alterations in vesicular monoamine transport can affect behavior (Sulzer, D. and S. Rayport (1990) Neuron 5:797-808).

Another family of molecules that appear to be important for neurotransmission are the choline-transporter-like CTL1 proteins. The prototypic CTL1 was identified in yeast as a suppressor of a choline transport mutation; however, mammalian homologues have been identified. The proteins comprise approximately ten putative transmembrane domains in addition to transporter-like motifs but do not appear to be canonical choline transporters. Choline transport is important to neurotransmission because choline is a precursor of acetylcholine, required in abundance by cholinergic neurons (O'Regan, S. et al. (2000) Proc. Nat]. Acad. Sci. U.S.A. 97:1835-1840).

Neuronal signals are transmitted across the neuromuscular junction (NMJ). Motor axons release the molecule agrin to induce the formation of the postsynaptic apparatus in muscle fibers. Proteins such as dystroglycan, MuSK, and rapsyn participate in the transduction of agrin signals. Agrin also functions in the upregulation of gene transcription in myonuclei and the control of presynaptic differentiation (Ruegg, M. A. and J. L. Bixby (1998) Trends Neurosci. 21:22-27).

Neurological Protein Domains

CNS-associated proteins can be phosphoproteins. For example, ARPP-21 (cyclic AMP-regulated phosphoprotein) is a cytosolic neuronal phosphoprotein that is highly enriched in the striatum and in other dopaminoceptive regions of the brain. The steady-state level of ARPP-21 mRNA is developmentally regulated. But, in the neonatal and mature animal, ARPP-21 mRNA is not altered following 6-hydroxydopamine lesions of the substantia nigra or by pharmacologic treatments that upregulate the D1- or D2-dopamine receptors (Ehrlich, M. E. et al. (1991) Neurochem. 57:1985-1991).

CNS-associated signaling proteins may contain PDZ domains. PDZ domains have been found in proteins which act as adaptors in the assembly of multifunctional protein complexes involved in signaling events at surfaces of cell membranes. PDZ domains are generally found in membrane-associated proteins including neuronal nitric oxide synthase (NOS) and several dystrophin-associated proteins (Ponting, C. P. et al. (1997) Bioessays 19:469-479). PSD-95/SAP90 is a membrane-associated guanylate kinase found in neuronal cells at the postsynaptic density (PSD) (Takeuchi, M. et al. (1997) J. Biol. Chem. 272:11943-11951). PSD-95/SAP90 contains three PDZ domains, one SH3 domain, and one guanylate kinase domain. The PDZ domains mediate interactions with NMDA receptors, Shaker-type potassium channels, and brain nitric oxide synthase. SAPAPs (AP90/PSD-95-Associated Proteins) promote localization of PSD-95/SAP90 at the plasma membrane.

CNS-associated proteins may also contain epidermal growth factor (EGF) domains. The Notch proteins are transmembrane proteins which contain extracellular regions of repeated EGF domains. Notch proteins, such as the Drosophila melanogaster neurogenic protein Notch, are generally involved in the inhibition of developmental processes. Other members of the Notch family are the lin-12 and glp-1 genes of Caenorhabditis elegans. Genetic studies indicate that the lin-12 and glp-1 proteins act as receptors in specific developmental cell interactions which may be involved in certain embyronic defects (Tax, F. E. et al. (1994) Nature 368:150-154). Pecanex, a maternal-effect neurogenic locus of D. melanogaster, is believed to encode a large transmembrane protein. In the absence of maternal expression of the pecanex gene, an embryo develops severe hyperneuralization similar to that characteristic of Notch mutant embryos (LaBonne, S. G. et al. (1989) Dev. Biol. 136:1-116).

Other CNS-associated signaling proteins contain WW domains. The WW domain is a protein motif with two highly conserved tryptophans. It is present in a number of signaling and regulatory proteins, including Huntingtin interacting protein. Several fibroblast growth factor (FGF) homologous factors (i.e., FHF polypeptides) have also been implicated in nervous system development based on mRNA expression patterns in mouse and human tissues. Members of the FHF family of polypeptides are structurally distinct from prototypic FGFs, consistent with the unusual role of these FGF-related proteins (Smallwood, P. M. et al. (1996) Proc. Natl. Acad. Sci. U.S.A. 93:9850-9857 and Hartung, H. et al. (1997) Mech. Dev. 64:31-39).

Disorders Associated with Neurological Processes

Alzheimer's disease (AD) is a degenerative disorder of the CNS which causes progressive memory loss and cognitive decline during mid to late adult life. AD is characterized by a wide range of neuropathologic features including amyloid deposits and intra-neuronal neurofibrillary tangles. Although the pathogenic pathway leading to neurodegeneration and AD is not well understood, at least three genetic loci that confer genetic susceptibility to the disease have been identified (Schellenberg, G. D. (1995) Proc. Natl. Acad. Sci. 92:8552-8559; Sherrington, R. et al. (1995) Nature 375:754-760).

Familial British dementia (FBD), is an autosomal dominant disease featuring amyloid plaques surrounded by astrocytes and microglia, neurofibrillary tangles, neuronal loss, and progressive dementia. The BRI gene on chromosome 13 encodes a 4 kD peptide, A-Bri. This membrane-anchored protein is a primary constituent of amyloid deposits, and its presence in lesions from the CNS of FBD patients maybe a contributive factor of this disease (El-Agnaf, O. M. A. et al. (2001) Biochemistry 40:3449-3457).

Astrocytomas, and the more malignant glioblastomas, are the most common primary tumors of the brain, accounting for over 65% of primary brain tumors. These tumors arise in glial cells of the astrocyte lineage. Following infection by pathogens, astrocytes function as antigen-presenting cells and modulate the activity of lymphocytes and macrophages. Astrocytomas constitutively express many cytolines and interleukins that are normally produced only after infection by a pathogen (de Micco, C. (1989) J. Neuroimmunol. 25:93-108). In the course of identifying genes related to astrocyte differentiation, one cDNA was isolated from an astrocytoma cDNA library that encodes a protein structurally related to the plant pathogenesis-related (PR) proteins (Murphy, E. V. et al. (1995) Gene 159:131-135). The glioma pathogenesis-related protein (GliPR) is highly expressed in glioblastoma, but not in fetal or adult brain, or in other nervous system tumors. PR proteins are a family of small (10-20 kDa), protease resistant proteins induced in plants by viral infections, such as tobacco mosaic virus. The synthesis of PR proteins is believed to be part of a primitive immunological response in plants (van Loon, L. C. (1985) Plant Mol. Biol. 4:111-116). GliPR shares up to 50% homology with the PR-1 protein family over a region that comprises almost two thirds of the protein, including a conserved triad of amino acids, His-Glu-His, appropriately spaced to form a metal-binding domain (Murphy et al., supra).

Signaling initiated by the Trk family receptors plays a dynamic role in neurogenic tumors. The proto-oncogene Trks encode the high-affinity receptor tyrosine kinases for nerve growth factor (NGF) neurotrophins. A rearranged Trk oncogene is often observed in non-neuronal neoplasms such as colon and papillary thyroid cancers. The proto-oncogene Trks regulates growth, differentiation and apoptosis of tumors of neuronal origin, such as neuroblastoma and medulloblastoma (Nakagawara, A. (2001) Cancer Lett.169:107-114).

Neuronal thread proteins (NTP) are a group of immunologically related molecules found in the brain and neuroectodermal tumor cell lines. NTP expression is increased in neuronal cells during proliferation, differentiation, brain development, in Alzheimer's disease (AD) brains, and in pathological states associated with regenerative nerve sprouting (de la Monte, S. M. et al. (1996) J. Neuropathol. Exp. Neurol. 55:1038-1050). Monoclonal antibodies generated to a recombinant NTP, AD7c-NTP, isolated from an end-stage AD brain library, showed high levels of NTP immunoreactivity in perikarya, neuropil fibers, and white matter fibers of AD brain tissue. In vitro studies also demonstrated NTP upregulation, phosphorylation, and translocation from the perikarya to cell processes and growth cones during growth factor-induced neuritic sprouting and neuronal differentiation. Additionally, increased NIP immunoreactivity was found in Down syndrome brains beginning in the second decade, prior to establishment of widespread AD neurodegeneration, and at an age when a low-level or an absence of NTP expression was observed in control brains. These findings indicated that abnormal expression and accumulation of NTP in brain may be an early marker of AD neurodegeneration in Down syndrome (de la Monte, S. M. et al. (1996) J. Neurol. Sci. 135:118-125). Furthermore, the increased expression and accumulation of NIP in AD brain tissue was paralleled by corresponding elevations of NTP in cerebrospinal fluid (CSF), and elevated levels of NTP were detectable in the CSF early in the course of the disease.

Fe65-like protein (Fe65L2), a new member of the Fe65 protein family, is one of the ligands that interacts with the cytoplasmic domain of Alzheimer beta-amyloid precursor protein (APP). Transgenic mice expressing APP simulate some of the prominent behavioral and pathological features of Alzheimer's disease, including age-related impairment in learning and memory, neuronal loss, gliosis, neuritic changes, amyloid deposition, and abnormal tau phosphorylation (Duilio, A. et al. (1998) Biochem. J. 330:513-519).

Amyotrophic lateral sclerosis (ALS) is characterized by motor neuron death, altered peroxidase activity of mutant SOD1, changes in intracellular copper homeostasis, protein aggregation, and changes in the function of glutamate transporters leading to excitotoxicity. Neurofilaments and peripherin appear to play some part in motor neuron degeneration. ALS is occasionally associated with mutations of the neurofilament heavy chain gene (Al-Chalabi, A. and P. N. Leigh (2000) Curr. Opin. Neurol. 13:397405). Cytoskeletal abnormalities such as abnormal inclusions containing neurofilaments (NFs) and/or peripherin, reduced mRNA levels for the NF light (NF-L) protein and mutations in the NF heavy (NF-H) gene have been observed in ALS. Intermediate filament inclusions containing peripherin may play a contributory role in ALS (Julien, J. P. and J. M. Beaulieu (2000) J. Neurol. Sci. 180:7-14).

Miller-Dieker syndrome (MDS) or isolated lissencephaly syndrome (ILS) are characterized by a smooth cerebral surface, a thickened cortex with four abnormal layers, and misplaced neurons. Both conditions may result from deletion or mutation in the LIS1 gene. The lissencephaly gene product Lis1 is a component of evolutionarily conserved intracellular multiprotein complexes essential for neuronal migration, and which may be components of the machinery for cell proliferation and intracellular transport (Leventer, R. J. et al. (2001) Trends Neurosci. 24:489-492). NudC, a nuclear movement protein, interacts with Lis1 (Morris, S. M. et al. (1998) Curr. Biol. 8:603-606).

NudC, a nuclear movement protein, interacts with the lissencephaly gene product Lis1, a protein involved in neuronal migration. People with Miller-Dieker syndrome (MDS) or isolated lissencephaly sequence (.S) have a hemizygous deletion or mutation in the LIS1 gene. Both conditions are characterized by a smooth cerebral surface, a thickened cortex with four abnormal layers, and misplaced neurons. LIS1 is highly expressed in the ventricular zone and the cortical plate. The interaction of Lis1 with NudC, in conjunction with the MDS and ILS phenotypes, raises the possibility that nuclear movement in the ventricular zone is closely related to neuronal fates and to cortical architecture. (Morris, S. M. et al. (1998) Curr. Biol. 8:603-606.)

Retinitis pigmentosa comprises a group of slowly progressive, inherited disorders of the retina that cause loss of night vision and peripheral visual field in adolescence. A recessive nonsense mutation in the Drosophila opsin gene causes photoreceptor degeneration. In some families, genes encoding rhodopsin and peripherin/RDS map very close to the disease loci. Rhodopsin and peripherin/RDS mutations have been found in approximately 30% of all autosomal dominant cases (Shastry, B. S. (1994) Am. J. Med. Genet 52:467-474).

Synaptic proteins are involved in Alzheimer's disease (AD) and other disorders including ischemia, a variety of disorders where synapse-associated proteins are abnormally accumulated in the nerve terminals or synaptic proteins are altered after denervation, and neoplastic disorders (Masliah, E. and R. Terry (1993) Brain Pathol. 3:77-85). Synaptophysin (SY), a major integral membrane protein of small synaptic vesicles, is on the X chromosome in subbands Xp11.22-p11.23, a region implicated in several inherited diseases including Wiskott-Aldrich syndrome, three forms of X-linked hypercalciuric nephrolithiaisis, and the eye disorders retinitis pigmentosa 2, congenital stationary night blindness, and Aland Island eye disease (Fisher, S. E. et al. (1997) Genomics 45:340-347).

Mutations in the BRI2 isoform of the BRI gene family are associated with dementia in humans (Vidal, R. et al. (2001) Gene 266:95-102).

Changes in the molecular and cellular components of neuronal signaling systems correlate with the effects on mood and cognition observed after long-term treatment with antidepressant drugs. Two serine/threonine kinases, Ca²⁺/calmodulin-dependent protein kinase II and cyclic AMP-dependent protein kinase, are activated in the brain following antidepressant treatment. Associated changes in the phosphorylation of selected protein substrates in subcellular compartments including presynaptic terminals and microtubules may contribute to the modulation of synaptic transmission observed with antidepressants (Popoli, M. et al. (2001) Pharmacol. Ther. 89:149-170). Reserpine can cause a syndrome resembling depression, indicating the importance of vesicular transport activity for the control of mood and behavior. The psychostimulant amphetamine also disrupts the storage of amines in secretory vesicles, further indicating that alterations in vesicular monoamine transport can affect behavior (Sulzer, D. and S. Rayport (1990) Neuron 5:797-808).

Decreased GABAergic neurotransmission has been implicated in the pathophysiology of CNS disorders such as epilepsy and schizophrenia. Impaired re-uptake of synaptic glutamate and a reduced expression of the glutamate transporter have been found in the motor cortex of patients with amyotrophic lateral sclerosis (ALS). The loss of glial glutamate transporters produces elevated extracellular glutamate levels, neurodegeneration characteristic of excitotoxicity, and a progressive paralysis. The loss of neuronal glutamate transporters produces mild neurotoxicity and results in epilepsy (Rothstein, J. D. et al. (1996) Neuron 16:675-686). GABA transporter function is reduced in epileptic hippocampi. Transporters for dopamine, norepinephrine, and serotonin have particular significance as targets for clinically relevant psychoactive agents including cocaine, antidepressants, and amphetamines. Cocaine and antidepressants are transporter antagonists that act with varying degrees of specificity to enhance synaptic concentrations of amines by limiting clearance. Amphetamines enhance transporter mediated efflux in concert with a depletion of vesicular amine stores (Barker, E. L. and R. D. Blakely (1995) Psychopharmacology 28:321-333; Sulzer, D. and S. Rayport (1990) Neuron 5:797-808; Wall, S. C. et al. (1995) Mol. Pharmacol. 47:544-550).

The μ-opioid receptor (MOR) mediates the actions of analgesic agents including morphine, codeine, methadone, and fentanyl as well as heroin. MOR is functionally coupled to a G-protein-activated potassium channel (Mestek A. et al. (1995) J. Neurosci. 15:2396-2406). A variety of MOR subtypes exist. Alternative splicing has been observed with MOR-1 as with a number of G protein-coupled receptors including somatostatin 2, dopamine D2, prostaglandin BP3, and serotonin receptor subtypes 5-hydroxytryptamine4 and 5-hydroxytryptamine7 (Pan, Y. X. et al. (1999) Mol. Pharm. 56:396-403).

The central nervous system regulates the innate immune system by elaborating anti-inflammatory hormone cascades in response to bacterial products and immune mediators. The central nervous system also responds via acetylcholine-mediated efferent signals carried through the vagus nerve. Nicotinic cholinergic receptors expressed on macrophages detect these signals and respond with a dampened cytokine response (Tracey K. J. et al. (2001) FASEB J. 15:1575-1576).

Dysferlin is the protein product of the gene mutated in patients with an autosomal recessive limb-girdle muscular dystrophy type 2B (LGMD2B) and a distal muscular dystrophy, Miyoshi myopathy. Dysferlin is homologous to a Caenorhabditis elegans spermatogenesis factor, FER-1. Otoferlin, another human FER-1-like protein (ferlin), is responsible for autosomal recessive nonsyndromic deafness (DFNB9). All the ferlins are characterized by sequences corresponding to multiple C2 domains that share the highest level of homology with the C2A domain of rat synaptotagmin III (Britton S. et al. (2000) Genomics 68:313-321).

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Atherosclerosis

Atherosclerosis and the associated coronary artery disease and cerebral stroke represent the most common cause of death in industrialized nations. Although certain key risk factors have been identified, a full molecular characterization that elucidates the causes and provide care for this complex disease has not been achieved. Molecular characterization of growth and regression of atherosclerotic vascular lesions requires identification of the genes that contribute to features of the lesion including growth, stability, dissolution, rupture and, most lethally, induction of occlusive vessel thrombus.

An early step in the development of atherosclerosis is formation of the “fatty streak”. Lipoproteins, such as the cholesterol-rich low-density lipoprotein (LDL), accumulate in the extracellular space of the vascular intima, and undergo modification. Oxidation of LDL occurs most avidly in the sub-endothelial space where circulating antioxidant defenses are less effective. The degree of LDL oxidation affects its interaction with target cells. “Minimally oxidized” LDL (MM-LDL) is able to bind to LDL receptor but not to the oxidized LDL (Ox-LDL) or “scavenger” receptors that have been identified, including scavenger receptor types A and B, CD36, CD68/macrosialin and LOX-1 (Navab et al. (1994) Arterioscler Thromb Vasc Biol 16:831-842; Kodama et al. (1990) Nature 343:531-535; Acton et al. (1994) J Biol Chem 269:21003-21009; Endemann et al. (1993) J Biol Chem 268:11811-11816; Ramprasad et al. (1996) Proc Natl Acad Sci 92:14833-14838; Kataoka et al. (1999) Circulation 99:3110-3117). MM-LDL can increase the adherence and penetration of monocytes, stimulate the release of monocyte chemotactic protein 1 (MCP-1) by endothelial cells, and induce scavenger receptor A (SRA) and CD36 expression in macrophages (Cushing et al. (1990) Proc Natl Acad Sci 87:5134-5138; Yoshida et al. (1998) Arterioscler Thromb Vasc Biol 18:794-802; Steinberg (1997) J Biol Chem 272:20963-20966). SRA and the other scavenger receptors can bind Ox-LDL and enhance uptake of lipoprotein particles.

Mononuclear phagocytes enter the intima, differentiate into macrophages, and ingest modified lipids including Ox-LDL. In most cell types, cholesterol content is tightly controlled by feedback regulation of LDL receptors and biosynthetic enzymes (Brown and Goldstein (1986) Science 232:34-47). In macrophages, however, the additional scavenger receptors lead to unregulated uptake of cholesterol (Brown and Goldstein (1983) Annu Rev Biochem 52:223-261) and accumulation of multiple intracellular lipid droplets producing a “foam cell” phenotype. Cholesterol-engorged and dead macrophages contribute most of the mass of early “fatty streak” plaques and typical “advanced” lesions of diseased arteries. Numerous studies have described a variety of foam cell responses that contribute to growth and rupture of atherosclerotic vessel wall plaques. These responses include production of multiple growth factors and cytokines, which promote proliferation and adherence of neighboring cells; chemokines, which further attract circulating monocytes into the growing plaque; proteins, which cause remodeling of the extracellular matrix; and tissue factor, which can trigger thrombosis (Ross (1993) Nature 362:801-809; Quin et al. (1987) Proc Natl Acad Sci 84:2995-2998). Thus, cholesterol-loaded macrophages which occur in abundance in most stages of the atherosclerotic plaque formation contribute to inception of the atherosclerotic process and to eventual plaque rupture and occlusive thrombus.

During Ox-LDL uptake, macrophages produce cytokines and growth factors that elicit further cellular events that modulate atherogenesis such as smooth muscle cell proliferation and production of extracellular matrix. Additionally, these macrophages may activate genes involved in inflammation including inducible nitric oxide synthase. Thus, genes differentially expressed during foam cell formation may reasonably be expected to be markers of the atherosclerotic process.

Lung Cancer

Lung cancer is the leading cause of cancer death for men and the second leading cause of cancer death for women in the U.S. Lung cancers are divided into four histopathologically distinct groups. Three groups (squamous cell carcinoma, adenocarcinoma, and large cell carcinoma) are classified as non-small cell lung cancers (NSCLCs). The fourth group of cancers is referred to as small cell lung cancer (SCLC). Deletions on chromosome 3 are common in lung cancer. Activating mutations in K-ras are commonly found in lung cancer and are the basis of one of the mouse models for the disease. Analysis of gene expression patterns associated with the development and progression of the disease will yield tremendous insight into the biology underlying this disease, and will lead to the development of improved diagnostics and therapeutics.

Ovarian Cancer

Ovarian cancer is the leading cause of death from a gynecologic cancer. The majority of ovarian cancers are derived from epithelial cells, and 70% of patients with epithelial ovarian cancers present with late-stage disease. As a result, the long-term survival rates for this disease is very low. Identification of early-stage markers for ovarian cancer would significantly increase the survival rate. Genetic variations involved in ovarian cancer development include mutation of p53 and microsatellite instability. Gene expression patterns likely vary when normal ovary is compared to ovarian tumors.

There is a need in the art for new compositions, including nucleic acids and proteins, for the diagnosis, prevention, and treatment of autoimmune/inflammatory, cardiovascular, neurological, developmental, cell proliferative, transport, psychiatric, metabolic, and endocrine disorders.

### SUMMARY OF THE INVENTION

Various embodiments of the invention provide purified polypeptides, neurotransmission-associated proteins, referred to collectively as ‘NTRAN’ and individually as ‘NTRAN-1’, ‘NTRAN-2’, ‘NTRAN-3', ‘NTRAN-4’, ‘NTRAN-5’, ‘NTRAN-6’, ‘NTRAN-7’, ‘NTRAN-8’, ‘NTRAN-9’, ‘NTRAN-10’, ‘NTRAN-11’, ‘NTRAN-12’, ‘NTRAN-13’, ‘NTRAN-14’, ‘NTRAN-15’, ‘NTRAN16’, ‘NTRAN-17’, ‘NTRAN-18’, ‘NTRAN-19’, ‘NTRAN-20’, ‘NTRAN-21’, ‘NTRAN-22’, ‘NTRAN-23’, ‘NTRAN-24’, and ‘NTRAN-25’, and methods for using these proteins and their encoding polynucleotides for the detection, diagnosis, and treatment of diseases and medical conditions. Embodiments also provide methods for utilizing the purified neurotransmission-associated proteins and/or their encoding polynucleotides for facilitating the drug discovery process, including determination of efficacy, dosage, toxicity, and pharmacology. Related embodiments provide methods for utilizing the purified neurotransmission-associated proteins and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. Another embodiment provides an isolated polypeptide comprising an amino acid sequence of SEQ ID NO:1-25.

Still another embodiment provides an isolated polynucleotide encoding a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. In another embodiment, the polynucleotide encodes a polypeptide selected from the group consisting of SEQ ID NO:1-25. In an alternative embodiment, the polynucleotide is selected from the group consisting of SEQ ID NO:26-50.

Still another embodiment provides a recombinant polynucleotide comprising a promoter sequence operably bilked to a polynucleotide encoding a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) abiologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25.

Still yet another embodiment provides an isolated polynucleotide selected from the group consisting of a) a polynucleotide comprising a polynucleotide sequence selected from the group consisting of SEQ ID NO:26-50, b) a polynucleotide comprising a naturally occurring polynucleotide sequence at least 90% identical or at least about 90% identical to a polynucleotide sequence selected from the group consisting of SEQ ID NO:26-50, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:26-50, b) a polynucleotide comprising a naturally occurring polynucleotide sequence at least 90% identical or at least about 90% identical to a polynucleotide sequence selected from the group consisting of SEQ ID NO:26-50, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:26-50, b) a polynucleotide comprising a naturally occurring polynucleotide sequence at least 90% identical or at least about 90% identical to a polynucleotide sequence selected from the group consisting of SEQ ID NO:26-50, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25, and a pharmaceutically acceptable excipient. In one embodiment, the composition can comprise an amino acid sequence selected from the group consisting of SEQ ID NO:1-25. Other embodiments provide a method of treating a disease or condition associated with decreased or abnormal expression of functional NTRAN, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional NTRAN, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional NTRAN, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) abiologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-25, b) a polypeptide comprising a naturally occurring amino acid sequence at least 90% identical or at least about 90% identical to an amino acid sequence selected from the group consisting of SEQ ID NO:1-25, c) a biologically active fragment of a polypeptide having an amino acid sequence selected from the group consisting of SEQ ID NO:1-25, and d) an immunogenic fragment of a polypeptide having an amino acid sequence selected from the group consisting of SEQ ID NO:1-25. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:26-50, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:26-50, ii) a polynucleotide comprising a naturally occurring polynucleotide sequence at least 90% identical or at least about 90% identical to a polynucleotide sequence selected from the group consisting of SEQ ID NO:26-50, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:26-50, ii) a polynucleotide comprising a naturally occurring polynucleotide sequence at least 90% identical or at least about 90% identical to a polynucleotide sequence selected from the group consisting of SEQ ID NO:26-50, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along With the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide sequences of the invention, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“NTRAN” refers to the amino acid sequences of substantially purified NTRAN obtained from any species, particularly a mammalian species, including bovine, ovine, porcine, murine, equine, and human, and from any source, whether natural, synthetic, semi-synthetic, or recombinant

The term “agonist” refers to a molecule which intensifies or mimics the biological activity of NTRAN. Agonists may include proteins, nucleic acids, carbohydrates, small molecules, or any other compound or composition which modulates the activity of NTRAN either by directly interacting with NTRAN or by acting on components of the biological pathway in which NTRAN participates.

An “allelic variant” is an alternative form of the gene encoding NTRAN. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding NTRAN include those sequences with deletions, insertions, or substitutions of different nucleotides, resulting in a polypeptide the same as NTRAN or a polypeptide with at least one functional characteristic of NTRAN. Included within this definition are polymorphisms which may or may not be readily detectable using a particular oligonucleotide probe of the polynucleotide encoding NTRAN, and improper or unexpected hybridization to allelic variants, with a locus other than the normal chromosomal locus for the polynucleotide encoding NTRAN. The encoded protein may also be “altered,” and may contain deletions, insertions, or substitutions of amino acid residues which produce a silent change and result in a functionally equivalent NTRAN. Deliberate amino acid substitutions may be made on the basis of one or more similarities in polarity, charge, solubility, hydrophobicity, hydrophilicity, and/or the amphipathic nature of the residues, as long as the biological or immunological activity of NTRAN is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of NTRAN. Antagonists may include proteins such as antibodies, anticalins, nucleic acids, carbohydrates, small molecules, or any other compound or composition which modulates the activity of NTRAN either by directly interacting with NTRAN or by acting on components of the biological pathway in which NTRAN participates.

The term “antibody” refers to intact immunoglobulin molecules as well as to fragments thereof, such as Fab, F(ab′)₂, and Fv fragments, which are capable of binding an epitopic determinant Antibodies that bind NTRAN polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions maybe double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-N₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers maybe specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic NTRAN, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding NTRAN or fragments of NTRAN may be employed as hybridization probes. The probes may be stored in freeze-dried form and may be associated with a stabilizing agent such as a carbohydrate. In hybridizations, the probe maybe deployed in an aqueous solution containing salts (e.g., NaCl), detergents (e.g., sodium dodecyl sulfate; SDS), and other components (e.g., Denhardt's solution, dry mil, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable laber” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of NTRAN or a polynucleotide encoding NTRAN which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments maybe preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:26-50 can comprise a region of unique polynucleotide sequence that specifically identifies SEQ ID NO:26-50, for example, as distinct from any other sequence in the genome from which the fragment was obtained. A fragment of SEQ ID NO:26-50 can be employed in one or more embodiments of methods of the invention, for example, in hybridization and amplification technologies and in analogous methods that distinguish SEQ ID NO:26-50 from related polynucleotides. The precise length of a fragment of SEQ ID NO:26-50 and the region of SEQ ID NO:26-50 to which the fragment corresponds are routinely determinable by one of ordinary skill in the art based on the intended purpose for the fragment.

A fragment of SEQ ID NO:1-25 is encoded by a fragment of SEQ ID NO:26-50. A fragment of SEQ ID NO:1-25 can comprise a region of unique amino acid sequence that specifically identifies SEQ ID NO:1-25. For example, a fragment of SEQ ID NO:1-25 can be used as an immunogenic peptide for the development of antibodies that specifically recognize SEQ ID NO:1-25. The precise length of a fragment of SEQ ID NO:1-25 and the region of SEQ ID NO:1-25 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “fall length” polynucleotide sequence encodes a “full length” polypeptide sequence.

“Homology” refers to sequence similarity or, alternatively, sequence identity, between two or more polynucleotide sequences or two or more polypeptide sequences.

The terms “percent identity” and “% identity,” as applied to polynucleotide sequences, refer to the percentage of identical residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm/nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters maybe, for example:

Matrix: BLOSUM62

Reward for match: 1

Penalty for mismatch: −2

Open Gap: S and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple-1, gap penalty=3, window=5, and “diagonals saved”=5. The PAM250 matrix is selected as the default residue weight table.

Alternatively the NCBI BLAST software suite maybe used. For example, for a pairwise comparison of two polypeptide sequences, one may use the “BLAST 2 Sequences” tool Version 2.0.12 (Apr. 21, 2000) with blastp set at default parameters. Such default parameters maybe, for example:

Matrix: BLOSUM62

Open Gap: 11 and Extension Gap: 1 penalties

Gap x drop-off 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., Cot or Rot analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of NTRAN which is capable of eliciting an immune response when introduced into a living organism, for example, a mammal. The term “immunogenic fragment” also includes any polypeptide or oligopeptide fragment of NTRAN which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of NTRAN. For example, modulation may cause an increase or a decrease in protein activity, binding characteristics, or any other biological, functional, or immunological properties of NTRAN.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences maybe in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and maybe pegylated to extend their lifespan in the cell.

“Post-translational modification” of an NTRAN may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of NTRAN.

“Probe” refers to nucleic acids encoding NTRAN, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989; Molecular Cloning: A Laboratory Manual, 2^{nd} ed., vol. 1-3, Cold Spring Harbor Press, Plainview N.Y.), Ausubel, F. M. et al. (1999; Short Protocols in Molecular Biology, 4^{th} ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids maybe part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UIRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing NTRAN, nucleic acids encoding NTRAN, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides.

The Invention

Various embodiments of the invention include new human neurotransmission-associated proteins (NTRAN), the polynucleotides encoding NTRAN, and the use of these compositions for the diagnosis, treatment, or prevention of autoimmune/inflammatory, cardiovascular, neurological, developmental, cell proliferative, transport, psychiatric, metabolic, and endocrine disorders.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown.

Table 2 shows sequences with homology to the polypeptides of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Columns shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are neurotransmission-associated proteins.

For example, SEQ ID NO:1 is 90% identical, from residue M1 to residue L686, to human amyloid A4 protein (GenBank ID g28721) as determined by the Basic Local Alignment Search Tool (BLAST). (See Table 2.) The BLAST probability score is 0.0, which indicates the probability of obtaining the observed polypeptide sequence alignment by chance. SEQ ID NO:1 also contains an amyloid A4 extracellular domain, and a Kunitz/bovine pancreatic trypsin inhibitor domain, as determined by searching for statistically significant matches in the hidden Markov model (HMM)-based PFAM database of conserved protein family domains. (See Table 3.) Data from BUMPS, BLAST, MOTIFS, and PROFILESCAN analyses provide further corroborative evidence that SEQ ID NO:1 is an amyloidogenic glycoprotein.

As another example, SEQ ID NO:4 is 92% identical, from residue M1 to residue Q162, and 98% identical, from residue R150 to residue V230, to human BRJ3 (GenBank ID g9588046) as determined by the Basic Local Alignment Search Tool (BLAST). (See Table 2.) The BLAST probability score is 5.8e-119, which indicates the probability of obtaining the observed polypeptide sequence alignment by chance. Data from additional BLAST analyses and MOTIFS analyses provide further corroborative evidence that SEQ ID NO:4 is a neurotransmission-associated protein.

As another example, SEQ ID NO:7 is 90% identical from residue Ml to residue V348, and 100% identical from residue G349 to residue A631, to human semaphorin B (GenBank ID g12248382) as determined by the Basic Local Alignment Search Tool (BLAST). (See Table 2.) The BLAST probability score is 0, which indicates the probability of obtaining the observed polypeptide sequence alignment by chance. SEQ ID NO:7 also contains a Sema domain as determined by searching for statistically significant matches in the hidden Markov model (HMM)-based PFAM database of conserved protein family domains. (See Table 3.) Data from BUMPS and MOTIFS analyses provide further corroborative evidence that SEQ ID NO:7 is a semaphorin.

As another example, SEQ ID NO:8 is 100% identical, from residue M1 to residue M98, to human divalent cation tolerant protein CUTA, a brain acetylcholinesterase putative membrane anchor (GenBank ID g6624588) as determined by the Basic Local Alignment Search Tool (BLAST). (See Table 2.) The BLAST probability score is 2.0e-46, which indicates the probability of obtaining the observed polypeptide sequence alignment by chance. SEQ ID NO:8 also contains a CutA1 divalent ion tolerance protein domain as determined by searching for statistically significant matches in the hidden Markov model (HMM)-based PFAM database of conserved protein family domains. (See Table 3.) Data from additional BLAST analyses provide further corroborative evidence that SEQ ID NO:8 is a divalent cation tolerance protein.

As another example, SEQ ID NO:9 is 97% identical, from residue Ml to residue F1115, to m-tomosyn (GenBank ID g3790³89) as determined by the Basic Local Alignment Search Tool (BLAST). (See Table 2.) The BLAST probability score is 0.0, which indicates the probability of obtaining the observed polypeptide sequence alignment by chance. SEQ ID NO:9 is localized to the subcellular region, has syntaxin-1 and WD gene function, and is a tomosyn protein, as determined by BLAST analysis using the PROTEOME database. SEQ ID NO:9 also contains a WD domain as determined by searching for statistically significant matches in the hidden Markov model (HMM)-based PFAM database of conserved protein family domains. (See Table 3.) Data from BLIMS, BLAST-PRODOM, BLAST-DOMO, and MOTIFS analyses provide further corroborative evidence that SEQ ID NO:9 is a syntaxin-binding protein molecule.

As another example, SEQ ID NO:10 is 99% identical, from residue E137 to residue T363, to FEZ1 (GenBank ID g1927202) as determined by the Basic Local Alignment Search Tool (BLAST). (See Table 2.) The BLAST probability score is 1.2e-1 14, which indicates the probability of obtaining the observed polypeptide sequence alignment by chance. SEQ ID NO:10 is localized to the subcellular region, has axonal outgrowth gene function, and is a FEZ protein, as determined by BLAST analysis using the PROTEOME database. Data from BLAST-PRODOM analysis provides further corroborative evidence that SEQ ID NO:10 is a FEZ molecule.

As another example, SEQ ID NO:12 is 98% identical, from residue Q183 to residue L505, and 93% identical, from residue S12 to residue U132, to Rattus norvegicus PSD-95/SAP90-associated protein-4 (GenBank ID g1864093) as determined by the Basic Local Alignment Search Tool (BLAST). (See Table 2.) The BLAST probability score is 5.4e-229, which indicates the probability of obtaining the observed polypeptide sequence alignment by chance. SEQ ID NO:12 also has homology to proteins that are localized to postsynaptic density, have signaling function, and are synaptic proteins that bind to the guanylate kinase-like domain of PSD-95/SAP90, as determined by BLAST analysis using the PROTEOME database. Data from BLAST analysis of the PRODOM database provide further corroborative evidence that SEQ ID NO: 12 is a neurotransmission-associated protein.

As another example, SEQ ID NO:18 is 98% identical, from residue K8 to residue K321, to human josephin MJD1 protein (GenBank ID g2262199) as determined by the Basic Local Alignment Search Tool (BLAST). (See Table 2.) The BLAST probability score is 2.3e-162, which indicates the probability of obtaining the observed polypeptide sequence alignment by chance. SEQ ID NO:18 also has homology to proteins that are localized to the carboxyl termini of MJD gene products, have nucleotide-excision repair and apoptotic function, and are josephin MJD proteins, as determined by BLAST analysis using the PROTEOME database. SEQ ID NO:18 also contains a josephin domain and a ubiquitin interaction motif domain as determined by searching for statistically significant matches in the hidden Markov model (HMM)-based PFAM database of conserved protein family domains. (See Table 3.) Data from BLAST analyses provide further corroborative evidence that SEQ ID NO:18 is a josephin protein.

As another example, SEQ ID NO:23 is 100% identical, from residue M1-E43 and 95% identical, from residue A31 to residue F234, to Mus musculus Ac39/physophilin (GenBank ID g1226235) as determined by the Basic Local Alignment Search Tool (BLAST). (See Table 2.) The BLAST probability score is 1.6e-124, which indicates the probability of obtaining the observed polypeptide sequence alignments by chance. SEQ ID NO:23 also has homology to proteins that are putative orthologs of human ATP6DV, which is subunit D of the vacuolar H(+)-ATPase proton pump, an accessory subunit that regulates ATP binding and hydrolysis by the A and B subunits, as determined by BLAST analysis using the PROTEOME database. SEQ ID NO:23 also contains an ATP synthase (C/AC39) subunit domain as determined by searching for statistically significant matches in the hidden Markov model (HMM)-based PFAM database of conserved protein family domains. (See Table 3.) Additional BLAST analyses against the PRODOM and DOMO databases provide further corroborative evidence that SEQ ID NO:23 is a synaptic transport protein

SEQ ID NO:2-3, SEQ ID NO:5-6, SEQ ID NO:11, SEQ ID NO:13-17, SEQ ID NO:19-22 and SEQ ID NO:24-25 were analyzed and annotated in a similar manner. The algorithms and parameters for the analysis of SEQ ID NO:1-25 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the fall length polynucleotide embodiments, and of fragments of the polynucleotides which are useful, for example, in hybridization or amplification technologies that identify SEQ ID NO:26-50 or that distinguish between SEQ ID NO:26-50 and related polynucleotides.

The polynucleotide fragments described in Column 2 of Table 4 may refer specifically, for example, to Incyte cDNAs derived from tissue specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N_{4—} represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm For example, a polynucleotide sequence identified as FLXXXXXX_gAAAAA_gBBBBB_{—}1_N is a “stretched” sequence, with XXXXXX being the Incyte project identification number, gAAAAA being the GemBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, | Exon prediction from genomic sequences using, for |
| ENST | example, GENSCAN (Stanford University, CA, USA) or |
| | FGENES (Computer Genomics Group, The Sanger Centre, |
| | Cambridge, UK). |
| GBI | Hand-edited analysis of genomic sequences. |
| FL | Stitched or stretched genomic sequences (see Example V). |
| INCY | Full length transcript and exon prediction from mapping of |
| | EST sequences to the genome. Genomic location and EST |
| | composition data are combined to predict the exons and |
| | resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide sequences of the invention, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP (SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses NTRAN variants. A preferred NTRAN variant is one which has at least about 80%, or alternatively at least about 90%, or even at least about 95% amino acid sequence identity to the NTRAN amino acid sequence, and which contains at least one functional or structural characteristic of NTRAN.

Various embodiments also encompass polynucleotides which encode NTRAN. In a particular embodiment, the invention encompasses a polynucleotide sequence comprising a sequence selected from the group consisting of SEQ ID NO:26-50, which encodes NTRAN. The polynucleotide sequences of SEQ ID NO:26-50, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding NTRAN. In particular, such a variant polynucleotide will have at least about 70%, or alternatively at least about 85%, or even at least about 95% polynucleotide sequence identity to a polynucleotide encoding NTRAN. A particular aspect of the invention encompasses a variant of a polynucleotide comprising a sequence selected from the group consisting of SEQ ID NO:26-50 which has at least about 70%, or alternatively at least about 85%, or even at least about 95% polynucleotide sequence identity to a nucleic acid sequence selected from the group consisting of SEQ ID NO:26-50. Any one of the polynucleotide variants described above can encode a polypeptide which contains at least one functional or structural characteristic of NTRAN.

In addition, or in the alternative, a polynucleotide variant of the invention is a splice variant of a polynucleotide encoding NTRAN. A splice variant may have portions which have significant sequence identity to a polynucleotide encoding NTRAN, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding NTRAN over its entire length; however, portions of the splice variant will have at least about 70%, or alternatively at least about 85%, or alternatively at least about 95%, or alternatively 100% polynucleotide sequence identity to portions of the polynucleotide encoding NTRAN. For example, a polynucleotide comprising a sequence of SEQ ID NO:43 and a polynucleotide comprising a sequence of SEQ ID NO:44 are splice variants of each other; a polynucleotide comprising a sequence of SEQ ID NO:29, a polynucleotide comprising a sequence of SEQ ID NO:31 and a polynucleotide comprising a sequence of SEQ ID NO:46 are splice variants of each other; a polynucleotide comprising a sequence of SEQ ID NO:32, a polynucleotide comprising a sequence of SEQ ID NO:49 and a polynucleotide comprising a sequence of SEQ ED NO:50 are splice variants of each other; and a polynucleotide comprising a sequence of SEQ ID NO:36, a polynucleotide comprising a sequence of SEQ ID NO:37 and a polynucleotide comprising a sequence of SEQ ID NO:45 are splice variants of each other. Any one of the splice variants described above can encode a polypeptide which contains at least one functional or structural characteristic of NTRAN.

It will be appreciated by those skilled in the art that as a result of the degeneracy of the genetic code, a multitude of polynucleotide sequences encoding NTRAN, some bearing minimal similarity to the polynucleotide sequences of any known and naturally occurring gene, maybe produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring NTRAN, and all such variations are to be considered as being specifically disclosed.

Although polynucleotides which encode NTRAN and its variants are generally capable of hybridizing to polynucleotides encoding naturally occurring NTRAN under appropriately selected conditions of stringency, it may be advantageous to produce polynucleotides encoding NTRAN or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding NTRAN and its derivatives without altering the encoded amino acid sequences include the production of RNA transcripts having more desirable properties, such as a greater half-life, than transcripts produced from the naturally occurring sequence.

The invention also encompasses production of polynucleotides which encode NTRAN and NTRAN derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding NTRAN or any fragment thereof.

Embodiments of the invention can also include polynucleotides that are capable of hybridizing to the claimed polynucleotides, and, in particular, to those having the sequences shown in SEQ ID NO:26-50 and fragments thereof, under various conditions of stringency (Wahl, G. M. and S. L. Berger (1987) Methods Enzymol. 152:399-407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding NTRAN maybe extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations maybe used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode NTRAN may be cloned in recombinant DNA molecules that direct expression of NTRAN, or fragments or functional equivalents thereof, in appropriate host cells. Due to the inherent degeneracy of the genetic code, other polynucleotides which encode substantially the same or a functionally equivalent polypeptides may be produced and used to express NTRAN.

The polynucleotides of the invention can be engineered using methods generally known in the art in order to alter NTRAN-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat Biotechnol. 17:259-264; and Crameri, A. et al. (1996) Nat Biotechnol. 14:315-319) to alter or improve the biological properties of NTRAN, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding NTRAN maybe synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, NTRAN itself or a fragment thereof may be synthesized using chemical methods known in the art. For example, peptide synthesis can be performed using various solution-phase or solid-phase techniques (Creighton, T. (1984) Proteins, Structures and Molecular Properties, W H Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of NTRAN, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active NTRAN, the polynucleotides encoding NTRAN or derivatives thereof maybe inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding NTRAN. Such elements may vary in their strength and specificity. Specific initiation signals may also be used to achieve more efficient translation of polynucleotides encoding NTRAN. Such signals include the ATG initiation codon and adjacent sequences, e.g. the Kozak sequence. In cases where a polynucleotide sequence encoding NTRAN and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding NTRAN and appropriate transcriptional and translational control elements. These methods include in vitro recombinant DNA techniques, synthetic techniques, and in vivo genetic recombination (Sambrook, J. et al. (1989) Molecular Cloning, A Laboratory Manual, Cold Spring Harbor Press, Plainview N.Y., ch. 4, 8, and 16-17; Ausubel et al., supra, ch. 1, 3, and 15).

A variety of expression vector/host systems may be utilized to contain and express polynucleotides encoding NTRAN. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Ge. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding NTRAN. For example, routine cloning, subcloning, and propagation of polynucleotides encoding NTRAN can be achieved using a multifunctional E. coli vector such as PBLUESCRIPT (Stratagene, La Jolla Calif.) or PSPORT1 plasmid (Invitrogen). Ligation of polynucleotides encoding NTRAN into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of NTRAN are needed, e.g. for the production of antibodies, vectors which direct high level expression of NTRAN may be used. For example, vectors containing the strong, inducible SP6 or T7 bacteriophage promoter maybe used.

Yeast expression systems may be used for production of NTRAN. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of NTRAN. Transcription of polynucleotides encoding NTRAN may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters maybe used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding NTRAN maybe ligated into an adenovirus transcription/translation complex consisting of the late promoter and tripartite leader sequence. Insertion in a non-essential E1 or E3 region of the viral genome may be used to obtain infective virus which expresses NTRAN inhost cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of NTRAN in cell lines is preferred. For example, polynucleotides encoding NTRAN can be transformed into cell lines using expression vectors which may contain viral origins of replication and/or endogenous expression elements and a selectable marker gene on the same or on a separate vector. Following the introduction of the vector, cells maybe allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk and apr cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding NTRAN is inserted within a marker gene sequence, transformed cells containing polynucleotides encoding NTRAN can be identified by the absence of marker gene function. Alternatively, a marker gene can be placed in tandem with a sequence encoding NTRAN under the control of a single promoter. Expression of the marker gene in response to induction or selection usually indicates expression of the tandem gene as well.

In general, host cells that contain the polynucleotide encoding NTRAN and that express NTRAN may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of NTRAN using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on NTRAN is preferred, but a competitive binding assay may be employed. These and other assays are well known in the art (Hampton, R. et al. (1990) Seroloyical Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding NTRAN include oligolabeling, nick translation, end-labeling, or PCR amplification using a labeled nucleotide. Alternatively, polynucleotides encoding NTRAN, or any fragments thereof, may be cloned into a vector for the production of an mRNA probe. Such vectors are known in the art, are commercially available, and maybe used to synthesize RNA probes in vitro by addition of an appropriate RNA polymerase such as T7, T3, or SP6 and labeled nucleotides. These procedures maybe conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemilmunescent, or chromogenic agents, as well as substrates, cofactors, inhibitors, magnetic particles, and the like.

Host cells transformed with polynucleotides encoding NTRAN maybe cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode NTRAN may be designed to contain signal sequences which direct secretion of NTRAN through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding NTRAN may be ligated to a heterologous sequence resulting in translation of a fusion protein in any of the aforementioned host systems. For example, a chimeric NTRAN protein containing a heterologous moiety that can be recognized by a commercially available antibody may facilitate the screening of peptide libraries for inhibitors of NTRAN activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the NTRAN encoding sequence and the heterologous protein sequence, so that NTRAN may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled NTRAN maybe achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

NTRAN, fragments of NTRAN, or variants of NTRAN maybe used to screen for compounds that specifically bind to NTRAN. One or more test compounds maybe screened for specific binding to NTRAN. In various embodiments, 1, 2, 3, 4, 5, 10, 20, 50, 100, or 200 test compounds can be screened for specific binding to NTRAN. Examples of test compounds can include antibodies, anticalins, oligonucleotides, proteins (e.g., ligands or receptors), or small molecules.

In related embodiments, variants of NTRAN can be used to screen for binding of test compounds, such as antibodies, to NTRAN, a variant of NTRAN, or a combination of NTRAN and/or one or more variants NTRAN. In an embodiment, a variant of NTRAN can be used to screen for compounds that bind to a variant of NTRAN, but not to NTRAN having the exact sequence of a sequence of SEQ ID NO:1-25. NTRAN variants used to perform such screening can have a range of about 50% to about 99% sequence identity to NTRAN, with various embodiments having 60%, 70%, 75%, 80%, 85%, 90%, and 95% sequence identity.

In an embodiment, a compound identified in a screen for specific binding to NTRAN can be closely related to the natural ligand of NTRAN, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor NTRAN (Howard, A. D. et al. (2001) Trends Pharmacol. Sci.22:132-140; Wise, A. et al. (2002) Drug Discovery Today 7:235-246).

In other embodiments, a compound identified in a screen for specific binding to NTRAN can be closely related to the natural receptor to which NTRAN binds, at least a fragment of the receptor, or a fragment of the receptor including all or a portion of the ligand binding site or binding pocket. For example, the compound may be a receptor for NTRAN which is capable of propagating a signal, or a decoy receptor for NTRAN which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to NTRAN, fragments of NTRAN, or variants of NTRAN. The binding specificity of the antibodies thus screened can thereby be selected to identify particular fragments or variants of NTRAN. In one embodiment, an antibody can be selected such that its binding specificity allows for preferential identification of specific fragments or variants of NTRAN. In another embodiment, an antibody can be selected such that its binding specificity allows for preferential diagnosis of a specific disease or condition having increased, decreased, or otherwise abnormal production of NTRAN.

In an embodiment, anticalins can be screened for specific binding to NTRAN, fragments of NTRAN, or variants of NTRAN. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit NTRAN involves producing appropriate cells which express NTRAN, either as a secreted protein or on the cell membrane. Preferred cells include cells from mammals, yeast, Diosophila, or E. coli. Cells expressing NTRAN or cell membrane fractions which contain NTRAN are then contacted with a test compound and binding, stimulation, or inhibition of activity of either NTRAN or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with NTRAN, either in solution or affixed to a solid support, and detecting the binding of NTRAN to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

NTRAN, fragments of NTRAN, or variants of NTRAN may be used to screen for compounds that modulate the activity of NTRAN. Such compounds may include agonists, antagonists, or partial or inverse agonists. In one embodiment, an assay is performed under conditions permissive for NTRAN activity, wherein NTRAN is combined with at least one test compound, and the activity of NTRAN in the presence of a test compound is compared with the activity of NTRAN in the absence of the test compound. A change in the activity of NTRAN in the presence of the test compound is indicative of a compound that modulates the activity of NTRAN. Alternatively, a test compound is combined with an in vitro or cell-free system comprising NTRAN under conditions suitable for NTRAN activity, and the assay is performed. In either of these assays, a test compound which modulates the activity of NTRAN may do so indirectly and need not come in direct contact with the test compound. At least one and up to a plurality of test compounds may be screened.

In another embodiment, polynucleotides encoding NTRAN or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding NTRAN may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding NTRAN can also be used to create “knockin” humanized animals (pigs) or transgenic animals (mice or rats) to model human disease. With knockin technology, a region of a polynucleotide encoding NTRAN is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress NTRAN, e.g., by secreting NTRAN in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of NTRAN and neurotransmission-associated proteins. In addition, the expression of NAP is closely associated with adrenal, brain, brain tumor, cervical, dorsal root ganglion tissue, fetal brain, spinal cord, testicular, tumor-associated stomach, and uterine tissue, and with bronchial epithelium cells, fetal prostate fibroblasts, as well as with polymicrogyria, gliosis, and cervical and testicular cancer. In addition, examples of tissues expressing NTRAN can be found in Table 6 and can also be found in Example XI. Therefore, NTRAN appears to play a role in autoimmune/inflammatory, cardiovascular, neurological, developmental, cell proliferative, transport, psychiatric, metabolic, and endocrine disorders. In the treatment of disorders associated with increased NTRAN expression or activity, it is desirable to decrease the expression or activity of NTRAN. In the treatment of disorders associated with decreased NTRAN expression or activity, it is desirable to increase the expression or activity of NTRAN.

Therefore, in one embodiment, NTRAN or a fragment or derivative thereof may be administered to a subject to treat or prevent a disorder associated with decreased expression or activity of NTRAN. Examples of such disorders include, but are not limited to, an autoimmune/inflammatory disorder such as acquired immunodeficiency syndrome (AIDS), Addison's disease, adult respiratory distress syndrome, allergies, annylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erydiroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, syndrome, systemic anaphylaxis, systemic lupus erythematosus, systemic sclerosis, thromb osteoarthritis, osteoporosis, pancreatitis, polymyositis, psoriasis, Reiter's syndrome, rheumatoid arthritis, scleroderma, Sjögren's ocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cardiovascular disorder such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery; a neurological disorder such as epilepsy, ischemic cerebrovascular disease, stroke, cerebral neoplasms, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kiru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a cell proliferative disorder such as actinic keratosis, arteriosclerosis, atherosclerosis, bursitis, cirrhosis, hepatitis, mixed connective tissue disease (MCID), myeloffbrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus and a cancer such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a transport disorder such as akinesia, amyotrophic lateral sclerosis, ataxia telangiectasia, cystic fibrosis, Becker's muscular dystrophy, Bell's palsy, Charcot-Marie Tooth disease, diabetes mellitus, diabetes insipidus, diabetic neuropathy, Duchenne muscular dystrophy, hyperkalemic periodic paralysis, normokalemic periodic paralysis, Parkinson's disease, malignant hyperthermia, multidrug resistance, myasthenia gravis, myotonic dystrophy, catatonia, tardive dyskinesia, dystonias, peripheral neuropathy, cerebral neoplasms, prostate cancer, cardiac disorders associated with transport, e.g., angina, bradyarrythmia, tachyarrythmia, hypertension, Long QT syndrome, myocarditis, cardiomyopathy, nemaline myopathy, centronuclear myopathy, lipid myopathy, mitochondrial myopathy, thyrotoxic myopathy, ethanol myopathy, dermatomyositis, inclusion body myositis, infectious myositis, polymyositis, neurological disorders associated with transport, e.g., Alzheimer's disease, amnesia, bipolar disorder, dementia, depression, epilepsy, Tourette's disorder, paranoid psychoses, and schizophrenia, and other disorders associated with transport, e.g., neurofibromatosis, postherpetic neuralgia, trigeminal neuropathy, sarcoidosis, sickle cell anemia, Wilson's disease, cataracts, infertility, pulmonary artery stenosis, sensorineural autosomal deafness, hyperglycemia, hypoglycemia, Grave's disease, goiter, Cushing's disease, Addison's disease, glucose-galactose malabsorption syndrome, hypercholesterolemia, adrenoleukodystrophy, Zellweger syndrome, Menkes disease, occipital horn syndrome, von Gierke disease, cystinuria, iminoglycinuria, Hartup disease, and Fanconi disease; a psychiatric disorder such as acute stress disorder, alcohol dependence, amphetamine dependence, anorexia nervosa, antisocial personality disorder, attention-deficit hyperactivity disorder, autistic disorder, anxiety, avoidant personality disorder, bipolar disorder, borderline personality disorder, brief psychotic disorder, bulimia nervosa, cannabis dependence, cocaine dependence, conduct disorder, cyclothymic disorder, delirium, delusional disorder, dementia, dependent personality disorder, depression, dysthymic disorder, hallucinogen dependence, histrionic personality disorder, inhalant dependence, manic depression, multi-infarct dementia, narcissistic personality disorder, nicotine dependence, obsessive-compulsive disorder, opioid dependence, oppositional defiant disorder, panic disorder, paranoid personality disorder, phencyclidine dependence, phobia, posttraumatic stress disorder, schizoaffective disorder, schizoid personality disorder, schizophrenia, sedative dependence, separation anxiety disorder, and sleep disorder; a metabolic disorder such as Addison's disease, cerebrotendinous xanthomatosis, congenital adrenal hyperplasia, coumarin resistance, cystic fibrosis, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, mannosidosis, neuraminidase deficiency, obesity, osteoporosis, phenylketonuria, pseudovitamin D-deficiency rickets, disorders of carbohydrate metabolism such as congenital type II dyserytbropoietic anemia, diabetes, insulin-dependent diabetes mellitus, non-insulin-dependent diabetes mellitus, galactose epimerase deficiency, glycogen storage diseases, lysosomal storage diseases, fructosuria, pentosuria, and inherited abnormalities of pyruvate metabolism, disorders of lipid metabolism such as fatty liver, cholestasis, primary biliary cirrhosis, carnitine deficiency, carnitine palmitoyltransferase deficiency, myoadenylate deaminase deficiency, hypertiglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, and lipid myopathies, and disorders of copper metabolism such as Menke's disease, Wilson's disease, and Ehlers-Danlos syndrome type IX diabetes; and an endocrine disorder such as a disorder of the hypothalamus and/or pituitary resulting from lesions such as a primary brain tumor, adenoma, infarction associated with pregnancy, hypophysectomy, aneurysm, vascular malformation, thrombosis, infection, immunological disorder, and complication due to head trauma, a disorder associated with hypopituitarism including hypogonadism, Sheehan syndrome, diabetes insipidus, Kallman's disease, Hand-Schuller-Christian disease, Letterer-Siwe disease, sarcoidosis, empty sella syndrome, and dwarfism, a disorder associated with hyperpituitarism including acromegaly, giantism, and syndrome of inappropriate antidiuretic hormone (ADH) secretion (SIADH) often caused by benign adenoma, a disorder associated with hypothyroidism including goiter, myxedema, acute thyroiditis associated with bacterial infection, subacute thyroiditis associated with viral infection, autoimmune thyroiditis (Hashimoto's disease), and cretinism, a disorder associated with hyperthyroidism including thyrotoxicosis and its various forms, Grave's disease, pretibial myxedema, toxic multinodular goiter, thyroid carcinoma, and Plummer's disease, a disorder associated with hyperparathyroidism including Conn disease (chronic hypercalemia), a pancreatic disorder such as Type I or Type II diabetes mellitus and associated complications, a disorder associated with the adrenals such as hyperplasia, carcinoma, or adenoma of the adrenal cortex, hypertension associated with alkalosis, amyloidosis, hypokalemia, Cushing's disease, Liddle's syndrome, and Arnold-Healy-Gordon syndrome, pheochromocytoma tumors, and Addison's disease, a disorder associated with gonadal steroid hormones such as: in women, abnormal prolactin production, infertility, endometriosis, perturbation of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis, and, in men, Leydig cell deficiency, male climacteric phase, and germinal cell aplasia, a hypergonadal disorder associated with Leydig cell tumors, androgen resistance associated with absence of androgen receptors, syndrome of 5 α-reductase, and gynecomastia.

In another embodiment, a vector capable of expressing NTRAN or a fragment or derivative thereof maybe administered to a subject to treat or prevent a disorder associated with decreased expression or activity of NTRAN including, but not limited to, those described above.

In a further embodiment, a composition comprising a substantially purified NTRAN in conjunction with a suitable pharmaceutical carrier may be administered to a subject to treat or prevent a disorder associated with decreased expression or activity of NTRAN including, but not limited to, those provided above.

In still another embodiment, an agonist which modulates the activity of NTRAN may be administered to a subject to treat or prevent a disorder associated with decreased expression or activity of NTRAN including, but not limited to, those listed above.

In a further embodiment, an antagonist of NTRAN may be administered to a subject to treat or prevent a disorder associated with increased expression or activity of NTRAN. Examples of such disorders include, but are not limited to, those autoimmune/inflammatory, cardiovascular, neurological, developmental, cell proliferative, transport, psychiatric, metabolic, and endocrine disorders described above. In one aspect, an antibody which specifically binds NTRAN may be used directly as an antagonist or indirectly as a targeting or delivery mechanism for bringing a pharmaceutical agent to cells or tissues which express NTRAN.

In an additional embodiment, a vector expressing the complement of the polynucleotide encoding NTRAN maybe administered to a subject to treat or prevent a disorder associated with increased expression or activity of NTRAN including, but not limited to, those described above.

In other embodiments, any protein, agonist, antagonist, antibody, complementary sequence, or vector embodiments maybe administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of NTRAN may be produced using methods which are generally known in the art. In particular, purified NTRAN may be used to produce antibodies or to screen libraries of pharmaceutical agents to identify those which specifically bind NTRAN. Antibodies to NTRAN may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit diner formation) are generally preferred for therapeutic use. Single chain antibodies (e.g., from camels or llamas) maybe potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with NTRAN or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to NTRAN have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of NTRAN amino acids may be fused with those of another protein, such as KLH, and antibodies to the chimeric molecule may be produced.

Monoclonal antibodies to NTRAN may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce NTRAN-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for NTRAN may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between NTRAN and its specific antibody. A two-site, monoclonal-based immunoassay utilizing monoclonal antibodies reactive to two non-interfering NTRAN epitopes is generally used, but a competitive binding assay may also be employed (Pound, supra).

Various methods such as Scatchard analysis in conjunction with radioimmunoassay techniques may be used to assess the affinity of antibodies for NTRAN. Affinity is expressed as an association constant, Kₐ, which is defined as the molar concentration of NTRAN-antibody complex divided by the molar concentrations of free antigen and free antibody under equilibrium conditions. The Kₐ determined for a preparation of polyclonal antibodies, which are heterogeneous in their affinities for multiple NTRAN epitopes, represents the average affinity, or avidity, of the antibodies for NTRAN. The Kₐ determined for a preparation of monoclonal antibodies, which are monospecific for a particular NTRAN epitope, represents a true measure of affinity. High-affinity antibody preparations with Kₐ ranging from about 10⁹ to 10¹² L/mole are preferred for use in immunoassays in which the NTRAN-antibody complex must withstand rigorous manipulations. Low-affinity antibody preparations with Kₐ ranging from about 10⁶ to 10⁷ L/mole are preferred for use in immunopurification and similar procedures which ultimately require dissociation of NTRAN, preferably in active form, from the antibody (Catty, D. (1988) Antibodies. Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of NTRAN-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding NTRAN, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding NTRAN. Such technology is well known in the art, and antisense oligonucleotides or larger fragments can be designed from various locations along the coding or control regions of sequences encoding NTRAN (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding NTRAN may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in NTRAN expression or regulation causes disease, the expression of NTRAN from an appropriate population of transduced cells may alleviate the clinical manifestations caused by the genetic deficiency.

In a further embodiment of the invention, diseases or disorders caused by deficiencies in NTRAN are treated by constructing mammalian expression vectors encoding NTRAN and introducing these vectors by mechanical means into NTRAN-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Récipon (1998) Curr. Opin. Biotechnol. 9:445-450).

Expression vectors that may be effective for the expression of NTRAN include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). NTRAN may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding NTRAN from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to NTRAN expression are treated by constructing a retrovirus vector consisting of (i) the polynucleotide encoding NTRAN under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding NTRAN to cells which have one or more genetic abnormalities with respect to the expression of NTRAN. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding NTRAN to target cells which have one or more genetic abnormalities with respect to the expression of NTRAN. The use of herpes simplex virus (HSV)-based vectors may be especially valuable for introducing NTRAN to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No.5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding NTRAN to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavinis RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for NTRAN into the alphavirus genome in place of the capsid-coding region results in the production of a large number of NTRAN-coding RNAs and the synthesis of high levels of NTRAN in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of NTRAN into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding NTRAN.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding NTRAN. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding NTRAN. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased NTRAN expression or activity, a compound which specifically inhibits expression of the polynucleotide encoding NTRAN may be therapeutically useful, and in the treatment of disorders associated with decreased NTRAN expression or activity, a compound which specifically promotes expression of the polynucleotide encoding NTRAN may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding NTRAN is exposed to at least one test compound thus obtained. The sample may comprise, for example, an intact or permeabilized cell, or an in vitro cell-free or reconstituted biochemical system. Alterations in the expression of a polynucleotide encoding NTRAN are assayed by any method commonly known in the art. Typically, the expression of a specific nucleotide is detected by hybridization with a probe having a nucleotide sequence complementary to the sequence of the polynucleotide encoding NTRAN. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of NTRAN, antibodies to NTRAN, and mimetics, agonists, antagonists, or inhibitors of NTRAN.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising NTRAN or fragments thereof. For example, liposome preparations containing a cell-impermeable macromolecule may promote cell fusion and intracellular delivery of the macromolecule. Alternatively, NTRAN or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example NTRAN or fragments thereof, antibodies of NTRAN, and agonists, antagonists or inhibitors of NTRAN, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind NTRAN may be used for the diagnosis of disorders characterized by expression of NTRAN, or in assays to monitor patients being treated with NTRAN or agonists, antagonists, or inhibitors of NTRAN. Antibodies useful for diagnostic purposes may be prepared in the same manner as described above for therapeutics. Diagnostic assays for NTRAN include methods which utilize the antibody and a label to detect NTRAN in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring NTRAN, including ELISAs, RIAs, and FACS, are known in the art and provide a basis for diagnosing altered or abnormal levels of NTRAN expression. Normal or standard values for NTRAN expression are established by combining body fluids or cell extracts taken from normal mammalian subjects, for example, human subjects, with antibodies to NTRAN under conditions suitable for complex formation. The amount of standard complex formation may be quantitated by various methods, such as photometric means. Quantities of NTRAN expressed in subject, control, and disease samples from biopsied tissues are compared with the standard values. Deviation between standard and subject values establishes the parameters for diagnosing disease.

In another embodiment of the invention, polynucleotides encoding NTRAN may be used for diagnostic purposes. The polynucleotides which may be used include oligonucleotides, complementary RNA and DNA molecules, and PNAs. The polynucleotides may be used to detect and quantify gene expression in biopsied tissues in which expression of NTRAN may be correlated with disease. The diagnostic assay may be used to determine absence, presence, and excess expression of NTRAN, and to monitor regulation of NTRAN levels during therapeutic intervention.

In one aspect, hybridization with PCR probes which are capable of detecting polynucleotides, including genomic sequences, encoding NTRAN or closely related molecules may be used to identify nucleic acid sequences which encode NTRAN. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding NTRAN, allelic variants, or related sequences.

Probes may also be used for the detection of related sequences, and may have at least 50% sequence identity to any of the NTRAN encoding sequences. The hybridization probes of the subject invention may be DNA or RNA and may be derived from the sequence of SEQ ID NO:26-50 or from genomic sequences including promoters, enhancers, and introns of the NTRAN gene.

Means for producing specific hybridization probes for polynucleotides encoding NTRAN include the cloning of polynucleotides encoding NTRAN or NTRAN derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²p or ³⁵S, or by enzymatic labels, such as alkaline phosphatase coupled to the probe via avidin/biotin coupling systems, and the like.

Polynucleotides encoding NTRAN may be used for the diagnosis of disorders associated with expression of NTRAN. Examples of such disorders include, but are not limited to, an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, syndrome, systemic anaphylaxis, systemic lupus erythematosus, systemic sclerosis, thromb osteoarthritis, osteoporosis, pancreatitis, polymyositis, psoriasis, Reiter's syndrome, rheumatoid arthritis, scleroderma, Sjögren's ocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cardiovascular disorder such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery; a neurological disorder such as epilepsy, ischemic cerebrovascular disease, stroke, cerebral neoplasms, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyclinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus and a cancer such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a transport disorder such as akinesia, amyotrophic lateral sclerosis, ataxia telangiectasia, cystic fibrosis, Becker's muscular dystrophy, Bell's palsy, Charcot-Marie Tooth disease, diabetes mellitus, diabetes insipidus, diabetic neuropathy, Duchenne muscular dystrophy, hyperkalemic periodic paralysis, normokalemic periodic paralysis, Parkinson's disease, malignant hyperthermia, multidrug resistance, myasthenia gravis, myotonic dystrophy, catatonia, tardive dyskinesia, dystonias, peripheral neuropathy, cerebral neoplasms, prostate cancer, cardiac disorders associated with transport, e.g., angina, bradyarrythmia, tachyarrytmia, hypertension, Long QT syndrome, myocarditis, cardiomyopathy, nemaline myopathy, centronuclear myopathy, lipid myopathy, mitochondrial myopathy, thyrotoxic myopathy, ethanol myopathy, dermatomyositis, inclusion body myositis, infectious myositis, polymyositis, neurological disorders associated with transport, e.g., Alzheimer's disease, amnesia, bipolar disorder, dementia, depression, epilepsy, Tourette's disorder, paranoid psychoses, and schizophrenia, and other disorders associated with transport, e.g., neurofibromatosis, postherpetic neuralgia, trigeminal neuropathy, sarcoidosis, sickle cell anemia, Wilson's disease, cataracts, infertility, pulmonary artery stenosis, sensorineural autosomal deafness, hyperglycemia, hypoglycemia, Grave's disease, goiter, Cushing's disease, Addison's disease, glucose-galactose malabsorption syndrome, hypercholesterolemia, adrenoleukodystrophy, Zellweger syndrome, Menkes disease, occipital horn syndrome, von Gierke disease, cystinuria, iminoglycinuria, Hartup disease, and Fanconi disease; a psychiatric disorder such as acute stress disorder, alcohol dependence, amphetamine dependence, anorexia nervosa, antisocial personality disorder, attention-deficit hyperactivity disorder, autistic disorder, anxiety, avoidant personality disorder, bipolar disorder, borderline personality disorder, brief psychotic disorder, bulimia nervosa, cannabis dependence, cocaine dependence, conduct disorder, cyclothymic disorder, delirium, delusional disorder, dementia, dependent personality disorder, depression, dysthymic disorder, hallucinogen dependence, histrionic personality disorder, inhalant dependence, manic depression, multi-infarct dementia, narcissistic personality disorder, nicotine dependence, obsessive-compulsive disorder, opioid dependence, oppositional defiant disorder, panic disorder, paranoid personality disorder, phencyclidine dependence, phobia, posttraumatic stress disorder, schizoaffective disorder, schizoid personality disorder, schizophrenia, sedative dependence, separation anxiety disorder, and sleep disorder; a metabolic disorder such as Addison's disease, cerebrotendinous xanthomatosis, congenital adrenal hyperplasia, coumarin resistance, cystic fibrosis, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, mannosidosis, neuraminidase deficiency, obesity, osteoporosis, phenylketonuria, pseudovitamin D-deficiency rickets, disorders of carbohydrate metabolism such as congenital type II dyserythropoietic anemia, diabetes, insulin-dependent diabetes mellitus, non-insulin-dependent diabetes mellitus, galactose epimerase deficiency, glycogen storage diseases, lysosomal storage diseases, fructosuria, pentosuria, and inherited abnormalities of pyruvate metabolism, disorders of lipid metabolism such as fatty liver, cholestasis, primary biliary cirrhosis, caroitine deficiency, carnitine palmitoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, and lipid myopathies, and disorders of copper metabolism such as Menke's disease, Wilson's disease, and Ehlers-Danlos syndrome type IX diabetes; and an endocrine disorder such as a disorder of the hypothalamus and/or pituitary resulting from lesions such as a primary brain tumor, adenoma, infarction associated with pregnancy, hypophysectomy, aneurysm, vascular malformation, thrombosis, infection, immunological disorder, and complication due to head trauma, a disorder associated with hypopituitarism including hypogonadism, Sheehan syndrome, diabetes insipidus, Kallman's disease, Hand-Schuller-Christian disease, Letterer-Siwe disease, sarcoidosis, empty sella syndrome, and dwarfism, a disorder associated with hyperpituitarism including acromegaly, giantism, and syndrome of inappropriate antidiuretic hormone (ADH) secretion (SIADH) often caused by benign adenoma, a disorder associated with hypothyroidism including goiter, myxedema, acute thyroiditis associated with bacterial infection, subacute thyroiditis associated with viral infection, autoimmune thyroiditis (Hashimoto's disease), and cretinism, a disorder associated with hyperthyroidism including thyrotoxicosis and its various forms, Grave's disease, pretibial myxedema, toxic multinodular goiter, thyroid carcinoma, and Plummer's disease, a disorder associated with hyperparathyroidism including Conn disease (chronic hypercalemia), a pancreatic disorder such as Type I or Type II diabetes mellitus and associated complications, a disorder associated with the adrenals such as hyperplasia, carcinoma, or adenoma of the adrenal cortex, hypertension associated with alkalosis, amyloidosis, hypokalemia, Cushing's disease, Liddle's syndrome, and Arnold-Healy-Gordon syndrome, pheochromocytoma tumors, and Addison's disease, a disorder associated with gonadal steroid hormones such as: in women, abnormal prolactin production, infertility, endometriosis, perturbation of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis, and, in men, Leydig cell deficiency, male climacteric phase, and germinal cell aplasia, a hypergonadal disorder associated with Leydig cell tumors, androgen resistance associated with absence of androgen receptors, syndrome of 5 α-reductase, and gynecomastia. Polynucleotides encoding NTRAN may be used in Southern or northern analysis, dot blot, or other membrane-based technologies; in PCR technologies; in dipstick, pin, and multiformat ELISA-like assays; and in microarrays utilizing fluids or tissues from patients to detect altered NTRAN expression. Such qualitative or quantitative methods are well known in the art.

In a particular aspect, polynucleotides encoding NTRAN may be used in assays that detect the presence of associated disorders, particularly those mentioned above. Polynucleotides complementary to sequences encoding NTRAN may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding NTRAN in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of NTRAN, a normal or standard profile for expression is established. This may be accomplished by combining body fluids or cell extracts taken from normal subjects, either animal or human, with a sequence, or a fragment thereof, encoding NTRAN, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding NTRAN may involve the use of PCR. These oligomers may be chemically synthesized, generated enzymatically, or produced in vitro. Oligomers will preferably contain a fragment of a polynucleotide encoding NTRAN, or a fragment of a polynucleotide complementary to the polynucleotide encoding NTRAN, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding NTRAN maybe used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (FSSCP) methods. In SSCP, oligonucleotide primers derived from polynucleotides encoding NTRAN are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of NTRAN include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, NTRAN, fragments of NTRAN, or antibodies specific for NTRAN may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for NTRAN to quantify the levels of NTRAN expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection maybe performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound: Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding NTRAN may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding NTRAN on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely to localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, NTRAN, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between NTRAN and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with NTRAN, or fragments thereof, and washed. Bound NTRAN is then detected by methods well known in the art. Purified NTRAN can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding NTRAN specifically compete with a test compound for binding NTRAN. In this manner, antibodies can be used to detect the presence of any peptide which shares one or more antigenic determinants with NTRAN.

In additional embodiments, the nucleotide sequences which encode NTRAN may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications, and publications mentioned above and below, including U.S. Ser. No. 60/322,180, U.S. Ser. No. 60/326,096, U.S. Ser. No. 60/327,446, U.S. Ser. No. 60/345,837, U.S. Ser. No. 60/343,903, U.S. Ser. No. 60/334,020, U.S. Ser. No. 60/340,226, U.S. Ser. No.60/345,008, U.S. Ser. No. 60/365,645, and U.S. Ser. No. 60/379,887, are hereby expressly incorporated by reference.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIP plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amershain Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch. 7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cereviviae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (M)-based protein family databases such as PFAM, INCY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALUGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:26-50. Fragments from about 20 to about 4000 nucleotides which are useful in hybridization and amplification technologies are described in Table 4, column 2.

IV. Identification and Editing of Coding Sequences from Genomic DNA

Putative neurotransmission-associated proteins were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode neurotransmission-associated proteins, the encoded polypeptides were analyzed by querying against PFAM models for neurotransmission-associated proteins. Potential neurotransmission-associated proteins were also identified by homology to Incyte cDNA sequences that had been annotated as neurotransmission-associated proteins. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example m. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example m were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of NTRAN Encoding Polynucleotides

The sequences which were used to assemble SEQ ID NO:26-50 were compared with sequences from the Incyte LIFESEQ database and public domain databases using BLAST and other implementations of the Smith-Waterman algorithm. Sequences from these databases that matched SEQ ID NO:26-50 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap '99” World Wide Web site (bttp://www.ncbi.nln.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook, supra, ch. 7; Ausubel et al., supra, ch.4).

Analogous computer techniques applying BLAST were used to search for identical or related molecules in databases such as GenBank or LUESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding NTRAN are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding NTRAN. cDNA sequences and cDNA library/tissue information are found in the LEFESEQ GOLD database (Incyte Genomics, Palo Alto Calif.).

VIII. Extension of NTRAN Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C, 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in NTRAN Encoding Polynucleotides

Common DNA sequence variants known as single nucleotide polymorphisms (SNPs) were identified in SEQ ID NO:26-50 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:26-50 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1 ×saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte Genomics). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an EBM-compatible PC computer. The digitized data are displayed as an image where the signal intensity is mapped using a linear 20-color transformation to a pseudocolor scale ranging from blue (low signal) to red thigh signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte Genomics). Array elements that exhibited at least about a two-fold change in expression, a signal-to-background ratio of at least 2.5, and an element spot size of at least 40% were identified as differentially expressed.

Expression

Histological and molecular evaluation of breast tumors has revealed that the development of breast cancer evolves through a multi-step process whereby pre-malignant mammary epithelial cells undergo a relatively defined sequence of events leading to tumor formation. An early even in tumor development is ductal hyperplasia. Cells undergoing rapid neoplastic growth gradually progress to invasive carcinoma and become metastatic to the lung, bone, and potentially other organs. Several factors participate in the process of tumor progression and malignant transformation, including genetic factors, environmental factors, growth factors, and hormones. Based on the complexity of this process, it is critical to study a population of human mammary epithelial cells undergoing the process of malignant transformation and to associate specific stages of progression with phenotypic and molecular changes.

SEQ ID NO:26 was differentially expressed in various breast tumor cell lines as compared to a pure human mammary epithelial cell line (HMEC). BT-474 is a breast ductal carcinoma cell line that was isolated from a solid, invasive ductal carcinoma of the breast obtained from a 60-year old female. BT-474 displays typical epithelial cellular structures such as desmosomes, microvilli, gap junctions, and tight junctions. BT-483 is a breast ductal carcinoma cell line that was isolated from a papillary invasive ductal tumor obtained from a 23-year old normal, menstruating, parous female with a family history of breast cancer. BT-483 displays characteristic epithelial cellular structures such as desmosomes, microvilli, tight junctions, and gap junctions. MCF7 is a nonmalignant breast adenocarcinoma cell line isolated from the pleural effusion of a 69-year old female. MCF7 has retained characteristics of the mammary epithelium such as the ability to process estradiol via cytoplasmic estrogen receptors and the capacity to form domes in culture. MCF-10A is a breast mammary gland (luminal ductal characteristics) cell line that was isolated from a 36-year old female with fibrocystic breast disease. MCF-10A expresses cytoplasmic keratins, epithelial sialomucins, and milkfat globule antigens. This cell line exhibits three-dimensional growth in collagen and forms domes in confluent culture. Hs 578T is a breast ductal carcinoma cell line that was isolated from a 74-year old female with breast carcinoma. These cells do not express any detectable estrogen receptors and do not form colonies in semi-solid culture medium. MDA-MB-468 is a breast adenocarcinoma cell line isolated from the pleural effusion of a 51-year old female with metastatic adeonocarcinoma of the breast. SK-BR-3 is a breast adenocarcinoma cell line isolated from a malignant pleural effusion of a 43-year old female. It forms poorly differentiated adeonocarcinoma when injected into nude mice. The expression of SEQ ID NO:26 was decreased by at least two-fold in all of these breast tumor cell lines as compared to HMEC cells.

In another experiment, the human breast tumor cells lines BT-474, BT-483, MCF7, and MCF-10A were grown in basal media in the absence of growth factors and hormones for 24 hours prior to comparison to HMEC cells. The expression of SEQ ID NO:26 was decreased by at least two-fold in all of these breast tumor cells lines as compared to the HMEC cells. Therefore, in various embodiments, SEQ ID NO:26 can be used for one or more of the following: i) monitoring treatment of breast cancer, ii) diagnostic assays for breast cancer, and iii) developing therapeutics and/or other treatments for breast cancer.

Prostate cancer develops through a multistage progression ultimately resulting in an aggressive tumor phenotype. The initial step in tumor progression involves the hyperproliferation of normal luminal and/or basal epithelial cells. Androgen responsive cells become hyperplastic and evolve into early-stage tumors. Although early-stage tumors are often androgen sensitive and respond to androgen ablation, a population of androgen independent cells evolve from the hyperplastic population. These cells represent a more advanced form of prostate tumor that may become invasive and potentially become metastatic to the bone, brain, or lung. In these experiments, prostate tumor cell lines were compared to a primary prostate epithelial cell line that was isolated from a normal donor (PrEC). LNCaP is a prostate carcinoma cell line isolated from a lymph node biopsy of a 50-year old male with metastatic prostate carcinoma. LNCaP cells express prostate specific antigens, produce prostatic acid phosphatase, and express androgen receptors. PC-3 is a prostate adenocarcinoma cell line that was isolated from a metastatic site in the bone of a 62-year old male with grade IV prostate adenocarcinoma. The expression of SEQ ID NO:26 was decreased by at least two-fold in the LNCaP and PC-3 cells under restrictive conditions (starved, e.g. in basal media in the absence of growth factors and hormones); and in LNCaP cells under optimal growth conditions (e.g. in the presence of growth factors and hormones). Therefore, in various embodiments, SEQ ID NO:26 can be used for one or more of the following: i) monitoring treatment of prostate cancer, ii) diagnostic assays for prostate cancer, and iii) developing therapeutics and/or other treatments for prostate cancer.

As another example, SEQ ID NO:32 is upregulated in HT29 colorectal carcinoma cells treated with 5-aza-2-deoxycytidine in comparison to untreated HT9 cells, as determined by microarray analysis. HT29 cells are derived from a Grade II adenocarcinoma of the colon obtained from a 44 year old Caucasian female. HT29 adenocarcinoma cells (American Type Culture Collection, Manassas Va.) are cultured in McCoy's medium supplemented with 10% fetal bovine serum (Life Technologies) at 37° C. and 5% CO₂. Treated cells are exposed to 500 nM 5-aza-2-deoxycytidine (Sigma-Aldrich) 24 hr after passage in complete culture medium. Control cultures are treated in parallel with phosphate buffered saline vehicle. After twenty-four hours, culture medium is replaced with drug-free medium. Control and 5-aza-2-deoxycytidine-treated cells are subcultured at equal densities at 1 and 5 days after the initial treatment, and proliferation was measured at the subsequent time point using a Coulter counter (Beckman Coulter, Inc., Fullerton Calif.). Therefore, in various embodiments, SEQ ID NO:32 can be used for one or more of the following: i) monitoring treatment of colorectal cancer, ii) diagnostic assays for colorectal cancer, and iii) developing therapeutics and/or other treatments for colorectal cancer.

As another example, SEQ ID NO:32 is upregulated in peripheral blood mononuclear cells (PBMC)s treated with lipopolysaccharide (LPS) compared to untreated PBMC cells, as determined by microarray analysis. PBMCs from 2 healthy volunteer donors were treated with LPS for 1, 2, 4, 24, and 72 hours. LPS-treated PBMCs were compared to untreated PBMCs from the same donors. Therefore, in various embodiments, SEQ ID NO:32 can be used for one or more of the following: i) monitoring treatment of immune disorders and related diseases and conditions, ii) diagnostic assays for immune disorders and related diseases and conditions, and iii) developing therapeutics and/or other treatments for immune disorders and related diseases and conditions.

As another example, SEQ ID NO:35 was differentially expressed in all of the human breast tumor cell lines evaluated in this experiment as compared to normal mammary epithelial cells (HMEC).

The following human breast tumor cell lines were compared to the HMEC line: BT-20 (breast carcinoma), BT-474 (breast ductal carcinoma), BT-483 (breast ductal carcinoma), Hs 578T (breast ductal carcinoma), MCF7 (nonmalignant breast adenocarcinoma), MCF-10A (breast mammary gland (luminal ductal characteristics) cell line), and MDA-MB-468 (breast adenocarcinoma). In the gene expression profile of control HMEC cells compared to that of various breast carcinoma lines at different stages of tumor progression, the breast tumor cells lines exhibited underexpression by at least two-fold. In a similar, separate experiment, HMEC cells were again compared to various human breast tumor cell lines. In five out of six (MCF7, T47D, Sk-BR-3, BT-20, and MDA-mb-435S) of the tumor cell lines SEQ ID NO:35 was underexpressed by at least two-fold as compared to the HMEC cells. Therefore, in various embodiments, SEQ ID NO:35 can be used for one or more of the following: i) monitoring treatment of breast cancer, ii) diagnostic assays for breast cancer, and iii) developing therapeutics and/or other treatments for breast cancer.

SEQ ID NO:35 was also differentially expressed in four lung tumor tissue samples, using a pair comparison study design in which normal lung tissue is compared to tumor lung tissue from the same donor. Lung cancers are divided into four histopathologically distinct groups. Three groups (squamous cell carcinoma, adenocarcinoma, and large cell carcinoma) are classified as non-small cell lung cancers (NSCLCs). The fourth group of cancers is referred to as small cell lung cancer (SCLC). Collectively, NSCLCs account for approximately 70% of cases while SCLCs account for approximately 18% of cases. The molecular and cellular biology underlying the development and progression of lung cancer are incompletely understood. Deletions on chromosome 3 are common in this disease and are thought to indicate the presence of a tumor suppressor gene in this region. Activating mutations in K-ras are commonly found in lung cancer and are the basis of one of the mouse models for the disease. Analysis of gene expression patterns associated with the development and progression of the disease will yield tremendous insight into the biology underlying this disease. SEQ ID NO:35 was underexpressed by at least two-fold in all of the lung tumor tissue samples tested as compared to the normal lung tissue from the same donors. These experiments indicate that SEQ ID NO:35 exhibits significant differential expression patterns using microarray techniques, and further establish the utility of SEQ ID NO:35 as a diagnostic marker or therapeutic agent which may be useful in a variety of conditions and diseases involving neurotransmission-associated proteins, including cancers. Therefore, in various embodiments, SEQ ID NO:35 can be used for one or more of the following: i) monitoring treatment of lung cancer, ii) diagnostic assays for lung cancer, and iii) developing therapeutics and/or other treatments for lung cancer.

As another example, both SEQ ID NO:36 and SEQ ID NO:37 were differentially expressed in specialized macrophage cells identified morphologically as “foam cells.” TBP-1 is a human promonocyte line derived from peripheral blood of a 1-year-old male with acute monocytic leukemia. TBP-1 cells can be differentiated to a macrophage-like phenotype by treatment with phorbol ester. Macrophages play a critical role in the initiation and maintenance of inflammatory immune responses. In atherosclerosis, macrophages localize in vascular lesions, accumulating lipids and taking on the morphology known as “foam cells.” Activated macrophages are also a major source of proinflammatory cytokines, and chronic inflammation is believed to be a major contributor to the development of atherosclerosis. SEQ ID NO:36 and SEQ ID NO:37 were underexpressed by at least two-fold in the differentiated (“foam cells”) as compared to the undifferentiated TBP-1 cells. Therefore, in various embodiments, SEQ ID NO:36 and SEQ ED NO:37 can be used for one or more of the following: i) monitoring treatment of atherosclerosis, ii) diagnostic assays for atherosclerosis, and iii) developing therapeutics and/or other treatments for atherosclerosis.

As another example, SEQ ID NO:45 was differentially expressed in specialized macrophage cells identified morphologically as “foam cells.” SEQ ID NO:45 was downregulated by at least two-fold in the differentiated “foam cells” as compared to the undifferentiated THP-1 cells. Therefore, in various embodiments, SEQ ID NO:45 can be used for one or more of the following: i) monitoring treatment of atherosclerosis, ii) diagnostic assays for atherosclerosis, and iii) developing therapeutics and/or other treatments for atherosclerosis.

As another example, SEQ ID NO:48 showed decreased expression in lung tumor tissue versus normal lung tissue as determined by microarray analysis. Normal lung tissue from a 68 year-old female (Roy Castle International Centre for Lung Cancer Research) was compared to lung tumor tissue from the same donor. Therefore, in various embodiments, SEQ D:) NO:48 can be used for one or more of the following: i) monitoring treatment of lung cancer, ii) diagnostic assays for lung cancer, and iii) developing therapeutics and/or other treatments for lung cancer.

XII. Complementary Polynucleotides

Sequences complementary to the NTRAN-encoding sequences, or any parts thereof, are used to detect, decrease, or inhibit expression of naturally occurring NTRAN. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of NTRAN. To inhibit transcription, a complementary oligonucleotide is designed from the most unique 5 ′ sequence and used to prevent promoter binding to the coding sequence. To inhibit translation, a complementary oligonucleotide is designed to prevent ribosomal binding to the NTRAN-encoding transcript.

XII. Expression of NTRAN

Expression and purification of NTRAN is achieved using bacterial or virus-based expression systems. For expression of NTRAN in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express NTRAN upon induction with isopropyl beta-D-thiogalactopyranoside (IPTG). Expression of NTRAN in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding NTRAN by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, NTRAN is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from NTRAN at specifically engineered sites. FLAG, an 8-amino acid peptide, enables immunoafffity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified NTRAN obtained by these methods can be used directly in the assays shown in Examples XVII and XVIII, where applicable.

XIV. Functional Assays

NTRAN function is assessed by expressing the sequences encoding NTRAN at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of NTRAN on gene expression can be assessed using highly purified populations of cells transfected with sequences encoding NTRAN and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding NTRAN and other genes of interest can be analyzed by northern analysis or microarray techniques.

XV. Production of NTRAN Specific Antibodies

NTRAN substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the NTRAN amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldricb, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-NTRAN activity by, for example, binding the peptide or NTRAN to a substrate, blocking with 1% BSA, reacting with rabbit antisera, washing, and reacting with radio-iodinated goat anti-rabbit IgG.

XVI. Purification of Naturally Occurring NTRAN Using Specific Antibodies

Naturally occurring or recombinant NTRAN is substantially purified by immunoaffinity chromatography using antibodies specific for NTRAN. An immunoaffinity column is constructed by covalently coupling anti-NTRAN antibody to an activated chromatographic resin, such as CNBr-activated SEPHAROSE (Amersham Biosciences). After the coupling, the resin is blocked and washed according to the manufacturer's instructions.

Media containing NTRAN are passed over the immunoaffinity column, and the column is washed under conditions that allow the preferential absorbance of NTRAN (e.g., high ionic strength buffers in the presence of detergent). The column is eluted under conditions that disrupt antibody/NTRAN binding (e.g., a buffer of pH 2 to pH 3, or a high concentration of a chaotrope, such as urea or thiocyanate ion), and NTRAN is collected.

XVII. Identification of Molecules Which Interact with NTRAN

NTRAN, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled NTRAN, washed, and any wells with labeled NTRAN complex are assayed. Data obtained using different concentrations of NTRAN are used to calculate values for the number, affinity, and association of NTRAN with the candidate molecules.

Alternatively, molecules interacting with NTRAN are analyzed using the yeast two-hybrid system as described in Fields, S. and O. Song (1989; Nature 340:245-246), or using commercially available kits based on the two-hybrid system, such as the MATCHMAKER system (Clontech).

NTRAN may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of NTRAN Activity

Measurements of NAP activity include tracer fluxes and electrophysiological approaches. Tracer fluxes are demonstrated by measuring uptake of labeled substrates into Xenopus laevis oocytes. Oocytes at stages V and VI are injected with NAP mRNA (10 ng per oocyte) and incubated for three days at 18° C. in OR2 medium (82.5 mM NaCl, 2.5 mM KCl, 1 mM CaCl₂, 1 mM MgCl₂, 1 mM Na₂HPO₄, 5 mM Hepes, 3.8 mM NaOH , 50 μg/ml gentamycin, pH 7.8) to allow expression of NAP protein. Oocytes are then transferred to standard uptake medium (100 mM NaCl, 2 mM KCl, 1 mM CaCl₂, 1 mM MgCl₂, 10 mM Hepes/Tris pH 7.5). Uptake of various neurotransmitters is initiated by adding a ³H substrate to the oocytes. After incubating for 30 minutes, uptake is terminated by washing the oocytes three times in Na⁺-free medium, measuring the incorporated ³H, and comparing with controls. NAP activity is proportional to the level of internalized ³H substrate.

An alternative assay for NTRAN activity measures the expression of NTRAN on the cell surface. cDNA encoding NTRAN is transfected into an appropriate mammalian cell line. Cell surface proteins are labeled with biotin as described (de la Fuente, M. A. et al. (1997) Blood 90:2398-2405). Immunoprecipitations are performed using NTRAN-specific antibodies, and immunoprecipitated samples are analyzed using sodium dodecyl sulfate polyacrylamide gel electrophoresis (SDS-PAGE) and immunoblotting techniques. The ratio of labeled immunoprecipitant to unlabeled immunoprecipitant is proportional to the amount of NTRAN expressed on the cell surface.

In the alternative, an assay for NTRAN activity is based on a prototypical assay for ligand/receptor-mediated modulation of cell proliferation. This assay measures the rate of DNA synthesis in Swiss mouse 3T3 cells. A plasmid containing polynucleotides encoding NTRAN is added to quiescent 3T3 cultured cells using transfection methods well known in the art. The transiently transfected cells are then incubated in the presence of [³H]thymidine, a radioactive DNA precursor molecule. Varying amounts of NTRAN ligand are then added to the cultured cells. Incorporation of [³H]thymidine into acid-precipitable DNA is measured over an appropriate time interval using a radioisotope counter, and the amount incorporated is directly proportional to the amount of newly synthesized DNA. A linear dose-response curve over at least a hundred-fold NTRAN ligand concentration range is indicative of receptor activity. One unit of activity per milliliter is defined as the concentration of NTRAN producing a 50% response level, where 100% represents maximal incorporation of [³H]thymidine into acid-precipitable DNA (McKay, I. and I. Leigh, eds. (1993) Growth Factors: A Practical Approach, Oxford University Press, New York N.Y., p.73.)

In a further alternative, the assay for NTRAN activity is based upon the ability of GPCR family proteins to modulate G protein-activated second messenger signal transduction pathways (e.g., cAMP; Gaudin, P. et al. (1998) J. Biol. Chem. 273:4990-4996). A plasmid encoding full length NTRAN is transfected into a mammalian cell line (e.g., Chinese hamster ovary (CHO) or human embryonic kidney (HEK-293) cell lines) using methods well-known in the art. Transfected cells are grown in 12-well trays in culture medium for 48 hours, then the culture medium is discarded, and the attached cells are gently washed with PBS. The cells are then incubated in culture medium with or without ligand for 30 minutes, then the medium is removed and cells lysed by treatment with 1 M perchloric acid. The cAMP levels in the lysate are measured by radioimmunoassay using methods well-known in the art. Changes in the levels of cAMP in the lysate from cells exposed to ligand compared to those without ligand are proportional to the amount of NTRAN present in the transfected cells.

To measure changes in inositol phosphate levels, the cells are grown in 24-well plates containing 1×10⁵ cells/well and incubated with inositol-free media and [³H]myoinositol, 2 mCi/well, for 48 hr. The culture medium is removed, and the cells washed with buffer containing 10 mM LiCl followed by addition of ligand. The reaction is stopped by addition of perchloric acid. Inositol phosphates are extracted and separated on Dowex AG1-X8 (Bio-Rad) anion exchange resin, and the total labeled inositol phosphates counted by liquid scintillation. Changes in the levels of labeled inositol phosphate from cells exposed to ligand compared to those without ligand are proportional to the amount of NTRAN present in the transfected cells.

In a further alternative, the ion conductance capacity of NTRAN is demonstrated using an electrophysiological assay. NTRAN is expressed by transforming a mammalian cell line such as COS7, HeLa or CHO with a eukaryotic expression vector encoding NTRAN. Eukaryotic expression vectors are commercially available, and the techniques to introduce them into cells are well known to those skilled in the art. A small amount of a second plasmid, which expresses any one of a number of marker genes such as β-galactosidase, is co-transformed into the cells in order to allow rapid identification of those cells which have taken up and expressed the foreign DNA. The cells are incubated for 48-72 hours after transformation under conditions appropriate for the cell line to allow expression and accumulation of NTRAN and β-galactosidase. Transformed cells expressing β-galactosidase are stained blue when a suitable colorimetric substrate is added to the culture media under conditions that are well known in the art. Stained cells are tested for differences in membrane conductance due to various ions by electrophysiological techniques that are well known in the art. Untransformed cells, and/or cells transformed with either vector sequences alone or β-galactosidase sequences alone, are used as controls and tested in parallel. The contribution of NTRAN to cation or anion conductance can be shown by incubating the cells using antibodies specific for either NTRAN. The respective antibodies will bind to the extracellular side of NTRAN, thereby blocking the pore in the ion channel, and the associated conductance. To study the dependence of NAP on external ions, sodium can be replaced by choline or N-methyl-D-glucamine and chloride by gluconate, NO₃, or SO₄ (Kavanaugh, M. P. et al. (1992) J. Biol. Chem. 267:22007-22009).

In a further alternative, NTRAN transport activity is assayed by measuring uptake of labeled substrates into Xenopus laevis oocytes. Oocytes at stages V and VI are injected with NTRAN mRNA (10 ng per oocyte) and incubated for 3 days at 18° C. in OR2 medium (82.5 mM NaCl, 2.5 mM KCl, 1 mM CaCl₂, 1 mM MgCl₂, 1 mM Na₂HPO₄, 5 mM Hepes, 3.8 mM NaOH, 50 μg/ml gentamycin, pH 7.8) to allow expression of NTRAN protein. Oocytes are then transferred to standard uptake medium (100 mM NaCl, 2 mM KCl, 1 mM CaCl₂, 1 mM MgCl₂, 10 mM Hepes/Tris pH 7.5). Uptake of various substrates (e.g., amino acids, sugars, drugs, and neurotransmitters) is initiated by adding a ³H substrate to the oocytes. After incubating for 30 minutes, uptake is terminated by washing the oocytes three times in Na⁺-free medium, measuring the incorporated ³H, and comparing with controls. NTRAN activity is proportional to the level of internalized ³H substrate.

In a further alternative, NTRAN activity can be demonstrated using an electrophysiological assay for ion conductance. Capped NTRAN mRNA transcribed with T7 polymerase is injected into defolliculated stage V Xenopus oocytes, similar to the previously described method. Two to seven days later, transport is measured by two-electrode voltage clamp recording. Two-electrode voltage clamp recordings are performed at a holding potential of 50 mV. The data are filtered at 10 Hz and recorded with the MacLab digital-to-analog converter and software for data acquisition and analysis (AD Instruments, Castle Hill, Australia). To study the dependence of NTRAN on external ions, sodium can be replaced by choline or N-methyl-D-glucamine and chloride by gluconate, NO₃, or SO₄ (Kavanaugh, M. P. et al. (1992) J. Biol. Chem. 267:22007-22009).

In the alternative, choline transporter activity or choline-transporter-hike CTL1 protein activity of NTRAN is determined by measuring choline uptake by yeast transformed with expression vectors harboring polynucleotides encoding NTRAN. The assay is performed in nitrogen-free medium at 30° C. for 10 or 30 min in the presence of 25 nM [³H]choline. The cells are then filtered, and washed. The amount of [³H]choline present in the cells is proportional to the activity of NTRAN in the cells (O'Regan, S. supra).

In a further alternative, NTRAN protein kinase (PK) activity is measured by phosphorylation of a protein substrate using gamma-labeled [³²P]-ATP and quantitation of the incorporated radioactivity using a gamma radioisotope counter. NTRAN is incubated with the protein substrate, [³²P]-ATP, and an appropriate kinase buffer. The ³²P incorporated into the product is separated from free [³²P]-ATP by electrophoresis and the incorporated ³²P is counted. The amount of ³²P recovered is proportional to the PK activity of NTRAN in the assay. A determination of the specific amino acid residue phosphorylated is made by phosphoamino acid analysis of the hydrolyzed protein.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| Incyte | | | | Incyte | Incyte Full |
|---|---|---|---|---|---|
| Project | Polypeptide | Incyte | Polynucleotide | Polynucleotide | Length |
| ID | SEQ ID NO: | Polypeptide ID | SEQ ID NO: | ID | Clones |
| 7500354 | 1 | 7500354CD1 | 26 | 7500354CB1 | |
| 3871329 | 2 | 3871329CD1 | 27 | 3871329CB1 | |
| 1681386 | 3 | 1681386CD1 | 28 | 1681386CB1 | |
| 7500938 | 4 | 7500938CD1 | 29 | 7500938CB1 | |
| 90055441 | 5 | 90055441CD1 | 30 | 90055441CB1 | |
| 7500936 | 6 | 7500936CD1 | 31 | 7500936CB1 | |
| 7500950 | 7 | 7500950CD1 | 32 | 7500950CB1 | |
| 7500854 | 8 | 7500854CD1 | 33 | 7500854CB1 | |
| 2754176 | 9 | 2754176CD1 | 34 | 2754176CB1 | |
| 7503408 | 10 | 7503408CD1 | 35 | 7503408CB1 | |
| 71086982 | 11 | 71086982CD1 | 36 | 71086982CB1 | |
| 7506367 | 12 | 7506367CD1 | 37 | 7506367CB1 | |
| 1414020 | 13 | 1414020CD1 | 38 | 1414020CB1 | |
| 7621128 | 14 | 7621128CD1 | 39 | 7621128CB1 | |
| 7505822 | 15 | 7505822CD1 | 40 | 7505822CB1 | |
| 71607945 | 16 | 71607945CD1 | 41 | 71607945CB1 | |
| 7505777 | 17 | 7505777CD1 | 42 | 7505777CB1 | |
| 7505818 | 18 | 7505818CD1 | 43 | 7505818CB1 | |
| 7505821 | 19 | 7505821CD1 | 44 | 7505821CB1 | |
| 7506685 | 20 | 7506685CD1 | 45 | 7506685CB1 | |
| 7500933 | 21 | 7500933CD1 | 46 | 7500933CB1 | |
| 7389203 | 22 | 7389203CD1 | 47 | 7389203CB1 | |
| 7506268 | 23 | 7506268CD1 | 48 | 7506268CB1 | |
| 7509159 | 24 | 7509159CD1 | 49 | 7509159CB1 | |
| 7512347 | 25 | 7512347CD1 | 50 | 7512347CB1 | |

TABLE 2

| Polypeptide | | GenBank ID NO: | | |
|---|---|---|---|---|
| SEQ | Incyte | or PROTEOME | Probability | |
| ID NO: | Polypeptide ID | ID NO: | Score | Annotation |
| 1 | 7500354CD1 | g28721 | 0.0 | [Homo sapiens] amyloid A4(751) protein (AA 1 - 751) |
| | | | | (Ponte, P. et al. (1988) Nature 331: 525-527) |
| 2 | 3871329CD1 | g6651019 | 8.7E−291 | [Mus musculus] semaphorin cytoplasmic domain-associated protein 3A |
| 3 | 1681386CD1 | g17861384 | 0.0 | [fl][Homo sapiens] nesprin-2 gamma |
| | 1681386CD1 | g10880799 | 0.0 | [Mus musculus] Syne-1B |
| | | | | (Apel, E. D. et al. (2000) J. Biol. Chem. 275: 31986-31995) |
| 4 | 7500938CD1 | g9588046 | 5.8E−119 | [Homo sapiens] BRI3 |
| | | | | (Vidal, R. et al. (2001) Gene 266: 95-102) |
| 5 | 90055441CD1 | g3851518 | 5.2E−135 | [Mus musculus] semaF cytoplasmic domain associated protein 2 |
| 6 | 7500936CD1 | g9588046 | 2.6E−114 | [Homo sapiens] BRI3 |
| | | | | Vidal, R et al. (supra) |
| 7 | 7500950CD1 | g12248382 | 0.0 | [Homo sapiens] SEMB |
| 8 | 7500854CD1 | g6624588 | 2.0E−46 | [Homo sapiens] dJ570F3.2 (LOC51596 (divalent cation tolerant protein CUTA)) |
| 9 | 2754176CD1 | g3790389 | 0.0 | [Rattus norvegicus] m-tomosyn |
| | | | | Fujita, Y. et al. Tomosyn: a syntaxin-1-binding protein that forms a novel complex |
| | | | | in the neurotransmitter release process. Neuron 20, 905-15 (1998). |
| | 2754176CD1 | 701814\|LOC81022 | 0.0 | [Rattus norvegicus] Tomosyn, a syntaxin-1-binding protein that may function in |
| | | | | neurotransmitter release by promoting SNARE complex formation. |
| | 2754176CD1 | 424148\|KIAA1006 | 0.0 | [Homo sapiens] Protein containing a WD domain (WD-40repeat), which may |
| | | | | mediate protein-protein interactions. |
| | | | | Nagase, T. et al. Prediction of the coding sequences of unidentified human genes. |
| | | | | XIII. The complete sequences of 100 new cDNA clones from brainwhich code for |
| | | | | large proteins in vitro. DNA Res. 6 (1), 63-70 (1999) |
| | 2754176CD1 | 340838\|LLGL2 | 4.6E−94 | [Homo sapiens] Protein with high similarity to LLGL1, which is a cytoskeletal |
| | | | | protein that associates with the heavy chain of nonmuscle myosin II and may be |
| | | | | associated with Smith-Magenis syndrome |
| 10 | 7503408CD1 | g1927202 | 1.2E−114 | [Homo sapiens] FEZ1 |
| | | | | Bloom, L. and Horvitz, H. R. The Caenorhabditis elegans gene unc-76 and its |
| | | | | human homologs define a new gene family involved in axonal outgrowth and |
| | | | | fasciculation. |
| | 7503408CD1 | 657931\|FEZ1 | 2.7E−53 | [Homo sapiens] Zygin 1, has similarity to C. elegans UNC-76 and may have a role |
| | | | | in axonal outgrowth. |
| | | | | Ishii, H. et al. The FEZ1 gene at chromosome 8p22 encodes a leucine-zipper |
| | | | | protein, and its expression is altered in multiple human tumors. Proc. Natl. Acad. |
| | | | | Sci. U.S.A. 96, 3928-33 (1999). |
| | 7503408CD1 | 568588\|FEZ2 | 9.8E−33 | [Homo sapiens] Fasciculation and elongation protein (zygin), functions in axonal |
| | | | | outgrowth, a member of a family of proteins related to C. elegans UNC-76 which |
| | | | | have a structural or signaling role in axonal bundle formation and maintenance. |
| 11 | 71086982CD1 | g1864093 | 4.6E−232 | [Rattus norvegicus] PSD-95/SAP90-associated protein-4 |
| | | | | Takeuchi, M. et al. (1997) SAPAPs. A family of PSD-95/SAP90-associated |
| | | | | proteins localized at postsynaptic density. J. Biol. Chem. 272: 11943-11951. |
| | 71086982CD1 | 331912\|Rn.11279 | 4.0E−233 | [Rattus norvegicus] Protein with high similarity to synaptic proteins that bind to |
| | | | | the guanylate kinase-like domain of PSD-95/SAP90, which is associated with |
| | | | | receptors and ion channels and may function in signaling. |
| | 71086982CD1 | 348264\|DLGAP2 | 9.8E−104 | [Homo sapiens] Protein with high similarity to guanylate kinase-associated protein |
| | | | | DLGAP1, which is a synaptic protein that binds to the guanylate kinase-like |
| | | | | domain of the PSD-95 family, may function in synapse organization and neuronal |
| | | | | cell signaling. |
| | | | | Ranta, S. et al. (2000) Positional cloning and characterization of the human |
| | | | | DLGAP2 gene and its exclusion in progressive epilepsy with mental retardation. |
| 12 | 7506367CD1 | g1864093 | 5.4E−229 | [Rattus norvegicus] PSD-95/SAP90-associated protein-4 |
| | | | | Takeuchi, M. et al. (supra) |
| | 7506367CD1 | 331912\|Rn.11279 | 4.8E−230 | [Rattus norvegicus] Protein with high similarity to synaptic proteins that bind to |
| | | | | the guanylate kinase-like domain of PSD-95/SAP90, which is associated with |
| | | | | receptors and ion channels and may function in signaling. |
| | 7506367CD1 | 348264\|DLGAP2 | 2.0E−103 | [Homo sapiens] Protein with high similarity to guanylate kinase-associated protein |
| | | | | DLGAP1, which is a synaptic protein that binds to the guanylate kinase-like |
| | | | | domain of the PSD-95 family, may function in synapse organization and neuronal |
| | | | | cell signaling. |
| 13 | 1414020CD1 | g1235591 | 8.0E−283 | [Rattus norvegicus] dendrin |
| | | | | Wisden, H. A. et al. (1997) Mol. Cell Neurosci. 8: 367-374. |
| | 1414020CD1 | 423733\|KIAA0749 | 0.0 | [Homo sapiens] Protein with high similarity to rat Rn.5444, dendrin, which is |
| | | | | localized to dendrites and expressed exclusively in the forebrain, and expression |
| | | | | of which is decreased after sleep deprivation. |
| | 1414020CD1 | 328690\|Rn.5444 | 7.0E−284 | [Rattus norvegicus][Endoplasmic reticulum; Cytoplasmic; Plasma membrane; |
| | | | | Dendrite] Dendrin, localized to dendrites and expressed exclusively in the |
| | | | | forebrain, may have a role in modulating synaptic plasticity, expression is |
| | | | | decreased after sleep deprivation. |
| 14 | 7621128CD1 | g17026376 | 0.0 | [fl][Mus musculus] muscle-derived protein MDP77 variant 2 |
| | 7621128CD1 | g7619884 | 6.8E−183 | [Gallus gallus] muscle derived protein. |
| | | | | Uyeda, A. et al. (2000) Biochem. Biophys. Res. Commun. 269: 564-569. |
| | 7621128CD1 | 335126\|EEA1 | 2.0E−14 | [Homo sapiens][Small molecule-bindingprotein][Endosome/Endosomal vesicles; |
| | | | | Nuclear; Cytoplasmic; Plasma membrane] Early endosome antigen 1, effector of |
| | | | | endosomal small GTPase RAB5, required for endosome fusion, may specify |
| | | | | transport directionality from the plasma membrane to early endosomes; |
| | | | | autoantigen associated with subacute cutaneous systemic lupus erythematosus. |
| | | | | Mu, F. t. et al. (1995) J. Biol. Chem. 270: 13503-13511. |
| 15 | 7505822CD1 | 569920/KIAA0063 | 1.6E−33 | [Homo sapiens] Member of the Josephin family, which contain triplet repeats that |
| | | | | are implicated in neurological diseases, has low similarity to a region of human |
| | | | | MJD Ataxin 3, which is associated with Machado-Joseph disease and induces |
| | | | | apoptosis when overexpressed. |
| 16 | 71607945CD1 | g193209 | 0.0 | [Mus musculus] phosphoprotein. |
| | | | | Lafer, E. et al. (1992) J. Neurosci. 12: 2144-2155. |
| | 71607945CD1 | 570248\|SNAP91 | 0.0 | [Homo sapiens] Synaptosomal-associated protein 91, aclathrin assembly protein |
| | | | | that helps regulate clathrin mediatedsynaptic vesicle recycling in synapses. |
| | | | | Yao, P. J. et al. (1999) Neuroscience 94: 389-394. |
| 17 | 7505777CD1 | g2589160 | 3.1E−88 | [Homo sapiens] DCRA. |
| | | | | Nakamura, A. (1997) J. Biochem. 122: 872-877. |
| | 7505777CD1 | 342972\|DSCR3 | 2.7E−89 | [Homo sapiens] Down syndrome criticial region gene 3, aubiquitously expressed |
| | | | | protein; the corresponding gene is located in the Down syndrome critical region of |
| | | | | chromosome 21. |
| | | | | Nakamura, A. et al. (1997) supra |
| 18 | 7505818CD1 | g2262199 | 2.3E−162 | [Homo sapiens] josephin MJD1. |
| | | | | Goto, J. (et al. (1997) Neurosci. Res. 28: 373-377. |
| | 7505818CD1 | 700748\|MJD | 7.2E−155 | [Homo sapiens][Nuclear; Cytoplasmic; Nuclear matrix]Machado-Joseph disease |
| | | | | (spinocerebellar ataxia 3), may be involved in nucleotide-excision repair; variants |
| | | | | with an expanded polyglutamine region are associated with Machado-Joseph |
| | | | | disease and induce apoptosis when overexpressed. |
| | | | | Paulson, H. L. et al. (1997) Ann. Neurol. 41: 453-462. |
| 19 | 7505821CD1 | g2262199 | 7.8E−182 | [Homo sapiens] josephin MJD1. |
| | | | | Goto, J. et al. (1997) supra |
| | 7505821CD1 | 700748\|MJD | 2.6E−174 | [Homo sapiens][Nuclear; Cytoplasmic; Nuclear matrix] Machado-Joseph disease |
| | | | | (spinocerebellar ataxia 3), may be involved in nucleotide-excision repair; variants |
| | | | | with an expanded polyglutamine region are associated with Machado-Joseph |
| | | | | disease and induce apoptosis when overexpressed. |
| | | | | Gaspar, C. et al. (2000) Hum. Mol. Genet. 9: 1957-1966. |
| 20 | 7506685CD1 | g1864093 | 1.4E−134 | [Rattus norvegicus] PSD-95/SAP90-associated protein-4. |
| | | | | Takeuchi, M. (1997) J. Biol. Chem. 272: 11943-11951. |
| | 7506685CD1 | 424074\|KIAA0964 | 3.2E−137 | [Homo sapiens] Protein with high similarity to Rn.37481, which is a synaptic |
| | | | | protein that binds to the guanylate kinase-likedomain of PSD-95/SAP90 and may |
| | | | | function in signaling. |
| 21 | 7500933CD1 | g9588046 | 2.7E−110 | [Homo sapiens] BRI3 |
| | | | | Vidal, R. et al. (supra) |
| | | 435996\|ITM2B | 8.9E−33 | [Homo sapiens][Unspecified membrane; Plasma membrane] Integral membrane |
| | | | | protein, a member of the type II integral membrane protein family; mutation in the |
| | | | | corresponding gene leads to production of an insoluble peptide fragment that |
| | | | | causes familial British dementia |
| | | | | Holton, J. L., et al. (2001) Am. J. Pathol. 158: 515-26 |
| | | | | Regional distribution of amyloid-Bri deposition and its association with |
| | | | | neurofibrillary degeneration in familial British dementia. |
| | | 583443\|Itm2b | 3.0E−32 | [Mus musculus][Unspecified membrane] Member of the type II integral |
| | | | | membrane protein family |
| 22 | 7389203CD1 | g10119916 | 7.4E−107 | [Homo sapiens] brain otoferlin long isoform |
| | | | | Yasunaga, S. et al. (2000) Am. J. Hum. Genet. 67: 591-600 |
| | | | | OTOF encodes multiple long and short isoforms: genetic evidence that the long |
| | | | | ones underlie recessive deafness DFNB9 |
| | | 710087\|Otof | 1.0E−107 | [Mus musculus] Otoferlin, may play a role in synaptic or other vesicle membrane |
| | | | | fusion; alteration of the human otoferlin gene is associated with nonsyndromic |
| | | | | prelingual deafness |
| | | 335100\|DYSF | 1.5E−47 | [Homo sapiens][Plasma membrane] Dysferlin, a protein necessary for normal |
| | | | | muscle function that may play a role in cell signaling; alterations of the |
| | | | | corresponding gene cause Miyoshi myopathy and limb girdle muscular dystrophy |
| | | | | type 2B |
| | | | | Piccolo, F. et al. (2000) Ann. Neurol. 48: 902-912 |
| | | | | Intracellular accumulation and reduced sarcolemmal expression of dysferlin in |
| | | | | limb-girdle muscular dystrophies. |
| 23 | 7506268CD1 | g1226235 | 1.6E−124 | [Mus musculus] Ac39/physophilin |
| | | | | Carrion-Vazquez, M. (1998) Eur. J. Neurosci. 10: 1153-1166 |
| | | | | Brain Ac39/physophilin: cloning, coexpression and colocalization with |
| | | | | synaptophysin. |
| | | 319226\|Atp6d | 2.2E−125 | [Mus musculus][Regulatory subunit; Active transporter, primary; Hydrolase; |
| | | | | Transporter; ATPase][Unspecified membrane; Plasma membrane] Putative |
| | | | | ortholog of human ATP6DV, which is subunit D of the vacuolar H(+)-ATPase |
| | | | | proton pump, an accessory subunit that regulates ATP binding and hydrolysis by |
| | | | | the A and B subunits |
| | | 356109\|ATP6D | 3.4E−103 | [Homo sapiens][Regulatory subunit; Active transporter, primary; Hydrolase; |
| | | | | Transporter; ATPase][Unspecified membrane; Plasma membrane] Vacuolar H+- |
| | | | | ATPase proton pump (subunit D), an accessory subunit in the peripheral catalytic |
| | | | | V1 complex, may be involved in coupling ATP hydrolysis (V1 complex) and |
| | | | | proton transport (V0 complex) |
| | | | | Forgac, M. (1998) FEBS Lett 440: 258-263. |
| | | | | Structure, function and regulation of the vacuolar (H+)-ATPases. |
| 24 | 7509159CD1 | g12248382 | 0.0 | [Homo sapiens] SEMB |
| | | 587311\|Sema4a | 0.0 | [Mus musculus] Semaphorin 4a(Semaphorin B), is in the transmembrane type |
| | | | | subfamily of semaphorins which is a family of proteins involved in neuronal axon |
| | | | | guidance during neural development |
| | | | | Puschel, A. W. et al. (1996) Mol. Cell Neurosci. 7: 419-431 |
| | | | | The sensory innervation of the mouse spinal cord may be patterned by differential |
| | | | | expression of and differential responsiveness to semaphorins. |
| | | 323692\|Sema4b | 2.4E−111 | [Mus musculus] [Unspecified membrane] Semaphorin 4b (Semaphorin C), is a |
| | | | | member of a family of proteins involved in neuronal growth cone guidance |
| 25 | 7512347CD1 | g12248382 | 3.3E−28 | [Homo sapiens] SEMB |

TABLE 3

| | | Ami- | | |
|---|---|---|---|---|
| | | no | | |
| SEQ | | Acid | | |
| ID | Incyte | Resi- | | Analytical Methods |
| NO: | Polypeptide | dues | Signature Sequences, Domains and Motifs | and Databases |
| 1 | 7500354CD1 | 733 | Signal_cleavage: M1-A17 | SPSCAN |
| | | | Signal Peptide: M1-A17; M1-E19; M1-T22 | HMMER |
| | | | Amyloid A4 extracellular domain: G24-P188 | HMMER_PFAM |
| | | | Kunitz/Bovine pancreatic trypsin inhibitor: C291-C341 | HMMER_PFAM |
| | | | Cytosolic domain: K687-N733; Transmembrane domain: A664-L686; Non-cytosolic domain: | TMHMMER |
| | | | M1-G663 | |
| | | | Pancreatic trypsin inhibitor (Kunitz) family proteins BL00280: G298-C341 | BLIMPS_BLOCKS |
| | | | Amyloidogenic glycoprotein extracellular domain proteins BL00319: T59-T107, T157-V182, | BLIMPS_BLOCKS |
| | | | D243-T276, T347-E396, V420-L461, Q462-A504, K687-Q732 | |
| | | | Amyloidogenic glycoprotein signatures: K161-V208; V698-N733 | PROFILESCAN |
| | | | Pancreatic trypsin inhibitor (Kunitz) family signature: P299-D357 | PROFILESCAN |
| | | | Amyloid A4 protein precursor signature PR00203: D177-N195, P363-R386, K662-K687, | BLIMPS_PRINTS |
| | | | E708-Q730 | |
| | | | Beta-amyloid peptide (beta-APP) signature PR00204: F638-L651, V652-A664, A664-A676 | BLIMPS_PRINTS |
| | | | Basic protease (Kunitz-type) inhibitor family signature PR00759: R288-A302, C316-G326, | BLIMPS_PRINTS |
| | | | G326-C341 | |
| | | | PROTEIN PRECURSOR AMYLOID TRANSMEMBRANE PROTEASE SIGNAL SERINE | BLAST_PRODOM |
| | | | INHIBITOR A4 GLYCOPROTEIN; PD003339: G4-V196; PD003344: I345-I518; PD003449: | |
| | | | E627-Q730; PD004634: E520-E617 | |
| | | | AMYLOIDOGENIC GLYCOPROTEIN EXTRACELLULAR DOMAIN | BLAST_DOMO |
| | | | DM02422;\|Q06481\|181-761: L165-Q732;\|P05067\|342-768: I345-L686;\|P51693\|187-619: | |
| | | | Y336-E550;\|A49414\|167-679: P353-E637 | |
| | | | Amyloidogenic glycoprotein extracellular domain signature: G181-P188 | MOTIFS |
| | | | Amyloidogenic glycoprotein intracellular domain signature: G719-K726 | MOTIFS |
| | | | Pancreatic trypsin inhibitor (Kunitz) family signature: F319-C337 | MOTIFS |
| | | | Potential Phosphorylation Sites: S159 S282 S351 S577 S595 S642 S660 T14 T61 T157 T266 | MOTIFS |
| | | | T278 T362 T489 T583 T614 T706 T724 Y336 | |
| | | | Potential Glycosylation Sites: D40N523 N552 | MOTIFS |
| 2 | 3871329CD1 | 1036 | Signal_cleavage: M1-R48 | SPSCAN |
| | | | PDZ domain (Also known as DHR or GLGF): T224-R313, E402-P486 | HMMER_PFAM |
| | | | Zinc finger, C3HC4 type (RING finger): C18-C56 | HMMER_PFAM |
| | | | Zinc finger, C3HC4 type BL00518: C33-C40 | BLIMPS_BLOCKS |
| | | | PDZ domain proteins PF00595: I447-N457 | BLIMPS_PFAM |
| | | | GLGF DOMAIN; DM00224\|P31016\|302-390: D217-V308 | BLAST_DOMO |
| | | | Zinc finger, C3HC4 type (RING finger), signature: C33-L42 | MOTIFS |
| | | | Potential Phosphorylation Sites: S91 S409 S433 S472 S561 S568 S581 S584 S650 S731 S744 | MOTIFS |
| | | | S786 S792 S837 S861 S879 S942 S966 S977 S991 T136 T188 T251 T291 T334 T422 T522 | |
| | | | T701 T712 T767 T902 T926 T927 T931 T1009 Y400 Y646 Y829 Y900 | |
| | | | Potential Glycosylation Sites: N231 N331 N578 N603 N711 N765 | MOTIFS |
| 3 | 1681386CD1 | 1847 | Spectrin repeat: N879-E981, R1098-H1204, K140-S236, K814-D876, E29-Q129, Q278-S344, | HMMER_PFAM |
| | | | Q1670-G1731, Q1513-Q1620, A984-E1095, N1207-R1293, K528-G553; | |
| | | | Leucine zipper pattern: L842-L863 | MOTIFS |
| | | | Potential Phosphorylation Sites: S23 S42 S88 S94 S133 S211 S222 S284 S312 S413 S470 | MOTIFS |
| | | | S517 S585 S747 S752 S769 S909 S946 S1108 S1133 S1237 S1239 S1323 S1339 S1366 | |
| | | | S1391 S1392 S1406 S1409 S1423 S1578 S1613 S1641 S1741 S1783 S1817 S1818 T41 T135 | |
| | | | T255 T274 T324 T329 T543 T595 T608 T624 T640 T669 T718 T719 T832 T1063 T1071 | |
| | | | T1096 T1153 T1196 T1224 T1231 T1259 T1310 T1327 T1532 T1567 T1770 Y248 | |
| | | | Potential Glycosylation Sites: N81 N375 N411 N606 N947 N992 N1061 N1125 N1169 | MOTIFS |
| | | | N1325 N1576 | |
| 4 | 7500938CD1 | 230 | Cytosolic domain: M1-G57; Transmembrane domain: V58-Y80; Non-cytosolic domain: R81-V230 | TMHMMER |
| | | | PROTEIN INTEGRAL MEMBRANE TRANSMEMBRANE SIGNAL ANCHOR 2A E25 2B | BLAST_PRODOM |
| | | | E316 E25B PD023945: D147-C227, M1-L155 | |
| | | | Potential Phosphorylation Sites: S30 S49 S172 T110 T153 T190 T198 Y126 | MOTIFS |
| 5 | 90055441CD1 | 315 | Signal_cleavage: M1-T54 | SPSCAN |
| | | | PDZ domain (Also known as DHR or GLGF): E117-P198; | HMMER_PFAM |
| | | | Similar C Elegans DNDA Clone CEMSH65R F44D12.4 Protein PD041259 A39-R312 | BLAST_PRODOM |
| | | | Potential Phosphorylation Sites: S62 S77 S152 S213 T54 T88 T130 T192 T224 T234 T268 | MOTIFS |
| 6 | 7500936CD1 | 220 | PROTEIN INTEGRAL MEMBRANE TRANSMEMBRANE SIGNAL ANCHOR 2A E25 2B | BLAST_PRODOM |
| | | | E316 E25B PD023945: M65-C217 | |
| | | | Potential Phosphorylation Sites: S30 S162 T63 T143 T180 T188 Y79 | MOTIFS |
| | | | Potential Glycosylation Sites: N122 | MOTIFS |
| 7 | 7500950CD1 | 631 | Sema domain: M1-V348 | HMMER_PFAM |
| | | | Cytosolic domain: A574-A631; Transmembrane domain: W551-V573; Non-cytosolic domain: | TMHMMER |
| | | | M1-Y550 | |
| | | | SEMAPHORIN B PRECURSOR SIGNAL IMMUNOGLOBULIN FOLD MULTIGENE | BLAST_PRODOM |
| | | | FAMILY NEUROGENESIS DEVELOPMENTAL PROTEIN PD116663: P413-A631 | |
| | | | SEMAPHORIN PROTEIN PRECURSOR RECEPTOR KINASE SIGNAL TYROSINE | BLAST_PRODOM |
| | | | HEPATOCYTE PD001844: V118-C280, I2-F146, G270-V348 | |
| | | | SEMAPHORIN; FASCICLIN; COLLAPSIN; II; DM01606 | BLAST_DOMO |
| | | | I48745\|1-619: M1-V348, L344-L491 | |
| | | | A49069\|1-646: I2-C280, P139-V348, V351-W461 | |
| | | | I48747\|1-646: I2-C280, P139-V348, V351-W461, P449-L482 | |
| | | | I48744\|1-639: I2-V348, V351-T474 | |
| | | | Bacterial regulatory proteins, araC family signature: R537-R581 | MOTIFS |
| | | | Potential Phosphorylation Sites: S7 S12 S20 S77 S153 S243 S286 S312 S368 S372 S403 S425 | MOTIFS |
| | | | S444 S608 S615 S617 T53 T151 T163 T185 T262 T267 T591 | |
| | | | Potential Glycosylation Sites: N21 N36 N366 N477 | MOTIFS |
| 8 | 7500854CD1 | 132 | Signal_cleavage: M1-P30 | SPSCAN |
| | | | Signal Peptide: M1-P30 | HMMER |
| | | | CutA1 divalent ion tolerance protein: V45-G116 | HMMER_PFAM |
| | | | PROTEIN DIVALENT CATION TOLERANCE PERIPLASMIC CUTA C-TYPE | BLAST_PRODOM |
| | | | CYTOCHROME BIOGENESIS PD009206: F49-M98 | |
| | | | Potential Phosphorylation Sites: S82 S105 | MOTIFS |
| 9 | 2754176CD1 | 1115 | Signal_cleavage: M1-S21 | SPSCAN |
| | | | WD domain, G-beta repeat: I187-D224, K431-D464, A230-N265, C91-N126, N493-R529, | HMMER_PFAM |
| | | | C49-G85, L621-Y657 | |
| | | | Lethal(2) giant larvae protein signature PR00962: T40-Y58, P308-M330, Q360-Q380, P448-Q471, | BLIMPS_PRINTS |
| | | | P775-L793, S634-L658 | |
| | | | LARVAE PROTEIN GIANT SUPPRESSOR LETHAL 2 P127 ANTI-ONCOGENE REPEAT | BLAST_PRODOM |
| | | | SIMILARITY SEVERAL PD007842: V298-D500, R144-Y363, E34-W125, L600-Y657, | |
| | | | D495-F530, L211-D224 | |
| | | | SIMILARITY TO SEVERAL TUMOR SUPPRESSOR PROTEINS SUCH AS MOUSE | BLAST_PRODOM |
| | | | MGL1 PD145797: N737-E844, S531-P677, D533-P566 | |
| | | | SIMILARITY SEVERAL TUMOR SUPPRESSOR PROTEINS SUCH AS MOUSE MGL1 | BLAST_PRODOM |
| | | | CODED PD040184: S864-F1115 | |
| | | | PROTEIN SNI1 SRO7 SNI2 SRO77 C1F3.03 CHROMOSOME I TRANSMEMBRANE | BLAST_PRODOM |
| | | | PD025667: I37-A239, D231-Y363, I451-K484, V880-Y982 | |
| | | | HUGL; LARVAE; GIANT; DM03976 | BLAST_DOMO |
| | | | \|S55474\|1-1015: P30-E550, L600-D852, I758-S983 | |
| | | | \|S54142\|1-1017: G196-V554, E34-L127, S749-L997, G612-E844 | |
| | | | \|P08111\|1-1032: G31-D490, N902-L980, P628-S692, I758-P797 | |
| | | | YPR032W; MEMBRANE; DM08120\|S54506\|36-1033: I37-D237, I451-L593 | BLAST_DOMO |
| | | | Trp-Asp (WD) repeats signature: L113-L127, I451-A465 | MOTIFS |
| | | | Potential Phosphorylation Sites: S137 S190 S227 S255 S343 S393 S429 S446 S459 S531 | MOTIFS |
| | | | S588 S684 S715 S719 S723 S869 S883 S939 S973 S1034 S1091 T51 T161 T235 T302 T349 | |
| | | | T400 T478 T748 T768 T999 Y234 Y948 Y1113 | |
| | | | Potential Glycosylation Sites: N638 N902 | MOTIFS |
| 10 | 7503408CD1 | 363 | UNC76 ZYGINI FEZ1T PD011714: D102-T363 | BLAST_PRODOM |
| | | | Potential Phosphorylation Sites: S7 S18 S42 S44 S55 S58 S116 S134 S169 S199 S272 S273 | MOTIFS |
| | | | S287 S306 S324 S327 T99 T308 | |
| | | | Potential Glycosylation Sites: N48 N132 N189 | MOTIFS |
| 11 | 71086982CD1 | 453 | Signal_cleavage: M1-A46 | SPSCAN |
| | | | PROTEIN PSD95/SAP90-ASSOCIATED DAP1 GUANYLATE KINASE-ASSOCIATED | BLAST_PRODOM |
| | | | BETA ALPHA PSD95 BINDING PD006399: A235-N433 | |
| | | | PSD95/SAP90-ASSOCIATED PROTEIN DAP1 GUANYLATE KINASE-ASSOCIATED | BLAST_PRODOM |
| | | | BETA ALPHA PSD95 BINDING PD007821: S12-P195 | |
| | | | PSD95/SAP90-ASSOCIATED PROTEIN 4 PD142277: D196-E234 | BLAST_PRODOM |
| | | | Potential Phosphorylation Sites: S41 S45 S76 S115 S126 S170 S171 S186 S190 S193 S224 | MOTIFS |
| | | | S251 S315 S352 S357 S410 S429 S434 T30 T49 T111 T175 T276 T338 | |
| | | | Potential Glycosylation Sites: N68 N183 N222 N294 | MOTIFS |
| 12 | 7506367CD1 | 505 | Signal_cleavage: M1-A46 | SPSCAN |
| | | | PROTEIN PSD95/SAP90-ASSOCIATED DAP1 GUANYLATE KINASE-ASSOCIATED | BLAST_PRODOM |
| | | | BETA ALPHA PSD95 BINDING PD006399: A287-N485 | |
| | | | PSD95/SAP90-ASSOCIATED PROTEIN DAP 1 GUANYLATE KINASE-ASSOCIATED | BLAST_PRODOM |
| | | | BETA ALPHA PSD95 BINDING PD007821: S12-I130, P180-P247 | |
| | | | PSD95/SAP90-ASSOCIATED PROTEIN 4 PD142277: D248-E286 | BLAST_PRODOM |
| | | | Potential Phosphorylation Sites: S41 S45 S76 S126 S134 S146 S222 S223 S238 S242 S245 | MOTIFS |
| | | | S276 S303 S367 S404 S409 S462 S481 S486 T30 T49 T111 T227 T328 T390 | |
| | | | Potential Glycosylation Sites: N68 N235 N274 N346 | MOTIFS |
| 13 | 1414020CD1 | 711 | DENDRIN PD146601: M55-K709 | BLAST_PRODOM |
| | | | Potential Phosphorylation Sites: S13 S46 S327 S389 S447 S515 S516 S539 S626 S644 S652 | MOTIFS |
| | | | S665 S666 S679 S688 T125 T242 T269 T433 T469 T540 T545 T622 T658 Y278 | |
| | | | Potential Glycosylation Sites: N68 | MOTIFS |
| 14 | 7621128CD1 | 684 | PROTEIN COILED COIL CHAIN MYOSIN REPEAT HEAVY ATP-BINDING FILAMENT | BLAST_PRODOM |
| | | | HEPTAD PD000002: V122-E353 | |
| | | | Muscle T22C1.6 PROTEIN PD075375: I285-E465 | BLAST_PRODOM |
| | | | TRICHOHYALIN DM03839 | BLAST_DOMO |
| | | | \|P37709\|632-1103: E103-E492 | |
| | | | \|P22793\|921-1475: K129-E492 | |
| | | | Potential Phosphorylation Sites: S82 S127 S212 S265 S284 S364 S464 S474 S529 S569 S603 | MOTIFS |
| | | | T105 T121 T157 T227 T253 T383 T391 T392 T404 T523 T588 T678 Y334 | |
| | | | Potential Glycosylation Sites: N4 N225 N481 | MOTIFS |
| 15 | 7505822CD1 | 146 | Josephin: G61-E137, T13-R49 | HMMER_PFAM |
| | | | PROTEIN T27A16.26 KIAA0063 HA1234 TRANSMEMBRANE PD108404: V59-L129, | BLAST_PRODOM |
| | | | V14-R49 | |
| | | | Potential Phosphorylation Sites: Y94 | MOTIFS |
| 16 | 71607945CD1 | 902 | ENTH domain: G19-M141 | HMMER_PFAM |
| | | | CLATHRIN COAT ASSEMBLY PROTEIN AP180 ASSOCIATED COATED PITS | BLAST_PRODOM |
| | | | ALTERNATIVE SPLICING PD037674: T284-A405 | |
| | | | PROTEIN CLATHRIN ASSEMBLY COAT AP180 ASSOCIATED COATED PITS | BLAST_PRODOM |
| | | | ALTERNATIVE SPLICING PD009526: Q4-R139 | |
| | | | CLATHRIN COAT ASSEMBLY PROTEIN AP180 ASSOCIATED COATED PITS | BLAST_PRODOM |
| | | | ALTERNATIVE SPLICING PD028037: E436-S524 | |
| | | | PROTEIN CLATHRIN ASSEMBLY COAT AP180 ASSOCIATED COATED PITS | BLAST_PRODOM |
| | | | ALTERNATIVE SPLICING PD014599: T549-W790 | |
| | | | APO POLYSIALO-GLYCOPROTEIN; SIALOGLYCOPROTEIN; DM05537 | BLAST_DOMO |
| | | | \|P12027\|1-541: G214-A731 | |
| | | | \|S08207\|1-540: E249-D718 | |
| | | | Potential Phosphorylation Sites: S128 S137 S273 S447 S475 S570 S649 S711 S750 S883 T5 | MOTIFS |
| | | | T7 T30 T62 T342 T413 T769 T770 T785 | |
| | | | Potential Glycosylation Sites: N50 N69 N105 | MOTIFS |
| 17 | 7505777CD1 | 172 | DOWN SYNDROME CRITICAL REGION PROTEIN A PD040389: K10-R171, M1-G19 | BLAST_PRODOM |
| | | | Potential Phosphorylation Sites: S28 T3 | MOTIFS |
| | | | Potential Glycosylation Sites: N58 | MOTIFS |
| 18 | 7505818CD1 | 321 | Josephin: P10-Q143, M1-Q9 | HMMER_PFAM |
| | | | Ubiquitin interaction motif: E188-S205, D168-I185, M294-V311 | HMMER_PFAM |
| | | | PROTEIN JOSEPHIN MJD1 MACHADO JOSEPH DISEASE POLYMORPHISM TRIPLET | BLAST_PRODOM |
| | | | REPEAT EXPANSION SPINOCEREBELLAR PD014018: K8-E235, M1-Q9 | |
| | | | SPINOCEREBELLAR ATAXIA TYPE 3 PD127646: Q258-K321 | BLAST_PRODOM |
| | | | Potential Phosphorylation Sites: S181 S205 S295 T67 T83 T152 T222 T310 | MOTIFS |
| | | | Potential Glycosylation Sites: N208 N220 | MOTIFS |
| 19 | 7505821CD1 | 362 | Signal_cleavage: M46-G73 | SPSCAN |
| | | | Josephin: Q63-Q183, M1-L62 | HMMER_PFAM |
| | | | Ubiquitin interaction motif: E228-S245, D208-I225, M335-V352 | HMMER_PFAM |
| | | | PROTEIN JOSEPHIN MJD1 MACHADO JOSEPH DISEASE POLYMORPHISM TRIPLET | BLAST_PRODOM |
| | | | REPEAT EXPANSION SPINOCEREBELLAR PD014018: M1-E275 | |
| | | | SPINOCEREBELLAR ATAXIA TYPE 3 PD127646: Q299-K362 | BLAST_PRODOM |
| | | | Potential Phosphorylation Sites: S29 S221 S245 S336 T54 T107 T123 T192 T262 T351 | MOTIFS |
| | | | Potential Glycosylation Sites: N248 N260 | MOTIFS |
| 20 | 7506685CD1 | 332 | Signal_cleavage: M1-A46 | SPSCAN |
| | | | PROTEIN PSD95/SAP90-ASSOCIATED DAP1 GUANYLATE KINASE-ASSOCIATED | BLAST_PRODOM |
| | | | BETA ALPHA PSD95 BINDING PD006399: A114-N312 | |
| | | | PSD95/SAP90-ASSOCIATED PROTEIN DAP1 GUANYLATE KINASE-ASSOCIATED | BLAST_PRODOM |
| | | | BETA ALPHA PSD95 BINDING PD007821: S12-S75 | |
| | | | PSD95/SAP90-ASSOCIATED PROTEIN 4 PD142277: T77-E113 | BLAST_PRODOM |
| | | | Potential Phosphorylation Sites: S41 S45 S76 S103 S130 S194 S231 S236 S289 S308 S313 | MOTIFS |
| | | | T30 T49 T155 T217 | |
| | | | Potential Glycosylation Sites: N68 N101 N173 | MOTIFS |
| 21 | 7500933CD1 | 214 | Cytosolic domain: M1-G57; Transmembrane domain: V58-Y80; Non-cytosolic domain: R81-V214 | TMHMMER |
| | | | PROTEIN INTEGRAL MEMBRANE TRANSMEMBRANE SIGNAL ANCHOR 2A E25 2B | BLAST_PRODOM |
| | | | E316 E25B | |
| | | | PD023945: I144-C211, M1-E147, I114-C211 | |
| | | | Potential Phosphorylation Sites: S30 S49 S156 T110 T174 T182 Y126 | MOTIFS |
| 22 | 7389203CD1 | 716 | C2 domain: V28-I106, L191-V285 | HMMER_PFAM |
| | | | Cell attachment sequence: R154-D156 | MOTIFS |
| | | | Potential Phosphorylation Sites: S150 S170 S254 S265 S292 S530 T31 T58 T60 T108 T371 | MOTIFS |
| | | | T383 T437 T536 | |
| 23 | 7506268CD1 | 234 | ATP synthase (C/AC39) subunit: Y15-P232 | HMMER_PFAM |
| | | | SUBUNIT V-ATPASE AC39 VACUOLAR ATP SYNTHASE HYDROLASE HYDROGEN | BLAST_PRODOM |
| | | | ION TRANSPORT PD008622: S52-F234, N35-G169 | |
| | | | AC39; ATP; VACUOLAR; SYNTHASE; | BLAST_DOMO |
| | | | DM03240\|P12953\|1-272: A31-F234 | |
| | | | DM03240\|P53659\|1-363: N35-F234, L7-E43 | |
| | | | DM03240\|P54641\|10-355: V11-F234 | |
| | | | DM03240\|P32366\|32-344: V37-F234 | |
| | | | Potential Phosphorylation Sites: S29 S52 S116 T86 T172 Y77 | MOTIFS |
| 24 | 7509159CD1 | 728 | Signal Peptide: M1-A31 | HMMER |
| | | | Sema domain (found in Plexins and Semaphorins): Y127-Q445, F64-E122 | HMMER_PFAM |
| | | | Cytosolic domain: A671-A728; Transmembrane domain: W648-V670; Non-cytosolic domain: | TMHMMER |
| | | | M1-Y647 | |
| | | | SEMAPHORIN B PRECURSOR SEM B SIGNAL IMMUNOGLOBULIN FOLD | BLAST_PRODOM |
| | | | MULTIGENE FAMILY NEUROGENESIS DEVELOPMENTAL PROTEIN | |
| | | | PD116663: P510-A728; PD107003: M1-D63 | |
| | | | Semaphorin protein precursor receptor kinase signal tyrosine family hepatocyte PD001844: | BLAST_PRODOM |
| | | | V184-C346, G336-A442, K118-T229, L67-P147 | |
| | | | SEMAPHORIN; FASCICLIN; COLLAPSIN; II; DM01606 | BLAST_DOMO |
| | | | 48745\|1-619: E122-L588, M1-L191; A49423\|1-619: D134-E544, L67-D125 | |
| | | | C49423\|1-643: G139-C490, F64-D91; A49069\|1-646: E133-L429, Y334-W558, F64-K118 | |
| | | | Potential Phosphorylation Sites: S106 S111 S119 S143 S219 S309 S352 S378 S465 S469 | MOTIFS |
| | | | S500 S522 S541 S705 S712 S714 T217 T229 T251 T328 T333 T688 | |
| | | | Potential Glycosylation Sites: N463 N574 | MOTIFS |
| | | | Bacterial regulatory proteins, araC family signature: R634-R678 | MOTIFS |
| 25 | 7512347CD1 | 72 | Signal_cleavage: M1-A31 | SPSCAN |
| | | | Signal Peptide: M1-A31 | HMMER |
| | | | SEMAPHORIN B PRECURSOR SEM B SIGNAL IMMUNOGLOBULIN FOLD | BLAST_PRODOM |
| | | | MULTIGENE FAMILY NEUROGENESIS DEVELOPMENTAL PROTEIN PD107003: M1-Q62 | |
| | | | SEMAPHORIN; FASCICLIN; COLLAPSIN; II; DM01606\|I48745\|1-619: M1-V69 | BLAST_DOMO |

TABLE 4

| Polynucleotide | |
|---|---|
| SEQ ID NO:/ | |
| Incyte ID/ | |
| Sequence | |
| Length | Sequence Fragments |
| 26/ | 1-256, 1-287, 1-581, 11-298, 13-254, 13-273, 13-281, 15-256, 16-328, 17-244, 17-260, 17-3469, 18-267, 18-280, 18-282, |
| 7500354CB1/ | 18-677, 19-693, 19-782, 20-263, 20-266, 20-268, 20-569, 20-610, 20-780, 21-831, 22-782, 23-244, 23-263, 23-274, |
| 3495 | 23-276, 23-280, 23-298, 23-299, 23-311, 23-319, 23-324, 23-326, 23-375, 23-479, 23-520, 23-549, 23-554, 23-584, |
| | 23-587, 23-601, 23-656, 23-668, 23-680, 24-249, 24-265, 24-281, 24-307, 24-308, 24-316, 24-447, 24-551, 24-563, 24-566, |
| | 24-587, 24-601, 24-619, 24-638, 24-661, 24-781, 24-782, 24-809, 25-261, 25-297, 25-353, 25-440, 25-536, 25-701, 25-835, |
| | 26-250, 26-677, 28-340, 29-570, 29-762, 30-298, 30-308, 30-726, 31-663, 33-326, 33-346, 33-620, 33-671, 34-279, |
| | 35-293, 39-231, 42-288, 42-292, 43-596, 45-340, 45-609, 45-656, 46-611, 49-295, |
| | 50-563, 52-712, 57-364, 58-687, 59-500, 66-393, 67-340, 67-415, 67-446, 68-658, 75-331, 76-808, 77-350, 80-582, 81-640, |
| | 82-591, 83-770, 90-351, 98-351, 98-693, 98-709, 113-316, 113-642, 119-404, 123-332, 127-390, 135-765, 147-747, 148-694, |
| | 148-820, 172-789, 173-424, 178-423, 178-429, 178-434, 181-451, 186-426, 187-585, 194-467, 194-480, |
| | 194-800, 201-782, 205-980, 208-730, 216-413, 221-402, 226-864, 229-497, 235-902, 239-574, 255-909, 258-568, 262-505, |
| | 266-643, 267-503, 270-833, 271-852, 282-460, 284-549, 293-601, 300-551, 303-669, 303-767, 309-661, 309-730, 309-971, |
| | 318-840, 318-931, 320-559, 322-434, 322-719, 323-933, 326-586, 328-968, 332-825, 338-772, 338-914, 344-775, 350-888, |
| | 354-878, 354-944, 358-624, 361-945, 365-614, 368-794, 369-621, 371-593, 383-464, 383-611, 383-617, 383-620, 383-847, |
| | 383-981, 387-920, 390-818, 390-975, 391-902, 393-891, 412-657, 418-588, 418-649, 418-661, 418-662, 418-663, 418-665, |
| | 418-672, 431-685, 435-970, 451-712, 453-1131, 454-757, 455-1124, 470-1046, 470-1062, 471-797, 471-1067, 473-722, |
| | 480-817, 480-1267, 482-822, 510-993, 513-757, 513-761, 513-939, 521-749, 521-765, 522-689, 523-729, 523-1010, |
| | 534-768, 537-767, 541-659, 541-792, 541-829, 541-1096, 548-745, |
| | 548-843, 549-849, 550-872, 558-954, 569-854, 579-817, 581-763, 597-1146, 604-1181, 606-888, 615-1322, 632-870, |
| | 652-781, 659-902, 661-1133, 663-906, 668-1303, 683-907, 687-1095, 777-1564, 796-1227, 808-1506, 823-1372, |
| | 867-1608, 876-1560, 877-1050, 879-1567, 883-1092, 886-1384, 901-1558, 902-1501, 904-1344, 917-1259, 925-1593, 927-1481, |
| | 933-1481, 942-1556, 943-1224, 943-1234, 943-1258, 943-1268, 943-1546, 943-1558, 949-1197, 952-1623, 958-1259, 961-1536, |
| | 961-1611, 961-1646, 971-1556, 978-1621, 986-1560, 987-1241, 987-1295, 990-1155, 993-1596, 1009-1609, 1027-1295, |
| | 1030-1469, 1034-1238, 1034-1560, 1034-1706, 1040-1712, 1041-1497, 1041-1498, 1050-1760, 1054-1617, 1057-1181, 1057-1408, |
| | 1065-1671, 1081-1427, 1085-1348, 1090-1724, 1097-1693, 1114-1307, 1115-1369, 1124-1792, 1126-1684, 1127-1501, 1127-1646, |
| | 1169-1820, 1174-1641, 1183-1549, 1184-1713, 1184-1827, 1195-1438, 1195-1717, 1199-1664, 1210-1810, 1221-1494, |
| | 1226-1892, 1229-1801, 1230-1724, 1234-1679, 1237-1854, 1238-1538, 1238-1841, 1242-1477, 1243-2005, 1246-1534, |
| | 1248-1728, 1248-1775, 1248-1816, 1250-1926, 1251-1499, 1253-1514, 1258-1539, 1261-1539, 1265-1902, 1269-1850, 1269-1880, |
| | 1271-1587, 1275-1528, 1277-1916, 1279-1825, 1284-1517, 1286-1568, 1294-1865, 1296-1848, 1300-1613, 1302-1500, 1307-1790, |
| | 1310-1734, 1311-1566, 1311-1577, 1311-1985, 1316-1841, 1318-1581, 1323-1605, 1325-1558, 1329-1887, |
| | 1334-1918, 1338-1628, 1338-1729, 1341-1761, 1345-1900, 1346-1829, 1349-1964, 1351-1812, 1353-1620, 1353-1912, 1359-1801, |
| | 1368-1994, 1374-1930, 1374-1947, 1380-1543, 1380-1599, 1380-1630, 1380-1935, 1381-1619, 1381-1620, 1381-1645, 1383-1997, |
| | 1384-1990, 1390-1667, 1390-1924, 1391-1668, 1391-1783, 1401-1685, 1402-1495, 1403-1849, 1403-2002, 1404-1548, 1404-1667, |
| | 1404-1973, 1405-1687, 1410-1609, 1412-1641, 1412-1668, 1412-1673, 1427-1695, 1428-1693, 1432-1717, 1442-1733, 1444-1674, |
| | 1455-1771, 1465-1897, 1467-2001, 1468-1942, 1476-1703, 1485-2127, 1502-1792, 1506-1782, 1513-1753, 1513-1820, 1515-1759, |
| | 1515-1789, 1522-1889, 1527-1826, 1528-1797, 1536-2089, 1540-1774, 1543-1792, 1546-1793, 1563-1859, 1563-1891, 1568-1755, |
| | 1573-1843, 1573-1868, 1575-1810, 1575-1823, 1575-1826, 1578-2230, 1581-1830, 1584-1777, 1606-1911, 1607-1777, 1607-1891, |
| | 1618-1915, 1622-1856, 1638-2268, 1653-2001, 1667-2278, |
| | 1681-1917, 1683-1916, 1708-1994, 1718-1987, 1721-1987, 1722-1995, 1722-1998, 1746-2255, 1756-2001, 1788-2107, 1794-2408, |
| | 1840-2008, 1860-1931, 1863-1990, 1892-2002, 1992-2369, 2000-2572, 2001-2588, 2001-2714, 2003-2246, 2004-2523, 2006-2145, |
| | 2015-2251, 2028-2666, 2031-2256, 2031-2630, 2040-2507, 2040-2663, 2051-2355, 2062-2349, 2065-2359, 2076-2402, 2076-2532, |
| | 2080-2530, 2081-2347, 2081-2371, 2081-2751, 2082-2670, 2083-2373, 2085-2355, 2086-2610, 2091-2436, 2094-2630, 2094-2686, |
| | 2094-2716, 2094-2721, 2097-2729, 2106-2382, 2109-2367, 2109-2391, 2115-2374, 2117-2369, 2117-2656, 2119-2376, 2126-2350, |
| | 2131-2753, 2133-2472, 2139-2402, 2140-2669, 2141-2421, 2145-2392, 2146-2386, 2146-2388, 2146-2409, 2150-2971, 2152-2418, |
| | 2153-2857, 2158-2400, 2158-2402, 2160-2480, 2162-2432, 2165-2443, 2166-2428, 2167-2736, 2170-2435, 2170-2483, 2170-2568, |
| | 2171-2488, 2176-2412, 2176-2753, 2176-2765, 2191-2463, 2196-2432, 2200-2731, 2205-2406, 2205-2481, 2206-2495, 2209-2729, |
| | 2210-2472, 2211-2467, 2215-2436, 2215-2490, 2221-2828, 2223-2505, |
| | 2223-2506, 2227-2505, 2232-2524, 2237-2542, 2240-2578, 2242-2727, 2245-2633, 2247-2507, 2247-2535, 2249-2509, 2250-2514, |
| | 2250-2579, 2250-2708, 2251-2533, 2253-2775, 2256-2440, 2256-2511, 2256-2704, 2256-2755, 2256-2864, 2259-2818, 2261-2931, |
| | 2268-2584, 2269-2507, 2270-2501, 2270-2534, 2276-2811, 2279-2559, 2279-2788, 2279-2960, 2279-3083, 2286-2567, 2288-2550, |
| | 2289-2539, 2290-2530, 2295-2569, 2295-2572, 2295-2909, 2297-2507, 2298-2845, 2300-2412, 2301-2565, 2302-2534, 2304-2894, |
| | 2310-2574, 2310-2806, 2311-2581, 2312-2590, 2316-2943, 2317-2629, 2318-2671, 2319-2472, 2324-2558, 2327-3018, 2329-2579, |
| | 2331-2522, 2331-2539, 2335-2882, 2337-2560, 2338-2605, 2338-2969, 2343-2608, 2343-2618, 2343-2872, 2344-2612, 2345-2574, |
| | 2347-2597, 2348-2637, 2348-2640, 2349-2607, 2349-2609, 2349-2610, 2350-2607, 2353-2709, 2365-2583, 2365-2612, 2366-2638, |
| | 2368-2614, 2372-2632, 2382-2845, 2384-2573, 2384-2637, 2385-2679, 2387-2657, 2387-2948, 2400-2852, 2407-2663, 2409-2676, |
| | 2414-2624, 2417-2664, 2417-2665, 2421-2647, 2427-2587, |
| | 2427-2735, 2432-2681, 2435-2664, 2436-2724, 2436-2753, 2437-2697, 2437-2719, 2437-2757, 2437-2905, 2438-2895, 2439-2671, |
| | 2440-2673, 2440-2691, 2440-2714, 2440-2715, 2440-2717, 2441-2723, 2443-2664, 2443-2682, 2443-2713, 2444-2833, 2447-2746, |
| | 2458-2747, 2459-2687, 2460-3158, 2465-2855, 2466-2669, 2466-2674, 2472-2702, 2472-2705, 2472-3030, 2474-2699, 2474-2715, |
| | 2474-2731, 2475-3016, 2477-2768, 2481-2723, 2484-2708, 2484-2712, 2484-2718, 2484-2726, 2484-2781, 2485-2767, 2486-2568, |
| | 2486-2709, 2486-2739, 2486-2766, 2489-2790, 2494-2787, 2499-2714, 2499-2773, 2500-3183, 2502-2751, 2502-2764, 2502-3016, |
| | 2508-2828, 2508-2892, 2511-2753, 2511-2780, 2513-2721, 2513-2750, 2513-2752, 2515-2735, 2515-2741, 2519-3079, 2520-2786, |
| | 2525-2802, 2528-2781, 2530-2773, 2532-2734, 2532-2766, 2532-3078, 2532-3219, 2538-2752, 2541-2791, 2543-2797, 2543-3093, |
| | 2546-2800, 2546-3180, 2549-2841, 2550-2844, 2553-2798, 2553-2805, 2554-2851, 2555-3201, 2561-3215, 2563-2813, 2563-2833, |
| | 2563-2836, 2566-2852, 2567-2862, 2569-2857, 2573-2846, 2573-2855, |
| | 2575-3222, 2584-3430, 2587-3008, 2587-3173, 2588-2835, 2592-3203, 2593-2825, 2593-2861, 2594-2842, 2594-2859, 2595-2848, |
| | 2595-2988, 2600-2808, 2602-2809, 2602-2813, 2602-2831, 2602-2855, 2602-2888, 2602-3002, 2602-3207, 2605-2844, 2605-2851, |
| | 2605-3177, 2606-3146, 2607-2864, 2608-2870, 2608-3116, 2609-3220, 2619-2859, 2619-2863, 2619-3219, 2621-2873, 2623-2883, |
| | 2624-2853, 2627-2876, 2629-2920, 2633-2859, 2633-2877, 2637-2879, 2639-3178, 2643-2903, 2648-3429, 2652-2886, 2652-2899, |
| | 2654-2931, 2655-3216, 2656-2884, 2656-3216, 2657-2944, 2659-2927, 2664-3219, 2665-3056, 2666-2926, 2667-3166, 2667-3213, |
| | 2668-2931, 2672-2934, 2675-2951, 2676-2910, 2676-2920, 2677-2918, 2681-3142, 2683-2953, 2683-2962, 2685-2969, 2687-3204, |
| | 2689-2951, 2689-2982, 2689-3211, 2692-2825, 2693-2988, 2694-2726, 2696-3416, 2697-3216, 2702-2949, 2702-2951, 2703-2950, |
| | 2703-2951, 2703-2954, 2704-2986, 2705-2955, 2707-2945, 2707-2980, 2707-2989, 2710-3217, 2712-3216, 2714-2887, 2714-2924, |
| | 2716-2882, 2716-3214, 2717-3216, 2718-3205, 2729-2999, |
| | 2729-3216, 2732-2961, 2732-2975, 2733-3219, 2734-3216, 2736-3039, 2737-3005, 2737-3145, 2738-3200, 2739-3003, 2739-3176, |
| | 2739-3216, 2741-3209, 2742-3000, 2743-3317, 2744-3216, 2745-2956, 2745-2988, 2745-3005, 2746-2983, 2746-2985, 2746-3214, |
| | 2749-2983, 2750-3162, 2751-3008, 2751-3042, 2751-3054, 2751-3101, 2753-3203, 2753-3217, 2755-3016, 2756-3067, 2757-2975, |
| | 2757-3009, 2757-3028, 2757-3216, 2760-3003, 2760-3216, 2761-3200, 2763-3142, 2763-3214, 2764-3066, 2764-3217, 2765-3213, |
| | 2765-3346, 2767-3064, 2767-3214, 2768-3042, 2768-3214, 2770-2925, 2773-3204, 2773-3209, 2774-2979, 2774-3015, 2774-3213, |
| | 2774-3422, 2776-3020, 2776-3027, 2776-3210, 2778-3406, 2779-3129, 2779-3202, 2780-3114, 2782-3214, 2782-3216, 2783-2967, |
| | 2783-2988, 2783-3055, 2783-3116, 2786-2947, 2786-3217, 2787-3214, 2788-3023, 2789-3216, 2790-3217, 2792-3068, 2792-3214, |
| | 2794-3051, 2795-3023, 2796-3205, 2796-3217, 2797-3216, 2798-3034, 2798-3213, 2799-3024, 2799-3396, 2801-3214, 2803-3205, |
| | 2804-3214, 2804-3430, 2805-3213, 2806-3322, 2808-3214, 2812-3209, |
| | 2813-3085, 2814-3039, 2814-3069, 2814-3071, 2814-3076, 2814-3080, 2814-3205, 2814-3216, 2815-3204, 2815-3216, 2816-3205, |
| | 2816-3216, 2819-3216, 2820-3204, 2820-3205, 2820-3214, 2821-3227, 2822-3204, 2822-3214, 2823-3216, 2823-3217, 2824-3205, |
| | 2826-3205, 2826-3209, 2826-3213, 2826-3214, 2827-3212, 2828-3068, 2828-3396, 2829-3211, 2829-3214, 2830-3090, 2830-3205, |
| | 2830-3216, 2832-3013, 2832-3078, 2832-3110, 2832-3210, 2833-3143, 2833-3145, 2833-3173, 2834-3075, 2835-3485, 2839-3137, |
| | 2841-2917, 2841-3082, 2841-3090, 2841-3390, 2842-3213, 2843-3082, 2845-3073, 2845-3131, 2846-3073, 2847-3081, 2847-3093, |
| | 2847-3135, 2848-3214, 2849-3205, 2850-3097, 2850-3113, 2851-3071, 2852-3071, 2855-3205, 2856-3153, 2858-3086, 2858-3088, |
| | 2858-3205, 2859-3108, 2860-3113, 2860-3176, 2861-3119, 2861-3146, 2861-3455, 2862-3116, 2862-3214, 2864-3205, 2864-3457, |
| | 2865-3214, 2866-3233, 2870-3106, 2870-3214, 2870-3382, 2871-3214, 2874-3211, 2874-3464, 2876-3131, 2876-3132, 2877-3140, |
| | 2877-3205, 2878-3125, 2878-3138, 2880-3436, 2883-3214, |
| | 2883-3365, 2888-3139, 2890-3183, 2891-3160, 2893-3175, 2893-3201, 2893-3211, 2893-3217, 2895-3216, 2896-3131, 2897-2995 |
| | 2900-3149, 2901-3214, 2904-3158, 2904-3204, 2906-3156, 2910-3211, 2911-3155, 2911-3205, 2911-3209, 2913-3209, 2914-3204, |
| | 2914-3214, 2915-3475, 2917-3214, 2920-3163, 2920-3214, 2922-3170, 2922-3200, 2922-3210, 2923-3182, 2923-3210, 2923-3218, |
| | 2923-3292, 2923-3495, 2924-3203, 2924-3216, 2925-3184, 2925-3209, 2925-3213, 2926-3185, 2926-3209, 2930-3169, 2931-3205, |
| | 2931-3215, 2936-3205, 2939-3163, 2939-3203, 2940-3158, 2940-3212, 2940-3399, 2941-3204, 2941-3214, 2943-3175, 2943-3191, |
| | 2943-3214, 2943-3216, 2947-3205, 2952-3170, 2955-3202, 2955-3206, 2955-3213, 2955-3253, 2956-3172, 2957-3174, 2961-3159, |
| | 2961-3216, 2961-3265, 2965-3031, 2965-3217, 2967-3216, 2967-3485, 2969-3204, 2969-3216, 2970-3191, 2972-3484, 2973-3214, |
| | 2983-3209, 2983-3214, 2986-3216, 2986-3414, 2987-3215, 2987-3216, 2987-3240, 2989-3258, 2990-3215, 2991-3312, 2991-3495, |
| | 2993-3214, 2994-3204, 2994-3214, 2994-3216, 2994-3226, 2996-3209, |
| | 2996-3214, 3000-3216, 3000-3267, 3005-3216, 3005-3411, 3010-3205, 3012-3186, 3012-3204, 3013-3181, 3015-3216, 3015-3266, |
| | 3015-3277, 3015-3347, 3020-3469, 3020-3489, 3020-3495, 3021-3216, 3022-3495, 3025-3216, 3028-3495, 3031-3226, 3031-3309, |
| | 3031-3465, 3033-3214, 3034-3268, 3034-3469, 3035-3151, 3035-3216, 3035-3261, 3038-3216, 3039-3265, 3039-3315, 3040-3217, |
| | 3040-3293, 3044-3315, 3045-3468, 3046-3247, 3047-3495, 3048-3326, 3050-3205, 3051-3214, 3051-3317, 3051-3331, 3051-3456, |
| | 3052-3474, 3057-3472, 3059-3468, 3059-3492, 3061-3214, 3061-3216, 3061-3468, 3061-3472, 3062-3324, 3063-3491, 3064-3491, |
| | 3070-3468, 3070-3470, 3075-3411, 3077-3214, 3077-3216, 3078-3312, 3078-3468, 3079-3194, 3083-3216, 3083-3324, 3083-3345, |
| | 3086-3469, 3088-3214, 3088-3266, 3090-3472, 3093-3200, 3093-3205, 3095-3398, 3095-3469, 3096-3471, 3097-3308, 3097-3329, |
| | 3098-3348, 3098-3383, 3098-3470, 3099-3216, 3099-3318, 3100-3199, 3100-3204, 3100-3215, 3100-3216, 3100-3331, 3100-3332, |
| | 3100-3341, 3100-3354, 3101-3369, 3102-3193, 3102-3211, |
| | 3102-3216, 3102-3217, 3102-3220, 3102-3232, 3102-3311, 3102-3325, 3102-3327, 3102-3328, 3102-3338, 3102-3343, 3102-3350, |
| | 3102-3358, 3102-3359, 3102-3360, 3102-3365, 3102-3368, 3102-3369, 3102-3370, 3102-3376, 3102-3380, 3102-3384, 3102-3394, |
| | 3102-3396, 3103-3214, 3103-3216, 3103-3217, 3104-3216, 3104-3357, 3105-3193, 3105-3216, 3105-3232, 3105-3335, 3105-3350, |
| | 3105-3354, 3105-3355, 3105-3362, 3105-3363, 3105-3368, 3105-3377, 3105-3386, 3105-3392, 3106-3215, 3106-3216, 3106-3217, |
| | 3106-3287, 3106-3337, 3106-3345, 3106-3357, 3106-3377, 3107-3210, 3107-3361, 3107-3363, 3107-3395, 3108-3468, 3108-3471, |
| | 3111-3366, 3112-3329, 3112-3338, 3112-3341, 3112-3342, 3112-3349, 3112-3357, 3112-3379, 3112-3380, 3112-3390, 3113-3216, |
| | 3114-3214, 3115-3210, 3115-3217, 3115-3370, 3115-3380, 3115-3388, 3115-3390, 3115-3471, 3116-3380, 3117-3354, 3118-3353, |
| | 3120-3319, 3121-3284, 3121-3355, 3121-3363, 3122-3362, 3122-3468, 3128-3328, 3128-3331, 3128-3362, 3128-3381, 3128-3410, |
| | 3130-3294, 3131-3216, 3135-3475, 3138-3476, 3138-3491, 3140-3384, |
| | 3140-3422, 3140-3470, 3143-3374, 3149-3480, 3149-3495, 3152-3205, 3158-3495, 3171-3432, 3173-3373, 3173-3430, 3173-3482, |
| | 3174-3442, 3177-3449, 3178-3420, 3179-3473, 3182-3469, 3183-3468, 3184-3463, 3184-3495, 3186-3468, 3187-3468, 3192-3469, |
| | 3195-3470, 3195-3471, 3196-3469, 3197-3471, 3198-3468, 3198-3470, 3199-3471, 3210-3478, 3218-3477, 3219-3333, 3219-3465, |
| | 3219-3476, 3220-3477, 3220-3480, 3227-3420, 3248-3482, 3248-3488, 3254-3487, 3255-3469, 3263-3489, 3265-3469, 3267-3471, |
| | 3268-3465, 3292-3463, 3292-3485, 3314-3468, 3322-3488, 3354-3482 |
| 27/ | 1-782, 183-649, 245-649, 643-1067, 669-1052, 696-844, 767-1097, 816-1097, 816-1101, 816-1103, 999-1097, 1034-1097, |
| 3871329CB1/ | 1034-1098, 1034-1102, 1034-1103, 1034-1244, 1034-1544, 1034-1545, 1034-1565, 1034-1647, 1034-1684, 1039-1080, |
| 3720 | 1204-1896, 1265-1801, 1345-2065, 1561-2216, 1635-1926, 1661-2317, 1676-2365, 1700-2253, 1727-2415, 1754-2375, |
| | 1765-2380, 1765-2409, 1791-2174, 1791-2473, 1919-2469, 1920-2720, 1974-2642, 2105-2701, 2123-2687, 2209-2891, 2344-3162, |
| | 2350-2927, 2647-3294, 2714-3546, 2733-3446, 2748-3422, 2765-3048, 2770-3433, 2781-3125, 2787-3407, 2817-3433, 2820-3365, |
| | 2820-3522, 2837-3522, 2851-3550, 2865-3515, 2873-3448, 2889-3590, 2904-3468, 2918-3538, 2965-3528, 2986-3714, |
| | 3008-3663, 3008-3717, 3046-3720 |
| 28/ | 1-550, 36-550, 105-548, 105-752, 338-569, 352-569, 416-781, 416-790, 470-951, 490-529, 557-625, 557-829, |
| 1681386CB1/ | 557-847, 557-982, 557-987, 557-1132, 557-1133, 563-1199, 575-1013, 649-974, 704-1401, 719-990, 719-1183, 914-1380, |
| 6119 | 929-1529, 1116-1403, 1138-1199, 1155-1723, 1276-1802, 1520-2076, 1563-1865, 1563-2117, 1572-1833, 1572-1837, 1615-2129, |
| | 1757-2161, 1763-1928, 1763-2298, 1775-2118, 1786-2272, 1808-2458, 1823-2082, 1823-2322, 1876-2576, 1887-2364, 1888-2364, |
| | 1909-2054, 1935-2551, 1979-2619, 2039-2619, 2088-2506, 2135-2867, 2135-2896, 2146-2709, 2150-2707, 2189-2591, 2193-2744, |
| | 2223-2952, 2231-2737, 2245-2494, 2258-2722, 2320-2572, 2320-2864, 2333-2927, 2342-2553, 2351-2888, 2379-2888, 2405-2915, |
| | 2426-3079, 2427-2938, 2579-2952, 2597-3052, 2669-2909, 2669-3073, 2669-3105, 2685-6060, 2693-2781, 2693-2982, 2835-3660, |
| | 2853-3660, 2906-3035, 2916-3182, 2945-3690, 3118-3697, 3132-3419, 3195-3749, 3221-3914, 3233-4042, 3255-3485, 3341-3522, |
| | 3342-4072, 3363-3807, 3404-4075, 3432-3978, 3432-4278, 3533-3703, 3534-3770, 3534-4124, 3613-4229, 3657-4100, |
| | 3714-4160, 3771-4399, 3776-4470, 3857-4462, 3885-4353, 3903-4549, 3937-4467, 3957-4576, 3987-4353, 3987-4603, 4028-4738, |
| | 4039-4589, 4075-4381, 4137-4695, 4146-4402, 4146-4630, 4146-4657, 4146-4658, 4146-4689, 4146-4694, 4146-4697, 4146-4702, |
| | 4146-4724, 4146-4743, 4159-4657, 4167-4574, 4235-4738, 4291-4955, 4318-4937, 4327-4787, 4341-4448, 4355-4926, 4432-4951, |
| | 4464-5228, 4521-5153, 4533-5098, 4570-5150, 4572-5150, 4639-5385, 4641-5194, 4684-5460, 4731-5133, 4731-5314, 4731-5372, |
| | 4743-5268, 4778-4994, 4800-5557, 4810-5332, 4810-5437, 4810-5440, 4814-5298, 4829-5075, 4834-5074, 4834-5124, 4835-5468, |
| | 4845-5385, 4848-5483, 4849-5474, 4852-5160, 4884-5384, 4884-5419, 4884-5443, 4884-5523, 4898-5509, 4915-5454, 4916-5444, |
| | 4928-5145, 4929-5170, 4943-5563, 4946-5570, 4951-5572, 4958-5622, 4961-5674, 4963-5527, 4982-5609, 4987-5582, 4997-5254, |
| | 5002-5139, 5014-5664, 5016-5660, 5018-5600, 5020-5640, 5027-5324, 5041-5750, 5046-5553, 5049-5649, 5050-5675, 5060-5533, |
| | 5060-5558, 5066-5099, 5067-5317, 5067-5697, 5070-5560, 5070-5566, |
| | 5070-5629, 5076-5500, 5083-5322, 5083-5375, 5087-5540, 5098-5629, 5101-5660, 5102-5203, 5107-5680, 5110-5677, 5110-5700, |
| | 5113-5511, 5116-5393, 5117-5698, 5123-5512, 5128-5309, 5131-5620, 5131-5824, 5131-5838, 5148-5693, 5161-5419, 5165-5671, |
| | 5167-5497, 5168-5782, 5172-5569, 5174-5582, 5178-5662, 5201-5320, 5201-5447, 5201-5494, 5201-5787, 5207-5836, 5211-5771, |
| | 5215-5590, 5219-5706, 5234-5308, 5236-5756, 5242-5784, 5257-5836, 5265-5436, 5265-5647, 5282-5947, 5290-5569, 5290-5604, |
| | 5307-5424, 5307-5924, 5312-5749, 5316-5554, 5316-5572, 5316-5583, 5316-5600, 5316-5602, 5316-5611, 5326-5902, 5334-5589, |
| | 5336-5602, 5355-5648, 5357-5978, 5360-5649, 5364-5675, 5365-5746, 5376-5940, 5391-5674, 5392-5969, 5392-6035, 5393-6047, |
| | 5402-5639, 5410-5660, 5415-6039, 5417-6019, 5424-5715, 5425-5991, 5426-5905, 5434-6072, 5435-6040, 5445-5637, 5447-6050, |
| | 5447-6051, 5451-6066, 5859-6119 |
| 29/ | 1-237, 1-557, 1-1073, 2-332, 4-240, 4-252, 4-271, 4-305, 8-224, 9-486, 12-344, 16-281, 18-272, 21-287, 26-317, |
| 7500938CB1/ | 27-291, 27-422, 27-510, 28-276, 28-297, 28-539, 29-248, 29-277, 29-488, 29-557, 30-585, 32-308, 32-557, 34-128, 34-286, |
| 1151 | 34-308, 34-316, 34-455, 37-346, 37-413, 37-424, 37-501, 37-511, 37-556, 37-557, 38-274, 38-282, 38-298, 38-337, 39-287, |
| | 39-313, 40-280, 40-282, 40-292, 40-313, 40-378, 41-285, 41-292, 42-271, 42-296, 42-355, 42-557, 43-301, 43-320, 43-321, |
| | 43-327, 43-350, 43-463, 43-554, 44-225, 44-255, 44-276, 44-280, 44-282, 44-288, 44-294, 44-300, 44-317, 44-331, 44-344, |
| | 44-357, 44-516, 45-291, 45-305, 47-311, 47-313, 48-286, 48-305, 48-321, 48-481, 49-263, 49-298, 49-299, 49-302, 49-324, |
| | 50-225, 50-244, 50-282, 50-284, 50-286, 50-293, 50-300, 50-306, 50-309, 50-315, 50-319, 50-331, 50-343, 50-357, 50-428, |
| | 50-518, 50-557, 52-314, 52-322, 52-339, 52-557, 54-283, 54-300, 54-325, 54-335, 54-338, 55-230, 55-311, 55-349, 55-479, |
| | 55-557, 56-302, 56-374, 57-292, 57-304, 57-305, 57-352, 57-382, 57-384, 57-557, 57-575, 58-294, 58-455, 61-278, 62-286, |
| | 63-300, 63-358, 63-373, 64-185, 64-297, 64-310, 65-557, 67-192, 67-197, 67-208, 67-281, 67-303, 67-326, 67-377, 68-324, |
| | 68-327, 70-317, 74-332, 75-316, 75-334, 75-345, 79-399, 81-391, 83-281, 83-515, 84-353, 90-329, 90-343, 90-557, 92-358, |
| | 96-329, 96-339, 96-346, 96-546, 97-557, 121-415, 122-353, 122-368, |
| | 123-358, 128-380, 152-327, 156-399, 169-390, 171-425, 179-515, 210-404, 211-514, 212-508, 224-484, 226-469, 246-494, |
| | 246-504, 259-484, 259-497, 259-506, 265-506, 265-536, 266-498, 269-540, 269-544, 270-538, 274-550, 274-559, 277-500, |
| | 281-486, 290-533, 297-519, 297-557, 301-518, 306-548, 309-517, 316-557, 337-554, 347-557, 356-546, 368-503, 376-546, |
| | 399-534, 556-781, 557-723, 557-767, 557-768, 557-776, 557-787, 557-795, 557-797, 557-808, 557-809, 557-810, 557-986, |
| | 557-1050, 557-1112, 560-1032, 562-800, 574-755, 578-835, 578-1095, 590-846, 594-840, 599-847, 600-822, 602-852, |
| | 603-850, 603-872, 606-849, 606-878, 606-888, 610-895, 611-835, 611-893, 618-804, 618-819, 618-885, 618-888, 618-898, |
| | 624-913, 627-883, 629-856, 629-901, 630-924, 634-890, 638-740, 638-841, 638-876, 656-924, 660-923, 661-907, |
| | 663-926, 665-811, 665-984, 666-884, 666-890, 666-904, 666-1084, 667-911, 670-859, 672-798, 672-1142, 674-907, |
| | 674-916, 675-910, 675-935, 679-935, 681-1061, 682-933, 687-1084, 696-1007, 696-1071, 700-860, 700-980, 713-1007, |
| | 716-979, 716-1147, 725-794, 729-791, 741-956, 742-1012, 744-814, 748-983, 750-1071, 755-993, 755-998, 755-1019, |
| | 764-1030, 771-1046, 775-993, 778-1004, 778-1013, 779-1055, 779-1073, |
| | 791-1072, 792-1017, 793-1078, 794-1036, 796-1016, 796-1090, 797-907, 797-1044, 797-1076, 799-1052, 803-1013, 803-1064, |
| | 811-1082, 814-985, 814-1088, 816-1044, 825-1074, 834-1006, 836-1082, 857-1076, 859-1085, 868-1127, 884-1086, 893-987, |
| | 899-1151, 901-1048, 901-1144, 921-1135, 944-1083 |
| 30/ | 1-670, 93-700, 346-639, 407-819, 407-1016, 407-1089, 407-1141, 407-1192, 407-1277, 408-1097, |
| 90055441CB1/ | 408-1199, 408-1273, 408-1276, 408-1277, 415-1277, 496-1277 |
| 1277 | |
| 31/ | 1-217, 1-1036, 19-255, 24-290, 26-310, 30-255, 30-296, 37-218, 43-218, 51-504, 51-505, 89-352, 132-385, 219-355, |
| 7500936CB1/ | 224-815, 230-1013, 234-871, 236-555, 238-851, 241-553, 244-883, 249-883, 251-386, 253-529, 258-527, 261-545, 262-540, |
| 1041 | 262-859, 266-919, 267-724, 270-738, 288-544, 288-577, 288-905, 295-594, 296-557, 301-569, 302-552, 305-549, 305-608, |
| | 308-551, 311-820, 311-905, 311-923, 312-565, 313-538, 313-539, 314-986, 318-709, 333-632, 338-1038, 343-547, 349-575, |
| | 349-588, 349-607, 349-670, 350-605, 355-611, 363-810, 364-614, 366-932, 369-606, 370-947, 370-1004, 376-625, 376-981, |
| | 378-637, 380-668, 382-530, 382-626, 382-979, 384-615, 384-659, 385-644, 412-990, 413-626, 414-955, 416-978, 419-697, |
| | 419-709, 420-657, 425-664, 426-668, 426-688, 427-691, 427-1023, 435-634, 435-924, 436-643, 436-689, 436-760, 438-1032, |
| | 445-626, 445-670, 448-742, 450-716, 450-841, 451-993, 455-688, 455-766, 461-765, 464-763, 464-771, 468-575, |
| | 468-697, 468-712, 468-735, 469-776, 471-755, 472-717, 472-731, 476-717, 476-756, 476-757, 481-739, 481-1041, |
| | 483-978, 484-988, 488-703, 488-947, 488-950, 491-683, 492-756, 493-611, 493-613, 496-758, 496-771, 496-949, |
| | 496-1041, 497-772, 498-686, 498-730, 498-1013, 502-739, 506-773, 515-760, 518-731, 520-744, 520-750, |
| | 523-995, 525-763, 537-718, 541-798, 541-1041, 553-809, 557-803, 562-810, 563-785, 565-815, 566-813, 569-812, |
| | 569-841, 569-851, 573-858, 574-798, 574-856, 581-767, 581-848, 587-876, 590-846, 592-819, 593-887, 601-703, |
| | 601-804, 601-839, 619-887, 623-886, 624-870, 628-774, 628-947, 629-867, 629-1041, 630-874, 635-761, 638-873, |
| | 638-898, 642-898, 645-896, 650-1041, 659-970, 659-1034, 663-943, 679-1041, 688-757, 692-754, 704-919, |
| | 707-777, 711-946, 713-1034, 741-976, 756-1041 |
| 32/ | 1-337, 1-2589, 205-859, 205-895, 233-376, 245-801, 290-1226, 291-1009, 299-1087, 301-464, 306-975, |
| 7500950CB1/ | 329-768, 378-514, 401-1058, 414-1115, 446-963, 449-1061, 461-1149, 467-956, 484-943, 490-1081, 528-814, |
| 2745 | 541-1239, 574-1165, 582-1189, 585-973, 602-902, 606-1267, 608-1145, 619-1129, 622-1263, 625-802, 637-910, |
| | 653-1227, 657-1193, 663-1262, 673-1381, 681-1069, 695-1285, 697-1165, 708-1089, 710-945, 710-1207, 748-1293, |
| | 753-1360, 758-1336, 768-1089, 773-1407, 781-1521, 788-1336, 804-1301, 854-1495, 877-1356, 925-1605, |
| | 1004-1227, 1201-2087, 1377-2195, 1419-2098, 1441-2204, 1460-1991, 1487-2116, 1512-2118, 1521-2168, 1524-2069, |
| | 1531-2151, 1541-2195, 1544-2190, 1547-2212, 1548-2043, 1558-2049, 1562-2239, 1576-2157, 1579-2201, 1583-2199, |
| | 1608-2087, 1608-2117, 1615-2280, 1637-1912, 1649-2089, 1650-1939, 1651-1884, 1651-2302, 1662-2311, 1692-2001, 1716-1959, |
| | 1739-2040, 1783-1991, 1796-2206, 1816-2389, 1834-2405, 1855-2539, 1860-2222, 1877-2329, 1888-2220, 1903-2426, 1906-2204, |
| | 1936-2224, 1954-2387, 1976-2539, 1989-2511, 1991-2451, 1993-2266, 2006-2575, |
| | 2018-2589, 2048-2547, 2059-2538, 2075-2601, 2079-2294, 2090-2427, 2153-2397, 2159-2299, 2162-2425, 2162-2429, 2171-2454, |
| | 2176-2299, 2180-2741, 2184-2650, 2235-2461, 2327-2535, 2476-2745 |
| 33/ | 1-191, 1-224, 1-239, 1-242, 1-253, 1-264, 1-268, 1-270, 1-278, 1-290, 1-292, 1-295, 1-344, 1-574, 2-250, 2-265, 3-237, 3-242, 7-279, |
| 7500854CB1/ | 8-244, 11-265, 13-277, 28-270, 45-330, 47-308, 56-318, 56-342, 57-276, 60-308, 67-268, 103-344, 105-340, 109-233, 120-571, |
| 627 | 344-583, 349-591, 363-587, 363-593, 372-595, 376-582, 384-627, 421-571, 432-569, 440-619, 479-571, 495-618, 507-571 |
| 34/ | 1-641, 61-616, 129-674, 456-815, 628-1074, 778-1369, 858-1096, 858-1464, 1070-1367, 1102-1255, 1333-1445, |
| 2754176CB1/ | 1333-1808, 1333-1920, 1333-1922, 1333-1939, 1333-1945, 1333-1960, 1336-1820, 1375-1910, 1402-1626, |
| 5899 | 1402-1834, 1415-1785, 1484-1935, 1507-2017, 1530-2002, 1639-1820, 1639-2125, 1673-2152, 1701-2292, 1757-1984, |
| | 1772-1993, 1801-2272, 1805-2372, 1810-1969, 1826-2042, 1844-2390, 1845-2092, 1894-2439, 1912-2135, 1928-2107, |
| | 1950-2366, 1957-2492, 1984-2626, 2012-2198, 2019-2477, 2034-2335, 2080-2254, 2118-2592, 2120-2720, 2205-2746, |
| | 2235-2328, 2263-2748, 2292-2507, 2331-2533, 2344-2742, 2347-2591, 2515-2719, |
| | 2523-2819, 2523-2967, 2523-3058, 2644-2991, 2647-3117, 2647-3138, 2663-2897, 2810-3302, 2830-3331, 2946-3312, 2981-3244, |
| | 2986-3189, 2989-3511, 3010-3163, 3023-3297, 3040-3314, 3074-3643, 3123-3393, 3146-3430, 3147-3419, 3202-3440, 3202-3759, |
| | 3234-3465, 3278-3485, 3318-3912, 3470-3679, 3476-4112, 3484-3741, 3485-4009, 3533-3767, 3571-3815, 3573-4021, 3584-4013, |
| | 3664-3934, 3669-3883, 3698-3960, 3755-4014, 3759-3992, 3773-4042, |
| | 3787-4129, 3824-4135, 3835-4238, 3850-4500, 3872-4144, 3924-4159, 3940-4129, 3980-4233, 4016-4508, 4027-4517, 4037-4508, |
| | 4040-4476, 4052-4327, 4086-4347, 4086-4570, 4089-4362, 4093-4476, 4094-4508, 4096-4508, 4105-4516, 4105-4517, 4138-4374, |
| | 4138-4509, 4143-4493, 4160-4511, 4218-4510, 4221-4533, 4270-4870, 4279-4478, 4279-4510, 4308-4651, 4312-4579, 4443-4988, |
| | 4462-5061, 4620-4894, 4731-4984, 4764-5017, 4764-5034, 4764-5341, 4770-5066, 4846-5111, 4887-5467, 5080-5693, 5169-5403, |
| | 5224-5422, 5233-5495, 5326-5580, 5326-5857, 5393-5658, 5393-5663, 5419-5696, 5444-5898, 5638-5899 |
| 35/ | 1-255, 1-284, 4-381, 6-1524, 20-275, 47-267, 47-331, 49-318, 67-236, 67-302, 91-281, 91-360, 116-471, |
| 7503408CB1/ | 145-389, 160-410, 161-441, 172-388, 187-499, 204-477, 283-503, 455-706, 463-716, 463-927, 470-761, 504-1107, 509-878, |
| 1542 | 511-1043, 518-1202, 519-815, 519-1176, 520-943, 520-1112, 521-744, 521-757, 529-992, 530-1022, 536-809, 536-1158, |
| | 538-919, 540-933, 545-1190, 567-1037, 569-822, 571-1055, 572-1478, 585-812, 586-867, 587-1154, 588-1328, 589-908, |
| | 595-836, 622-877, 629-1154, 630-837, 630-1084, 630-1154, 640-864, 651-955, 653-911, 659-1112, 660-1136, 661-924, |
| | 663-1314, 665-1277, 667-910, 669-924, 672-963, 673-939, 676-758, 676-1380, 677-949, 677-1247, 680-1309, 686-982, |
| | 686-1233, 693-1356, 697-962, 697-1070, 699-1398, 715-1415, 719-1018, |
| | 727-1535, 729-1044, 731-1010, 736-1271, 736-1291, 752-1522, 753-1024, 754-1179, 756-1291, 756-1354, 757-1242, 769-1514, |
| | 775-867, 791-1017, 791-1358, 796-1033, 803-1382, 809-1453, 820-1380, 821-1087, 822-1137, 829-1469, 831-1091, |
| | 834-1142, 839-1112, 839-1143, 842-1094, 844-1421, 844-1463, 845-1122, 845-1366, 847-1281, 853-1115, 857-1385, |
| | 859-1000, 859-1089, 861-1036, 865-1494, 874-1140, 874-1261, 874-1506, 877-1519, 886-1540, 887-1469, 888-1100, 892-1477, |
| | 892-1536, 894-1539, 896-1153, 897-1542, 902-1189, 902-1190, 913-1527, 917-1510, 923-1202, 924-1510, 930-1201, |
| | 930-1477, 935-1537, 939-1277, 944-1172, 950-1104, 961-1262, 962-1246, 965-1519, 967-1251, 967-1314, 974-1513, |
| | 987-1271, 992-1315, 1011-1259, 1012-1139, 1024-1539, 1029-1254, 1029-1366, 1029-1514, 1031-1540, 1032-1297, 1038-1530, |
| | 1055-1525, 1058-1525, 1067-1530, 1077-1527, 1078-1527, 1081-1528, 1097-1542, 1100-1540, 1102-1452, 1108-1158, |
| | 1180-1473, 1209-1539, 1209-1542, 1286-1527, 1326-1527, 1398-1542 |
| 36/ | 1-161, 1-301, 1-336, 10-803, 46-342, 46-840, 54-161, 63-324, 67-787, 69-258, 91-160, 159-270, 160-391, |
| 71086982CB1/ | 190-840, 221-594, 265-431, 268-1076, 310-764, 310-1014, 311-795, 312-1053, 320-1089, 343-1095, 381-637, 381-946, |
| 2997 | 423-958, 425-1085, 470-1083, 535-618, 541-669, 541-813, 541-825, 541-989, 566-1194, 585-1141, 589-1229, 618-1370, |
| | 635-1225, 660-853, 690-1157, 707-1001, 713-929, 717-1249, 726-943, 726-1248, 737-1017, 743-904, 749-1013, 749-1020, |
| | 768-1047, 780-1025, 780-1193, 801-1338, 809-1363, 820-1064, 845-1093, 1095-1790, 1203-1775, 1231-1849, 1236-1693, |
| | 1273-1921, 1274-1543, 1281-1898, 1284-1944, 1292-1865 |
| | 1302-1844, 1302-1908, 1316-1965, 1319-1868, 1320-1665, 1321-1775, 1324-1952, 1328-1964, 1329-1912, 1340-1493, 1346-2005, |
| | 1347-1966, 1357-1577, 1359-1602, 1366-1948, 1372-1592, 1382-1638, 1408-1790, 1412-1898, 1416-1712, 1419-2016, 1430-1627, |
| | 1464-1804, 1506-2025, 1529-1927, 1555-2076, 1562-2119, 1566-2177, 1580-1868, 1591-2184, 1594-2089, 1596-2183, 1597-2131, |
| | 1597-2177, 1618-1806, 1619-2191, 1622-1887, 1626-1894, 1628-1906, 1630-1904, 1630-2138, |
| | 1630-2193, 1640-1874, 1640-2360, 1644-2224, 1645-1821, 1645-2153, 1645-2226, 1660-1940, 1671-1913, 1674-2217, 1676-1977, |
| | 1677-2310, 1687-1874, 1694-1889, 1715-2226, 1730-2270, 1739-2332, 1745-1908, 1748-2217, 1771-2094, 1772-2224, 1775-2203, |
| | 1776-2317, 1786-2195, 1791-2036, 1792-2095, 1792-2341, 1793-2309, 1796-2277, 1802-2203, 1802-2205, 1802-2213, 1805-2325, |
| | 1820-1894, 1827-2181, 1831-2219, 1837-2212, 1842-2221, 1843-2093, 1847-2180, 1853-2462, 1877-2102, 1904-2110, 1906-2198, |
| | 1927-2228, 1951-2188, 1957-2214, 2190-2778, 2552-2997, 2587-2837, 2616-2934, 2630-2909, 2720-2997 |
| 37/ | 1-3383, 69-258, 660-1427, 869-1785, 1197-1785, 1347-1437, 1347-1501, 1347-1696, 1347-1762, 1347-1767, 1347-1769, |
| 7506367CB1/ | 1347-1874, 1350-1721, 1355-1598, 1359-1931, 1364-1656, 1365-1760, 1387-2005, 1429-2077, 1430-1699, 1437-2054, 1438-1476, |
| 3383 | 1440-2100, 1448-2021, 1454-1922, 1458-2000, 1458-2064, 1472-2121, 1475-2024, 1476-1821, 1477-1931, 1477-2004, |
| | 1480-2108, 1484-2120, 1485-2068, 1496-1649, 1502-2161, 1503-2122, 1513-1733, 1515-1758, 1522-2104, 1528-1748, 1538-1794, |
| | 1548-1957, 1564-1946, 1568-2054, 1571-2259, 1572-1868, 1575-2172, 1586-1783, 1620-1960, 1662-2181, 1685-2083, |
| | 1711-2232, 1718-2275, 1722-2333, 1736-2024, 1747-2299, 1747-2340, 1750-2245, 1752-2339, 1753-2287, 1753-2333, 1774-1962, |
| | 1775-2347, 1778-2043, 1782-2050, 1784-2062, 1786-2060, 1786-2294, 1786-2349, 1793-2153, 1796-2030, 1796-2515, 1800-2380, |
| | 1801-1977, 1801-2309, 1801-2382, |
| | 1816-2096, 1827-2069, 1830-2373, 1832-2133, 1833-2466, 1843-2030, 1850-2045, 1871-2382, 1878-2460, 1886-2426, 1895-2487, |
| | 1904-2373, 1927-2250, 1928-2380, 1931-2359, 1932-2473, 1942-2351, 1947-2192, |
| | 1948-2251, 1948-2496, 1949-2465, 1952-2433, 1958-2361, 1961-2481, 1976-2050, 1983-2337, 2009-2616, 2062-2354, 2083-2384 |
| | 2107-2344, 2244-2751, 2346-2931, 2349-3031, 2367-3104, 2376-3123, 2387-3221, 2407-3282, 2419-3063, 2437-3012, 2472-3048, |
| | 2537-3107, 2537-3114, 2542-3219, 2547-3126, 2549-3205, 2554-3081, 2558-3066, 2565-3254, 2578-3142, 2580-3229, 2619-3271, |
| | 2630-3300, 2633-3102, 2633-3254, 2639-3248, 2643-3252, 2644-3200, 2645-3124, 2655-3185, 2655-3379, 2659-3255, 2663-3295, |
| | 2674-3338, 2679-3383, 2685-3327, 2699-3184, 2732-3189, 2740-2990, 2755-3262, 2761-3216, 2769-3087, 2783-3062, 2873-3150 |
| 38/ | 1-309, 16-225, 55-722, 71-663, 82-681, 152-737, 201-748, 225-530, 306-844, 627-1123, 852-1461, 942-1469, |
| 1414020CB1/ | 976-1201, 1117-1717, 1365-1947, 1833-2310, 1984-2205, 1994-2336, 2026-2279, 2026-2721, 2027-2377, 2132-2550, 2147-2894, |
| 3789 | 2267-2504, 2300-2570, 2300-2572, 2339-2786, 2358-2611, 2358-2865, 2411-2862, 2442-2950, 2455-2795, 2458-2743, |
| | 2483-2756, 2488-3004, 2494-3152, 2496-2759, 2499-3151, 2500-2792, 2502-3182, 2517-2777, 2522-2812, 2522-2815, |
| | 2522-2825, 2541-2784, 2541-3024, 2541-3152, 2548-2824, 2557-2622, 2567-3111, 2572-2854, 2572-2855, 2574-2994, |
| | 2599-3296, 2604-3242, 2612-3152, 2623-2861, 2624-2898, 2627-2769, 2646-3243, 2669-3287, 2683-3316, 2683-3317, |
| | 2724-3298, 2738-3072, 2749-2952, 2756-2971, 2756-3310, 2761-3388, 2770-3398, 2770-3431, 2791-3051, 2792-2990, |
| | 2793-3409, 2800-3277, 2800-3283, 2811-3255, 2821-3364, 2830-3391, 2835-3444, 2841-3051, 2844-3519, 2858-3153, |
| | 2869-3439, 2869-3555, 2871-3123, 2871-3286, 2871-3414, 2879-3557, 2892-3511, 2923-3473, |
| | 2925-3294, 2936-3225, 2941-3176, 2947-3466, 2948-3194, 2967-3241, 2992-3515, |
| | 2999-3649, 3006-3222, 3006-3550, 3015-3511, 3017-3728, 3034-3533, 3038-3503, 3047-3551, 3053-3537, 3069-3715, 3071-3731 |
| | 3086-3728, 3095-3728, 3098-3351, 3102-3739, 3105-3326, 3109-3724, 3110-3763, 3125-3739, 3141-3375, 3141-3567, 3141-3735, |
| | 3141-3743, 3142-3399, 3142-3626, 3142-3672, 3144-3711, 3144-3763, 3152-3756, 3155-3731, 3195-3513, 3196-3421, 3196-3764, |
| | 3209-3467, 3259-3606, 3264-3553, 3268-3761, 3279-3761, 3308-3789, 3320-3553, 3342-3581, 3348-3545, 3388-3757, 3474-3717 |
| 39/ | 1-294, 209-711, 215-462, 215-721, 215-727, 215-781, 215-800, 215-801, 215-817, 215-828, 215-863, 215-876, |
| 7621128CB1/ | 215-883, 215-902, 215-941, 232-477, 232-768, 239-508, 239-514, 239-800, 239-816, 247-596, 295-508, 295-526, 295-548, |
| 4174 | 295-606, 314-1056, 321-1059, 334-870, 339-978, 385-909, 403-1047, 414-700, 417-1046, 426-1034, 524-1139, 535-993, |
| | 549-1256, 592-1023, 640-1188, 688-1265, 725-1464, 735-2360, 736-1470, 749-1642, 750-1300, 761-1639, 780-1642, |
| | 806-1642, 879-1642, 881-1451, 949-1642, 960-1405, 1018-1475, 1040-1274, 1040-1315, 1040-1317, 1042-1446, 1092-1475, |
| | 1108-1475, 1114-1475, 1165-1475, 1230-1475, 1240-1464, 1488-1768, 1555-2112, 1555-2217, 1692-1916, 1711-1907, |
| | 2112-2705, 2125-2781, 2179-2944, 2179-2973, 2308-2605, 2308-2851, |
| | 2308-2951, 2328-2622, 2361-2920, 2361-2972, 2445-3215, 2487-3025, 2517-2719, 2554-3215, 2724-3379, 2785-3401, 2785-3413, |
| | 2790-3329, 2993-3650, 3112-3212, 3384-3888, 3386-3648, 3403-3674, 3525-3748, 3546-4074, 3647-3946, 3672-3985, 3740-3978, |
| | 3740-4159, 3815-4174 |
| 40/ | 1-263, 3-222, 5-744, 12-248, 12-288, 12-611, 12-655, 12-659, 12-681, 13-312, 25-197, 29-137, 32-464, 35-218, |
| 7505822CB1/ | 35-224, 37-238, 37-240, 38-210, 42-255, 44-267, 48-283, 57-262, 57-272, 57-285, 61-285, 64-285, 66-285, 66-316, 82-348, |
| 811 | 104-284, 109-219, 149-688, 192-444, 200-416, 209-455, 209-575, 209-741, 225-658, 282-562, 282-750, 282-757, 286-750, |
| | 288-687, 288-771, 290-753, 294-766, 302-553, 305-750, 314-748, 334-441, 341-631, 356-751, 364-589, 382-748, 386-748, |
| | 390-811, 402-750, 424-757, 426-750, 443-756, 477-732, 477-747, 482-567, 482-756, 503-748, 503-753, 504-755, |
| | 512-669, 593-675, 593-686, 633-722 |
| 41/ | 1-518, 36-621, 90-772, 267-589, 284-941, 385-1059, 429-721, 721-981, 721-1219, 773-1105, 822-1387, |
| 71607945CB1/ | 958-1335, 976-1508, 1032-1294, 1043-1571, 1069-1728, 1085-1550, 1090-1796, 1182-1210, 1183-1838, 1196-1746, |
| 4430 | 1215-1827, 1221-1854, 1260-1792, 1277-1847, 1283-1834, 1287-1938, 1309-1892, 1398-1811, 1412-2018, 1417-1844, |
| | 1429-1970, 1443-2064, 1453-2104, 1461-2098, 1469-1986, 1469-1989, 1478-1994, 1503-2117, 1557-1840, 1606-2051, |
| | 1624-2253, 1635-2349, 1771-2370, 1800-2303, 1826-2263, 1861-2356, 1875-2430, 1890-2108, 1939-2357, 1946-2555, |
| | 1954-2396, 1960-2500, 1968-2269, 2043-2629, 2046-2692, 2119-2722, 2194-2483, 2345-3051, 2386-2629, 2386-2891, |
| | 2464-2960, 2488-2765, 2491-2693, 2491-3211, 2497-3121, 2550-2858, 2550-3067, 2552-3121, 2558-3258, 2583-3119, |
| | 2590-2828, 2600-3327, 2606-3327, 2621-2871, 2644-3227, 2653-3367, 2655-2952, 2662-3335, 2666-3184, 2718-3212, |
| | 2810-3482, 2825-3380, 2827-3485, 2847-3444, 2884-3463, 2889-3452, 2930-3497, 2947-3450, 3017-3609, 3049-3641, |
| | 3056-3546, 3056-3716, 3060-3609, 3061-3719, 3064-3526, 3081-3125, |
| | 3082-3245, 3082-3246, 3083-3585, 3095-3325, 3110-3354, 3110-3579, 3116-3718, 3118-3388, 3118-3391, 3121-3337, 3127-3354, |
| | 3134-3381, 3135-3691, 3149-3702, 3154-3409, 3164-3897, 3180-3410, 3183-3917, 3210-3892, 3226-3383, 3250-3874, 3268-3930, |
| | 3300-3589, 3385-3897, 3386-4105, 3395-3984, 3458-4033, 3512-3775, 3517-3817, 3528-3794, 3559-4188, 3566-3828, 3572-3821, |
| | 3573-3828, 3581-3827, 3600-4077, 3608-3852, 3608-3858, 3608-4147, 3608-4183, 3615-3834, 3621-3890, 3646-3903, 3671-3917, |
| | 3693-4312, 3722-4214, 3726-4368, 3728-3979, 3730-4410, 3790-4340, 3793-4015, 3793-4037, 3856-4406, 3861-4115, 3865-4136, |
| | 3873-4116, 3890-4122, 3908-4373, 3921-4189, 3928-4171, 3928-4176, 3928-4188, 3928-4340, 3928-4387, 3972-4224, 3984-4250, |
| | 3997-4430, 4023-4255, 4072-4378, 4087-4330, 4098-4415, 4128-4430, 4132-4430, 4133-4429, 4136-4430, 4205-4368, 4207-4430, |
| | 4208-4429, 4217-4420, 4225-4412 |
| 42/ | 1-237, 1-238, 1-1216, 3-280, 142-669, 142-713, 368-914, 441-1138, 488-1144, 511-1138, 542-884, 551-1087, |
| 7505777CB1/ | 552-884, 616-1001, 677-934, 677-982, 765-1059, 781-969, 781-1216, 800-1061, 822-1094, |
| 1216 | 822-1099, 822-1101, 865-1065, 905-1030 |
| 43/ | 1-234, 1-1266, 60-315, 66-300, 138-705, 155-399, 156-1134, 202-797, 217-1134, 227-501, 231-1134, 235-796, |
| 7505818CB1/ | 236-1134, 250-803, 257-1133, 271-786, 291-476, 323-825, 325-1134, 374-705, 385-929, 475-741, 475-850, 482-741, |
| 1269 | 509-765, 509-1159, 521-1005, 561-1192, 756-1013, 841-1216, 841-1252, 853-1252, 856-1252, 861-1250, 863-1251, |
| | 868-1269, 873-1267, 903-1251, 931-1181, 931-1266, 1022-1240, 1025-1253, 1040-1253, |
| | 1065-1267, 1092-1225, 1140-1250 |
| 44/ | 1-705, 1-848, 1-1350, 74-518, 219-786, 236-480, 237-1218, 283-916, 308-582, 316-877, 331-884, 352-867, 404-906, |
| 7505821CB1/ | 406-1218, 455-786, 466-1013, 556-822, 556-1097, 563-822, 590-846, 590-1243, 832-1143, 1015-1349, 1050-1324, 1109-1336, |
| 1423 | 1124-1336, 1149-1423, 1176-1309, 1217-1334 |
| 45/ | 1-2864, 46-258, 69-258, 831-1283, 1041-1130, 1041-1355, 1045-1427, 1067-1264, 1725-2231, 1901-2544, 2017-2588, 2029-2686, |
| 7506685CB1/ | 2099-2752, 2113-2583, 2165-2437, 2264-2543, 2269-2619 |
| 2864 | |
| 46/ | 1-237, 1-1025, 2-332, 4-240, 4-252, 4-271, 4-305, 8-224, 9-486, 12-344, 16-281, 18-272, 21-287, 26-317, 27-291, |
| 7500933CB1/ | 27-422, 27-510, 28-276, 28-297, 28-539, 29-248, 29-277, 29-488, 32-308, 34-128, 34-286, 34-308, 34-316, 34-455, 37-346, |
| 1025 | 37-374, 37-377, 37-413, 37-424, 37-498, 37-501, 37-511, 37-544, 38-274, 38-282, 38-298, 38-337, 39-287, 39-313, 40-280, |
| | 40-282, 40-292, 40-313, 40-378, 41-285, 41-292, 42-271, 42-296, 42-355, 43-301, 43-320, 43-321, 43-327, 43-350, 43-463, |
| | 43-544, 44-225, 44-255, 44-276, 44-280, 44-282, 44-288, 44-294, 44-300, 44-317, 44-331, 44-344, 44-357, 44-516, 45-291, |
| | 45-305, 47-311, 47-313, 48-286, 48-305, 48-321, 48-481, |
| | 49-263, 49-298, 49-299, 49-302, 49-324, 50-225, 50-244, 50-264, 50-282, 50-284, 50-286, 50-293, 50-300, 50-306, 50-309, |
| | 50-315, 50-319, 50-331, 50-343, 50-357, 50-428, 50-518, 50-544, 52-314, 52-322, 52-339, 52-544, 54-283, 54-300, 54-325, |
| | 54-335, 54-338, 55-230, 55-311, 55-349, 55-479, 56-302, 56-374, 57-292, 57-304, 57-305, 57-352, 57-382, 57-384, 57-544, |
| | 58-294, 58-455, 61-278, 62-286, 63-300, 63-358, 63-373, 64-185, 64-297, 64-310, 65-544, 67-192, 67-197, 67-208, |
| | 67-281, 67-303, 67-326, 67-377, 68-324, 68-327, 70-317, 74-332, 75-316, 75-334, 75-345, 79-399, 81-391, 83-281, |
| | 83-515, 84-353, 90-329, 90-343, 92-358, 96-329, 96-339, 96-346, 96-544, 97-544, 121-415, 122-353, 122-368, 123-361, |
| | 128-380, 152-327, 156-399, 158-412, 159-415, 168-413, 169-390, 171-425, 179-515, 210-404, 211-514, 212-508, 224-484, |
| | 226-469, 233-393, 246-494, 246-504, 259-484, 259-497, 259-506, 265-506, 265-536, 266-498, 269-540, 269-544, 270-538, |
| | 274-544, 277-500, 281-486, 290-533, 297-519, 297-544, 301-518, 304-544, 306-544, 308-455, 309-517, 316-544, 337-544, |
| | 356-544, 368-503, 376-541, 399-534, 464-703, 535-707, 535-720, 535-733, 535-739, 535-749, 535-752, 535-787, 535-984, |
| | 535-1025, 542-798, 546-792, 551-799, 551-915, 552-774, 554-804, |
| | 555-802, 555-824, 558-801, 558-830, 558-840, 562-847, 563-787, 563-845, 570-756, 570-771, 570-837, 570-840, 570-850, |
| | 576-865, 579-835, 581-808, 581-853, 582-876, 586-842, 590-692, 590-793, 590-828, 608-876, 612-875, 613-859, 615-878, |
| | 617-763, 617-936, 618-836, 618-842, 618-856, 618-1020, 619-863, 622-811, 624-750, 624-1014, 626-859, |
| | 626-868, 627-862, 627-887, 631-887, 633-1013, 634-885, 639-1025, 648-959, 648-1023, 652-812, 652-932, 665-959, |
| | 668-931, 668-1023, 677-746, 68 1-743, 693-908, 694-964, 696-766, 700-935, 702-1023, 707-945, 707-950, 707-971, |
| | 716-982, 723-998, 727-945, 730-956, 730-965, 731-1007, 731-1025, 743-1024, 744-969, 745-1025, 746-988, 748-968, |
| | 748-1025, 749-859, 749-996, 749-1025, 751-1004, 755-965, 755-1016, 763-1013, 766-937, 766-1025, 768-996, |
| | 777-1025, 786-958, 788-1025, 809-1025, 811-1025, 820-1025, 836-1025, 845-939, 851-1025, 853-1000, |
| | 853-1025, 873-1025, 896-1025 |
| 47/ | 1-547, 1-551, 1-636, 1-828, 1-2531, 76-537, 106-758, 373-1008, 379-554, 421-758, 445-1008, 894-1580, |
| 7389203CB1/ | 950-1580, 985-1091, 1209-1549, 1216-1588, 1217-1558, 1235-1584, 1273-1591, 1371-1677, 1461-1588, 1582-1823, 1582-2001, |
| 3048 | 1582-2071, 1588-1696, 1588-2014, 1592-2194, 1592-2313, 1627-2061, 1627-2479, 1631-1867, 1709-2539, 1777-2555, |
| | 1777-2577, 1856-2728, 1933-2800, 1933-2816, 2113-3048, 2154-2974, 2211-2789, 2305-2986, 2324-2905, |
| | 2391-2799, 2401-2820, 2404-2801, 2411-2905 |
| 48/ | 1-222, 10-221, 16-221, 17-278, 17-542, 17-713, 24-1289, 36-221, 38-222, 39-208, 39-222, 41-163, 53-222, |
| 7506268CB1/ | 62-132, 74-222, 81-222, 88-217, 222-436, 222-457, 222-463, 222-471, 222-475, 222-486, 222-501, 222-531, 222-705, |
| 1299 | 222-720, 222-856, 226-488, 228-476, 228-721, 228-831, 228-890, 233-527, 236-496, 240-546, 241-458, 243-490, 248-546, |
| | 256-553, 257-495, 257-619, 262-525, 263-849, 268-502, 268-515, 273-466, 274-881, 277-569, 285-532, 286-540, 288-563, |
| | 293-570, 298-412, 298-578, 302-501, 302-546, 302-715, 302-800, 309-531, 309-566, 312-538, 315-607, 319-848, 322-562, |
| | 324-566, 330-966, 332-978, 347-621, 347-899, 348-967, 351-595, 352-567, 354-667, 355-983, 356-924, 357-626, 358-618, |
| | 360-583, 367-678, 368-839, 375-635, 378-581, 379-622, 379-623, 379-635, 382-567, 384-635, 386-586, 388-681, 391-890, |
| | 396-646, 396-887, 404-1028, 405-700, 411-685, 414-668, 418-517, 422-688, 426-1132, 430-645, 430-659, 430-771, |
| | 431-705, 432-652, 432-672, 432-725, 432-1017, 433-672, 433-890, 433-917, 433-919, 433-940, |
| | 435-887, 436-1033, 437-677, 437-711, 439-924, 443-683, 445-975, 448-731, 450-708, |
| | 450-740, 451-687, 459-697, 472-1143, 473-717, 475-713, 475-812, 475-902, 479-726, 486-779, 487-742, 489-787, 493-781, |
| | 498-642, 498-725, 498-1186, 502-891, 515-963, 520-835, 521-781, 525-821, 532-1126, 536-791, 536-792, 545-835, |
| | 545-978, 546-806, 546-812, 549-1249, 555-745, 571-846, 582-810, 582-870, 583-737, 584-1095, 586-804, 586-811, |
| | 586-849, 586-856, 586-869, 586-879, 588-861, 591-852, 594-841, 604-864, 604-1261, 604-1294, 607-805, 607-897, |
| | 619-874, 623-1255, 626-895, 632-1130, 636-885, 640-920, 641-891, 652-903, 654-783, 654-978, 658-941, 661-927, |
| | 663-825, 664-908, 668-889, 670-1021, 674-1287, 685-806, 704-1285, 709-1030, 725-1094, 735-1287, 744-1014, |
| | 746-1287, 761-1233, 766-1291, 769-1294, 775-852, 778-1029, 782-1047, 787-1287, |
| | 790-1226, 791-1235, 794-1068, 794-1294, 797-1006, 800-956, 804-1052, 805-1098, 812-1294, 813-1136, 814-1238, 817-1033, |
| | 819-1096, 820-967, 820-1063, 822-1076, 823-1083, 833-1064, 834-880, 836-1100, 838-1100, 840-1054, 841-1111, 853-1150, |
| | 858-1294, 859-1091, 859-1113, 859-1115, 861-1143, 866-1294, 867-1144, 870-1287, 872-1294, 873-1294, 874-1100, |
| | 874-1145, 874-1294, 875-1086, 875-1294, 877-1151, 877-1294, 880-1096, 880-1103, 882-1191, 883-1294, 884-1105, 884-1133, |
| | 885-1294, 886-1131, 899-1053, 901-1170, 901-1187, 902-1211, 904-1299, 905-1291, 909-1294, 910-1294, 912-1294, 915-1294, |
| | 918-1162, 920-1294, 922-1192, 925-1147, 941-1294, 942-1197, 947-1294, 966-1217, 970-1291, 971-1294, 973-1243, |
| | 977-1232, 978-1211, 978-1266, 981-1294, 982-1294, 983-1294, 984-1294, 991-1294, 992-1294, 993-1258, 993-1294, |
| | 994-1262, 994-1279, 994-1294, 995-1247, 995-1286, 995-1294, 996-1167, 996-1294, 997-1294, 1000-1283, 1000-1294, |
| | 1001-1294, 1002-1294, 1008-1294, 1009-1294, 1014-1294, 1016-1188, 1019-1275, 1028-1244, 1038-1294, 1040-1294, 1050-1294, |
| | 1054-1294, 1065-1233, 1069-1294, 1077-1235, 1078-1294, 1091-1294, 1099-1294, 1107-1294, 1108-1294, 1114-1294, 1116-1192, |
| | 1125-1294, 1128-1294, 1139-1294, 1142-1294, 1162-1294, 1167-1294, 1179-1203, 1186-1286 |
| 49/ | 1-259, 1-367, 1-444, 1-534, 1-535, 1-536, 1-542, 2-3146, 3-275, 4-275, 161-824, 169-275, 170-275, |
| 7509159CB1/ | 181-275, 189-460, 195-275, 198-418, 199-430, 206-507, 404-547, 563-1015, 563-1028, 563-1153, 600-886, |
| 3146 | 613-1311, 646-1237, 654-1261, 657-1045, 676-973, 678-1339, |
| | 680-1217, 691-1201, 694-1335, 709-982, 725-1299, 731-1265, 735-1334, 753-1141, 767-1357, 769-1237, 780-1161, 782-1017, |
| | 782-1279, 802-1384, 820-1365, 825-1432, 830-1408, 840-1161, 848-1409, 860-1408, 860-1518, 876-1373, 903-1557, 905-1449, |
| | 916-1505, 919-1456, 930-1496, 939-1535, 948-1653, 949-1428, 960-1377, 965-1518, 989-1507, 992-1536, 1026-1624, 1027-1621, |
| | 1043-1752, 1050-1595, 1063-1574, 1065-1226, 1065-1265, 1070-1226, 1076-1226, 1122-1801, 1169-1660, 1174-1793, 1176-1595, |
| | 1176-1736, 1178-1466, 1182-1684, 1196-1454, 1202-1664, 1245-1511, 1245-1869, 1302-1801, 1316-1602, 1322-1981, 1323-1509, |
| | 1337-1808, 1342-1631, 1343-1694, 1357-1829, 1367-1592, 1412-2007, 1424-1919, 1502-1625, 1522-2100, 1530-1917, 1536-2106, |
| | 1539-2396, 1542-2360, 1562-2401, 1563-2401, 1565-2063, 1567-2401, 1570-2074, |
| | 1573-2401, 1580-2104, 1580-2122, 1584-2263, 1585-2175, 1598-2401, 1606-2369, 1608-2401, 1612-2401, 1613-2172, 1625-2156, |
| | 1644-2142, 1652-2281, 1653-2133, 1669-2172, 1677-2283, 1684-2401, 1686-2333, 1689-2234, 1692-2348, 1696-2210, 1706-2360, |
| | 1709-2355, 1712-2377, 1713-2208, 1715-2401, 1720-2401, 1722-2545, 1723-2214, 1727-2404, 1731-2336, 1741-2322, 1744-2366, |
| | 1748-2364, 1759-2397, 1773-2252, 1773-2282, 1780-2445, 1797-2343, 1802-2077, 1813-2401, 1814-2098, 1814-2254, 1815-2104, |
| | 1815-2135, 1816-2049, 1816-2467, 1819-2305, 1824-2315, 1827-2476, 1829-2503, 1829-2543, 1830-2328, 1834-2299, 1855-2216, |
| | 1857-2166, 1864-2098, 1880-1999, 1880-2075, 1881-2124, 1890-2212, 1892-2127, 1892-2145, 1923-2625, 1925-2663, 1932-2606, |
| | 1936-2512, 1948-2156, 1961-2371, 1981-2554, 1992-2347, 1997-2145, 1999-2242, 1999-2570, 2005-2743, 2018-2373, 2020-2704, |
| | 2025-2387, 2042-2494, 2053-2385, 2068-2591, 2071-2369, 2081-2527, 2101-2389, 2102-2553, 2107-2763, 2110-2642, 2119-2552, |
| | 2141-2704, 2146-2789, 2154-2676, 2156-2616, 2158-2431, 2161-2765, |
| | 2171-2740, 2183-2754, 2193-2756, 2200-2791, 2205-2789, 2213-2712, 2215-3004, 2224-2703, 2224-2788, 2240-2634, 2244-2459, |
| | 2255-2592, 2288-2785, 2294-2782, 2302-2871, 2308-3013, 2318-2562, 2323-3013, 2324-2464, 2327-2590, 2327-2594, 2336-2619, |
| | 2339-2789, 2341-2464, 2345-2730, 2348-2490, 2348-2726, 2366-2834, 2377-2701, 2400-2626, 2425-3076, 2487-3130, 2492-2700, |
| | 2496-2759, 2500-3146, 2524-3142, 2544-2825, 2546-3136, 2548-2826, 2571-2976, 2571-3146, 2579-3143, 2601-2868, 2616-2860, |
| | 2620-3095, 2640-3094, 2641-2830, 2641-2908, 2641-2923, 2641-3132, 2645-2951, 2655-2936, 2658-2936, 2658-2937, 2658-2939, |
| | 2658-2941, 2658-2942, 2658-2943, 2658-2948, 2658-2950, 2663-2884, 2673-3133, 2677-3133, 2691-3123, 2699-2953, 2700-3094, |
| | 2704-3132, 2709-3133, 2718-3074, 2723-3133, 2725-3094, 2730-3084, 2736-3140, 2739-3146, 2752-3133, 2756-3133, 2757-2915, |
| | 2778-3133, 2784-2927, 2784-3132, 2792-3037, 2794-3133, 2809-3030, 2815-3133, 2824-3133, 2850-3130, 2856-3127, 2860-3125, |
| | 2861-3094, 2862-3102, 2884-3133, 2938-3132, 2940-3072, 2945-3132, 2966-3131, 3003-3132, 3013-3133, 3021-3146, 3025-3132 |
| 50/ | 1-721, 9-2238, 503-1538, 575-1521, 652-1651, 662-1486, 670-1590, 672-1521, 677-1642, 680-1571, 1446-2238 |
| 7512347CB1/ | |
| 2238 | |
| 29/ | 1-237, 1-557, 1-1073, 2-332, 4-240, 4-252, 4-271, 4-305, 8-224, 9-486, 12-344, 16-281, 18-272, |
| 7500938CB1/ | 21-287, 26-317, 27-291, 27-422, 27-510, 28-276, 28-297, 28-539, 29-248, 29-277, 29-488, 29-557, 30-585, 32-308, 32-557, |
| 1151 | 34-128, 34-286, 34-308, 34-316, 34-455, 37-346, 37-413, 37-424, 37-501, 37-511, 37-556, 37-557, 38-274, 38-282, 38-298, |
| | 38-337, 39-287, 39-313, 40-280, 40-282, 40-292, 40-313, 40-378, 41-285, 41-292, 42-271, 42-296, 42-355, 42-557, 43-301, |
| | 43-320, 43-321, 43-327, 43-350, 43-463, 43-554, 44-225, 44-255, 44-276, 44-280, 44-282, 44-288, 44-294, 44-300, 44-317, |
| | 44-331, 44-344, 44-357, 44-516, 45-291, 45-305, 47-311, 47-313, 48-286, 48-305, 48-321, 48-481, 49-263, 49-298, 49-299, |
| | 49-302, 49-324, 50-225, 50-244, 50-282, 50-284, 50-286, 50-293, 50-300, 50-306, 50-309, 50-315, 50-319, 50-331, 50-343, |
| | 50-357, 50-428, 50-518, 50-557, 52-314, 52-322, 52-339, 52-557, 54-283, 54-300, 54-325, |
| | 54-335, 54-338, 55-230, 55-311, 55-349, 55-479, 55-557, 56-302, 56-374, 57-292, 57-304, 57-305, 57-352, 57-382, |
| | 57-384, 57- 65-557, 67-192, 67-197, 67-208, 67-281, 67-303, 67-326, 67-377, 68-324, 68-327, 70-317, 74-332, |
| | 75-316, 75-334, 75-345, 79-399, 81-391, 83-281, 83-515, 84-353, 90-329, 90-343, 90-557, 92-358, 96-329, 96-339, |
| | 96-346, 96-546, 97-557, 121-415, 122-353, 122-368, 123-358, 128-380, 152-327, 156-399, 169-390, 171-425, 179-515, |
| | 210-404, 211-514, 212-508, 224-484, 226-469, 246-494, 246-504, 259-484, 259-497, 259-506, 265-506, 265-536, 266-498, |
| | 269-540, 269-544, 270-538, 274-550, 274-559, 277-500, 281-486, 290-533, 297-519, 297-557, 301-518, 306-548, 309-517, |
| | 316-557, 337-554, 347-557, 356-546, 368-503, 376-546, 399-534, 556-781, 557-723, |
| | 557-767, 557-768, 557-776, 557-787, 557-795, 557-797, 557-808, 557-809, 557-810, 557-986, 557-1050, 557-1112, 560-1032, |
| | 562-800, 574-755, 578-835, 578-1095, 590-846, 594-840, 599-847, 600-822, 602-852, 603-850, 603-872, 606-849, 606-878, |
| | 606-888, 610-895, 611-835, 611-893, 618-804, 618-819, 618-885, 618-888, 618-898, 624-913, 627-883, 629-856, |
| | 629-901, 630-924, 634-890, 638-740, 638-841, 638-876, 656-924, 660-923, 661-907, 663-926, 665-811, 665-984, |
| | 666-884, 666-890, 666-904, 666-1084, 667-911, 670-859, 672-798, 672-1142, 674-907, 674-916, 675-910, 675-935, |
| | 679-935, 681-1061, 682-933, 687-1084, 696-1007, 696-1071, 700-860, 700-980, 713-1007, 716-979, 716-1147, |
| | 725-794, 729-791, 741-956, 742-1012, 744-814, 748-983, 750-1071, 755-993, 755-998, 755-1019, 764-1030, 771-1046, |
| | 775-993, 778-1004, 778-1013, 779-1055, 779-1073, 791-1072, 792-1017, 793-1078, 794-1036, 796-1016, 796-1090, |
| | 797-907, 797-1044, 797-1076, 799-1052, 803-1013, 803-1064, 811-1082, 814-985, 814-1088, 816-1044, 825-1074, |
| | 834-1006, 836-1082, 857-1076, 859-1085, 868-1127, 884-1086, 893-987, 899-1151, |
| | 901-1048, 901-1144, 921-1135, 944-1083 |
| 30/ | 1-670, 93-700, 346-639, 407-819, 407-1016, 407-1089, 407-1141, 407-1192, 407-1277, 408-1097, |
| 90055441CB1/ | 408-1199, 408-1273, 408-1276, |
| 1277 | 408-1277, 415-1277, 496-1277 |

TABLE 5

| Polynucleotide SEQ | | Representative |
|---|---|---|
| ID NO: | Incyte Project ID: | Library |
| 26 | 7500354CB1 | BEPINOT01 |
| 27 | 3871329CB1 | DRGCNOT02 |
| 28 | 1681386CB1 | FIBPFEN06 |
| 29 | 7500938CB1 | BRAITUT21 |
| 30 | 90055441CB1 | STOMTDE01 |
| 31 | 7500936CB1 | BRAITUT21 |
| 32 | 7500950CB1 | EOSITXT01 |
| 33 | 7500854CB1 | SCORNON02 |
| 34 | 2754176CB1 | ADRENOT08 |
| 35 | 7503408CB1 | BRAINOT09 |
| 36 | 71086982CB1 | TESTTUT02 |
| 37 | 7506367CB1 | CERVNOT01 |
| 38 | 1414020CB1 | BRAINOT12 |
| 39 | 7621128CB1 | KIDNFET01 |
| 40 | 7505822CB1 | LUNGTUT03 |
| 41 | 71607945CB1 | BRAINOT11 |
| 42 | 7505777CB1 | BMARTXE01 |
| 43 | 7505818CB1 | PROSNON01 |
| 44 | 7505821CB1 | THP1TXT03 |
| 45 | 7506685CB1 | BRAZDIT04 |
| 46 | 7500933CB1 | BRAITUT21 |
| 47 | 7389203CB1 | LIVRFEE02 |
| 48 | 7506268CB1 | MUSCDIN06 |
| 49 | 7509159CB1 | LIVRFEA01 |

TABLE 6

| Library | Vector | Library Description |
|---|---|---|
| ADRENOT08 | pINCY | Library was constructed using RNA isolated from adrenal tissue removed from a 20-year-old Caucasian male, who died |
| | | from head trauma. |
| BEPINOT01 | PSPORT1 | Library was constructed using RNA isolated from a bronchial epithelium primary cell line derived from a 54-year-old |
| | | Caucasian male. |
| BMARTXE01 | pINCY | This 5′ biased random primed library was constructed using RNA isolated from treated SH-SY5Y cells derived from a |
| | | metastatic bone marrow neuroblastoma, removed from a 4-year-old Caucasian female (Schering AG). The medium was |
| | | MEM/HAM'S F12 with 10% fetal calf serum. After reaching about 80% confluency cells were treated with 6- |
| | | Hydroxydopamine (6-OHDA) at 100 microM for 8 hours. |
| BRAINOT09 | pINCY | Library was constructed using RNA isolated from brain tissue removed from a Caucasian male fetus, who died at 23 |
| | | weeks gestation. |
| BRAINOT11 | pINCY | Library was constructed using RNA isolated from brain tissue removed from the right temporal lobe of a 5-year-old |
| | | Caucasian male during a hemispherectomy. Pathology indicated extensive polymicrogyria and mild to moderate gliosis |
| | | (predominantly subpial and subcortical), consistent with chronic seizure disorder. Family history included a cervical |
| | | neoplasm. |
| BRAINOT12 | pINCY | Library was constructed using RNA isolated from brain tissue removed from the right frontal lobe of a 5-year-old |
| | | Caucasian male during a hemispherectomy. Pathology indicated extensive polymicrogyria and mild to moderate gliosis |
| | | (predominantly subpial and subcortical), which are consistent with chronic seizure disorder. Family history included a |
| | | cervical neoplasm. |
| BRAITUT21 | pINCY | Library was constructed using RNA isolated from brain tumor tissue removed from the midline frontal lobe of a 61-year- |
| | | old Caucasian female during excision of a cerebral meningeal lesion. Pathology indicated subfrontal meningothelial |
| | | meningioma with no atypia. One ethmoid and mucosal tissue sample indicated meningioma. Family history included |
| | | cerebrovascular disease, senile dementia, hyperlipidemia, benign hypertension, atherosclerotic coronary artery disease, |
| | | congestive heart failure, and breast cancer. |
| BRAZDIT04 | pINCY | Library was constructed using RNA isolated from diseased striatum and globus pallidus tissue removed from a |
| | | 70-year-old female who died from metastatic adenocarcinoma. Pathology indicated moderate Alzheimer disease and |
| | | mild carotid and cerebral atherosclerosis. The cerebral hemispheres, frontal and temporal lobes, white matter, |
| | | and hippocampus showed mild atrophy, bilaterally. There were numerous neurofibrillary tangles, neuritic and |
| | | diffuse amyloid plaques deposited throughout most neocortical areas. Most of the diffuse plaques were in |
| | | the superficial layers, with more core and neuritic amyloid plaques in the deep cortical layers. Most of the tangles |
| | | were found in small interneurons, rather than in the large pyramidal neurons. The areas that were most involved |
| | | with plaques and tangles were the entorhinal cortex, temporal cortex, and superior parietal lobes. There was |
| | | marked vacuolization of the superficial layers throughout all neocortical areas examined. The hippocampus |
| | | contained numerous neurofibrillary tangles (predominantly in the CA-1 field), diffuse degeneration |
| | | within the pyramidal cell neurons. There were neuritic plaques with scattered neurofibrillary tangles within |
| | | the amygdala. The thalamus had scattered diffuse plaques. There was mild pigment incontinence in the substantia nigra |
| | | compacta. The periaqueductal gray showed mild gliosis. Diffuse plaques were found within the superior colliculus. |
| | | Neurofibrillary tangles were found within the pons. The neurons of the locus ceruleus were ballooned and contain |
| | | eosinophilic foamy material with very little neuromelanin pigment. |
| CERVNOT01 | PSPORT1 | Library was constructed using RNA isolated from the uterine cervical tissue of a 35-year-old Caucasian female during a |
| | | vaginal hysterectomy with dilation and curettage. Pathology indicated mild chronic cervicitis. Family history included |
| | | atherosclerotic coronary artery disease and type II diabetes. |
| DRGCNOT02 | pINCY | Library was constructed using RNA isolated from dorsal root ganglion tissue removed from the cervical spine of a 32- |
| | | year-old Caucasian male who died from acute pulmonary edema, acute bronchopneumonia, bilateral pleural effusions, |
| | | pericardial effusion, and malignant lymphoma (natural killer cell type). The patient presented with pyrexia of unknown |
| | | origin, malaise, fatigue, and gastrointestinal bleeding. Patient history included probable cytomegalovirus infection, liver |
| | | congestion, and steatosis, splenomegaly, hemorrhagic cystitis, thyroid hemorrhage, respiratory failure, pneumonia of the |
| | | left lung, natural killer cell lymphoma of the pharynx, Bell's palsy, and tobacco and alcohol abuse. Previous surgeries |
| | | included colonoscopy, closed colon biopsy, adenotonsillectomy, and nasopharyngeal endoscopy and biopsy. Patient |
| | | medications included Diflucan (fluconazole), Deltasone (prednisone), hydrocodone, Lartab, Aiprazolam, Reazodone, |
| | | ProMace-Cytabom, Etoposide, Cisplatin, Cytarabine, and dexamethasome. The patient received radiation therapy and |
| | | multiple blood transfusions. |
| EOSITXT01 | pINCY | Library was constructed using RNA isolated from eosinophils stimulated with IL-5. |
| FIBPFEN06 | pINCY | The normalized prostate stromal fibroblast tissue libraries were constructed from 1.56 million independent clones from a |
| | | prostate fibroblast library. Starting RNA was made from fibroblasts of prostate stroma removed from a male fetus, who |
| | | died after 26 weeks' gestation. The libraries were normalized in two rounds using conditions adapted from Soares et al., |
| | | PNAS (1994) 91: 9228 and Bonaldo et al., Genome Research (1996) 6: 791, except that a significantly longer (48- |
| | | hours/round)reannealing hybridization was used. The library was then linearized and recircularized to select for insert |
| | | containing clones as follows: plasmid DNA was prepped from approximately 1 million clones from the normalized |
| | | prostate stromal fibroblast tissue libraries following soft agar transformation. |
| KIDNFET01 | pINCY | Library was constructed using RNA isolated from kidney tissue removed from a Caucasian female fetus, who died at 17 |
| | | weeks' gestation from anencephalus. |
| LIVRFEA01 | PSPORT1 | This amplified library was constructed using RNA isolated from liver tissue removed from a Caucasian male fetus who |
| | | died from fetal demise. |
| LIVRFEE02 | pINCY | This 5′ biased random primed library was constructed using RNA isolated from liver tissue removed from a Caucasian |
| | | male fetus who died from fetal demise. Serologies were negative. |
| LUNGTUT03 | PSPORT1 | Library was constructed using RNA isolated from lung tumor tissue removed from the left lower lobe of a 69-year-old |
| | | Caucasian male during segmental lung resection. Pathology indicated residual grade 3 invasive squamous cell carcinoma. |
| | | Patient history included acute myocardial infarction, prostatic hyperplasia, malignant skin neoplasm, and tobacco use. |
| MUSCDIN06 | pINCY | This normalized diseased thigh muscle tissue library was constructed from 6.76 million independent clones from a |
| | | diseased thigh muscle tissue library. Starting RNA was made from RNA isolated from diseased thigh muscle tissue |
| | | removed from a 74-year-old Caucasian female who died from respiratory arrest due to amyotrophic lateral |
| | | sclerosis (ALS). Patient history included amyotrophic lateral sclerosis, hypertension, and arthritis. The library |
| | | was normalized in two rounds using conditions adapted from Soares et al., PNAS (1994) 91: 9228-9232 and Bonaldo |
| | | et al., Genome Research (1996) 6: 791, except that a significantly longer (48 hours/round) reannealing |
| | | hybridization was used. |
| PROSNON01 | PSPORT1 | This normalized prostate library was constructed from 4.4 M independent clones from a prostate library. |
| | | Starting RNA was made from prostate tissue removed from a 28-year-old Caucasian male who died from a self-inflicted |
| | | gunshot wound. The normalization and hybridization conditions were adapted from Soares, M. B. et al. (1994) |
| | | Proc. Natl. Acad. Sci. USA 91: 9228-9232, using a longer (19 hour) reannealing hybridization period. |
| SCORNON02 | PSPORT1 | This normalized spinal cord library was constructed from 3.24M independent clones from the a spinal cord tissue library. |
| | | RNA was isolated from the spinal cord tissue removed from a 71-year-old Caucasian male who died from respiratory |
| | | arrest. Patient history included myocardial infarction, gangrene, and end stage renal disease. The normalization and |
| | | hybridization conditions were adapted from Soares et al.(PNAS (1994) 91: 9228). |
| STOMTDE01 | PCDNA2.1 | This 5′ biased random primed library was constructed using RNA isolated from stomach tissue removed from a 61-year- |
| | | old Caucasian male during a partial esophagectomy, proximal gastrectomy, pyloromyotomy, and regional lymph node |
| | | excision. Pathology for the associated tumor indicated an invasive grade 3 adenocarcinoma in the esophagus, |
| | | extending distally to involve the gastroesophageal junction. The tumor extended through the muscularis to involve |
| | | periesophageal and perigastric soft tissues. One perigastric and two periesophageal lymph nodes were positive for |
| | | tumor. There were multiple perigastric and periesophageal tumor implants. The patient presented with deficiency |
| | | anemia and myelodysplasia. Patient history included hyperlipidemia, and tobacco and alcohol abuse in remission. |
| | | Previous surgeries included adenotonsillectomy, rhinoplasty, vasectomy, and hemorrhoidectomy. A previous bone |
| | | marrow aspiration found the marrow to be hypercellular for age and had a cellularity-to-fat ratio of 95:5. |
| | | The marrow was focally densely fibrotic. Granulocytic precursors were slightly increased with normal maturation. |
| | | The estimate of blast cells was greater than 5%. Megakaryocytes were increased and appeared atypical in clusters. |
| | | Storage cells and granulomata were absent. Patient medications included Epoetin, Danocrine, Berocca Plus |
| | | tablets, Selenium, vitamin B6 phosphate, vitamins E & C, and beta carotene. Family history included alcohol |
| | | abuse, atherosclerotic coronary artery disease, type II diabetes, chronic liver disease, and primary cardiomyopathy |
| | | in the father; and benign hypertension and cerebrovascular disease in the mother. |
| TESTTUT02 | pINCY | Library was constructed using RNA isolated from testicular tumor removed from a 31-year-old Caucasian male during |
| | | unilateral orchiectomy. Pathology indicated embryonal carcinoma. |
| THP1TXT03 | pINCY | Library was constructed using RNA isolated from treated THP-1 cells. THP-1 is a human promonocyte line derived from |
| | | the peripheral blood of a 1-year-old Caucasian male with acute monocytic leukemia (ref: Int. J. Cancer (1980) 26: 171). |
| | | The THP-1 cultured cells were differentiated with PMA(100 ng/ml) for 48 hours, incubated with Mycobacteria |
| | | tuberculosis, strain H37Rv, for 4 hours at 37 C, washed and RNA extracted. |

TABLE 7

| | | | Parameter |
|---|---|---|---|
| Program | Description | Reference | Threshold |
| ABI | A program that removes vector sequences and | Applied Biosystems, Foster City, CA. | |
| FACTURA | masks ambiguous bases in nucleic acid sequences. | | |
| ABI/ | A Fast Data Finder useful in comparing and | Applied Biosystems, Foster City, CA; | Mismatch |
| PARACEL | annotating amino acid or nucleic acid sequences. | Paracel Inc., Pasadena, CA. | <50% |
| FDF | | | |
| ABI | A program that assembles nucleic acid sequences. | Applied Biosystems, Foster City, CA. | |
| AutoAssembler | | | |
| BLAST | A Basic Local Alignment Search Tool useful in | Altschul, S. F. et al. (1990) J. Mol. Biol. | ESTs: |
| | sequence similarity search for amino acid and | 215: 403-410; Altschul, S. F. et al. (1997) | Probability |
| | nucleic acid sequences. BLAST includes five | Nucleic Acids Res. 25: 3389-3402. | value = 1.0E−8 |
| | functions: blastp, blastn, blastx, tblastn, and tblastx. | | or less Full |
| | | | Length |
| | | | sequences: |
| | | | Probability |
| | | | value = |
| | | | 1.0E−10 or less |
| FASTA | A Pearson and Lipman algorithm that searches for | Pearson, W. R. and D. J. Lipman (1988) Proc. | ESTs: fasta E |
| | similarity between a query sequence and a group of | Natl. Acad Sci. USA 85: 2444-2448; Pearson, | value = |
| | sequences of the same type. FASTA comprises as | W. R. (1990) Methods Enzymol. 183: 63-98; | 1.06E−6; |
| | least five functions: fasta, tfasta, fastx, tfastx, and | and Smith, T. F. and M. S. Waterman (1981) | Assembled |
| | ssearch. | Adv. Appl. Math. 2: 482-489. | ESTs: fasta |
| | | | Identity = 95% |
| | | | or greater and |
| | | | Match length = |
| | | | 200 bases or |
| | | | greater; fastx E |
| | | | value = 1.0E−8 |
| | | | or less; Full |
| | | | Length |
| | | | sequences: |
| | | | fastx score = |
| | | | 100 or greater |
| BLIMPS | A BLocks IMProved Searcher that matches a | Henikoff, S. and J. G. Henikoff (1991) Nucleic | Probability |
| | sequence against those in BLOCKS, PRINTS, | Acids Res. 19: 6565-6572; Henikoff, J. G. and | value = 1.0E−3 |
| | DOMO, PRODOM, and PFAM databases to search | S. Henikoff (1996) Methods Enzymol. | or less |
| | for gene families, sequence homology, and structural | 266: 88-105; and Attwood, T. K. et al. (1997) J. | |
| | fingerprint regions. | Chem. Inf. Comput. Sci. 37: 417-424. | |
| HMMER | An algorithm for searching a query sequence against | Krogh, A. et al. (1994) J. Mol. Biol. | PFAM, INCY, |
| | hidden Markov model (HMM)-based databases of | 235: 1501-1531; Sonnhammer, E. L. L. et al. | SMART, or |
| | protein family consensus sequences, such as PFAM, | (1988) Nucleic Acids Res. 26: 320-322; | TIGRFAM |
| | INCY, SMART, and TIGRFAM. | Durbin, R. et al. (1998) Our World View, in a | hits: |
| | | Nutshell, Cambridge Univ. Press, pp. 1-350. | Probability |
| | | | value = 1.0E−3 |
| | | | or less |
| | | | Signal peptide |
| | | | hits: Score = 0 |
| | | | or greater |
| ProfileScan | An algorithm that searches for structural and sequence | Gribskov, M. et al. (1988) CABIOS 4: 61-66; | Normalized |
| | motifs in protein sequences that match sequence patterns | Gribskov, M. et al. (1989) Methods Enzymol. | quality score ≧ |
| | defined in Prosite. | 183: 146-159; Bairoch, A. et al. (1997) | GCG specified |
| | | Nucleic Acids Res. 25: 217-221. | “HIGH” value |
| | | | for that |
| | | | particular |
| | | | Prosite motif. |
| | | | Generally, |
| | | | score = |
| | | | 1.4-2.1. |
| Phred | A base-calling algorithm that examines automated | Ewing, B. et al. (1998) Genome Res. | |
| | sequencer traces with high sensitivity and probability. | 8: 175-185; Ewing, B. and P. Green | |
| | | (1998) Genome Res. 8: 186-194. | |
| Phrap | A Phils Revised Assembly Program including SWAT and | Smith, T. F. and M. S. Waterman (1981) Adv. | Score = 120 or |
| | CrossMatch, programs based on efficient implementation | Appl. Math. 2: 482-489; Smith, T. F. and M. S. | greater; |
| | of the Smith-Waterman algorithm, useful in searching | Waterman (1981) J. Mol. Biol. 147: 195-197; | Match length = |
| | sequence homology and assembling DNA sequences. | and Green, P., University of Washington, | 56 or greater |
| | | Seattle, WA. | |
| Consed | A graphical tool for viewing and editing Phrap assemblies. | Gordon, D. et al. (1998) Genome Res. 8: 195-202. | |
| SPScan | A weight matrix analysis program that scans protein | Nielson, H. et al. (1997) Protein Engineering | Score = 3.5 or |
| | sequences for the presence of secretory signal peptides. | 10: 1-6; Claverie, J. M. and S. Audic (1997) | greater |
| | | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos (1996) | |
| | determine orientation. | Protein Sci. 5: 363-371. | |
| TMHMMER | A program that uses a hidden Markov model (HMM) to | Sonnhammer, E. L. et al. (1998) Proc. Sixth Intl. | |
| | delineate transmembrane segments on protein sequences | Conf. on Intelligent Systems for Mol. Biol., | |
| | and determine orientation. | Glasgow et al., eds., The Am. Assoc. for Artificial | |
| | | Intelligence (AAAI) Press, Menlo Park, CA, | |
| | | and MIT Press, Cambridge, MA, pp. 175-182. | |
| Motifs | A program that searches amino acid sequences for patterns | Bairoch, A. et al. (1997) Nucleic Acids | |
| | that matched those defined in Prosite. | Res. 25: 217-221; Wisconsin Package | |
| | | Program Manual, version 9, page M51-59, | |
| | | Genetics Computer Group, Madison, WI. | |

TABLE 8

| | | | | | | | | | | | African | Asian | | +HL,64 |
|---|---|---|---|---|---|---|---|---|---|---|---|---|---|---|
| SEQ | | | | | | | | | | Caucasian | Allele 1 | Allele 1 | Hispanic | |
| ID | | | | EST | CB1 | EST | Allele | Allele | Amino | Allele 1 | fre- | fre- | Allele 1 | |
| NO: | PID | EST ID | SNP ID | SNP | SNP | Allele | 1 | 2 | Acid | frequency | quency | quency | frequency | |

| | | | | | | | | | | | | | |
|---|---|---|---|---|---|---|---|---|---|---|---|---|---|
| 47 | 7389203 | 7347224H1 | SNP00094502 | 309 | 701 | C | C | A | N44 | n/d | n/a | n/a | n/a |
| | | | | | | | | | coding | | | | |
| 48 | 7506268 | 008160H1 | SNP00008075 | 97 | 973 | C | C | G | non- | n/d | n/a | n/a | n/a |
| | | | | | | | | | coding | | | | |
| 48 | 7506268 | 1657745H1 | SNP00033953 | 99 | 1272 | C | C | T | non- | n/a | n/a | n/a | n/a |
| | | | | | | | | | coding | | | | |
| 48 | 7506268 | 6114153H1 | SNP00008075 | 113 | 1180 | C | C | G | non- | n/d | n/a | n/a | n/a |
| | | | | | | | | | coding | | | | |
| 49 | 7509159 | 1388790H1 | SNP00008723 | 116 | 2924 | T | C | T | non- | n/a | n/a | n/a | n/a |
| | | | | | | | | | coding | | | | |
| 49 | 7509159 | 2191204H1 | SNP00151397 | 22 | 2720 | C | C | T | non- | n/a | n/a | n/a | n/a |
| | | | | | | | | | coding | | | | |
| 49 | 7509159 | 3542239H1 | SNP00151397 | 104 | 2716 | C | C | T | non- | n/a | n/a | n/a | n/a |
| | | | | | | | | | coding | | | | |
| 49 | 7509159 | 4407957H1 | SNP00151397 | 64 | 2718 | C | C | T | non- | n/a | n/a | n/a | n/a |
| | | | | | | | | | coding | | | | |
| 49 | 7509159 | 4728316H1 | SNP00008723 | 232 | 2920 | T | C | T | non- | n/a | n/a | n/a | n/a |
| | | | | | | | | | coding | | | | |

1. An isolated polypeptide selected from the group consisting of:
a) a polypeptide comprising an amino acid sequence selected from the group consisting of SEQ ID NO:1-10, and SEQ ID NO: 14-25,
b) a polypeptide consisting essentially of an amino acid sequence selected from the group consisting of SEQ ID NO:11-13,
c) a polypeptide comprising a naturally occurring amino acid sequence at least 90% identical to an amino acid sequence selected from the group consisting of SEQ ID NO:2-3, SEQ ID NO:5, SEQ ID NO:8, SEQ ID NO: 14, SEQ ID NO:22, and SEQ ID NO:25,
d) a polypeptide comprising a naturally occurring amino acid sequence at least 96% identical to an amino acid sequence selected from the group consisting of SEQ ID NO:7, SEQ ID NO:13, SEQ ID NO: 15, and SEQ ID NO:24,
e) a polypeptide comprising a naturally occurring amino acid sequence at least 97% identical to the amino acid sequence of SEQ ID NO:20,
f) a polypeptide comprising a naturally occurring amino acid sequence at least 98% identical to the amino acid sequence of SEQ ID NO:9,
g) a polypeptide comprising a naturally occurring amino acid sequence at least 99% identical to an amino acid sequence selected from the group consisting of SEQ ID NO:11 and SEQ ID NO:18-19,
h) a polypeptide consisting essentially of a naturally occurring amino acid sequence at least 90% identical to an amino acid sequence selected from the group consisting of SEQ ID NO:12, SEQ ID NO:16-17, SEQ ID NO:21, and SEQ ID NO:23,
i) a biologically active fragment of a polypeptide having an amino acid sequence selected from the group consisting of SEQ ID NO: 1-25, and
j) an immunogenic fragment of a polypeptide having an amino acid sequence selected from the group consisting of SEQ ID NO: 1-25.

2. An isolated polypeptide of claim 1 selected from the group consisting of:
a) a polypeptide comprising an amino acid sequence selected from the group consisting of SEQ ID NO: 1-10 and SEQ ID NO: 14-25, and
b) a polypeptide consisting essentially of an amino acid sequence selected from the group consisting of SEQ ID NO:11-13.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:26-50.

6. A recombinant polynucleotide comprising a promoter sequence operably linked to a polynucleotide of claim 3.

7. A cell transformed with a recombinant polynucleotide of claim 6.

8. (Canceled)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO: 1-25.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:26-50,
b) a polynucleotide comprising a naturally occurring polynucleotide sequence at least 90% identical to a polynucleotide sequence selected from the group consisting of SEQ ID NO:26-32, SEQ ID NO:34-37, SEQ ID NO:39-40, SEQ ID NO:42, SEQ ID NO:45-48, SEQ ID NO:50
c) a polynucleotide comprising a naturally occurring polynucleotide sequence at least 91% identical to the polynucleotide sequence of SEQ ID NO:33,
d) a polynucleotide comprising a naturally occurring polynucleotide sequence at least 94% identical to the polynucleotide sequence of SEQ ID NO:44,
e) a polynucleotide comprising a naturally occurring polynucleotide sequence at least 95% identical to the polynucleotide sequence of SEQ ID NO:49,
f) a polynucleotide comprising a naturally occurring polynucleotide sequence at least 97% identical to the polynucleotide sequence of SEQ ID NO:38,
f) a polynucleotide comprising a naturally occurring polynucleotide sequence at least 99% identical to a polynucleotide sequence selected from the group consisting of SEQ ID NO:41 and SEQ ID NO:43,
h) a polynucleotide complementary to a polynucleotide of a),
i) a polynucleotide complementary to a polynucleotide of b),
j) a polynucleotide complementary to a polynucleotide of c),
k) a polynucleotide complementary to a polynucleotide of d),
l) a polynucleotide complementary to a polynucleotide of e),
m) a polynucleotide complementary to a polynucleotide of f),
n) a polynucleotide complementary to a polynucleotide of g), and
o) an RNA equivalent of a)-n).

13. (Canceled)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (Canceled)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide is selected from the group consisting of:
a) a polypeptide comprising an amino acid sequence selected from the group consisting of SEQ ID NO: 1-10 and SEQ ID NO: 14-25, and
b) a polypeptide consisting essentially of an amino acid sequence selected from the group consisting of SEQ ID NO:11-13.

19. (Canceled)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21-22. (Canceled)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24-25. (Canceled)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (Canceled)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-105. (Canceled)


SEQUENCE LISTING (50 sequences)

SEQ ID NO: 1 - protein, 733 aa, Homo sapiens
  misc_feature: Incyte ID No 7500354CD1
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAIPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 1036 aa, Homo sapiens
  misc_feature: Incyte ID No 3871329CD1
MGFALERFAEAVDPALECKLCGQVLEEPLCTPCGHVFCASCLLPWAVRRRRCPLQCQPLAPGELYRVLPLRSLIQKLRVQCDYRARGCGHSVRLHELEAHVEHCDFGPARRLRSRGGCASGLGGGEVPARGGCGPTPRAGRGGGARGGPPGGRWGRGRGPGPRVLAWRRREKALLAQLWALQGEVQLTARRYQEKFTQYMAHVRNFVGDLGGGHRRDGEHKPFTIVLERENDTLGFNIIGGRPNQNNQEGTSTEGIYVSKILENGPADRADGLEIHDKIMEVNGKDLSKATHEEAVEAFRNAKEPIVVQVLRRTPLSRPAYGMASEVQLMNASTQTDITFEHIMALAKLRPPTPPVPDICPFLLSDSCHSLHPMEHEFYEDNEYISSLPADADRTEDFEYEEVELCRVSSQEKLGLTVCYRTDDEEDTSIYVSEVDPNSIAAKDGRIREGDRILQINGEDVQNREEAVALLSNDECKRIVLLVARPEIQLDEGWLEDERNEFLEELNLEMLEEEHNEAMQPTANEVEQPKKQEEEEGTTDTATSSSNNHEKDSGVGRTDESLRNDESSEQENAAEDPNSTSLKSKRDLGQSQDTLGSVELQYNESLVSGEYIDSDCIGNPDEDCERFRQLLELKCKIRNHGEYDLYYSSSTIECNQGEQEGVEHELQLLNEELRNIELECQNIMQAHRLQKVTDQYGDIWTLHDGGFRNYNTSIDMQRGKLDDIMEHPEKSDKDSSSAYNTAESCRSTPLTVDRSPDSSLPRVINLTNKKNLRSTMAATQSSSGQSSKESTSTKAKTTEQGCSAESKEKGLEGSKLPDQEKAVSEHIPYLSPYHSSSYRYANIPAHARHYQSYMQLIQQKSAVEYAQSQLSLVSMCKESQKCSEPKMEWKVKIRSDGTRYITKRPVRDRILKERALKIKEERSGMTTDDDTMSEMKMGRYWSKEERKQHLVRAKEQRRRREFMMRSRLECLKESPQSGSEGKKEINIIELSHKKMMKKRNKKILDNWMTIQELMTHGAKSPDGTRVHNAFLSVTTV

SEQ ID NO: 3 - protein, 1847 aa, Homo sapiens
  misc_feature: Incyte ID No 1681386CD1
MLITQLPDIQEKLHQLQMEKLPSRKAITEMISWMNNVEHQTSDEDSVHSPSSASQVKHLLQKHKEFRMEMDYKQWIVDFVNQSLLQLSTCDVESKRYERTEFAEHLGEMNRQWHRVHGMLNRKIQHLEQLLESITESENKIQILNNWMEAQEERLKTLQKPESVISVQKLLLDCQDIENQLAIKSKALDELKQSYLTLESGAVPLLEDTASRIDELFQKRSSVLTQVNQLKTSMQSVLQEWKIYDQLYDEVNMMTIRFWYCMEHSKPVVLSLETLRCQVENLQSLQDEAESSEGSWEKLQEVIGKLKGLCPSVAEIIEEKCQNTHKRWTQVNQAIADQLQKAQSLLQLWKAYSNAHGEAAARLKQQEAKFQQLANISMSGNNLAEILPPALQDIKELQHDVQKTKEAFLQNSSVLDRLPQPAESSTHMLLPGPLHSLQRAAYLEKMLLVKANEFEFVLSQFKDFGVRLESLKGLIMHEEENLDRLHQQEKENPDSFLNHVLALTAQSPDIEHLNEVSLKLPLSDVAVKTLQNMNRQWIRATATALERCSELQGIGLNEKFLYCCEKWIQLLEKIEEALKVDVANSLPELLEQQKTYKMLEAEVSINQTIADSYVTQSLQLLDTTEIENRPEFITEFSKLTDRWQNAVQGVRQRKGDVDGLVRQWQDFTTSVENLFRFLTDTSHLLSAVKGQERFSLYQTRSLIHELKNKEIHFQRRRTTCALTLEAGEKLLLTTDLKTKESVGRRISQLQDSWKDMEPQLAEMIKQFQSTVETWDQCEKKIKELKSRLQVLKAQSEDPLPELHEDLHNEKELIKELEQSLASWTQNLKELQTMKADLTRHVLVEDVMVLKEQIEHLHRQWEDLCLRVAIRKQEIEDRLNTWVVFNEKNKELCAWLVQMENKVLQTADISIEEMIEKLQKDCMEEINLFSENKLQLKQMGDQLIKASNKSRAAEIDDKLNKINDRWQHLFDVIGSRVKKLKETFAFIQQLDKNMSNLRTWLARIESELSKPVVYDVCDDQEIQKRLAEQQDLQRDIEQHSAGVESVFNICDVLLHDSDACANETECDSIQQTTRSLDRRWRNICAMSMERRMKIEETWRLWQKFLDDYSRFEDWLKSAERTAACPNSSEVLYTSAKEELKRFEAFQRQIHERLTQLELINKQYRRLARENRTDTASRLKQMVHEGNQRWDNLQRRVTAVLRRLRHFTNQREEFEGTRESILVWLTEMDLQLTNVEHFSESDADDKMRQLNGFQQEITLNTNKIDQLIVFGEQLIQKSEPLDAVLIEDELEELHRYCQEVFGRVSRFHRRLTSCTPGLEDEKEASENETDMEDPREIQTDSWRKRGESEEPSSPQSLCHLVAPGHERSGCETPVSVDSIPLEWDHTGDVGGSSSHEEDEEGPYYSALSGKSISDGHSWHVPDSPSCPEHHYKQMEGDRNVPPVPPASSTPYKPPYGKLLLPPGTDGGKEGPRVLNGNPQQEDGGLAGITEQQSGAFDRWEMIQAQELHNKLKIKQNLQQLNSDISAITTWLKKTEAELEMLKMAKPPSDIQEIELRVKRLQEILKAFDTYKALVVSVNVSSKEFLQTESPESTELQSRLRQLSLLWEAAQGAVDSWRGGLRQSLMQCQDFHQLSQNLLLWLASAKNRRQKAHVTDPKADPRALLECRRELMQLEKELVERQPQVDMLQEISNSLLIKGHGEDCIEAEEKVHVIEKKLKQLREQVSQDLMALQGTQNPASPLPSFDEVDSGDQPPATSVPAPRAKQFRAVRTTEGEEETESRVPGSTRPQRSFLSRVVRAALPLQLLLLLLLLLACLLPSSEEDYSCTQANNFARSFYPMLRYTNGPPPT

SEQ ID NO: 4 - protein, 230 aa, Homo sapiens
  misc_feature: Incyte ID No 7500938CD1
MVKISFQPAVAGIKGDKADKASASAPAPASATEILLTPAREEQPPQHRSKRGGSVGGVCYLSMGMVVLLMGLVFASVYIYRYFFLAQLARDNFFRCGVLYEDSLSSQVRTQMELEEDVKIYLDENYERINVPVPQFGGGDPADIIHDFQRRGTYLPQTYIIQEEMVVTEHVSDKEALGSFIYHLCNGKDTYRLRRRATRRRINKRGAKNCNAIRHFENTFVVETLICGVV

SEQ ID NO: 5 - protein, 315 aa, Homo sapiens
  misc_feature: Incyte ID No 90055441CD1
MPLKLRGKKKAKSKETAGLVEGEPTGAGGGSLSASRAPARRLVFHAQLAHGSATGRVEGFSSIQELYAQIAGAFEISPSEILYCTLNTPKIDMERLLGGQLGLEDFIFAHVKGIEKEVNVYKSEDSLGLTITDNGVGYAFIKRIKDGGVIDSVKTICVGDHIESINGENIVGWRHYDVAKKLKELKKEELFTMKLIEPKKAFEIEPRSKAGKSSGEKIGCGRATLRLRSKGPATVEEMPSETKAKAIEKIDDVLELYMGIRDIDLATTMFEAGKDKVNPDEFAVALDETLGDFAFPDEFVFDVWGVIGDAKRRGL

SEQ ID NO: 6 - protein, 220 aa, Homo sapiens
  misc_feature: Incyte ID No 7500936CD1
MVKISFQPAVAGIKGDKADKASASAPAPASATEILLTPARLARDNFFRCGVLYEDSLSSQVRTQMELEEDVKIYLDENYERINVPVPQFGGGDPADIIHDFQRGLTAYHDISLDKCYVIELNTTIVLPPRNFWELLMNVKRGTYLPQTYIIQEEMVVTEHVSDKEALGSFIYHLCNGKDTYRLRRRATRRRINKRGAKNCNAIRHFENTFVVETLICGVV

SEQ ID NO: 7 - protein, 631 aa, Homo sapiens
  misc_feature: Incyte ID No 7500950CD1
MIPWPASDRKKSECAFKKKSNETQCFNFIRVLVSYNVTHLYTCGTFAFSPACTFIELQDSYLLPISEDKVMEGKGQSPFDPAHKHTAVLVDGMLYSGTMNNFLGSEPILMRTLGSQPVLKTDNFLRWLHHDASFVAAIPSTQVVYFFFEETASEFDFFERLHTSRVARVCKNDVGGEKLLQKKWTTFLKAQLLCTQPGQLPFNVIRHAVLLPADSPTAPHIYAVFTSQWQVGGTRSSAVCAFSLLDIERVFKGKYKELNKETSRWTTYRGPETNPRPGSCSVGPSSDKALTFMKDHFLMDEQVVGTPLLVKSGVEYTRLAVETAQGLDGHSHLVMYLGTTTGSLHKAVGAVFVGFSGGVWRVPRANCSVYESCVDCVLARDPHCAWDPESRTCCLLSAPNLNSWKQDMERGNPEWACASGPMSRSLRPQSRPQIIKEVLAVPNSILELPCPHLSALASYYWSHGPAAVPEASSTVYNGSLLLIVQDGVGGLYQCWATENGFSYPVISYWVDSQDQTLALDPELAGIPREHVKVPLTRVSGGAALAAQQSYWPHFVTVTVLFALVLSGALIILVASPLRALRARGKVQGCETLRPGEKAPLSREQHLQSPKECRTSASDVDADNNCLGTEVA

SEQ ID NO: 8 - protein, 132 aa, Homo sapiens
  misc_feature: Incyte ID No 7500854CD1
MPALLPVASRLLLLPRVLLTMASGSPPTQPSPASDSGSGYVPGSVSAAFVTCPNEKVAKEIARAVVEKRLAACVNLIPQITSIYEWKGKIEEDSEVLMFCAPLRSGRGNCIACGTGELSVPAVGAPGHRVSF

SEQ ID NO: 9 - protein, 1115 aa, Homo sapiens
  misc_feature: Incyte ID No 2754176CD1
MRKFNIRKVLDGLTAGSSSASQQQQQQHPPGNREPEIQETLQSEHFQLCKTVRHGFPYQPSALAFDPVQKILAVGTQTGALRLFGRPGVECYCQHDSGAAVIQLQFLINEGALVSALADDTLHLWNLRQKRPAILHSLKFCRERVTFCHLPFQSKWLYVGTERGNIHIVNVESFTLSGYVIMWNKAIELSSKSHPGPVVHISDNPMDEGKLLIGFESGTVVLWDLKSKKADYRYTYDEAIHSVAWHHEGKQFICSHSDGTLTIWNVRSPAKPVQTITPHGKQLKDGKKPEPCKPILKVEFKTTRSGEPFIILSGGLSYDTVGRRPCLTVMHGKSTAVLEMDYSIVDFLTLCETPYPNDFQEPYAVVVLLEKDLVLIDLAQNGYPIFENPYPLSIHESPVTCCEYFADCPVDLIPALYSVGARQKRQGYSKKEWPINGGNWGLGAQSYPEIIITGHADGSVKFWDASAITLQVLYKLKTSKVFEKSRNKDDRPNTDIVDEDPYAIQIISWCPESRMLCIAGVSAHVIIYRFSKQEVITEVIPMLEVRLLYEINDVETPEGEQPPPLPTPVGGSNPQPIPPQSHPSTSSSSSDGLRDNVPCLKVKNSPLKQSPGYQTELVIQLVWVGGEPPQQITSLAVNSSYGLVVFGNCNGIAMVDYLQKAVLLNLGTIELYGSNDPYRREPRSPRKSRQPSGAGLCDISEGTVVPEDRCKSPTSAKMSRKLSLPTDLKPDLDVKDNSFSRSRSSSVTSIDKESREAISALHFCETFTRKTDSSPSPCLWVGTTLGTVLVIALNLPPGGEQRLLQPVIVSPSGTILRLKGAILRMAFLDTTGCLIPPAYEPWREHNVPEEKDEKEKLKKRRPVSVSPSSSQEISENQYAVICSEKQAKVISLPTQNCAYKQNITETSFVLRGDIVALSNSICLACFCANGHIMTFSLPSLRPLLDVYYLPLTNMRIARTFCFTNNGQALYLVSPTEIQRLTYSQETCENLQEMLGELFTPVETPEAPNRGFFKGLFGGGAQSLDREELFGESSSGKASRSLAQHIPGPGGIEGVKGAASGVVGELARARLALDERGQKLGDLEERTAAMLSSAESFSKHAHEIMLKYKDKKWYQF

SEQ ID NO: 10 - protein, 363 aa, Homo sapiens
  misc_feature: Incyte ID No 7503408CD1
MEAPLVSLDEEFEDLRPSCSEDPEEKPQCFYGSSPHHLEDPSLSELENFSSEIISFKSMEDLVNEFDEKLNVCFRNYNAKTENLAPVKNQLQIQEEEETLQDEEVWDALTDNYIPSLSEDWRDPNIEALNGNCSDTEVIEEIEEMMQNSPDPEEEEEVLEEEDGGETSSQADSVLLQEMQALTQTFNNNWSYEGLRHMSGSELTELLDQVEGAIRDFSEELVQQLARRDELEFEKEVKNSFITVLIEVQNKQKEQRELMKKRRKEKGLSLQSSRIEKGNQMPLKRFSMEGISNILQSGIRQTFGSSGTDKQYLNTVIPYEKKASPPSVEDLQMLTNILFAMKEDNEKVPTLLTDYILKVLCPT

SEQ ID NO: 11 - protein, 453 aa, Homo sapiens
  misc_feature: Incyte ID No 71086982CD1
MALCLELLKQCSSCLVAYKKTPPPVPPRTTSKPFISVTVQSSTESAQDTYLDSQDHKSEVTSQSGLSNSSDSLDSSTRPPSVTRGGVAPAPEAPEPPPKHAALKSEQGTLTSSESHPEAAPKRKLSSIGIQVDCIQPVPKEEPSPATKFQSIGVQVEDDWRSSVPSHSMSSRRDTDSDTQDANDSSCKSSERSLPDCTPHPNSISIDAGPRQAPKIAQIKRNLSYGDNSDPALEASSLPPPDPWLETSSSSPAEPAQPGACRRDGYWFLKLLQAETERLEGWCCQMDKETKENNLSEEVLGKVLSAVGSAQLLMSQKFQQFRGLCEQNLNPDANPRPTAQDLAGFWDLLQLSIEDISMKFDELYHLKANSWQLVETPEKRKEEKKPPPPVPKKPAKSKPAVSRDKASDASDKQRQEARKRLLAAKRAASVRQNSATESADSIEIYVPEAQTRL

SEQ ID NO: 12 - protein, 505 aa, Homo sapiens
  misc_feature: Incyte ID No 7506367CD1
MALCLELLKQCSSCLVAYKKTPPPVPPRTTSKPFISVTVQSSTESAQDTYLDSQDHKSEVTSQSGLSNSSDSLDSSTRPPSVTRGGVAPAPEAPEPPPKHAALKSEQGTLTSSESPPRAAPKRKLSSIGIQVSSGAEAIAPLGGRSSMEHRRCWARGPGPRALEPWGLLKGNFAQSPLGPWGQVDCIQPVPKEEPSPATKFQSIGVQVEDDWRSSVPSHSMSSRRDTDSDTQDANDSSCKSSERSLPDCTPHPNSISIDAGPRQAPKIAQIKRNLSYGDNSDPALEASSLPPPDPWLETSSSSPAEPAQPGACRRDGYWFLKLLQAETERLEGWCCQMDKETKENNLSEEVLGKVLSAVGSAQLLMSQKFQQFRGLCEQNLNPDANPRPTAQDLAGFWDLLQLSIEDISMKFDELYHLKANSWQLVETPEKRKEEKKPPPPVPKKPAKSKPAVSRDKASDASDKQRQEARKRLLAAKRAASVRQNSATESADSIEIYVPEAQTRL

SEQ ID NO: 13 - protein, 711 aa, Homo sapiens
  misc_feature: Incyte ID No 1414020CD1
MLDGPLFSEGPDSPRELQDEESGSCLWVQKSKLLVIEVKTISCHYSRRAPSRQPMDFQASHWARGFQNRTCGPRPGSPQPPPRRPWASRVLQEATNWRAGPLAEVRAREQEKRKAASQEREAKETERKRRKAGGARRSPPGRPRPEPRNAPRVAQLAGLPAPLRPERLAPVGRAPRPSAQPQSDPGSAWAGPWGGRRPGPPSYEAHLLLRGSAGTAPRRRWDRPPPYVAPPSYEGPHRTLGTKRGPGNSQVPTSSAPAATPARTDGGRTKKRLDPRIYRDVLGAWGLRQGQGLLGGSPGCGAARARPEPGKGVVEKSLGLAAADLNSGSDSHPQAKATGSAGTEIAPAGSATAAPCAPHPAPRSRHHLKGSREGKEGEQIWFPKCWIPSPKKQPPRHSQTLPRPWAPGGTGWRESLGLGEGAGPETLEGWKATRRAHTLPRSSQGLSRGEGVFVIDATCVVIRSQYVPTPRTQQVQLLPSGVTRVVGDSPSQSKPGKEEGEGATVFPSPCQKRLSSSRLLHQPGGGRGGEAEGGRPGDSTLEERTFRILGLPAPEVNLRDAPTQPGSPEHQALGPAASGAQGRAEGSEVAVVQRRAGRGWARTPGPYAGALREAVSRIRRHTAPDSDTDEAEELSVHSGSSDGSDTEAPGASWRNERTLPEVGNSSPEEDGKTAELSDSVGEILDVISQTEEVLFGVRDIRGTQQGNRKRQ

SEQ ID NO: 14 - protein, 684 aa, Homo sapiens
  misc_feature: Incyte ID No 7621128CD1
MEANHSEQLSAERQSTPPGDSSSLPSHNGLEKEDGQDSPTPVQPPEKEASVHPDISEELNRQLEDIINTYGSAASTAGKEGSARASEQPENAESPDNEDGDCEETTEEAGREPVASGEPPTVKEPVSNKEQKLEKKILKGLGKEANLLMQNLNKLQTPEEKFDFLFKKYAELLDEHRTEQKKLKLLQKKQVQIQKEKDQLQGEHSRAILARSKLESLCRELQRHNKTLKEEALQRAREEEEKRKEITSHFQSTLTDIQGQIEQQSERNMKLCQENTELAEKLKSIIDQYELREEHLDKIFKHRELQQKLVDAKLEQAQEMMKEAEERHKREKEYLLNQAAEWKLQAKVLKEQETVLQAQLTLYSGRFEEFQSTLTKSNEVFATFKQEMDKTTKKMKKLEKDTATWKARFENCNKALLDMIEEKALRAKEYECFVMKIGRLENLCRALQEERNELHKKIRDAEISEKDDQSQHNSDEEPESNVSVDQEIDAEEVNSVQTAVKNLATAFMIIHHPESTPHQSKETQPEIGSSQESADAALKEPEQPPLIPSRDSESPLPPLTPQAEAEGGSDAEPPSKASNSPAGLGAETQCEGLPVGAQADQASWKPEAEASGQAPQAPTEASLQKMEADVPAPACAAEEHVAAMVPACEPSRQPPRAAAEELPVGASAGPQPRNVADTNLEGVD

SEQ ID NO: 15 - protein, 146 aa, Homo sapiens
  misc_feature: Incyte ID No 7505822CD1
MSQAPGAQPSPPTVYHERQRLELCAVHALNNVLQQQLFSQEAADEICKRPLSQLALPQVLGLILNLPSPVSLGLLSLPLRRRHWVALRQVDGVYYNLDSKLRAPEALGDEDGVRAFLAAALAQGLCEVLLVVTKEVEEKGSWLRTD

SEQ ID NO: 16 - protein, 902 aa, Homo sapiens
  misc_feature: Incyte ID No 71607945CD1
MSGQTLTDRIAAAQYSVTGSAVARAVCKATTHEVMGPKKKHLDYLIQATNETNVNIPQMADTLFERATNSSWVVVFKALVTTHHLMVHGNERFIQYLASRNTLFNLSNFLDKSGSHGYDMSTFIRRYSRYLNEKAFSYRQMAFDFARVKKGADGVMRTMAPEKLLKSMPILQGQIDALLEFDVHPNELTNGVINAAFMLLFKDLIKLFACYNDGVINLLEKFFEMKKGQCKDALEIYKRFLTRMTRVSEFLKVAEQVGIDKGDIPDLTQAPSSLMETLEQHLNTLEGKKPGNNEGSGAPSPLSKSSPATTVTSPNSTPAKTIDTSPPVDLFATASAAVPVSTSKPSSDLLDLQPDFSSGGAAAAAAPAPPPPAGGATAWGDLLGEDSLAALSSVPSEAQISDPFAPEPTPPTTTAEIATASASASTTTTVTAVTAEVDLFGDAFAASPGEAPAASEGAAAPATPTPVAAALDACSGNDPFAPSEGSAEAAPELDLFAMKPPETSVPVVTPTASTAPPVPATAPSPAPAVAAAAAATTAATAAATTTTTTSAATATTAPPALDIFGDLFESTPEVAAAPKPDAAPSIDLFSTDAFSSPPQGASPVPESSLTADLLSVDAFAAPSPATTASPAKVDSSGVIDLFGDAFGSSASEPQPASQAASSSSASADLLAGFGGSFMAPSPSPVTPAQNNLLQPNFEAAFGTTPSTSSSSSFDPSGDLLMPTMAPAGQPAPVSMVPPSPAMAASKALGSDLDSSLASLVGNLGISGTTTKKGDLQWNAGEKKLTGGANWQPKVAPATWSAGVPPSAPLQGAVPPTSSVPPVAGAPSVGQPGAGFGMPPAGTGMPMMPQQPVMFAQPMMRPPFGAAAVPGTQLSPSPTPASQSPKKPPAKDPLADLNIKDFL

SEQ ID NO: 17 - protein, 172 aa, Homo sapiens
  misc_feature: Incyte ID No 7505777CD1
MGTALDIKIKRANKVYHAGPQKGKFTPSPVDFTITPETLQNVKERALLPKFLLRGHLNSTNCVITQPLTGELVVESSEAAIRSVELQLVRVETCGCAEGYARDATEIQNIQIADGDVCRGLSVPIYMVFPRLFTCPTLETTNFKVEFEVNIVVLLHPDHLITENFPLKLCRI

SEQ ID NO: 18 - protein, 321 aa, Homo sapiens
  misc_feature: Incyte ID No 7505818CD1
MESIFHEKQPSGNMDDSGFFSIQVISNALKVWGLELILFNSPEYQRLRIDPINERSFICNYKEHWFTVRKLGKQWFNLNSLLTGPELISDTYLALFLAQLQQEGYSIFVVKGDLPDCEADQLLQMIRVQQMHRPKLIGEELAQLKEQRVHKTDLERMLEANDGSGMLDEDEEDLQRALALSRQEIDMEDEEADLRRAIQLSMQGSSRNISQDMTQTSGTNLTSEELRKRREAYFEKQQQKQQQQQQQQQQQQQQQQQQQQQQQQQGDLSGQSSHPCERPATSSGALGSDLGDAMSEEDMLQAAVTMSLETVRNDLKTEGKK

SEQ ID NO: 19 - protein, 362 aa, Homo sapiens
  misc_feature: Incyte ID No 7505821CD1
MESIFHEKQEGSLCAQHCLNNLLQGEYFSPVELSSIAHQLDEEERMRMAEGGVTSEDYRTFLQVISNALKVWGLELILFNSPEYQRLRIDPINERSFICNYKEHWFTVRKLGKQWFNLNSLLTGPELISDTYLALFLAQLQQEGYSIFVVKGDLPDCEADQLLQMIRVQQMHRPKLIGEELAQLKEQRVHKTDLERMLEANDGSGMLDEDEEDLQRALALSRQEIDMEDEEADLRRAIQLSMQGSSRNISQDMTQTSGTNLTSEELRKRREAYFEKQQQKQQQQQQQQQQQQQQQQQQQQQQQQQQGDLSGQSSHPCERPATSSGALGSDLGDAMSEEDMLQAAVTMSLETVRNDLKTEGKK

SEQ ID NO: 20 - protein, 332 aa, Homo sapiens
  misc_feature: Incyte ID No 7506685CD1
MALCLELLKQCSSCLVAYKKTPPPVPPRTTSKPFISVTVQSSTESAQDTYLDSQDHKSEVTSQSGLSNSSDSLDSSTRPPNSISIDAGPRQAPKIAQIKRNLSYGDNSDPALEASSLPPPDPWLETSSSSPAEPAQPGACRRDGYWFLKLLQAETERLEGWCCQMDKETNENNLSEEVLGKVLSAVGSAQLLMSQKFQQFRGLCEQNLNPDANPRPTAQDLAGFWDLLQLSIEDISMKFDELYHLKANSWQLVETPEKRKEEKKPPPPVPKKPAKSKPAVSRDKASDASDKQRQEARKRLLAAKRAASVRQNSATESADSIEIYVPEAQTRL

SEQ ID NO: 21 - protein, 214 aa, Homo sapiens
  misc_feature: Incyte ID No 7500933CD1
MVKISFQPAVAGIKGDKADKASASAPAPASATEILLTPAREEQPPQHRSKRGGSVGGVCYLSMGMVVLLMGLVFASVYIYRYFFLAQLARDNFFRCGVLYEDSLSSQVRTQMELEEDVKIYLDENYERINVPVPQFGGGDPADIIQEEMVVTEHVSDKEALGSFIYHLCNGKDTYRLRRRATRRRINKRGAKNCNAIRHFENTFVVETLICGVV

SEQ ID NO: 22 - protein, 716 aa, Homo sapiens
  misc_feature: Incyte ID No 7389203CD1
MFSPLKSRARALAHGDPFQVSRAQDFQVGVTVLEAQKLVGVNINPYVAVQVGGQRRVTATQRGTSCPFYNEYFLFEFHDTRLRLQDLLLEITAFHSQTLPFMATRIGTFRMDLGIILDQPDGQFYQRWVPLHDPRDTRAGTKGFIKVTLSVRARGDLPPPMLPPAPGHCSDIEKNLLLPRGVPAERPWARLRVRLYRAEGLPALRLGLLGSLVRALHDQRVLVEPYVRVSFLGQEGETSVSAEAAAPEWNEQLSFVELFPPLTRSLRLQLRDDAPLVDAALATHVPDLRRISHPGRAAGFNPTFGPAWVPLYGSPPGAGLRDSLQGLNEGVGQGIWFRGRLLLAVSMQVLEGRAEPEPPQAQQGSTLSRLTRKKKKKARRDQTPKAVPQHLDASPGAEGPEIPRAMEVEVEELLPLPENVLAPCEDFLLFGVLFEATMIDPTVASQPISFEISIGRAGRLEEQLGRGSRAGEGTEGAAVEAQPLLGARPEEEKEEEELGTPAQRPEPMDGSGPYFCLPLCHCKPCMHVWSCWEDHTWRLQSSNCVRKVAERLDQGLQEVERLQRKPGPGACAQLKQALEVLVAGSRQFCHGAERRTMTRPNALDRCRGKLLVHSLNLLAKQGLRLLRGLRRRNVQKKVALAKKLLAKLRFLAEEAPGAAPGEVCAKLELFLRLGLGKQAKACTSELPPDLLPEPSAGLPSSLHRDGPGADAEPSVG

SEQ ID NO: 23 - protein, 234 aa, Homo sapiens
  misc_feature: Incyte ID No 7506268CD1
MSFFPELYFNVDNGYLEGLVRGLKAGVLSQADYLNLVQCETLEAAFFQDCISEQDLDEMNIEIIRNTLYKAYLESFYKFCTLLGGTTADAMCPILEFEADRRAFIITINSFGTELSKEDRAKLFPHCGRLYPEGLAQLARADDYEQVKNVADYYPEYKLLFEGAGSNPGDKTLEDRFFEHEVKLNKLAFLNQFHFGVFYAFVKLKEQECRNIVWIAECIAQRHRAKIDNYIPIF

SEQ ID NO: 24 - protein, 728 aa, Homo sapiens
  misc_feature: Incyte ID No 7509159CD1
MALPALGLDPWSLLGLFLFQLLQLLLPTTTAGGGGQGPMPRVRYYAGDERRALSFFHQKGLQDFDTLLLSGDGNTLYVGAREAILALDIQDPGVPRLKNMIPWPASDRKKSECAFKKKSNEELQDSYLLPISEDKVMEGKGQSPFDPAHKHTAVLVDGMLYSGTMNNFLGSEPILMRTLGSQPVLKTDNFLRWLHHDASFVAAIPSTQVVYFFFEETASEFDFFERLHTSRVARVCKNDVGGEKLLQKKWTTFLKAQLLCTQPGQLPFNVIRHAVLLPADSPTAPHIYAVFTSQWQVGGTRSSAVCAFSLLDIERVFKGKYKELNKETSRWTTYRGPETNPRPGSCSVGPSSDKALTFMKDHFLMDEQVVGTPLLVKSGVEYTRLAVETAQGLDGHSHLVMYLGTTTGSLHKAVVSGDSSAHLVEEIQLFPDPEPVRNLQLAPTQGAVFVGFSGGVWRVPRANCSVYESCVDCVLARDPHCAWDPESRTCCLLSAPNLNSWKQDMERGNPEWACASGPMSRSLRPQSRPQIIKEVLAVPNSILELPCPHLSALASYYWSHGPAAVPEASSTVYNGSLLLIVQDGVGGLYQCWATENGFSYPVISYWVDSQDQTLALDPELAGIPREHVKVPLTRVSGGAALAAQQSYWPHFVTVTVLFALVLSGALIILVASPLRALRARGKVQGCETLRPGEKAPLSREQHLQSPKECRTSASDVDADNNCLGTEVA

SEQ ID NO: 25 - protein, 72 aa, Homo sapiens
  misc_feature: Incyte ID No 7512347CD1
MALPALGLDPWSLLGLFLFQLLQLLLPTTTAGGGGQGPMPRVRYYAGDERRALSFFHQKGLQAKEHDTVASQ

SEQ ID NO: 26 - DNA, 3495 bp, Homo sapiens
  misc_feature: Incyte ID No 7500354CB1
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaaaaaaaaaa

SEQ ID NO: 27 - DNA, 3720 bp, Homo sapiens
  misc_feature: Incyte ID No 3871329CB1
gaaatcaggggacctagcaggagcctgaaaacttcaagccaaacaaacagtgagatcacacctcccacccgccacctccctccactgccgccgccgcgagacggctgccccgggggtggcccggggaaggcaggggggctcggagaagacggactctgctttcgctccccctttcttccccatccctaacatgggctttgccctggagcgcttcgcagaagccgtggacccggctctggagtgcaaactgtgcggccaggtgcttgaagagcccctgtgcacgccgtgcgggcacgtcttctgcgccagctgcctgttgccctgggcggtgcggaggcgccggtgcccgctgcagtgccagcccttggcgcccggcgagctgtaccgggtgctgccgctgcgcagcctcatccagaagctgcgagtccagtgcgactaccgcgcccgcggctgcggccactcggtcaggctgcacgagctggaggcgcacgtcgagcactgcgacttcggccctgcccgccggctccgcagccgcgggggctgcgcttcggggctgggcggtggtgaggtgcccgcgcgggggggctgcggtccgacacccagggctggccggggcgggggcgcgcgcggggggccgccgggcggccgctggggccgcgggcggggacccgggcctcgggtcctcgcctggaggcggcgcgagaaggcgctgctggcgcagctctgggcgctgcagggcgaggtgcagctcacggcgcgcaggtaccaggagaagttcacccaatacatggctcacgtccgcaacttcgtcggcgacctcggtggcggccaccgcagggatggagagcataagccattcactattgtgttagaaagagaaaatgacactttgggattcaatattataggaggtcgaccaaatcagaataatcaggaaggaacatcgactgaaggaatttacgtttcaaaaattttagaaaatggacctgctgacagagcagatggcctggagattcatgacaaaatcatggaggtcaatgggaaggatctttcaaaggccactcatgaagaggcagtggaagcttttcgcaatgccaaggagcccattgtggtgcaggtgttaaggcgaacacctcttagtagaccagcctatgggatggcttcagaagtgcagcttatgaatgccagcactcagacggacatcaccttcgaacacatcatggctctggccaagcttcgtccacctacccctccagtgccagacatctgtccattcctgctctcagacagctgccattctctacatccaatggagcatgaattttatgaggacaatgagtatatttccagcttgcctgctgatgcagacagaacagaagactttgaatatgaggaggtcgagttgtgtcgtgttagcagtcaagagaagctgggcctgacagtctgttaccgaacagatgatgaagaagacaccagcatttatgtcagcgaggttgacccaaatagcattgctgccaaagacggccggattcgagaaggggatcggattttgcaaataaatggggaagatgtccagaatcgagaagaagcagtggccttgctgtctaacgatgagtgtaagagaatcgtgctgcttgttgcaaggccagagattcagctggatgaaggctggctggaagatgaaaggaatgaattcttagaggagttaaacttggagatgttggaagaagagcataatgaagcaatgcagcccactgccaatgaggtggagcagccaaaaaagcaagaagaagaagaaggcacaacagacactgcaacatcctcatccaacaaccatgagaaggacagtggagtaggacgtacagatgaaagcttgcgaaatgatgagagctcagagcaggagaatgcagccgaggaccccaatagcacatctttgaagagcaagagagacctggggcagagccaagacactctgggaagtgttgaacttcagtacaatgagagcctcgtatctggtgaatacattgactcagactgcattggcaacccagatgaggactgtgaaagattcaggcagctcttggagctcaaatgcaagattcgaaatcatggagagtatgacctgtattactcaagcagcacaattgaatgcaatcaaggggagcaagagggagtggagcatgagctacagttgcttaatgaagaactgagaaacattgagcttgagtgtcagaatatcatgcaggctcacaggctccagaaagtgacagaccagtatggagacatctggacattgcatgatggaggattccggaattataacaccagcatagatatgcaaaggggaaagctagatgacatcatggagcatccagaaaagtctgacaaggacagttctagtgcttacaacacagctgagagctgcagaagtactccgctcactgtagaccgttcccctgacagttcccttccaagggtgatcaacctcaccaataagaaaaacctgagaagcacaatggcagccacccagtcctcttccggacagagcagtaaagagtcgacctccaccaaagccaaaaccactgagcaaggttgtagcgctgaaagcaaggagaagggtttagaaggcagcaagcttcctgatcaagagaaggcagtcagcgaacacatcccttacctctctccttaccacagctcctcatatagatatgcaaacatcccagcacacgcccggcattatcaaagctacatgcagttaattcaacagaaatctgcagtcgagtatgctcagagtcagctcagcttggtgagcatgtgcaaggagtctcagaagtgttcagagcccaagatggaatggaaggtgaaaattaggagcgacgggacacggtacatcacaaagagacccgtgcgagaccgaatcctgaaggaacgtgccttaaagatcaaggaagagcggagtggcatgaccacagacgatgacaccatgagcgagatgaaaatggggcgctactggagcaaagaggagagaaagcagcacctggttagggccaaagagcagcgccgtcgccgtgagttcatgatgcgaagcaggttagagtgtctcaaggagagccctcagagcggcagtgagggcaagaaggagatcaatatcattgaactgagtcacaaaaagatgatgaaaaagagaaacaagaaaattttggacaactggatgacaatccaagaactgatgacccatggggccaagtctccagatggcacgagagtccataatgccttcttgtcggtgaccactgtatgaccgaatgaatggaatgcatgcgactgattttaggaggatgctaccagtttcggtagagtatgattgcctcgttcaatgtggcgtttttatatatattttgtgactctttatagtttaaattttttgtaagcaaaaaatacctggtaatttttcatttgtttttcatatactggtaccttctttttggctgagatctttcttttacttgtgatatattcatattactcgtttataaaaaatcaaaaacaaaaggaaagaaaacaaaaaaaacttgcacaaaaatactgaggagccaattaatttcctacttcaatgtatctaatgtaagtgaaaatctggatttatttctctagtttacttattttctactttaataataactcaatgccaaatatttctatgcttgtttcttggcataa

SEQ ID NO: 28 - DNA, 6119 bp, Homo sapiens
  misc_feature: Incyte ID No 1681386CB1
tccgacgctgtatacggcgccagtgtgctggaaaggttggtggcgtctgtgagctgtcctgaattagagggccagatcgcaaaactggaagagcagtggttgtccctgaacaagaaaattgaccatgagctccacaggctgcaagctcttctcaagcatctgctcagttataacagagattcggatcagttaaccaagtggttggaatcttcccagcatactctgaattactggaaagaacagtccctcaatgtgtctcaggacttggatacaatcagaagcaacatcaacaatttttttgagttttcaaaagaagttgatgaaaaatcctccttgaagactgccgttatcagtatcgggaaccagcttcttcacctgaaagaaactgatacagctacactgagagcttctttagcacagtttgaacaaaaatggacaatgctcataactcaacttccagatattcaagaaaaacttcaccagcttcaaatggagaaattgccgtctcgtaaagcaatcacagaaatgattagctggatgaacaatgtggagcatcaaacttcagatgaagactccgtgcattcaccaagttctgcatctcaagttaaacatcttcttcagaagcacaaggagtttagaatggaaatggactataaacagtggatagttgacttcgttaaccagtcattacttcagctaagcacctgtgatgtagaaagcaagcgctatgaaagaacggagtttgcagagcacctgggggagatgaaccgccagtggcaccgtgtacatggaatgctgaatagaaagatacaacatttagaacaacttctagaaagtatcactgagagtgaaaataaaatacagattttgaacaactggatggaagcacaagaagagagactgaaaactttacaaaaacctgaaagtgtgatctcagtgcagaagctgctcctggactgtcaggatatagaaaatcaacttgcaattaaatccaaagcactagatgagttgaaacaaagttatctgactttggagagtggggcagtgccattgttagaagatacagcatcccgaattgatgagttatttcaaaagagaagcagtgttctcactcaggtcaatcagctcaaaacctccatgcagtcagttttacaggagtggaagatttatgatcaactctatgatgaagtgaatatgatgacaatccgattctggtactgcatggaacacagcaagcctgtggtgttatcattggagaccttgagatgccaggtggagaaccttcagtctctgcaagatgaagctgagagcagtgaagggagttgggagaaactccaggaggttatcggcaaactcaaaggtctctgcccctctgttgctgaaataatcgaagagaaatgccaaaatactcataaaaggtggactcaggtgaaccaagccattgcagaccagttgcagaaggcccagagtctgctccagctctggaaggcctatagcaatgctcatggtgaagctgccgcaaggctgaagcagcaggaagcaaagtttcaacagctcgcaaacatcagcatgtctggaaacaacctggcagagatcctgcccccagccctgcaggacataaaggagctgcagcatgatgtgcagaaaacaaaagaagcctttctccaaaattccagtgtcctggatcgactcccacaacccgcagagtccagcacccacatgctcctcccgggccccctgcactctctccagagggctgcttatttggaaaagatgctgcttgtgaaagcaaatgaatttgagtttgttctctcacagtttaaggattttggagtccggctggaatctttaaaaggtcttattatgcatgaagaagagaatttggatagacttcaccaacaggaaaaagaaaatcctgactcattcctgaatcatgtgctggcactgacagcccaatcacctgatattgaacatttgaatgaagtgagcctcaagctcccacttagtgacgtagctgtgaagacgttacaaaatatgaaccggcaatggattcgggccacggccacggcactggagcgctgcagtgagcttcagggaattggattgaatgaaaagtttctttattgctgtgaaaagtggatccaacttttggagaagatagaagaagcactcaaagtggatgtggctaacagccttcctgagctcctggagcagcagaaaacctataagatgttagaagctgaagtttctataaaccagacaattgctgattcctatgtcacccagtccttacaactcctggacacaacagaaatagagaacagaccagaatttattacagaattctcaaagctgacggatcggtggcagaatgctgtccagggtgttcggcagaggaagggtgacgttgatgggctggtgaggcagtggcaagatttcactacttctgtggagaacttgtttcgcttcctcactgacaccagccacctgctatctgcagtgaagggccaggagcgcttcagcctataccaaaccagaagtctgatccatgagctgaagaataaagaaattcattttcaaaggaggcgaactacctgtgccctaaccttggaagctggagaaaagttactgctcacaactgacctgaaaactaaagagtctgtgggtaggagaatcagtcaacttcaggacagctggaaagacatggagccccagctggcagagatgattaagcagttccagagcactgtagagacctgggaccagtgtgaaaagaaaatcaaggagttgaaaagcaggctgcaagttttaaaggcacaaagtgaagatcctcttccagagcttcacgaggacctccataacgaaaaagagctgattaaggaactagaacagtctttggctagctggactcagaacttgaaagaacttcaaactatgaaggcggacttaacccggcacgttctcgtggaagatgtgatggttttgaaggagcaaatagagcatttgcacagacaatgggaggacctctgcttaagggtggccatacgtaaacaggagattgaagacagactcaatacatgggttgtattcaatgaaaaaaataaagagttgtgtgcctggctggtgcagatggaaaacaaagttctacagacagcggacattagtattgaagaaatgattgaaaagttacagaaggactgcatggaagaaataaacttgtttagtgaaaacaagttacagttaaagcagatgggtgaccagttgatcaaggccagcaacaaatcaagagcagctgagatcgatgacaagctcaacaaaattaacgatcgttggcaacatctttttgatgtcatcggatcaagggtgaagaagctgaaggagacctttgcttttattcagcagttggacaaaaacatgagcaaccttcgcacctggttggctcgaattgagtctgagctttccaagcctgttgtttatgatgtctgcgatgatcaagagatccagaagaggctcgctgagcagcaggatctacagcgagatattgaacaacacagcgcaggggtggagtccgtgtttaacatctgtgacgtcctactgcacgactccgatgcctgtgcaaatgagaccgagtgtgactcgatccagcagaccaccaggagcctggacagacgctggaggaacatttgtgccatgtccatggagcggcgcatgaaaatcgaggagacgtggcgcctgtggcagaagtttttagacgactattctcgctttgaggactggctcaagtcagctgagaggacggcagcctgcccaaattcctcagaggtgttgtacacgagtgccaaagaggaactgaagaggtttgaggcctttcagcggcagattcatgagcggctcactcagctggagctcatcaacaagcagtaccggcggctggcccgggagaaccgcacagacacggccagcaggctgaagcagatggtccacgagggcaaccagcgctgggacaaccttcagaggcgggtcacagccgtcctgcggagactcaggcatttcaccaaccagagggaagaatttgagggcaccagggagagcattctggtgtggctcacagagatggacctgcagctgaccaacgtggagcacttctcagagagtgacgccgatgacaagatgcgccaactgaatggcttccaacaggaaattacattaaataccaacaagattgatcagctcattgtgtttggggagcagctgattcagaagagcgagcccctggatgctgtgctgattgaggatgagctggaggaactccaccgctactgccaggaggtgtttggaagggtctcccggttccaccggcggctcacctcctgcactccgggcttggaagatgaaaaggaggcctctgagaatgaaacagacatggaagaccccagagaaatccagactgattcttggcgtaaacggggagagagcgaggaaccgtcatctcctcagtccctgtgtcatctagtggccccagggcacgagcggtctggctgcgagacccctgtcagcgtggactccatccccctggagtgggaccacacaggcgacgtggggggctcctcctctcacgaagaggacgaggagggcccatactacagcgcactgtcaggtaaatccatttcggatggccactcgtggcatgttcccgacagcccttcctgtcccgagcatcactacaagcaaatggaaggtgacaggaatgttccacctgttccccctgcgtccagcaccccttataaaccaccctatggaaagctactattacctccaggcacggatggtggcaaagaaggcccgcgagtcctgaatggcaacccacagcaggaagacgggggactggccggtatcacagagcagcagtcaggtgccttcgacagatgggagatgattcaagcacaggagcttcacaataagctcaaaataaaacaaaatttgcaacagctgaactctgatatcagcgccatcactacttggctgaaaaaaactgaagcagagctggaaatgttaaagatggcaaagcctccctctgatatccaggaaatagaactgagagtgaagagacttcaggagatactgaaagcctttgacacttacaaggcattagtggtctctgtcaacgtgagcagcaaggaatttctgcaaaccgagagccccgaatccacagagctccaaagtagactccgccagctgagcctgctctgggaagcagcacagggcgcagtggacagctggagagggggcttacgacagtcgctcatgcagtgccaggacttccaccagttgagtcaaaatctgctgctgtggttagcgagtgccaagaaccggaggcagaaggctcatgtcaccgatccaaaggcagacccccgggctctcctagagtgtcggagggaactaatgcaactggaaaaggagctggtagaacgtcaacctcaagtggacatgttacaggagatttcaaacagccttctcattaagggacatggagaagactgtattgaagctgaagaaaaggtgcatgttattgagaagaaactcaaacagttacgggagcaagtgtcccaagatttaatggccttgcagggaacccagaacccagcctcacccctgcccagcttcgacgaggtagactcgggggaccagcctcctgcaacatccgtgccagctccccgagcaaagcagttcagagcagtgagaactacagaaggcgaggaggagacagagagcagggtccccggcagcacacggccacagcgctccttcctctcaagggtggtccgggcagccctacccctgcagctgctcctcctgctgctgctgctcctggcctgcctgctgccctcctccgaagaagactacagctgcactcaggccaacaactttgcccggtccttttaccccatgctgaggtacaccaatgggccaccccccacatagagggcatagctggccacagtgctacaccacctgcctgattgccaagggtgcccagcacgtggccccagaccaatctgagtgacttagtgtttgcaagggggatccactagttctaagcgccgcaccccgcgtgctcc

SEQ ID NO: 29 - DNA, 1151 bp, Homo sapiens
  misc_feature: Incyte ID No 7500938CB1
cgagcgggatccaaacttccggtgcctgcagagctcggagcggcggaggcagagaccgaggctgcaccggcagaggctgcggggcggacgcgcgggccggcgcagccatggtgaagattagcttccagcccgccgtggctggcatcaagggcgacaaggctgacaaggcgtcggcgtcggcccctgcgccggcctcggccaccgagatcctgctgacgccggctagggaggagcagcccccacaacatcgatccaagagggggggctcagtgggcggcgtgtgctacctgtcgatgggcatggtcgtgctgctcatgggcctcgtgttcgcctctgtctacatctacagatacttcttccttgcgcagctggcccgagataacttcttccgctgtggtgtgctgtatgaggactccctgtcctcccaggtccggactcagatggagctggaagaggatgtgaaaatctacctcgacgagaactacgagcgcatcaacgtgcctgtgccccagtttggcggcggtgaccctgcagacatcatccatgacttccagcggagggggacctacctgccgcagacgtacatcatccaggaggagatggtggtcacggagcatgtcagtgacaaggaggccctggggtccttcatctaccacctgtgcaacgggaaagacacctaccggctccggcgccgggcaacgcggaggcggatcaacaagcgtggggccaagaactgcaatgccatccgccacttcgagaacaccttcgtggtggagacgctcatctgcggggtggtgtgaggccctcctcccccagaaccccctgccgtgttcctcttttcttctttccggctgctctctggccctcctccttccccctgcttagcttgtactttggacgcgtttctatagaggtgacatgtctctccattcctctccaaccctgcccacctccctgtaccagagctgtgatctctcggtggggggcccatctctgctgacctgggtgtggcggagggagaggcgatgctgcaaagtgttttctgtgtcccactgtcttgaagctgggcctgccaaagcctgggcccacagctgcaccggcaggcccaagggggaaggaccgtttgggggagccgggcatgtgaaggccct

SEQ ID NO: 30 - DNA, 1277 bp, Homo sapiens
  misc_feature: Incyte ID No 90055441CB1
cggaagcgcggctgccattggaggctgcttttacctgcgcggggcccggggcgcaaagtccgaggcgccggggggaggaggcggcggacggcagcgcaggtgggcccgcgctctcggccctgcaagatgcccctgaagctgcgggggaagaagaaggccaagtccaaggagaccgccgggctggtggagggcgagccgacgggcgcgggcggcgggagcctctcagcgtcccgggctcccgcacgcaggctggtcttccacgcgcagctggcgcacggtagtgccacgggccgagtggagggcttctccagcatccaggagctctacgcccagatcgcgggcgcgtttgaaatctcgccgtcggagatcttatattgcactttaaacacacctaaaattgacatggaaagactcttaggaggacaactaggactagaagatttcatatttgcccatgtgaaaggaatcgaaaaagaagtgaatgtgtataaatctgaggattcacttggtctcaccattacagataatggtgttggctatgcttttataaagagaattaaagatggtggtgttattgactcagttaaaacaatctgtgttggggatcatattgaatccataaatggagaaaatattgttgggtggcgtcactatgatgttgctaagaagttaaaggaattaaaaaaggaggaactctttactatgaagttaatagaacctaagaaggcatttgaaatagagccgaggtcaaaggctggaaagtcatcaggagaaaaaattggttgtggaagggcaacacttcgcctgagatcaaaaggtcctgccaccgtggaagaaatgccttctgaaaccaaagcaaaggcaattgaaaagattgatgatgttcttgagttgtacatgggaattcgagatattgatttagccaccacaatgtttgaagctggaaaggacaaagtaaatccagatgaatttgctgtggcacttgacgaaactcttggagactttgcgttcccagacgaatttgtctttgatgtttggggagtcattggtgatgccaaacgaagaggattatgatgtgtacactccatctctgaagaaacaacccatcgttcttttttttctcttttttaaaaagtcctataagatctgtttttggacacctttactaactctggtttaatttcatgtgtatggaatatattctttgaaatataattttggtaattttgatttctgggcactttttaacattgctgatgtagtatgcttaagagaaa

SEQ ID NO: 31 - DNA, 1041 bp, Homo sapiens
  misc_feature: Incyte ID No 7500936CB1
gatccaaacttccggtgcctgcagagctcggagcggcggaggcagagaccgaggctgcaccggcagaggctgcggggcggacgcgcgggccggcgcagccatggtgaagattagcttccagcccgccgtggctggcatcaagggcgacaaggctgacaaggcgtcggcgtcggcccctgcgccggcctcggccaccgagatcctgctgacgccggctaggctggcccgagataacttcttccgctgtggtgtgctgtatgaggactccctgtcctcccaggtccggactcagatggagctggaagaggatgtgaaaatctacctcgacgagaactacgagcgcatcaacgtgcctgtgccccagtttggcggcggtgaccctgcagacatcatccatgacttccagcggggtctgactgcgtaccatgatatctccctggacaagtgctatgtcatcgaactcaacaccaccattgtgctgccccctcgcaacttctgggagctcctcatgaacgtgaagagggggacctacctgccgcagacgtacatcatccaggaggagatggtggtcacggagcatgtcagtgacaaggaggccctggggtccttcatctaccacctgtgcaacgggaaagacacctaccggctccggcgccgggcaacgcggaggcggatcaacaagcgtggggccaagaactgcaatgccatccgccacttcgagaacaccttcgtggtggagacgctcatctgcggggtggtgtgaggccctcctcccccagaaccccctgccgtgttcctcttttcttctttccggctgctctctggccctcctccttccccctgcttagcttgtactttggacgcgtttctatagaggtgacatgtctctccattcctctccaaccctgcccacctccctgtaccagagctgtgatctctcggtggggggcccatctctgctgacctgggtgtggcggagggagaggcgatgctgcaaagtgttttctgtgtcccactgtcttgaagctgggcctgccaaag

SEQ ID NO: 32 - DNA, 2745 bp, Homo sapiens
  misc_feature: Incyte ID No 7500950CB1
gcatgatgggcacctggagggccgcactcccgttccagccaggctgagccttctgtcccctgcctctggggcctgggaaccccccttcttctttctcctgaatggcacccccgccctagaatccagacaccgagtttcccactgtggctggttcaagggtatgtgaggggatgaacgtagggcacttagcttcttccaccagaagggcctccaggattttgacactctgctcctgagtggtgatggaaatactctctacgtgggggctcgagaagccattctggccttggatatccaggatccaggggtccccaggctaaagaacatgataccgtggccagccagtgacagaaaaaagagtgaatgtgcctttaagaagaagagcaatgagacacagtgtttcaacttcatccgtgtcctggtttcttacaatgtcacccatctctacacctgcggcaccttcgccttcagccctgcttgtaccttcattgaacttcaagattcctacctgttgcccatctcggaggacaaggtcatggagggaaaaggccaaagcccctttgaccccgctcacaagcatacggctgtcttggtggatgggatgctctattctggtactatgaacaacttcctgggcagtgagcccatcctgatgcgcacactgggatcccagcctgtcctcaagaccgacaacttcctccgctggctgcatcatgacgcctcctttgtggcagccatcccttcgacccaggtcgtctacttcttcttcgaggagacagccagcgagtttgacttctttgagaggctccacacatcgcgggtggctagagtctgcaagaatgacgtgggcggcgaaaagctgctgcagaagaagtggaccaccttcctgaaggcccagctgctctgcacccagccggggcagctgcccttcaacgtcatccgccacgcggtcctgctccccgccgattctcccacagctccccacatctacgcagtcttcacctcccagtggcaggttggcgggaccaggagctctgcggtttgtgccttctctctcttggacattgaacgtgtctttaaggggaaatacaaagagttgaacaaagaaacttcacgctggactacttataggggccctgagaccaacccccggccaggcagttgctcagtgggcccctcctctgataaggccctgaccttcatgaaggaccatttcctgatggatgagcaagtggtggggacgcccctgctggtgaaatctggcgtggagtatacacggcttgcagtggagacagcccagggccttgatgggcacagccatcttgtcatgtacctgggaaccaccacagggtcgctccacaaggctgtgggtgcagtgtttgtaggcttctcaggaggtgtctggagggtgccccgagccaactgtagtgtctatgagagctgtgtggactgtgtccttgcccgggacccccactgtgcctgggaccctgagtcccgaacctgttgcctcctgtctgcccccaacctgaactcctggaagcaggacatggagcgggggaacccagagtgggcatgtgccagtggccccatgagcaggagccttcggcctcagagccgcccgcaaatcattaaagaagtcctggctgtccccaactccatcctggagctcccctgcccccacctgtcagccttggcctcttattattggagtcatggcccagcagcagtcccagaagcctcttccactgtctacaatggctccctcttgctgatagtgcaggatggagttgggggtctctaccagtgctgggcaactgagaatggcttttcataccctgtgatctcctactgggtggacagccaggaccagaccctggccctggatcctgaactggcaggcatcccccgggagcatgtgaaggtcccgttgaccagggtcagtggtggggccgccctggctgcccagcagtcctactggccccactttgtcactgtcactgtcctctttgccttagtgctttcaggagccctcatcatcctcgtggcctccccattgagagcactccgggctcggggcaaggttcagggctgtgagaccctgcgccctggggagaaggccccgttaagcagagagcaacacctccagtctcccaaggaatgcaggacctctgccagtgatgtggacgctgacaacaactgcctaggcactgaggtagcttaaactctaggcacaggccggggctgcggtgcaggcacctggccatgctggctgggcggcccaagcacagccctgactaggatgacagcagcacaaaagaccacctttctcccctgagaggagcttctgctactctgcatcactgatgacactcagcagggtgatgcacagcagtctgcctcccctatgggactcccttctaccaagcacatgagctctctaacagggtgggggctacccccagacctgctcctacactgatattgaagaacctggagaggatccttcagttctggccattccagggaccctccagaaacacagtgtttcaagagaccctaaaaaacctgcctgtcccaggaccctatggtaatgaacaacaaacatctaaacaatcatatggctaacatgccaatcctggaaactccactctgtaagctgccggctttgagaccaacactgccttcttccagggtcatgcagggatctgctcccttctgctttccttaccagtcgtgcaccgctga

SEQ ID NO: 33 - DNA, 627 bp, Homo sapiens
  misc_feature: Incyte ID No 7500854CB1
cccagaggttggccccctgaggtgcctctctgctcctgtcttttgtttggatgccggcgctgctgcctgtggcctcccgccttttgttgctaccccgagtcttgctgaccatggcctctggaagccctccgacccagccctcgccggcctcggattccggctctggctacgttccgggctcggtctctgcagcctttgttacttgccccaacgagaaggtcgccaaggagatcgccagggccgtggtggagaagcgcctagcagcctgcgtcaacctcatccctcagattacatccatctatgagtggaaagggaagatcgaggaagacagtgaggtgctgatgttctgtgcacccttacgaagtggccgaggtaattgcattgcctgtggaacaggggaactttccgtacctgcagtgggtgcgccaggtcacagagtcagtttctgactctatcacagtcctgccatgatgagccctgttcctgctcatcatgaagatccccgcgatacttcaacgccttctgacttccaggtgatgactgggcccccaataaatcccgtctttgggtctctctgaaaaaaaaaaaaaaaaaaaaaaaaaaaaaaaaaaaaaaaaaaaaaaaacc

SEQ ID NO: 34 - DNA, 5899 bp, Homo sapiens
  misc_feature: Incyte ID No 2754176CB1
cgctcttgtcgtcgcagtatttcctgttcggattatctttggcttccccccagctgcctccttaccctcacactcccactcctccgtttccgcggtcgaagctgccttcggccccgggtggtctcccccgcccggggaccccctgtgcctcccctcccgggctgcgggggagcccctccgagaccatgaggaaattcaacatcaggaaggtgctggacggcctgaccgccggctcgtcctcggcgtcgcagcagcaacagcagcagcatccgcctgggaaccgggagccggagatccaggaaacgctccagtccgagcactttcagctctgcaagactgttcgccatggatttccctatcaaccctcagccctggcctttgatcctgtacagaagatcctggcagtgggaactcagactggtgctttaaggctctttggtcgtccaggagtagaatgttattgccagcatgacagtggagctgcagtaatccagctccagttcctgattaatgagggagcgcttgtgagtgccttggctgatgacaccttacacttatggaatttacgtcagaagaggcctgccatactacattcgcttaaattttgcagagaaagggttacattttgccatctgcctttccagagtaagtggctctatgtgggcactgaacgaggtaatatacatattgtcaatgtggagtccttcacactctcaggctacgtcattatgtggaataaagccattgaactgtcatctaaatctcacccaggacctgtggtccatataagtgataatccaatggacgagggaaagcttttgattggctttgaatctggaacagtagttttatgggacctcaaatcaaagaaagccgactacagatacacatatgatgaggctatccactctgttgcttggcatcatgaaggaaaacaatttatttgcagtcattcagatggcaccttgactatatggaatgtaaggtcccctgctaaaccagtacagacaatcactccacatggaaaacagttaaaggatgggaagaagccagaaccatgcaaacctatcctcaaggtggaattcaaaacgactagatctggggagccttttattattttatcaggaggtttgtcatatgatactgtaggaagaagaccttgcttaacagtgatgcatgggaaaagcactgctgtgctagaaatggactattcaattgttgattttctaacgctgtgtgaaacaccatacccaaatgattttcaagaaccatatgctgtggttgttcttctagaaaaggatttagtacttatagaccttgcacaaaatggatatcctatatttgaaaatccctaccctttgagtatacatgagtcccctgttacatgttgcgaatattttgcggattgtcctgtggaccttattcctgcactttattctgttggagctagacagaaacgtcaaggttacagcaaaaaggaatggcccatcaacggaggtaattggggcttgggtgctcaaagttacccagaaataattattacagggcatgctgatgggtcagttaagttctgggatgcttctgcaataactctacaagtattatataagctaaagacatctaaagtatttgaaaagtcaagaaataaagatgacaggccaaacacagacattgtagatgaagatccatatgccattcagatcatctcctggtgtccagaaagtagaatgctgtgcatcgctggagtttcagctcatgtcattatttatagattcagcaagcaggaagtaatcacagaagtcattccgatgcttgaagttcgattattatatgagataaatgatgtggaaactccggagggtgagcagccaccacctttgccaacacccgtgggagggtccaaccctcagcccatccctcctcagtctcatccatctaccagtagcagttcatctgatgggcttcgtgataatgtaccttgtttaaaagttaaaaactcaccacttaaacagtctccaggttatcaaacagaactagttattcagttggtttgggtgggtggagaaccaccacaacaaataaccagcctggcagtcaattcttcctatggactggtggtttttggcaattgcaatggcattgctatggttgactacctccagaaagcagtgctgctcaacctgggcactattgaattatatggctctaatgatccttatcggagagaaccccgatctcctcgtaaatctcgacagccttcaggagccggtctgtgtgatattagtgaagggactgttgttccagaggatcgctgcaaatctccaacctctgcaaagatgtcaaggaagttaagcttacctactgacctaaagcctgatttagatgtaaaggataactcctttagccgatcacggagttcaagtgtaacaagcattgacaaagaatcccgagaagcgatctccgctcttcatttctgtgaaacgtttactcgaaagacggactcgtccccttccccttgtctatgggttggaacaacgctaggaacagtgcttgtcattgcactgaaccttcccccagggggagagcaaagacttcttcagccagtaattgtgtctccaagtggtactatattgaggttaaaaggtgcaatcttgagaatggcatttctggataccacaggctgcttaataccacctgcgtatgaaccctggagagagcacaatgttcctgaagaaaaagacgaaaaggagaaattgaaaaaacggcggcctgtctcagtatccccctcctcttctcaggaaattagtgaaaaccagtatgcagtgatatgttctgaaaagcaagcaaaagtaatctcactgccaacccagaactgtgcttataagcaaaatattacagagacctcgtttgtgcttcgtggagatattgtagcattgagtaacagtatctgccttgcctgtttctgtgccaatggacatataatgacttttagtttgccaagtttaagacctctgttggatgtgtattacttgccccttaccaatatgcggatagccagaacgttctgctttaccaacaatggacaagcattataccttgtttcacctacagaaatccagagacttacttatagtcaagagacctgtgaaaatcttcaggaaatgttgggtgaactcttcactcctgtagaaacacctgaagcaccaaacaggggattctttaaaggcttatttggaggtggtgcacaatctcttgacagagaagaactatttggagaatcgtcctcaggaaaggcttcaaggagccttgcacagcatattcctggccctggtggcattgaaggcgtaaaaggggcagcatctggagttgttggtgaattagcacgagccaggctggcactagatgaaagagggcagaaacttggcgatctggaagaaagaactgcggccatgttatcaagtgcagagtcattttctaaacatgctcatgagattatgttgaaatacaaagataagaagtggtaccagttctgacaaccagaatccaataagtccaacttcagccagaaggaaaaaagttttccatttttattacattctttaggaaagttaacgttaaagggatgttcgtcactgaatactgttctttcctagcacagtcatgcactgttttacctcagtcatgtggctttaactgaggagtgttcacacgcactcgaaatggagtatatggtgtgtgccagttatgagttgaccatttgggaattaaacaggtcacacgtgacagatgaagaaaccaagggggctgctgaggagacctggtgcagggactaatcctggatcattcctgtattaaactttcatatgccaaaagggtttgtgccgttttatctgccatcagtgtttgacctgtttagggcagaggcaataagtcagaagtcttgaagttgaaatagttatatgtgtgtcattggactggattataaacagctgtcttggactttccctctcttaacactgactggtcatcagtatcattagtgaaaaagaaacaaattgtttgttatcatctctttagacagataagctgaatggtgggctttaaataataaaaacatacacatagttgacttgtgtatgagctactcttggattcttgttattatagacttgtatttagttcatattttgtcaaaagcaaaacaagaagatacatcacttttcattgaaaagaaaagtgtagagcatgactgaattgctcatcattctgggagtttccatgtagtggctatgcagtgtggaaagtgagaaaaacctccattgtggtgaggagaatacttcaatgtcccttgtccttgttctcattaattcagctaaaggtggatttgaccaaaataatgctggttaaatttgtagaaatgttgaaattggctgtgttttaaattttgcttgaatttttataaaatgtttaaccaaatgtacctttgcattctttaattaaaattgcttaaaaaaaaactttcattatttcagtaaaatgctcagctcccttttcaaaatgccctttatttgctaactgttccaatacactcaggtgatgagccaattaaatgttagtgcacatgctatcgcatggaaaattacaagcatctgttgtcaataattatataagagtttagtctgatgacctacaaaatattttgtgtagaaatatactataaatctgtacatatcctttcaaggttttaaaaaaccaaaaagaaaggaaaatatgtacactgtgataactaaattatttctgtattggaatataatacaatttgagaccagcaatggacaatgaatgattgtttcatagaatagcattacgggcaggaaagaaaccacctagtagatctagaagtaagcaatctcagaatacagactaatctgagattattattgattgttgttaaagcaacccagcaaaagcaaaatggaagcattaaaattaatgttaatgaaatttaaatattgtcttctataaaaaattcctttaaatgattttgttttttcattaagagataatcagagcacaaattgatagtaaacaggatggttgtttttctattctatatgatcattaaaggaatatgtaggacatcttaactttttcatacagtctgttatgcatattttcctctacttttcattaataaagcttttatgtttacattttataacatgcactactagatgcaaaagtttacatcaaagtttactttaaatatcatttggtagggactaaatgtgtgtgatagagataattgatcaagccaaaagaaatattttttaaataagcccttttcaaaagtttttgaatttatagaaagcaccaatgaataacatatttctgtttcgttaatgtcagctgcctgaacattcagcagtttataaattgcttaatttgtgttatctattatccagtaaacccatagttccatgatatgtcacaggaattgttaggtcctattttaaaggtacagttttgtgaatgtcatcaataaaatcaacagttatggatttgaagaagttgggaaagcattatgtagattaatatactggttggttccctatctatgtggaaggtcatattagctgcaattatttaatttgctgtgttattttgtgttatataacacaaatatatttgtatattaacttcatttttactgtcatttttcctgttgtatacaaaatgaactaatcttgtaattattttcaaatatagaagtatatacattagatggatttccaagattttgtaagaaaatcttaaatcagtgttttgagttatttaatttttaaattaatctacaaattatgcacaacaaactagggactccattagggttaggaac

SEQ ID NO: 35 - DNA, 1542 bp, Homo sapiens
  misc_feature: Incyte ID No 7503408CB1
tccggctgagccccgggatccgcctccctccgccaggacccgcacagataaactcatcctgaaagtcgctgttgttctcctgctgagcaagaatggaggccccactggtgagtctggatgaagagtttgaggaccttcgaccctcctgctcggaggacccggaggagaagccccagtgtttctatggttcatctccccaccatctcgaggacccctccctctccgagcttgagaatttttcttccgaaataatcagcttcaagtccatggaggacctcgtaaatgaatttgatgagaagctcaatgtctgctttcggaactacaacgccaagaccgagaacctagctcccgtgaagaaccagttacagatccaagaggaggaggagacccttcaggacgaggaggtttgggatgctctgacagacaattacatcccttcactctcagaagactggagggatccaaacatcgaggctctgaatggcaactgctctgacactgaggtaattgaggagattgaggaaatgatgcagaactccccagaccctgaggaagaagaggaggttctggaagaagaggatggaggagaaacttcctcccaggcagactcggtcctcctgcaggagatgcaggcattgacacagaccttcaacaacaactggtcctatgaagggctgaggcacatgtctgggtctgagctgaccgagctgctggaccaggtggagggtgccatccgtgacttctcggaggagctggtgcagcagctggcccgccgggacgagctggagtttgagaaggaagtgaagaactcctttatcacggtgcttattgaggttcagaacaagcagaaggagcagcgagaactgatgaaaaagaggcggaaagagaaagggctgagcctgcagagcagccggatagagaagggaaaccagatgcctctcaagcgcttcagcatggaaggcatctccaacattctgcagagtggcatccgccagacctttggctcctcaggaactgacaaacagtatctgaacacagtcattccttacgagaagaaagcctctcctccctcagtggaagacctgcagatgctgacaaacattctctttgccatgaaggaggataatgagaaggtgcctactttgctaacggactacattttaaaagtgctctgccctacctaaccttgccctttggagcagcctcgctgcaggaggtcactgagcaagagtcattccatcacagggactgcatgagaccatgtaacctccgacatgtatttaaacgtgtatagcttaacctggattaaacacgagcaagcgcgcggggtcctttgccgttggcttctagtgctagtaatcattggatgcatgatggggcagggccggtgatggtgcctcccccttgctggtgtcaggagaggggaaggcagccgctttcaccgctcattatgtagtctggctacagccctcaaaaacagcttatactcttaagactaattttgaaataaaaccttcatttaattaaaaaaaaaaaaaaagg

SEQ ID NO: 36 - DNA, 2997 bp, Homo sapiens
  misc_feature: Incyte ID No 71086982CB1
cgcgcttccgtcctgtccagccgccagtcctccagcccgtgtccccgctccgcccgctttgtctctccccggctcgctgtctctttgtctctgccctcgcgctcctccgcagcccctccctcgctccccatctcgggtccccttctcagagcgctcttcagccctcagagccgccctttctgggcgaccccactcttcgggactccccctcagagcgcccccagatcttttggggttccccttgagaacaccttccactctccccaagggctccccgtgagtttcttgcacatcctttgggggttctgcaccccaagtcgctggggtctcgcctcctctgaacccccattgcccctgggctttccctcttctgggtgttccccatatccactgggagctcctaggtcccaagttggggtctttctccttgggaccccccaatatgtcctcagctccctgacttcaggagctcctctctgctttcccctggtttgtcccctgtcgctgtctctccttgttctctctcaggtctccgagcacccccacttctcgggatcggggtcccctgctttgctctccctgcccctctgtgcccccacatctgtctcggtggtctcgccactctgtgcctcttgcgctgaaggcccgcctttgagcctgcttctttgcctggggcccttggcccccccttgctttttcagccctagccccctgtctccccttctctctgctccttgtctccctctccctttttctgtctttgccgggtctctgggtctctgacccccatccggccctcatggctttgtgtctggagctcttgaagcaatgttcatcatgcctagtggcgtataagaagaccccgccaccggtccctccacgcaccacttcaaagccgttcatctcagtcacagtccagagcagtactgagtctgcccaggacacctacctggacagccaggaccacaagagcgaggtgactagccagtcgggcctgagcaactcgtcggacagcctggacagcagtacccgaccgcccagcgtgacacggggtggagtcgccccagcccctgaggccccagagccacccccaaaacatgcagctctgaaaagtgaacaagggacgctgaccagctctgagtcccaccccgaggccgcccccaaaaggaaactgtcatcgataggaatacaagttgactgcattcagccagtgccaaaagaggagcccagtcccgctaccaaattccagtccatcggggttcaggtagaggacgactggcgaagcagcgtcccctctcacagtatgtcctcccgacgggacacagactcggatacccaggatgccaatgactcaagctgtaagtcatctgagaggagcctcccggactgtacccctcaccccaactccatcagcatcgatgccggtccccggcaggcccccaagattgcccagatcaagcgcaacctctcctatggagacaacagcgaccctgccctagaggcgtcctcgctgcccccacccgacccctggctcgagacctcctccagctccccagcagagccggcacagccaggggcctgccgccgagacggctactggttcctaaagctactgcaggcagaaacagagcggctggaaggctggtgctgccagatggacaaggagaccaaagagaacaacctctctgaagaagtcttaggaaaagtcctcagtgctgtgggcagtgcccagctactgatgtcccagaaattccagcagttccggggcctctgtgagcaaaacttgaaccctgatgccaacccacgccccacagcccaggacctggcagggttctgggacctgctacagctgtccatcgaggatatcagcatgaagttcgatgaactctaccacctcaaggccaacagctggcagctggtggagacccccgagaagaggaaggaagagaagaaaccaccccctccggtcccaaagaagccagccaaatccaagccggcagtgagccgcgacaaggcctcagacgccagcgacaagcagcgccaggaggcccgcaagagactcctggcggccaagcgggcagcttctgtgcggcagaactcagccaccgagagcgcagacagcatcgagatttatgtcccggaggcccagaccaggctctgagaccatgcaggaggaaagaaacgattttaaatcattaaaaacacaaaaactaagtgcgaacggaacagagttttctcaacctttgctatggttattctgtctagagaccctgagccaactttcaaattgacgcatacaagggctcacaatttggcttttttggggtccctcccagctttaggttatgaagattttactcacaaaaaaaatcaacaaaaatcacgaaactagaaaactttttttttcctcttgctgggccgtggtggactagatagatggacgtcggcaactcccggcccagcctccatactgcggtctttttactcgttctatctgatgagaactcacactagctggtttacaagatgacgacagtccaagggcagcctgtgggcacctgccatgtccctcctttccccagctatccccgctctgaccttgattttcattcttatgtttttctcttttcccttcagagctcacacagtggtcaccattgtggcaagcggctttctgggtctcagccctctctgcggttgagggcccagaggacagagagatggacatgcgtcccctccctccccccgccaagtgctcacacacaacctcacgcgcacacacacacacgcagatggaggcgcctcactgggaggtgccccgccagccctgggcagtgtcaggcaggactcactcaccgctgagcagatgagagaagttttagtcttggcgggtggaaatgagacgaagccacagttatcacactccagactcctgcccttttattttctccagccccttcttccttcagcaaaatctaggactcccgagtggcttcc

SEQ ID NO: 37 - DNA, 3383 bp, Homo sapiens
  misc_feature: Incyte ID No 7506367CB1
cgcgcttccgtcctgtccagccgccagtcctccagcccgtgtccccgctccgcccgctttgtctctccccggctcgctgtctctttgtctctgccctcgcgctcctccgcagcccctccctcgctccccatctcgggtccccttctcagagcgctcttcagccctcagagccgccctttctgggcgaccccactcttcgggactccccctcagagcgcccccagatcttttggggttccccttgagaacaccttccactctccccaagggctccccgtgagtttcttgcacatcctttgggggttctgcaccccaagtcgctggggtctcgcctcctctgaacccccattgcccctgggctttccctcttctgggtgttccccatatccactgggagctcctaggtcccaagttggggtctttctccttgggaccccccaatatgtcctcagctccctgacttcaggagctcctctctgctttcccctggtttgtcccctgtcgctgtctctccttgttctctctcaggtctccgagcacccccacttctcgggatcggggtcccctgctttgctctccctgcccctctgtgcccccacatctgtctcggtggtctcgccactctgtgcctcttgcgctgaaggcccccctttgagcctgcttctttgcctggggcccttggcccccccttgctttttcagccctagccccctgtctccccttctctctgctccttgtctccctctccctttttctgtctttgccgggtctctgggtctctgacccccatccggctctcatggctttgtgtctggagctcttgaagcaatgttcatcatgcctagtggcgtataagaagaccccgccaccggtccctccacgcaccacttcaaagccgttcatctcagtcacagtccagagcagtactgagtctgcccaggacacctacctggacagccaggaccacaagagcgaggtgactagccagtcgggcctgagcaactcgtcggacagcctggacagcagtacccgaccgcccagcgtgacacggggtggagtcgccccagcccctgaggccccagagccacccccaaaacatgcagctctgaaaagtgaacaagggacgctgaccagctctgagtccccaccccgagccgcccccaaaaggaaactgtcatcgataggaatacaagtcagctcgggtgcggaggccatagccccgcttggcggcaggagcagcatggagcatagacgctgttgggccaggggcccaggacccagggcccttgagccatgggggttgctcaaggggaactttgcccagagccctctcgggccttggggacaggttgactgcattcagccagtgccaaaagaggagcccagtcccgctaccaaattccagtccatcggggttcaggtagaggacgactggcgaagcagcgtcccctctcacagtatgtcctcccgacgggacacagactcggatacccaggatgccaatgactcaagctgtaagtcatctgagaggagcctcccggactgtacccctcaccccaactccatcagcatcgatgccggtccccggcaggcccccaagattgcccagatcaagcgcaacctctcctatggagacaacagcgaccctgccctagaggcgtcctcgctgcccccacccgacccctggctcgagacctcctccagctccccagcagagccggcacagccaggggcctgccgccgagacggctactggttcctaaagctactgcaggcagaaacagagcggctggaaggctggtgctgccagatggacaaggagaccaaagagaacaacctctctgaagaagtcttaggaaaagtcctcagtgctgtgggcagtgcccagctactgatgtcccagaaattccagcagttccggggcctctgtgagcaaaacttgaaccctgatgccaacccacgccccacagcccaggacctggcagggttctgggacctgctacagctgtccatcgaggatatcagcatgaagttcgatgaactctaccacctcaaggccaacagctggcagctggtggagacccccgagaagaggaaggaagagaagaaaccaccccctccggtcccaaagaagccagccaaatccaagccggcagtgagccgcgacaaggcctcagacgccagcgacaagcagcgccaggaggcccgcaagagactcctggcggccaagcgggcagcttctgtgcggcagaactcagccaccgagagcgcagacagcatcgagatttatgtcccggaggcccagaccaggctctgagaccatgcaggaggaaagaaacgattttaaatcattaaaaacacaaaaactaagtgcgaacggaacagagttttctcaacctttgctatggttattctgtctagagaccctgagccaactttcaaattgacgcatacaagggctcacaatttggcttttttgggtccctcccagctttaggttatgaagattttactcacaaaaaaaatcaacaaaaatcacgaaactagaaaactttttttttcctcttgctggccgtggtggactagatagatggacgtcggcaactcccggcccagcctccatactgcggtctttttactcgttctatctgatgagaactcacactagcttgtttacaagatgacgacagtccaagggcagccttgggcacctgccatgtccctcctttccccagctatccccgctctgaccttgattttcattcttatgtttttctcttttcccttcagagctcacacagtggtcaccattgtggcaagcggctttctgggtctcagccctctctgcggttgagggcccagaggacagagagatggacatgcgtcccctccctccccccgccaagtgctcacacacaacctcacgcgcacacacacacacgcagatggaggcgcctcactgggaggtgccccgccagccctgggcagtgtcaggcaggactcactcaccgctgagcagatgagagaagttttagtcttggcgggtggaaatgagacgaagccacagttatcacactccagactcctgcccttttattttctccagccccttcttccttcagcaaaatctaggactcccgagtggcttccagggggccgtcagtcctcagccgcgcctgtgtccggtgcccgaggggcgggcggcggtgtctgtatgtatgtgtacatatgcacatagaccttagagtgtatagttaacaaacgcccatctgctcacccatgcccacccagtgccgccgccgctggctctcggggcacctggcaggaggcgggtgtgtgaatagcatatatttttacatgtactatatctaggtgtgtgtaca

SEQ ID NO: 38 - DNA, 3789 bp, Homo sapiens
  misc_feature: Incyte ID No 1414020CB1
gtggggtggggcaggatgctggatggcccactgttctccgaggggcctgacagcccccgggagctccaggatgaggagtctggcagctgcctctgggtgcagaagtccaagctattggtgatagaagtgaagactatttcctgtcattatagtcgccgcgccccttctcgacagcccatggacttccaggccagccactgggctcgcgggttccagaaccgcacgtgtgggccgcgcccgggatccccacagccgccgccccgccggccctgggcctccagggtgctgcaggaggcgaccaactggcgggcggggcccctggccgaggtccgagctcgggagcaagagaaaaggaaagcggcgtcgcaggagcgggaggccaaggagaccgagcgaaaaaggcgcaaggctggtggggcccgacggagccccccgggtcgaccccgcccggagccccgcaacgcccctcgggtggcccagctggcagggctccctgctcccttgcggccggagcgcctggcgcctgtggggcgagcgccccgtccatccgcgcagccgcagagcgacccagggtcggcgtgggcggggccctggggaggtcggcggcccgggcccccaagctacgaggctcacctgctgctgagaggttctgccgggaccgccccacgacgccgctgggaccggccgccaccctacgtggctccaccttcttacgaaggcccccataggaccttggggactaagagaggccccgggaactctcaggtgcccacttcatcagccccagctgcgactccagccaggacagacggagggcgcacaaagaagaggctggatcctcggatctaccgggacgtcctcggggcttggggtctccgacaggggcaaggtctcttggggggatccccaggctgtggagcggccagagcaaggccagagcccggcaagggggtcgtggagaaaagcctggggctggctgctgctgacctgaacagtggtagcgacagccatccccaagccaaagctacagggagcgcaggcaccgagatagctcctgcggggtctgcaactgcggctccctgtgccccgcatcccgctcccagatccaggcaccacctcaagggctcgagggaagggaaagaaggagaacagatctggtttcccaaatgctggattccctcccctaaaaagcagccgccccgccatagccagacactccccagaccctgggctcccggaggcaccggatggagagaatctctgggtcttggagagggggcaggaccggagaccctggagggttggaaggcgacccgccgtgcccacaccttgccccgcagttcccagggcctgtcccgtggggaaggcgtctttgtcattgacgccacgtgcgtagtgatacgatcccaatatgttccaaccccccgaacccagcaggtgcagcttttgccctctggggtgacacgcgtggtgggggattcccccagccaatcgaagcccggcaaggaggagggtgaaggggccacggtctttccttccccttgtcaaaagcggctgtcgagcagtcgccttttacaccagcccggcgggggccgcgggggcgaagctgagggcgggaggccgggggactccacactggaggagcgcactttccgcatcttggggctcccggcccccgaagtaaacctgcgggacgcccccacgcagccaggtagcccagagcaccaagccttaggcccagcagcttcgggagcccagggcagagccgaggggtcggaagtggcggtggtccagcggcgcgccggccggggctgggcgcggaccccagggccctacgccggggccctgcgagaagccgtgtcccgtatccgccgccacacagcccctgactcggacacggacgaagctgaggagctcagcgtccatagcggctcctctgatggaagcgacacagaagccccgggcgcctcctggcggaatgagaggaccctgcccgaggttggaaacagttcgccagaggaagatgggaagacagcggaactgagcgacagtgtcggggagatcctagatgtcataagccaaaccgaggaggtcctcttcggggtgagggacatcagagggacccaacagggaaataggaagaggcagtgagaggccccttcttgtatttgtgtccccaacgcatccatccttgggtccactggtccccattcttccccacagacttcctttgcttctcttttccttgtatctttacccatacctgttctcatccttgaaatataaatgaaaggaagggaagcatatgcccattaatgattttgtttcaggagaggtgagaatgagcagatttaattaatgtctgttatgttcagggcacaagggtgagctcttcgcaggggctgatgcactgggtgtggagctgagcagagaggcctaaccaggatcaggcaggagggcagggatggtggcagccataggagggcagggtagggtagggcctctgaggaggagggaaaaagtgaaggagaggctttggacctggtgacagagtgatcagatgacagaggggttcttgggagaagaggcataggtccagcaacaaccaacaaagcagaaggagggctcaccttggtgtcacaagtcttggatttcaatcccaactctgccactgagttgctggttgactgaggccagtcactttccctctccaggcctccaggcctcctggtatataaaatgatggtattctaaggtccatccttccgtctctgacattttgagatctttggaaaggactctatctcatcctcccctcgacaagccaagaatgagaattgggaataagtgaacagagtttgagggtttctgggcggcctccgtgtcacccaaagtcatgatcaattcaggagactgcccaaggcttgcagaagaggtaagggagtgaggcactcctatcccagtctcccaggtttggttgagggctccccaaggcagggcaagatagcggccctgtcactgaccctggcctgtggtggtctgagctggggagggaaggacaccaatgaatcagcttgggacctctttaggccttccccttttcctccaccccgatgctccttagtgatgctctgaggcgtggccacgatctccctcccaggtggtatcgcccacctgaaaaaatcctgagaatttctcccatcttggcctcttccagaaaccggccaggcaaggaaagaggccggtcaccagaagccagcaggcgtggggtgtgatactctctatagccactacagggcgcgcgcaggtcgcggatctccccagttgctaatcccggctctgccactcaatcctatccctagttcccgagcgcgggtcccccgccttgcagtctccagccgtgcggggccgggagcaggcctccggcctcccagacttctagagcccgccgggcccatctttgtactcatccaccccagccggcttgggactcagacaccgaagtcttttttttttttctctccgatccttggacacctcctctgtctgccatttattagccatgtgaacttggccacatcacttcacctccctgagcctcagtttcctcatctgtcaaatgggggtttataaacacctacctcgcagggttgttgtgaggatttaatgcgataatgtatgtaaagcgccttgcacactgcctggcacacagtaggcgctcaataaatctaagcttccctttaaaaaaaaaaaaaaaaaaaaaaattctgcggcgcaagaattcctggc

SEQ ID NO: 39 - DNA, 4174 bp, Homo sapiens
  misc_feature: Incyte ID No 7621128CB1
aacagctagggaagcttgggtgtattttccccttgctgtgtcatatgatgttcctttccctacccccacctcctccagactgctgcctccatcagtgcctgtggcgtgtgtgtgtgtgtgtgtgtgtgcgcgcgcgtgcgtgcacgtgtgtatgtgtgtgtgtgtgtattgggtttctctctcccttgtaagaacacagccagcccgccctctcctgctgttgctgcagctctgacttgctttttcctgcctccttcctctcctctctcttcttgcttagcttcttgccttctgatactactcccaagatggaggctaatcactctgaacagctctcagcggaacgacagtcaacacctccaggtgacagttcatcattacccagtcacaatggcctggagaaggaagatggccaggattctccaaccccagtccaaccaccagagaaagaggcaagtgtgcaccccgatatctctgaagagctgaatcgacagctggaagacatcattaacacttatgggtctgctgccagcacagcagggaaagagggctctgccagggccagtgagcagcctgagaatgcagaatcacctgacaacgaggatggggactgtgaggaaacaactgaagaggctggaagagaacccgttgcttctggagagccacccactgtcaaagagcccgtcagcaataaggagcaaaaattggaaaagaaaatcctaaaaggattaggcaaagaagccaacctgctaatgcaaaatctgaacaagttgcaaacaccggaagaaaagtttgattttttattcaagaagtatgctgaattgctggatgaacatcgtactgagcaaaagaagttaaagctcctccaaaagaaacaggtacaaattcaaaaagaaaaggaccagttacaaggtgaacacagcagagctatcctcgctcgaagcaaattggagagtctgtgccgggagctgcagagacacaacaagactctgaaggaagaggcgcttcagcgggcacgtgaggaagaagagaaaaggaaggaaatcacaagccatttccagagtaccctcacggacatccagggccagatcgagcagcagagtgagcgaaatatgaagctctgtcaggagaacacagagcttgcagaaaagctgaaaagcatcatcgatcagtatgagctcagagaggagcatctggacaaaatatttaaacacagagaactgcagcagaagctggtggatgcaaagcttgagcaggcccaagaaatgatgaaggaagcggaggagcgacacaaacgagaaaaggaatatttgctgaaccaggcagcagagtggaaacttcaggcgaaagtgctgaaggagcaagagacagtcctgcaggctcagctcactctctactcaggaaggtttgaagaattccagagcacactaactaaaagcaacgaggtgtttgccacgttcaaacaggaaatggacaaaacaactaagaaaatgaagaagctggaaaaggacacagccacatggaaagcccgatttgagaactgtaacaaagctctgttggacatgattgaagagaaagcactgagagctaaagaatatgagtgctttgtgatgaaaatcgggaggctagagaacctctgccgtgctttacaagaagagagaaacgaactccacaaaaaaatcagagacgcagaaatatctgaaaaggatgaccaaagtcagcacaactccgatgaagagccagagtcaaacgtctctgtggatcaagagattgacgcagaggaggttaatagtgtccaaaccgccgtgaaaaatctggccacagccttcatgataattcatcatccagagtcaaccccgcaccagtccaaagaaacccaacccgaaataggcagttctcaggagagtgctgacgccgctctcaaggagccagagcaaccccctctgatcccttcacgggattcagagagtcccctgcctcccctaactcctcaggctgaagccgaaggaggcagtgatgctgaacctccctccaaggccagtaattctcctgccgggttgggagcagaaacccaatgcgagggtctccctgttggagcacaggctgatcaggcgtcctggaagccagaggcagaagcttccggtcaggccccacaggctcccaccgaggcctccctacagaagatggaggcagatgtgcctgctccagcatgcgcagcagaagagcacgttgcagccatggtgcctgcatgcgagcccagtaggcagcccccacgagcagcagcagaggagctgccagtaggggcctcagctgggccccagccgcgcaacgtggctgacaccaatctggaaggcgtcgactaagcctcaccgtgccttcagaggcttcttcctgcctctttgcatattcagcataacagcttgtcttccgaaaaaggcattaagggctagagatgtcaaatggaagagacttaggatcaagacattttttaatgttaggcagaacacattcattcagactttgctatttgttgtcaatttatagctgatttgaaggttttctatttaacactggttgacagtataattttcccaaagggcaacaaaacatacagatagcaaatttatattccttttctgttaagataatacttaaatttgttaatcaccagtaagatttgatgtttaaagacttccactgcaatatataaatactgaaaatgtgatgttctgcttatttggatatatagtttaacaagttccatagtaatttatggatgcccaggttacatttaaactatacatatacataatatatacatacgtgtgtacttattatatgtatatatacacgtatgtatagatgtgtgaatatttgtttatatacatatatgcacgtatctatatgtatatataaatgcattattacacatacatttctctcaccccttaatgcatttttctcaatgcagaatatttagatgctaatcaaaaaaatgatctcttacttgcacttgagactatggttgactaatgctctataaatccgaagagagttcggactataagtatttaggctatcattatgttgggcaaaaataagtaacccaatggtagataaatgaattcaaccagttgatcaaatggcagaaaagcagttagactacaatctgtgcagacagcatggacacaaagatgaccacaaggggcccaaaacagaaaagagaaacacaagctcacctcttggagctgcttctcctacacttctcagcccattgctttgccaccccctcatgtgccagggcatcccattccaggcttcctgcaaggaaacggttggaagtgggaaaggggagctagggattgaggggttaagggacctcacactaagaaggggctgtgctttgatcccctgcctcttgcactaccaatgtctcaagacataatattcatctcttgctgtcagacccattctatattctaaaagcttctgctccttccttcccaatttctcctttgtagcaggaaattacacccagccctcatctcaattaatgctaaataaagctattgtttttccaaacacaaatctacactgggtctcaatatcagtgatgaggcttacaaaccaacacgttttctgccatgaggatttctctttaggccagaagtacaaaacaaaaaaaccaatggattttaaccaaaatgatttgaaatataggtgaggattcaggagaaggcaaaagctagaaacacttggggttgtcaacatgagtattacattaacattgcttgatgagaacctctaatgatactgacaacataaattacctagggtaaaggatagctgcaacaatgaaacaggaaagaagagagggagagagaggaaagggaaggaagaaaggaaggagggagaagggaagaaagaaacaatgtctaacccaaccctatcttgaaagttgaactcaagtagaaaaatggatagaaacaaaattctctagtactcatccaggaaaccattcttcaatgttgcatgtggctgtttgccaaggcacacaaagtgcttgtaggcagcaaccatatgctacaagaattgtaaactgcatacagtttgtttgaagtagacagtgaggtattacaaagttgctaggcaggaaaaatcaggaaatagcttt

SEQ ID NO: 40 - DNA, 811 bp, Homo sapiens
  misc_feature: Incyte ID No 7505822CB1
gtagtccgtcccgcctgcccagtcagcgcggtgttgcccgccccgcactcggagcccagagccgccgcccaggaaggggatgcggaaacccctggctcggtggagcggagaggcaggcgggcaggagccgaggacggcatgtcccaggccccgggagcacagccgagcccacccaccgtgtaccacgaacggcagcgcctggagctgtgtgctgtccacgccctcaacaacgttctgcagcagcagctctttagccaggaggctgccgatgagatctgcaagaggcccctgtcccagctggccctgccccaggtactggggctgatcctgaacctgccctcgcccgtgtcgctggggctgctgtcactgccgctgcgccggcggcactgggtggccctgcgccaggtggacggtgtctactacaacctggactccaagctgcgggcgcccgaggccctgggggatgaggacggagtcagggccttcctggcggctgcgctggcccagggcctgtgcgaggtgctgctggtagtgaccaaggaggtggaggagaagggcagctggctgcggacagactgaccatggctgaccatcggcgcccacagcgcagtccctgcgcatccccctccggctgcgcacactgcatgcctgggaaaggccagcacttcatggaccctggggaggccccgccccctccccacacccctgctccccactgccgctgctgcctcaataaatctgctgatttgcaaaaaaaaaaaaaaaaaaaaaaaaaaaaaaaaaaaaaaaaaaaaaaaaaaaaaaagatcgg

SEQ ID NO: 41 - DNA, 4430 bp, Homo sapiens
  misc_feature: Incyte ID No 71607945CB1
cggggatggggacgcagctcggagcgctagagagacgcggcggcgctggcagaagaggcggcggcgggcgggtactggcttctggggccagggccagggcggtgggcgccgggaccgcggagctgaggagcggggcccggccagggctggagactttgcgcccgggggcaccggggctgcgcgcggtcgcacacatccaccggcgcggcttccctcggcggcccgggctccgctcatcctgcggcgggcggcgccgctcaggggcgggaagaggaggcggtagacgcgaccacagaagatgtcgggccaaacgctcacggatcggatcgccgccgctcagtacagcgttacaggctctgctgtagcaagagcggtctgcaaagccactactcatgaagtaatgggccccaagaaaaagcacctggactatttgatccaggctaccaacgagaccaatgttaatattcctcagatggccgacactctctttgagcgggcaacaaacagtagctgggtggttgtgtttaaggctttagtgacaacacatcatctcatggtgcatggaaatgagagatttattcaatatttggcttctagaaatacactattcaatctcagcaattttttggacaaaagtggatcccatggttatgatatgtctaccttcataaggcgctatagtagatatttgaatgaaaaggctttttcttacagacagatggcctttgattttgccagggtgaagaaaggggccgatggtgtaatgaggacaatggctcccgaaaagctgctaaagagtatgccaatactacagggacaaattgatgcactgcttgaatttgatgtgcatccaaatgaactaacaaatggtgtcataaatgcagcatttatgcttcttttcaaagatcttatcaaactttttgcttgctacaatgatggtgttattaacttactcgaaaagttttttgaaatgaagaaaggacaatgtaaagatgctctagaaatttacaaacgatttctaactagaatgacacgagtgtctgaatttctcaaggttgcagagcaagttggtattgataaaggtgacattcctgacctcacacaggctcccagcagtcttatggagacgcttgaacagcatctaaatacattagaaggaaagaaacctggaaacaatgaaggatctggtgctccctctccattaagtaagtcttctccagccacaactgttacgtctcctaattctacaccagctaaaactattgacacatccccaccggttgatttatttgcaactgcatctgcggctgtcccagtcagcacttctaaaccatctagtgatctcctggacctccagccagacttttcctctggaggggcagcagcagccgcagcaccagcaccaccaccacctgctggaggagccactgcatggggagaccttttgggagaggattctttggctgcactttcctctgttccctctgaagcacagatttcagatccatttgcaccagaacctacccctcctactacaactgctgaaattgcaactgcctcagcttctgcctccactactacaactgttactgctgtcactgctgaagtggatctctttggagatgcctttgcagcttctcctggggaggcccctgcagcatccgaaggggccgccgcaccagctaccccaacccctgtagcagcagcacttgatgcatgttcaggaaatgacccctttgccccgtctgaaggtagtgcagaggctgcacctgagctggacctctttgcaatgaagccacctgagaccagtgttcctgtagttacccctacagctagcacagcccctccggttcccgcaactgctccttctcctgctcctgccgttgcagctgctgctgctgccactactgctgccaccgccgctgccaccaccactaccaccacctccgctgccaccgccaccactgctcctcctgctctagatatctttggtgatttatttgagtccactcctgaagttgctgcagcgcctaagccagatgctgctcctagcatagacctgtttagtacagatgctttctcctctccaccacaaggggcctctcctgtgcctgagagttctctcactgctgacctcttatctgtggatgcatttgcagcaccatctcctgcaaccactgcctcgccagcaaaggtggattcttcaggtgtcatagacctttttggggatgcatttggaagtagcgcttctgaaccccaacctgcatctcaggctgcttctagttcatcagcatcggcagacctactagctggatttgggggttctttcatggcgccttccccatctccagtgactccagctcagaataacctgctacagcccaattttgaggcagcttttgggacaacgccttcaacttccagcagcagctcctttgatccatcaggtgatcttttgatgccaaccatggcaccagctgggcagcctgcacctgtctcaatggtaccacccagtcctgcaatggcagccagcaaagcccttggaagtgatcttgattcatctcttgccagcttagtaggcaatcttggaatttctggtaccacaacaaaaaagggagatcttcagtggaatgctggagagaaaaagttgactggtggagccaactggcagcctaaagtagctccagcaacctggtcagcaggcgttccaccaagtgcacctttgcaaggagctgtacctccaaccagttcagttcctcctgttgccggggccccatcggttggacaacctggagcaggatttggaatgcctcctgctgggacaggcatgcccatgatgcctcagcagccggtcatgtttgcacagcccatgatgaggcccccctttggagctgccgctgtacctggcacgcagctttctccaagccctacacctgccagtcagagtcccaagaaacctccagcaaaggacccattagcggatcttaacatcaaggatttcttgtaaacaatttaagctgcaatatttgtgactgaataggaaaataaatgagtttggagacttcaaataagattgatgctgagtttcaaagggagccaccagtaccaaacccaatacttactcataacttctcttccaaaatgtgtaacacagccgtgaaagtgaacattaggaatatgtactaccttagctgttatccctactcttgaaattgtagtgtatttggattatttgtgtattgtacgatgtaaacaatgaatggatgttactgatgccgttagtgcttttttggacttcacctgaggacagatgatgcagctgttgtgtggcgagctatttggaaagacgtctgtgtttttgaaggtttcaatgtacatataacttttgaacaaaccccaaactcttcccataaattatcttttcttctgtatctctgttacaagcgtagtgtgataataccagataataaggaaaacactcataaatatacaaaactttttcagtgtggagtacatttttccaatcacaggaacttcaactgttgtgagaaatgtttatttttgtggcactgtatatgttaagaaattttattttaaaaaatataaaggttaacgtccataataaatacttctctttgaagctaccttatcaagaacgaaaaatcgtatgggaagaatcccctatttatcactgctatattaaaatatatatattttaattatatttgacaggttttgcatctaaattgacctatttattcattcttgattaaatgcactgaaaagtaaagggtctgtttgtgtcatgttcatgaaaatgcggttagagaggtgctattcaagtgattctgaaggcaccccaaggtatatctgtaatttaaagattactgcaaatatctttactttactgtgggtttttagtacatctgttaatttagtgtttctttgtgtgttttgtagactagtgttcttccatccttcaactgagctcaaagtaggttttgttgtaacattgtgattaggatttaaactaattcagagaattgtatcttttactgtacatactgtattctttaagttttaatttgttgtcatactgtctgtgctgatggcttggcttaagattttgatgcataaatgaggtcactgttgatcagtgttgctagtagcttggcagctcttcataaaagcatattgggttggaaaggtgtttgcctatttttcaaattatttaatagatgtatggtaccatttaaaagtggttgtatctgaatttactgtggggataacatacactgtaatggggaaaaattacctaaaaccaatttcaaaatggctttctttgtatttcagtttaaaaacccagtgcatgtacgccctctgagatgcaataaacaccttgaacaaagaaatgcaaaaaaaaaaaaaaa

SEQ ID NO: 42 - DNA, 1216 bp, Homo sapiens
  misc_feature: Incyte ID No 7505777CB1
gcgcttcggcccgcactaaggccggctcttgtgccggaaggaggaaggcgtggggcattcgcccctcggagctagggagtgtgtgcgacgccgctgcgaggtcacgtgagccactgccggcagagagggaaaggggcggggcccagaacgaagcggggaggcgccccttgtttccctggggtcacgcgcagccggaagtggcggctgctgcggagaattggagatggggaccgccctggacatcaagattaaaagagcgaataaagtttatcacgccgggcctcagaaggggaagtttactcccagtcccgtggacttcacgattacacctgaaaccttacagaacgtcaaagagagagctttgcttcccaaatttctccttcgaggacatctcaactcaacaaactgtgtcatcacgcagccactaacgggagagctggtggtggagagctcggaagccgccatcagaagcgtggagctgcagctggtgcgcgtggagacgtgcgggtgtgcagaaggctatgcccgcgacgccacggagattcagaacattcagatcgccgacggggatgtgtgcaggggcctctctgtccccatctacatggtcttccctaggctgttcacctgccctacactggagaccaccaacttcaaagtggaatttgaggttaacatcgtggtgctgcttcaccctgaccacctcatcacggagaacttcccgctgaagctctgcaggatatagcccggaggagggaagcatagagaacgggagtggccatctggaaatccagctggttatccaaatcctaaggggagctacagccagcggcatatacttgtttttgtgattattctgtatcagaaatgaaacagaccctcaaattaactttccttcctcatttcttgaggcttctgcttccaacaggcacctctaatcagaccttttctttgaaattcaacaagatttcttaatgctatttgccaagaccatttcacagaaaacattgactgtggctcttgccttatctgttcctttttaggtacagtaaaacaattgtgacagcagtttgagcttgctggagagtggcatcatggggacaaaaggaaacctctgacttgctaatggatgtagccagggactccccatagcaaagggtctgtggccagttgacatccaggatggctgcaagcgcacttgatggtcaggaagtttgca

SEQ ID NO: 43 - DNA, 1269 bp, Homo sapiens
  misc_feature: Incyte ID No 7505818CB1
cagggggcggactggagggggtggttcggcgtgggggccgttggctccagacaaataaacatggagtccatcttccacgagaaacagccttctggaaatatggatgacagtggttttttctctattcaggttataagcaatgccttgaaagtttggggtttagaactaatcctgttcaacagtccagagtatcagaggctcaggatcgatcctataaatgaaagatcatttatatgcaattataaggaacactggtttacagttagaaaattaggaaaacagtggtttaacttgaattctctcttgacgggtccagaattaatatcagatacatatcttgcacttttcttggctcaattacaacaggaaggttattctatatttgttgttaagggtgatctgccagattgcgaagctgaccaactcctgcagatgattagggtccaacagatgcatcgaccaaaacttattggagaagaattagcacaactaaaagagcaaagagtccataaaacagacctggaacgaatgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtagttccagaaacatatctcaagatatgacacagacatcaggtacaaatcttacttcagaagagcttcggaagagacgagaagcctactttgaaaaacagcagcaaaagcagcaacagcagcagcagcagcagcagcagcagcagcagcagcagcagcagcagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataatacctttaaaaaataatttagatattcatactttccaacattatcctgtgtgattacagcatagggtccactttggtaatgtgtcaaagagatgaggaaataagacttttagcggtttgcaaacaaaatgatgggaaagtggaacaatgcgtcggttgtaggactaaataatgatcttccaaatattagccaaagaggcattcagcaattaaagacatttaaaatagaaaaaaaaaaaaaaaa

SEQ ID NO: 44 - DNA, 1423 bp, Homo sapiens
  misc_feature: Incyte ID No 7505821CB1
gttggctccagacaaataaacatggagtccatcttccacgagaaacaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacaggttataagcaatgccttgaaagtttggggtttagaactaatcctgttcaacagtccagagtatcagaggctcaggatcgatcctataaatgaaagatcatttatatgcaattataaggaacactggtttacagttagaaaattaggaaaacagtggtttaacttgaattctctcttgacgggtccagaattaatatcagatacatatcttgcacttttcttggctcaattacaacaggaaggttattctatatttgttgttaagggtgatctgccagattgcgaagctgaccaactcctgcagatgattagggtccaacagatgcatcgaccaaaacttattggagaagaattagcacaactaaaagagcaaagagtccataaaacagacctggaacgaatgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtagttccagaaacatatctcaagatatgacacagacatcaggtacaaatcttacttcagaagagcttcggaagagacgagaagcctactttgaaaaacagcagcaaaagcagcaacagcagcagcagcagcagcagcagcagcagcagcagcagcagcagcagcagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataatacctttaaaaaataatttagatattcatactttccaacattatcctgtgtgattacagcatagggtccactttggtaatgtgtcaaagagatgaggaaataagacttttagcggtttgcaaacaaaatgatgggaaagtggaacaatgcgtcggttgtaggactaaataatgatcttccaaatattagccaaagaggcattcagcaattaaagacatttaaaataaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 45 - DNA, 2864 bp, Homo sapiens
  misc_feature: Incyte ID No 7506685CB1
cgcgcttccgtcctgtccagccgccagtcctccagcccgtgtccccgctccgcccgctttgtctctccccggctcgctgtctctttgtctctgccctcgcgctcctccgcagcccctccctcgctccccatctcgggtccccttctcagagcgctcttcagccctcagagccgccctttctgggcgaccccactcttcgggactccccctcagagcgcccccagatcttttggggttccccttgagaacaccttccactctccccaagggctccccgtgagtttcttgcacatcctttgggggttctgcaccccaagtcgctggggtctcgcctcctctgaacccccattgcccctgggctttccctcttctgggtgttccccatatccactgggagctcctaggtcccaagttggggtctttctccttgggaccccccaatatgtcctcagctccctgacttcaggagctcctctctgctttcccctggtttgtcccctgtcgctgtctctccttgttctctctcaggtctccgagcacccccacttctcgggatcggggtcccctgctttgctctccctgcccctctgtgcccccacatctgtctcggtggtctcgccactctgtgcctcttgcgctgaaggcccccctttgagcctgcttctttgcctggggcccttggcccccccttgctttttcagccctagccccctgtctccccttctctctgctccttgtctccctctccctttttctgtctttgccgggtctctgggtctctgacccccatccggccctcatggctttgtgtctggagctcttgaagcaatgttcatcatgcctagtggcgtataagaagaccccgccaccggtccctccacgcaccacttcaaagccgttcatctcagtcacagtccagagcagtactgagtctgcccaggacacctacctggacagccaggaccacaagagcgaggtgactagccagtcgggcctgagcaactcgtcggacagcctggacagcagtacccgaccgcccaactccatcagcatcgatgccggtccccggcaggcccccaagattgcccagatcaagcgcaacctctcctatggagacaacagcgaccctgccctagaggcgtcctcgctgcccccacccgacccctggctcgagacctcctccagctccccagcagagccggcacagccaggggcctgccgccgagacggctactggttcctaaagctactgcaggcagaaacagagcggctggaaggctggtgctgccagatggacaaggagaccaacgagaacaacctctctgaagaagtcttaggaaaagtcctcagtgctgtgggcagtgcccagctactgatgtcccagaaattccagcagttccggggcctctgtgagcaaaacttgaaccctgatgccaacccacgccccacagcccaggacctggcagggttctgggacctgctacagctgtccatcgaggatatcagcatgaagttcgatgaactctaccacctcaaggccaacagctggcagctggtggagacccccgagaagaggaaggaagagaagaaaccaccccctccggtcccaaagaagccagccaaatccaagccggcagtgagccgcgacaaggcctcagacgccagcgacaagcagcgccaggaggcccgcaagagactcctggcggccaagcgggcagcttctgtgcggcagaactcagccaccgagagcgcagacagcatcgagatttatgtcccggaggcccagaccaggctctgagaccatgcaggaggaaagaaacgattgttaaatcattaaaaacacaaaaactaagtgcgaacggaacagagttttctcaacctttgctatggttattctgtctagagaccctgagcccactttcaattgacgcatacaagggctcacaatttggcttttttgggtccctcccagctttaggttatgaagattttactcacaaaaaaaatcaacaaaatcacgaaactagaaaactttttttttcctcttgctggccgtggtggactagatagatggacgtcggcaactcccggcccagcctccatactgcggtctttttactcgttctatctgatgagaactcacactagcttgtttacaagatgacgacagtccaagggcagccttgggcacctgccatgtccctcctttccccagctatccccgctctgaccttggattttcattcttatgtttttctcttttcccttcagagctcacacagtggtcaccattgtggcaagcggctttctgggtctcagccctctctgcggttgagggcccagaggacagagagatggacatgcgtcccctccctccccccgccaagtgctcacacacaacctcacgcgcacacacacacacgcagatggaggcgcctcactgggaggtgccccgccagccctgggcagtgtcaggcaggactcactcaccgctgagcagatgagggaagttttagtcttggcgggtggaaatgagacgaagccacagttatcacactccagactcctgcccttttattttctccagccccttcttccttcagcaaaatctaggactcccgagtggcttccagggggccgtcagtcctcagccgcgcctgtgtccggtgcccgaggggcgggcggcggtgtctgtatgtatgtgtacatatgcacatagaccttagagtgtatagttaacaaacgcccatctgctcacccatgcccacccagcgccgccgccgctggctctcggggcacctggcaggaggcgggtgtgtgaatagcatatatttttacatgtactatatctaggtgtgtgtaca

SEQ ID NO: 46 - DNA, 1025 bp, Homo sapiens
  misc_feature: Incyte ID No 7500933CB1
cgagcgggatccaaacttccggtgcctgcagagctcggagcggcggaggcagagaccgaggctgcaccggcagaggctgcggggcggacgcgcgggccggcgcagccatggtgaagattagcttccagcccgccgtggctggcatcaagggcgacaaggctgacaaggcgtcggcgtcggcccctgcgccggcctcggccaccgagatcctgctgacgccggctagggaggagcagcccccacaacatcgatccaagagggggggctcagtgggcggcgtgtgctacctgtcgatgggcatggtcgtgctgctcatgggcctcgtgttcgcctctgtctacatctacagatacttctttcttgcacagctggcccgagataacttcttccgctgtggtgtgctgtatgaggactccctgtcctcccaggtccggactcagatggagctggaagaggatgtgaaaatctacctcgacgagaactacgagcgcatcaacgtgcctgtgccccagtttggcggcggtgaccctgcagacatcatccaggaggagatggtggtcacggagcatgtcagtgacaaggaggccctggggtccttcatctaccacctgtgcaacgggaaagacacctaccggctccggcgccgggcaacgcggaggcggatcaacaagcgtggggccaagaactgcaatgccatccgccacttcgagaacaccttcgtggtggagacgctcatctgcggggtggtgtgaggccctcctcccccagaaccccctgccgtgttcctcttttcttctttccggctgctctctggccctcctccttccccctgcttagcttgtactttggacgcgtttctatagaggtgacatgtctctccattcctctccaaccctgcccacctccctgtaccagagctgtgatctctcggtggggggcccatctctgctgacctgggtgtggcggagggagaggcgatgctgcaaagtgttttctgtgtcccactgtcttgaagctgggcctgc

SEQ ID NO: 47 - DNA, 3048 bp, Homo sapiens
  misc_feature: Incyte ID No 7389203CB1
ccggcacccacgaccgacaagtgaagctcacctttcgaggctttacccagaaaacaagaaaaattcactgtggtccagaagcagatatcggtgagctgttccgatggccccactatggggctccactggctggggagtgtctgtctgtgcaggtggtcaactgcagccgtgtattcagccttaggcctctagggaccctggtgatctccctgcagcagctacagaatgctgggcatttggtgctacgggaagccctagtggatgagaatcttcaagtgtccccgatccaggtggagcttgacctgaagtaccagcccccagagggcgctactggagcctggtcagaggaggactttggggcacccatccaggacagcttcgagttaatcatccccaatgtgggcttccaggaactggagcctggggaggcccagctggagcggcgggcagtggctctaggccgcaggctagctcgaagtctaggccagcaggacgatgaagagaatgagctggagcttgagctggagcaggacctggatgatgagcctgacgtggaactttctggtgttatgttcagccccctcaagagccgcgccagggccctggcccatggggatcccttccaggtgtccagagctcaagacttccaggtgggagtcactgtgctggaagcccagaaactggtgggagtcaacattaacccctatgtggccgtgcaagtgggggggcagcgccgtgtgaccgccacacagcgtgggaccagttgccccttctacaatgagtacttcttgttcgaatttcatgacacgcggcttcgtctccaagacttgctgctggagatcacggctttccattcgcagaccctcccctttatggccacccggataggcaccttcaggatggacctgggcatcatcttggaccagccagatggccagttctaccaaagatgggttccgctgcatgatccccgagacacccgcgccgggaccaagggtttcattaaggtcaccttgtccgtgagggcgcgcggggacctgccccctccaatgctacccccggccccagggcactgttcggacatcgagaagaacctgctcctgccgcgcggggtgcccgccgagaggccatgggcgcggctccgcgtgcgcctgtaccgcgccgaggggcttcccgcgctgcgcctggggctgctgggcagcctggtccgcgccctgcacgaccagcgcgtcctggtggagccctatgtgcgggtgtctttcctggggcaggagggcgagacgtcggtgagcgccgaggcggcggcgcccgaatggaacgagcagctgagcttcgtggagctcttcccgccgctgacgcgcagcctccgcctgcagctgcgggacgacgcgcccctggtcgacgcggcactcgctacgcacgtgccggacctgaggcggatctcccatccgggccgcgcggcggggtttaaccctaccttcggcccggcctgggtgcccctctatggctcgccccccggcgcggggctccgggatagtcttcaaggtctcaacgaaggcgttggccaaggcatttggttccgcggccgccttctgctggctgtgtccatgcaggtgttggaagggagagctgaacctgagcctccccaggcccagcaggggtccacgttgtcccggctcacccgaaagaagaaaaagaaagccagaagggatcagaccccaaaggcggttccgcagcacttggacgccagccccggtgccgaggggcctgagatcccccgtgccatggaggtggaggtggaggagctgctgccgctgccagagaatgtcctggcgccctgtgaagatttcctgcttttcggtgtgctcttcgaggccaccatgatcgaccccaccgtggcctcccagcccatcagcttcgagatctccattggtcgcgcaggccgtctggaggagcaattgggccgagggtccagggctggggagggaactgagggtgcagccgtggaggctcagcctctgctgggagccaggccagaggaggagaaagaggaggaagaactggggacccctgctcagcggcctgagcccatggacggcagcgggccatacttctgcttgcccctctgtcactgcaagccatgcatgcatgtgtggagttgctgggaggaccacacctggcgcctgcagagcagcaactgcgtgcgcaaagtggccgagaggctggaccaggggctgcaggaggttgagagactgcagcgcaagccggggcctggcgcctgtgcacagctcaagcaggcactggaagtgctggtggctgggagcagacagttttgccacggtgccgagcgcaggacgatgacccggcccaatgccctggatcgatgccgagggaaactcctggtgcacagcctgaaccttttggctaagcaaggactgcgacttctacgcggcctgagacggcgcaatgtgcaaaagaaggtggcactggccaagaagctcctggcaaaactgcgctttctggctgaggaggcacccggggcagcccctggtgaggtctgtgccaagctggagctcttcctgcggctgggcctgggcaagcaagccaaggcctgcacctctgagctgcccccggatttgctgcccgagccctcagccgggctgccctccagcctacaccgggacggtcctggagcagacgctgagccctctgtgggatgaactcctggtatttgagcagttgatcgtggatgggaggagggagcacctgcaggaggagcctccattagtgatcatcaatgtatttgaccacaataagtttgtgagtgtggcctgggccctccctgggttcctggccaggagtttcccccttgatgcccaccttcctggctcctgagcctcttcccctttgtcttcactgcctgctccccctagggcccccccgtgttcctgggcagggcactgccgccccaagggtaaagctgatgaaggaccacaggccaacgcccagagttaccttaccagcacagtgcgatgatatcaatcagcc

SEQ ID NO: 48 - DNA, 1299 bp, Homo sapiens
  misc_feature: Incyte ID No 7506268CB1
gcccgctgaggacgcagcgtcagctgacctggggagtcgcgattcgtgccggccggtcctggttctccggtcccgccgctcccgcagcagccatgtcgttcttcccggagctttactttaacgtggacaatggctacttggagggactggtgcgcggcctgaaggccggggtgctcagccaggccgactacctcaacctggtgcagtgcgagacgctagaggcggcttttttccaggactgcatttcagagcaggaccttgacgagatgaacatcgagatcatccgcaacaccctctacaaggcctacctggagtccttctacaagttctgcaccctactgggcgggactacggctgatgccatgtgccccatcctggagtttgaagcagaccgccgcgccttcatcatcaccatcaattctttcggcacagagctgtccaaagaggaccgtgccaagctctttccacactgtgggcggctctaccctgagggcctggcgcagctggctcgggctgacgactatgaacaggtcaagaacgtggccgattactacccggagtacaagctgctcttcgagggtgcaggtagcaaccctggagacaagacgctggaggaccgattctttgagcacgaggtaaagctgaacaagttggccttcctgaaccagttccactttggtgtcttctatgccttcgtgaagctcaaggagcaggagtgtcgcaacatcgtgtggatcgctgaatgtatcgcccagcgccaccgcgccaaaatcgacaactacatccctatcttctagcgtcctggcccaaggctctcaattgcactctttgtgtgtgtgtgtgtgtgtgtgcgcgtgtgtgtgcgtgtgtgtgtatgtggtctgtgacaagcctgtggctcacctgcctgtccggggtgtagtacgctgtcctagcggctgcccagttctcctgaccctcttagagactgttcttaggcctgaaaaggggctgggcacccccccccaccaaggatggacgaagaccccctccagagcaaggaggccccctcagccctgtggttacagccgctgatgtatctaagaagcatgtcactttcatgttcctccctaactccctgacctgagaaccctggggcctgggggcagtttgagcctcctctcccttctgtgggtcgctcccagagccatggcccatgggaaggacagagtgtgtgtgtccttggggcctggggggatgttgctcctcagctccctccctcagccctgcccctctgagacaataaaactgccctaaaaa

SEQ ID NO: 49 - DNA, 3146 bp, Homo sapiens
  misc_feature: Incyte ID No 7509159CB1
ttggcatgatgggcacctggagggccgcactcccgttccagccaggctgagccttctgtcccctgcctctggggcctgggaaccccccttcttctttctcctgaatggcacccccgccctagaatccagacaccgagtttcccactgtggctggttcaagggtatgtgagagctccctggtgacagtctgtggctgagcatggccctcccagccctgggcctggacccctggagcctcctgggccttttcctcttccaactgcttcagctgctgctgccgacgacgaccgcggggggaggcgggcaggggcccatgcccagggtcagatactatgcaggggatgaacgtagggcacttagcttcttccaccagaagggcctccaggattttgacactctgctcctgagtggtgatggaaatactctctacgtgggggctcgagaagccattctggccttggatatccaggatccaggggtccccaggctaaagaacatgataccgtggccagccagtgacagaaaaaagagtgaatgtgcctttaagaagaagagcaatgaggaacttcaagattcctacctgttgcccatctcggaggacaaggtcatggagggaaaaggccaaagcccctttgaccccgctcacaagcatacggctgtcttggtggatgggatgctctattctggtactatgaacaacttcctgggcagtgagcccatcctgatgcgcacactgggatcccagcctgtcctcaagaccgacaacttcctccgctggctgcatcatgacgcctcctttgtggcagccatcccttcgacccaggtcgtctacttcttcttcgaggagacagccagcgagtttgacttctttgagaggctccacacatcgcgggtggctagagtctgcaagaatgacgtgggcggcgaaaagctgctgcagaagaagtggaccaccttcctgaaggcccagctgctctgcacccagccggggcagctgcccttcaacgtcatccgccacgcggtcctgctccccgccgattctcccacagctccccacatctacgcagtcttcacctcccagtggcaggttggcgggaccaggagctctgcggtttgtgccttctctctcttggacattgaacgtgtctttaaggggaaatacaaagagttgaacaaagaaacttcacgctggactacttataggggccctgagaccaacccccggccaggcagttgctcagtgggcccctcctctgataaggccctgaccttcatgaaggaccatttcctgatggatgagcaagtggtggggacgcccctgctggtgaaatctggcgtggagtatacacggcttgcagtggagacagcccagggccttgatgggcacagccatcttgtcatgtacctgggaaccaccacagggtcgctccacaaggctgtggtaagtggggacagcagtgctcatctggtggaagagattcagctgttccctgaccctgaacctgttcgcaacctgcagctggcccccacccagggtgcagtgtttgtaggcttctcaggaggtgtctggagggtgccccgagccaactgtagtgtctatgagagctgtgtggactgtgtccttgcccgggacccccactgtgcctgggaccctgagtcccgaacctgttgcctcctgtctgcccccaacctgaactcctggaagcaggacatggagcgggggaacccagagtgggcatgtgccagtggccccatgagcaggagccttcggcctcagagccgcccgcaaatcattaaagaagtcctggctgtccccaactccatcctggagctcccctgcccccacctgtcagccttggcctcttattattggagtcatggcccagcagcagtcccagaagcctcttccactgtctacaatggctccctcttgctgatagtgcaggatggagttgggggtctctaccagtgctgggcaactgagaatggcttttcataccctgtgatctcctactgggtggacagccaggaccagaccctggccctggatcctgaactggcaggcatcccccgggagcatgtgaaggtcccgttgaccagggtcagtggtggggccgccctggctgcccagcagtcctactggccccactttgtcactgtcactgtcctctttgccttagtgctttcaggagccctcatcatcctcgtggcctccccattgagagcactccgggctcggggcaaggttcagggctgtgagaccctgcgccctggggagaaggccccgttaagcagagagcaacacctccagtctcccaaggaatgcaggacctctgccagtgatgtggacgctgacaacaactgcctaggcactgaggtagcttaaactctaggcacaggccggggctgcggtgcaggcacctggccatgctggctgggcggcccaagcacagccctgactaggatgacagcagcacaaaagaccacctttctcccctgagaggagcttctgctactctgcatcactgatgacactcagcagggtgatgcacagcagtctgcctcccctatgggactcccttctaccaagcacatgagctctctaacagggtgggggctacccccagacctgctcctacactgatattgaagaacctggagaggatccttcagttctggccattccagggaccctccagaaacacagtgtttcaagagaccctaaaaaacctgcctgtcccaggaccctatggtaatgaacaccaaacatctaaacaatcatatgctaacatgccactcctggaaactccactctgaagctgccgctttggacaccaacactcccttctcccagggtcatgcagggatctgctccctcctgcttcccttaccagtcgtgcaccgctgactcccaggaagtcttccctgaagtctgaccacctttcttcttgcttcagttggggcagactctgatcccttctgccctggcagaatggcaggggtaatctgagccttcttcactcctttaccctagctgaccccttcacctctccccctcccttttcctttgttttgggattcagaaaactgcttgtcagagactgtttattttttattaaaaatataaggcttaaaaaaaaaaaaaa

SEQ ID NO: 50 - DNA, 2238 bp, Homo sapiens
  misc_feature: Incyte ID No 7512347CB1
ggtatgtgagagctccctggtgacagtctgtggctgagcatggccctcccagccctgggcctggacccctggagcctcctgggccttttcctcttccaactgcttcagctgctgctgccgacgacgaccgcggggggaggcgggcaggggcccatgcccagggtcagatactatgcaggggatgaacgtagggcacttagcttcttccaccagaagggcctccaggctaaagaacatgataccgtggccagccagtgacagaaaaaagagtgaatgtgcctttaagaagaagagcaatgagacacagtgtttcaacttcatccgtgtcctggtttcttacaatgtcacccatctctacacctgcggcaccttcgccttcagccctgcttgtaccttcattgaacttcaagattcctacctgttgcccatctcggaggacaaggtcatggagggaaaaggccaaagcccctttgaccccgctcacaagcatacggctgtcttggtggatgggatgctctattctggtactatgaacaacttcctgggcagtgagcccatcctgatgcgcacactgggatcccagcctgtcctcaagaccgacaacttcctccgctggctgcatcatgacgcctcctttgtggcagccatcccttcgacccaggtcgtctacttcttcttcgaggagacagccagcgagtttgacttctttgagaggctccacacatcgcgggtggctagagtctgcaagaatgacgtgggcggcgaaaagctgctgcagaagaagtggaccaccttcctgaaggcccagctgctctgcacccagccggggcagctgcccttcaacgtcatccgccacgcggtcctgctccccgccgattctcccacagctccccacatctacgcagtcttcacctcccagtggcaggttggcgggaccaggagctctgcggtttgtgccttctctctcttggacattgaacgtgtctttaaggggaaatacaaagagttgaacaaagaaacttcacgctggactacttataggggccctgagaccaacccccggccaggcagttgctcagtgggcccctcctctgataaggccctgaccttcatgaaggaccatttcctgatggatgagcaagtggtggggacgcccctgctggtgaaatctggcgtggagtatacacggcttgcagtggagacagcccagggccttgatgggcacagccatcttgtcatgtacctgggaaccaccacagggtcgctccacaaggctgtggtaagtggggacagcagtgctcatctggtggaagagattcagctgttccctgaccctgaacctgttcgcaacctgcagctggcccccacccagggtgcagtgtttgtaggcttctcaggaggtgtctggagggtgccccgagccaactgtagtgtctatgagagctgtgtggactgtgtccttgcccgggacccccactgtgcctgggacctgagtcccgaacctgttgcctcctgcctgcccccaacctgaactcctggaagcaggacatggagcgggggaacccagagtgggcatgtgccagtggccccatgagcaggagccttcggcctcagagccgcccgcaaatcattaaagaagtcctggctgtccctaactccatcctggagctcccctgcccccacctgtcagccttggcctcttattattggagtcatggcccagcagcagtcccagaagcctcttccactgtctacaatggctccctcttgctgatagtgcaggatggagttgggggtctctaccagtgctgggcaactgagaatggcttttcataccctgtgatctcctactgggtggacagccaggaccagaccctggccctggatcctgaactggcaggcatcccccgggagcatgtgaaggtcccgttgaccagggtcagtggtggggccgccctggctgcccagcagtcctactggccccactttgtcactgtcactgtcctctttgccttagtgctttcaggagccctcatcatcctcgtggcctccccattgagagcactccgggctcggggcaaggttcagggctgtgagaccctgcgccctggggagaaggccccgttaagcagagagcaacacctccagtctcccaaggaatgcaggacctctgccagtgatgtggacgctgacaacaactgcctaggcactgaggtagcttaaactctaggcacaggc